(19) United States
(12) Patent Application Publication
(10) Pub. No.: US 2010/0249123 A1
(43) Pub. Date: Sep. 30, 2010

(54) NOVEL BENZAMIDE DERIVATIVES AS MODULATORS OF THE FOLLICLE STIMULATING HORMONE

(75) Inventors: Beatrice Bonnet, Plan-les-Ouates (CH); Brice Campo, Plan-les-Ouates (CH); Luca Raveglia, Milan (IT); Mauro Riccaboni, Milan (IT)

Correspondence Address: EDWARDS ANGELL PALMER & DODGE LLP, P.O. BOX 55874, BOSTON, MA, 02205, US

(73) Assignee: ADDEX PHARMA SA, PLAN-LES-OUATES (CH)

(21) Appl. No.: 12/532,831

(22) PCT Filed: Mar. 19, 2008
(86) PCT No.: PCT/IB08/00985

§ 371 Date: May 19, 2010

(30) Foreign Application Priority Data
Mar. 23, 2007 (GB) 0705656.7

(51) Int. Cl. A61K 31/166; C07C 255/44; A61K 31/277; C07C 255/46; C07D 319/16; A61K 31/357; C07D 213/56; A61K 31/44; C07C 271/10; A61K 31/27; C07D 271/06; A61K 31/4245; C07C 237/48; C07D 265/36; A61K 31/538; C07D 295/185; A61K 31/5375; C07D 317/68; A61K 31/36; C07C 275/26; A61K 31/17; C07D 263/04; A61K 31/421; C07D 231/14; A61K 31/415; C07D 231/56; A61K 31/416; C07D 333/24; A61K 31/381; C07D 239/26; A61K 31/505; C07D 471/04; A61K 31/437; A61P 35/00; A61P 19/10; A61P 15/00

(52) U.S. Cl. 514/230.5; 558/392; 514/521; 549/362; 514/452; 546/337; 514/357; 560/32; 514/506; 548/131; 514/364; 564/158; 514/616; 544/105; 544/169; 514/237.5; 549/436; 514/466; 564/47; 514/595; 564/179; 514/622; 548/231; 514/376; 548/374.1; 514/406; 548/356.5; 549/77; 514/438; 544/335; 514/256; 546/121; 514/300; 546/113

(57) ABSTRACT

The present invention provides new compounds of formula I, wherein Q, R₁, R₂, R₄, R₅, R₆, Xᵢ, R₇, R₈, M and G¹ₙ are defined as in formula I; invention compounds are modulators of follicle-stimulating hormone—(“FSH”) which are useful for male and female contraception as well as other disorders modulated by FSH receptor.

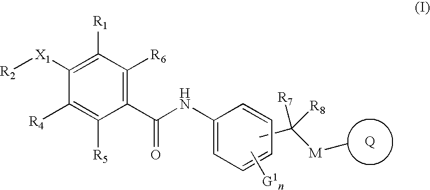

### SUMMARY OF THE INVENTION

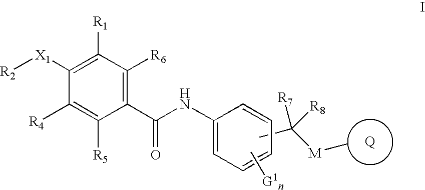

The present invention provides new compounds of formula I, wherein Q, R₁, R₂, R₄, R₅, R₆, X₁, R₇, R₈, M and G¹ₙ are defined as in formula I; invention compounds are modulators of follicle-stimulating hormone—(“FSH”) which are useful for male and female contraception as well as other disorders modulated by FSH receptor.

### BACKGROUND OF THE INVENTION

The invention relates to a compound having negative allosteric modulator activity on Follicle Stimulating Hormone (FSH) receptor, in particular compounds of formula I, to a pharmaceutical composition containing the same, as well as the use of said compound in medical therapy.

Gonadotropins serve a variety of important bodily functions including metabolism, temperature regulation, bone maintenance and the reproductive process. Normal function of both the ovary and the testis is long recognized to be dependent on the pituitary-synthesized gonadotropins (Luteinizing hormone (LH), Thyrotropin hormone (TSH) and FSH). These pituitary hormones are glycoprotein dimers, which share a common α-subunit, and with an average molecular weight of ˜30 kDa (Combarnous, Endocrine Review, 13, 670-691, 1992). Their action is mediated via specific plasma membrane receptors that are members of the large family of G protein coupled receptors (GPCR), and lead to activation of the adenyl cyclase system and elevation of intracellular levels of the second messenger cAMP (Mukherjee et al., Endocrinology, 137, 3234, 1966).

In women, reproduction depends upon the dynamic interaction of several compartments of the female reproductive system. Glycoprotein hormones, and in particular LH and FSH, act directly on the ovary to promote the development of selected follicles by inducing granulosa and theca cells proliferation and differentiation. More precisely, upon LH-mediated stimulation of the LH receptor, present on ovarian Theca cells, testosterone is generated. In a parallel manner, FSH-mediated activation of the FSH receptor, present on ovarian granulose cells, leads to the production of the enzyme aromatase. Aromatase converts testosterone into estradiol, required for follicle growth, ovulation, and endometrium proliferation (For review see Hsueh et al., Rec. Prog. Horm. Res., 45, 209-227, 1989). The discovery of such regulatory mechanisms has opened some new opportunities for the development of effective contraceptive methods. Moreover, Female FSHβ (mutation on the β subunit of the FSH peptide) gene knock-out mice are infertile because of a block in folliculogenesis (Kumar et al., Nat. Gen., 15, 201-204, 1997). In the same manner women with resistant ovary syndrome are infertile. The infertility experienced by these women is the result of non-functional FSH receptors (Aittomaki et al., Cell, 82, 959-968, 1995), re-enforcing the hypothesis that an antagonist of FSH receptor would act to limit proliferation of follicular granulosa cells in the ovary, therefore acting as a contraceptive. The control of fertility is a major public health issue and contraceptive choices for women have not increased since the development of the steroid based contraception. These compounds act via the nuclear estrogen or progesterone receptors, which are present in variety of tissues and could lead to unwanted side effects. Due to their specific action on ovarian tissue without impacting peripheral and central tissues, FSH receptor antagonists will therefore represent a novel non-steroidal approach for contraception.

Because of the controlling function of FSH on estrogen synthesis, a FSH antagonist may also be effective in the treatment of estrogen-related disorders such as uterine fibroids, endometriosis, polycystic ovarian disease, dysfunctional uterine bleeding, breast cancer and ovarian cancer. Moreover, because the proliferation of follicular granulosa cells also impacts the health and development of the oocytes, FSH antagonists may be useful in preventing depletion of oocytes, a common side effect of chemotherapy or similar treatments designed to treat rapidly dividing cells.

More recently, a study by Sun et al. examined the role of FSH in the regulation of bone mass in women having postmenopausal osteoporosis. They propose that an excess of FSH during menopause and ovarian deficiency might explain the accompanying bone loss in these states. In fact, neither FSHβ nor FSH receptor null mice have bone loss despite severe hypogonadism (Sun et al., Cell, 125, 247-260, 2006). These data suggests that FSH antagonists may also be useful in the prevention and in the treatment of osteoporosis.

In males, FSH is responsible for the integrity of the seminiferous tubules and acts on Sertoli cells for the maturation of sperm cells. Male FSHβ null mice have small testes and have reduced (75%) epididymal sperm (Kumar et al., Nat. Gen., 15, 201-204, 1997) while idiopathic men infertility seems to be related to a reduction in FSH binding sites. In addition, men with selective FSH deficiency are oligo- or azoospermic with normal testosterone levels and present normal virilization (Lindstedt et al., Clin. Lab. Med., 36, 664, 1998). There are currently some studies attempting to develop steroid-based male contraceptives but these are not orally active and might induce secondary effects (Peterson et al., Mol. Cell. Endocrin., 160, 203-217, 2000; Liu et al., Endocrine, 13, 361-367, 2000). Therefore, low molecular weight (LMW) FSH antagonists may proyide a novel method for male contraception. They also have the potential to modify the rate of germ cell division in male. Because chemotherapy is known to deplete rapidly dividing cells such as spermatocytes, an FSH antagonist may be useful in a planned chemotherapy regimen to prevent spermatocyte depletion.

A new avenue for developing selective compounds acting at GPCRs is to identify molecules that act through allosteric mechanisms, modulating the receptor by binding to a site different from the highly conserved orthosteric binding site. This concept has assumed a greater importance in the pharmacology of GPCR in general. For example, allosteric modulators have been described for Ca²⁺-sensing receptors (Nemeth et al, U.S. Pat. No. 6,031,003. Prior WO 93/04373), for metabotropic glutamate receptors (reviewed in Mutel, Expert Opin. Ther. Patents 12:1-8, 2000), for GABAB receptors (Urwyler et al, Mol. Pharmacol., 60, 963-971, 2001), or for adenosine receptors (Gao et al., Mini. Rev. Med. Chem., 5, 545-553, 2005). These ligands do not activate the receptor by themselves but either increase or decrease both the potency and/or the efficacy of the endogenous agonist (For review see T. Kenakin, Mol. Interv., 4, 223-229, 2004; Christopoulos and Kenakin, Pharmacol. Review., 54, 323-374, 2002; May et al, Annu. Rev. Pharmacol. Toxicol., 47, 1-51, 2007). As a therapeutic principle, negative allosteric modulators are expected to have several advantages over compounds acting at the orthosteric binding site which behave as competitive antagonists. Due to the non competition between agonist and antagonist, (i) less compound is necessary to induce inhibition therefore avoiding possible problems of overdosing rendering negative allosteric modulators safer and allowing higher doses of compound to be administered; (ii) they produce saturable antagonism and therefore dissociate magnitude from duration of the effect; and (iii) because they bind to a site on the receptor that is distinct for each receptor subtype of the same family, they offer high selectivity or even specificity.

Negative allosteric modulators of FSH receptors have emerged recently in WO 04/056779, WO 04/056780 (Tetrahydroquinolines) and WO 02/70493 (Bisaryls) as novel pharmacological entitites. Substituted tetrahydroquinoline derivatives FSH-R antagonists have been disclosed in WO 03/004028. Thiazolidinone FSH-R agonists and antagonists have been described in WO 02/09705 and WO 02/09706. Aryl sulfonic acid FSH-R antagonists have been disclosed in WO 00/58276 and WO 00/58277. Substituted aminoalkylamide derivative FSH-R antagonists have been described in WO 01/47875. FSH-R agonist activity was disclosed in WO 03/020726 (Thienopyrimidine); WO 01/87287 (pyrazoles) and WO 06/117370 (Hexahydroquinolines). Examples of FSH-R agonists are described by others in the field in WO 05/087765 (Thiazoles). FSH receptor antagonists are disclosed in WO 06/135687 (Pyrrolobenzodiazepines) and in WO 07/017289 (Acyltryptophanols).

International Patent Publication WO03/103655 discloses N-phenylsalicylamide having a hydroxyl group in ortho position as NF-κ_{b} inhibitors for therapeutic treatment of cancers. Certain para di-substituted phenylamides containing a gem-dialkyl group in combination with a cyano, a terminal aminomethyl or a terminal aminocarbonyl are claimed as agents used in treatment of heart and circulatory diseases (EP 0358118). International Patent Publication WO03/004467 describes amino-thiazole benzamides derivatives as inhibitors of the cellular proliferation. In WO04108133, VR1 receptor modulators are presented, containing a carbonyl group in a hetero-bicyclic ring and connected to a substituted phenyl by an amide bond.

None of the specifically disclosed compounds are structurally related to the compounds of the present invention.

### DETAILED DESCRIPTION OF THE INVENTION

According to the present invention, there are provided new compounds of the general formula I

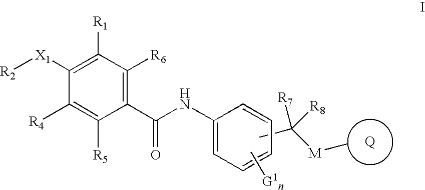

Or a pharmaceutically acceptable salt, hydrate or solvate of such compound

Wherein:
X₁ is independently selected from O, NR₃;
R₃ is independently selected from the group consisting of hydrogen, an optionally substituted (C₁-C₆)alkyl, (C₂-C₆)alkynyl, (C₂-C₆)alkenyl, (C₃-C₇)cycloalkyl, (C₂-C₆)alkylhalo, (C₁-C₆)alkyl-CN, (C₂-C₆)alkyl-O—(C₁-C₆)alkyl, (C₂-C₆)alkyl-O—(C₂-C₆)alkynyl, (C₂-C₆)alkyl-O—(C₂-C₆)alkenyl, (C₂-C₆)alkyl-O—(C₃-C₇)cycloalkyl or (C₂-C₆)alkyl-O-alkylcycloalkyl;
R₁ represent independently hydrogen, OH, an optionally substituted O—(C₀-C₆)alkyl, O—(C₂-C₆)alkynyl, O—(C₂-C₆)alkenyl, O—(C₃-C₇)cycloalkyl, O-alkylcycloalkyl, (C₁-C₆)alkyl, (C₂-C₆)alkynyl, (C₂-C₆)alkenyl, (C₃-C₇)cycloalkyl, (C₀-C₆)alkylhalo or (C₀-C₆)alkyl-CN;
R₂ represent independently hydrogen, an optionally substituted (C₁-C₆)alkyl, (C₂-C₆)alkynyl, (C₂-C₆)alkenyl, (C₃-C₇)cycloalkyl, (C₄-C₁₀)alkylcycloalkyl, (C₁-C₆)heterocycloalkyl, (C₁-C₆)alkyl-heteroaryl, (C₁-C₆)alkyl-aryl or (C₁-C₆)alkyl-CN;
   R₁ and R₂ according to the above definitions can be combined to form a heterocycloalkyl ring;
R₄ is independently selected from group consisted of hydrogen, OH, (C₀-C₆)alkyl-CN, (C₁-C₆)alkyl, (C₀-C₆)alkylhalo, (C₃-C₆)cycloalkyl, (C₁-C₆)alkyl -(C₃-C₈)cyclo alkyl, (C₂-C₆)alkenyl, (C₂-C₆)alkynyl, (C₀-C₆)alkyl-OR₉, (C₁-C₆)alkyl-NR₉R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O)R₉, (C₀-C₆)alkyl-S (═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀)R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-)cycloalkyl) or N((C₀-C₆)alkyl)(aryl) substituents;
R₅, R₆ are each independently selected from group consisted of hydrogen, (C₀-C₆)alkyl-CN, (C₁-C₆)alkyl, (C₀-C₆)alkylhalo, (C₃-C₆)cycloalkyl, (C₁-C₆)alkyl-(C₃-C₈)cycloalkyl, (C₂-C₆)alkenyl, (C₂-C₆)alkynyl, (C₀-C₆)alkyl-OR₉, (C₁-C₆)alkyl-NR₉R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(=O)R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀)R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-)cycloalkyl) or N((C₀-C₆)alkyl)(aryl) substituents;
G¹ is independently selected from a group consisting of hydrogen, OH, (C₀-C₆)alkyl-CN, (C₁-C₆)alkyl, (C₀-C₆)alkylhalo, (C₃-C₆)cycloalkyl, (C₀-C₆)alkyl-(C₃-C₈)cycloalkyl, (C₂-C₆)alkenyl, (C₂-C₆)alkynyl, (C₀-C₆)alkyl-OR₉, (C₀-C₆)alkyl-NR₉R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O)R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀)R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-)cycloalkyl) or N((C₀-C₆)alkyl)(aryl) substituents, wherein optionally two substituents are combined to the intervening atoms to form a bicyclic heterocycloalkyl, aryl or heteroaryl ring; wherein each ring is optionally further substituted with 1-5 independent halogen, CN, (C₁-C₆)alkyl, O—(C₀-C₆)alkyl, O-alkylcycloalkyl, O(aryl), O(heteroaryl), O-arylalkyl, O-heteroarylalkyl, N((—C₀-C₆)alkyl)((C₀-C₃)arylalkyl) or N((C₀-C₆)alkyl)(heteroarylalkyl) groups;
   R₉, R₁₀, R₁₁ each independently is hydrogen, (C₁-C₆)alkyl, (C₃-C₆)cycloalkyl, (C₁-C₆)alkyl-(C₃-C₈)cycloalkyl, (C₂-C₆)alkenyl, (C₂-C₆)alkynyl, (C₁-C₆)alkylhalo, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), N(C₀-C₆-alkyl)₂, —N((C₀-C₆)alkyl)((C₃-C₇-)cycloalkyl) or N((C₀-C₆)alkyl)(aryl) substituents;
n is an integer from 1 to 4, provided that when n>1, the G¹ groups may be equal or different from each other;
R₇ and R₈ represent independently an optionally substituted (C₁-C₄)alkyl, (C₁-C₆)alkylhalo, (C₀-C₆)alkyl-aryl, (C₁-C₆)alkyl-O—(C₀-C₆)-alkyl, (C₀-C₆)alkyl-heteroaryl, (C₀-C₆)alkyl-heterocycloalkyl, (C₀-C₆)alkyl-(C₃-C₇)cycloalkyl or R⁷ and R⁸ can together form a (C₃-C₆)cycloalkyl or an heterocycloalkyl group of formula:

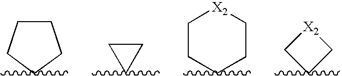

X₂ is independently selected from the group consisting of CH₂, O, S, SO₂;
M is independently selected from the group of consisting of a bond, an optionally substituted (C₁-C₆)alkyl, (C₂-C₆)alkynyl, (C₁-C₆)alkylhalo, (C₂-C₆)alkenyl, (C₁-C₆)alkyl-O—(C₀-C₆)alkyl, (C₁-C₆)-alkylhalo-O—(C₀-C₆)alkyl, (C₃-C₆)alkynyl-O—(C₀-C₆)alkyl, (C₃-C₆)alkenyl-O—(C₀-C₆)alkyl, (C₀-C₆)alkyl-S—(C₀-C₆)alkyl, (C₀-C₆)alkyl-S(═O)—(C₀-C₆)alkyl, (C₀-C₆)alkyl-S(═O)₂—(C₀-C₆)alkyl, (C₀-C₆)alkyl-NR₁₂—(C₀-C₆)alkyl, (C₀-C₆)alkyl-S(═O)₂NR₁₂—(C₀-C₆)alkyl, (C₀-C₆)alkyl-NR₁₂—S(═O)₂—(C₀-C₆)alkyl, (C₀-C₆)alkyl-C(═O)—NR₁₂—(C₀-C₆)alkyl, (C₀-C₆)alkyl-C(═O)—NR₁₂—(C₀-C₆)alkyl-O, (C₀-C₆)alkyl-C(═O)—NR₁₂—(C₀-C₆)alkyl-S, (C₀-C₆)alkyl-NR₁₂C(═O)—(C₀-C₆)alkyl, (C₀-C₆)alkyl-NR₁₂C(═O)—(C₂-C₆)alkyl-O, (C₀-C₆)alkyl-NR₁₂C(═O)—(C₂-C₆)alkyl-S, (C₀-C₆)alkyl-OC(═O)—(C₀-C₆)alkyl, (C₀-C₆)alkyl-C(═O)—O—(C₀-C₆)alkyl, (C₀-C₆)alkyl-C(═O)—(C₀-C₆)alkyl, (C₀-C₆)alkyl-NR₁₂—C(═O)—O—(C₀-C₆)alkyl, (C₀-C₆)alkyl-O—C(═O)—NR₁₂—(C₀-C₆)alkyl or (C₀-C₆)alkyl-NR₁₂—C(═O)—NR₁₃—(C₀-C₆)alkyl substituents;
   wherein optionally two substituents are combined to the intervening atoms to form a cycloalkyl, heterocycloalkyl or heteroaryl ring; wherein each ring is optionally further substituted with 1-5 independent hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₃-C₇)cycloalkyl, O—(C₁-C₆)alkyl-heteroaryl, O—(C₁-C₆)alkylaryl, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl;
   R₁₂ and R₁₃ are each independently selected from the group consisting of hydrogen, an optionally substituted (C₁-C₆)alkyl, (C₀-C₆)alkylhalo, (C₂-C₆)alkynyl, (C₂-C₆)alkenyl, (C₃-C₇)cycloalkyl, (C₁-C₆)alkyl-hetero aryl, (C₁-C₆)alkyl-aryl, aryl, heterocycloalkyl, heteroaryl ring; wherein each substitutable carbon atom in R₁₂, R₁₃ is optionally further substituted with hydrogen, OH, (C₁-C₆)alkyl, (C₀-C₄)alkyl-CN, (C₁-C₆)alkylhalo, OR₁₄, SR₁₄, NR₁₄R₁₅, NR₁₄C(═O)—R₁₅; C(═O)—NR₁₄R₁₅, S(═O)₂—NR₁₄R₁₅, NR₁₄S(═O)₂—R₁₅, C(═O)—OR₁₄; C(═NR₁₄)—NR₁₅, wherein R₁₄ and R₁₅ are each independently selected from H, (C₁-C₄)alkyl or (C₁-C₄)alkylhalo;
Q represent independently H, an optionally substituted (C₁-C₆)alkyl, (C₀-C₆)alkyl-CN, (C₁-C₆)alkylhalo, (C₃-C₇)cycloalkyl, (C₃-C₇)heterocycloalkyl or one of the following aryl or heteroaryl:

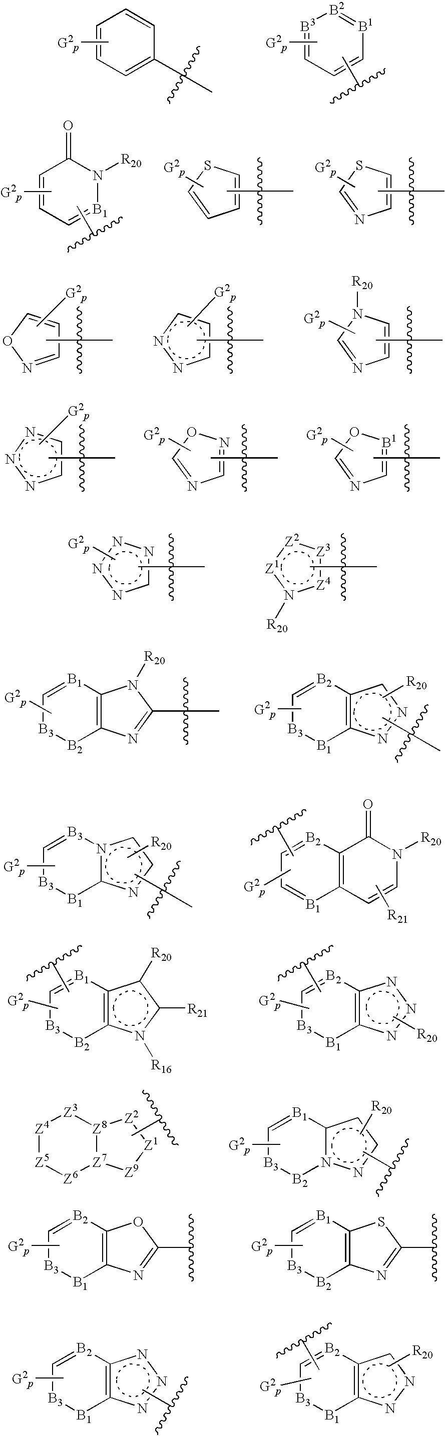

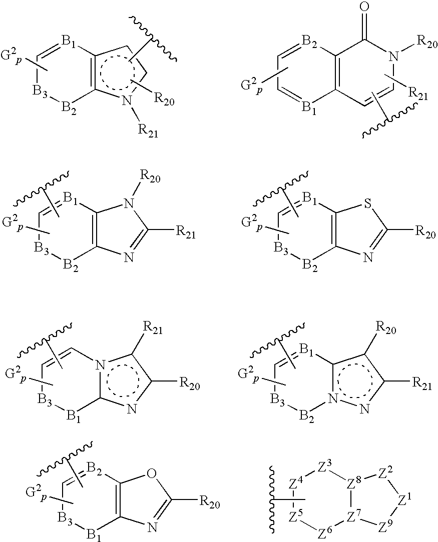

G² groups are each independently selected from the group consisting of hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₂-C₆)alkyl-OR₁₄, O—(C₃-C₇)cycloalkyl, O—(C₁-C₆)alkyl-heteroaryl, O—(C₁-C₆)alkyl-aryl, (C₀-C₆)alkyl-OR₁₄, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl, (C₁-C₆)alkyl-aryl, (C₁-C₆)alkylhalo-OR₁₇, (C₃-C₆)alkynyl-OR₁₇, (C₃-C₆)alkenyl-OR₁₇, (C₀-C₆)alkyl-S—R₁₇, O—(C₂-C₆)alkyl-S—R₁₇, (C₀-C₆)alkyl-S(═O)—R₁₇, O—(C₂-C₆)alkyl-S(═O)—R₁₇, (C₀-C₆)alkyl-S(═O)₂—R₁₇, O—(C₁-C₆)alkyl—S(═O)₂—R₁₇, (C₀-C₆)alkyl-NR₁₇R₁₈, O—(C₂-C₆)alkyl-NR₁₇R₁₈, (C₀-C₆)alkyl-S(═O)₂NR₁₇R₁₈, (C₀-C₆)alkyl-NR₁₇—S(═O)₂R₁₈, O—(C₁-C₆)alkyl-S(═O)₂NR₁₇R₁₈, O—(C₂-C₆)alkyl-NR₁₇—S(═O)₂R₁₈, (C₀-C₆)alkyl-C(═O)—NR₁₇R₁₈, (C₀-C₆)alkyl-NR₁₇C(═O)—R₁₈, O—(C₁-C₆)alkyl-C(═O)—NR₁₇R₁₈, O—(C₂-C₆)alkyl-NR₁₇C(═O)—R₁₈, (C₀-C₆)alkyl-OC(═O)—R₁₇, (C₀-C₆)alkyl-C(═O)—OR₁₇, O—(C₂-C₆)alkyl-OC(═O)—R₁₇, O—(C₁-C₆)alkyl-C(═O)—OR₁₇, (C₀-C₆)alkyl-C(═O)—R₁₇, O—(C₁-C₆)alkyl-C(═O)—R₁₇, (C₀-C₆)alkyl-NR₁₇—C(═O)—OR₁₈, (C₀-C₆)alkyl-O—C(═O)—NR₁₇R₁₈ or (C₀-C₆)alkyl-NR₁₇—C(═O)—NR₁₈R₁₉ substituents;
wherein optionally two substituents are combined to the intervening atoms to form a bicyclic aryl, cycloalkyl, heterocycloalkyl or heteroaryl ring; wherein each ring is optionally further substituted with 1-5 independent hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₃-C₇)cycloalkyl, O—(C₁-C₆)alkyl-heteroaryl, O—(C₁-C₆)alkylaryl, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl;
p is an integer that is selected from the group consisting of 1,2, 3, 4 and 5 provided that when p>1, the G² groups may be equal or different from each other;
R₁₆, R₁₇, R₁₈, R₁₉, R₂₀ and R₂₁ are each independently selected from the group consisting of hydrogen, an optionally substituted (C₁-C₆)alkyl, (C₁-C₆)alkylhalo, (C₁-C₆)alkyl-CN, (C₁-C₆)alkyl-O—(C₀-C₆)alkyl, (C₁-C₆)alkyl-N((C₀-C₆)alkyl)₂, (C₁-C₆)alkyl-C(═O)—O—(C₀-C₆)alkyl, (C₁-C₆)alkyl-heterocycloalkyl, (C₂-C₆)alkynyl, (C₂-C₆)alkenyl, (C₃-C₇)cycloalkyl, (C₃-C₇)cycloalkyl-(C₁-C₆)alkyl, heteroaryl, (C₁-C₆)alkyl-heteroaryl, aryl;
Z¹, Z², Z³, Z⁴, Z⁵, Z⁶, Z⁷, Z⁸ and Z⁹ are each independently selected from the group consisting of bond, —C═, —C═C—, —C(═O)—, —C(═S)—, —C—, —O—, —N═, —N— or —S— which may further be substituted by G²ₚ groups;
B¹, B² and B³ are each selected independently from —C—, —N—, —O— or —S— which may further be substituted by one G²ₚ group;
Any N or S bearing ring may be depicted in its N-oxide, S-oxide or S-dioxide form;

It being understood that:
When

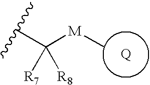

stands in the para-position of the phenyl ring with R₇ and R₈ are each independently selected from an optionally substituted (C₁-C₄)alkyl, or can together form a (C₃-C₆)cycloalkyl or an heterocycloalkyl group of formula:

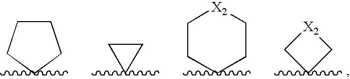

and G¹ₙ are hydrogens, then

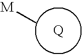

can not be

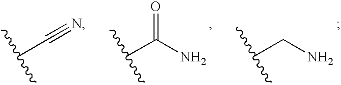

If X₁ is O, then R₁ is represented by O—(C₁-C₆)alkyl, O—(C₂-C₆)alkynyl, O—(C₂-C₆)alkenyl, O—(C₃-C₇)cycloalkyl, O-alkylcycloalkyl;
X₁—R₂ and R₁ may not represent at the same time OH;
R₇ and R₈ may not represent at the same time (C₀-C₆)alkyl-aryl, (C₀-C₆)alkyl-heteroaryl;
If R₅ or R₆ are represented by (C₀-C₆)alkyl-OR₉, then R₉ may not represent an hydrogen;
G¹ₙ groups may not represent at the same time OH;
If R₇, R₈ and M represent at the same time an optionally substituted (C₁-C₄)alkyl, then Q can not be H;
If R₇, R₈ represent

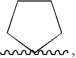

then compounds of the following list are excluded from the present invention:
3,4-dimethoxy-N-[4-[1-[[(4-methoxyphenyl)amino]carbonyl]cyclopentyl]phenyl]-benzamide
N-[4-(1-cyanocyclopentyl)phenyl]-3,4-dimethoxy-benzamide.

Definition of Terms

For the avoidance of doubt it is to be understood that in this specification “(C₁-C₆)” means a carbon group having 1, 2, 3, 4, 5 or 6 carbon atoms. “(C₀-C₆)” means a carbon group having 0, 1, 2, 3, 4, 5 or 6 carbon atoms.

In this specification “C” means a carbon atom.

In the above definition, the term “(C₁-C₆)alkyl” includes group such as methyl, ethyl, propyl, isopropyl, butyl, isobutyl, sec-butyl, tert-butyl, pentyl, isopentyl, neopentyl, tert-pentyl, hexyl or the like.

“(C₂-C₆)alkenyl” includes group such as ethenyl, 1-propenyl, allyl, isopropenyl, 1-butenyl, 3 -butenyl, 4-pentenyl and the like.

“(C₂-C₆)alkynyl” includes group such as ethynyl, propynyl, butynyl, pentynyl and the like.

“Cycloalkyl” refers to an optionally substituted carbocycle containing no heteroatoms, includes mono-, bi-, and tricyclic saturated carbocycles, as well as fused ring systems. Such fused ring systems can include on ring that is partially or fully unsaturated such as a benzene ring to form fused ring systems such as benzo fused carbocycles. Cycloalkyl includes such fused ring systems as spirofused ring systems. Examples of cycloalkyl include cyclopropyl, cyclobutyl, cyclopentyl, cyclohexyl, decahydronaphthalene, adamantane, indanyl, fluorenyl, 1,2,3,4-tetrahydronaphthalene and the like.

“Alkyl cycloalkyl” includes (C₁-C₁₀)alkyl-(C₃-C₈)cycloalkyl group such methylcyclohexyl group, isopropylcyclopentyl group, isobutylcyclopentane group or the like.

In this specification, unless state otherwise, the term “halo” and “halogen” may be fluoro, chloro, bromo or iodo.

In this specification, unless state otherwise, the term “alkylhalo” means an alkyl group as defined above, which is substituted with an halo as described above. The term (C₂-C₆)alkylhalo may include, but is not limited to fluoromethyl or bromopropyl.

“Heterocycloalkyl” refers to an optionally substituted carbocycle containing at least one heteroatom selected independently from O, N, S. It includes mono-, bi-, and tricyclic saturated carbocycles, as well as fused ring systems. Such fused ring systems can include one ring that is partially or fully unsaturated such as a benzene ring to form fused ring systems such as benzo fused carbocycles. Examples of heterocycloalkyl include piperidine, piperazine, morpholine, tetrahydrothiophene, indoline, isoquinoline and the like.

“Aryl” includes (C₆-C₁₀)aryl group such as phenyl, 1-naphtyl, 2-naphtyl and the like.

“Arylalkyl” includes (C₆-C₁₀)aryl-(C₁-C₃)alkyl group such as benzyl group, 1-phenylethyl group, 2-phenylethyl group, 1-phenylpropyl group, 2-phenylpropyl group, 3-phenylpropyl group, 1-naphtylmethyl group, 2-naphtylmethyl group or the like.

“Heteroaryl” includes 5-10 membered heterocyclic group containing 1 to 4 heteroatoms selected from oxygen, nitrogen or sulphur to form a ring such as furyl (furan ring), benzofuranyl (benzofuran ring), thienyl (thiophene ring), benzothiophenyl (benzothiophene ring), pyrrolyl (pyrrole ring), imidazolyl (imidazole ring), pyrazolyl (pyrazole ring), thiazolyl (thiazole ring), isothiazolyl (isothiazole ring), triazolyl (triazole ring), tetrazolyl (tetrazole ring), pyridil (pyridine ring), pyrazynyl (pyrazine ring), pyrimidinyl (pyrimidine ring), pyridazinyl (pyridazine ring), indolyl (indole ring), isoindolyl (isoindole ring), benzoimidazolyl (benzimidazole ring), purinyl group (purine ring), quinolyl (quinoline ring), phtalazinyl (phtalazine ring), naphtyridinyl (naphtyridine ring), quinoxalinyl (quinoxaline ring), cinnolyl (cinnoline ring), pteridinyl (pteridine ring), oxazolyl (oxazole ring), isoxazolyl (isoxazole ring), benzoxazolyl (benzoxazole ring), benzothiazolyly (benzothiaziole ring), furazanyl (furazan ring) and the like.

“Heteroarylalkyl” includes heteroaryl-(C₁-C₃-alkyl) group, wherein examples of heteroaryl are the same as those illustrated in the above definition, such as 2-furylmethyl group, 3-furylmethyl group, 2-thienylmethyl group, 3-thienylmethyl group, 1-imidazolylmethyl group, 2-imidazolylmethyl group, 2-thiazolylmethyl group, 2-pyridylmethyl group, 3-pyridylmethyl group, 1-quinolylmethyl group or the like.

“Solvate” refers to a complex of variable stoechiometry formed by a solute (e.g. a compound of formula I) and a solvent. The solvent is a pharmaceutically acceptable solvent as water preferably; such solvent may not interfere with the biological activity of the solute.

“Optionally” means that the subsequently described event(s) may or may not occur, and includes both event(s), which occur, and events that do not occur.

The term “substituted” refers to substitution with the named substituent or substituents, multiple degrees of substitution being allowed unless otherwise stated.

Preferred compounds of the present invention are compounds of formula I-A depicted below

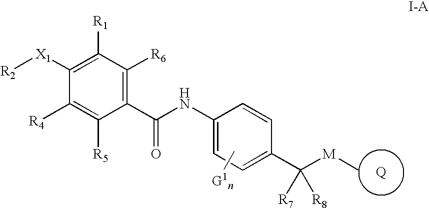

Or a pharmaceutically acceptable salt, hydrate or solvate of such compound

Wherein:
X₁ is selected from O, NR₃;
R₃ is independently selected from the group consisting of hydrogen, an optionally substituted (C₁-C₆)alkyl, (C₂-C₆)alkynyl, (C₂-C₆)alkenyl, (C₃-C₇)cycloalkyl, (C₂-C₆)alkylhalo, (C₁-C₆)alkyl-CN, (C₂-C₆)alkyl-O—(C₁-C₆)alkyl, (C₂-C₆)alkyl-O—(C₂-C₆)alkynyl, (C₂-C₆)alkyl-O—(C₂-C₆)alkenyl, (C₂-C₆)alkyl-O—(C₃-C₇)cycloalkyl or (C₂-C₆)alkyl-O-alkylcycloalkyl;
R₁ represent independently hydrogen, OH, an optionally substituted O—(C₀-C₆)alkyl, O—(C₂-C₆)alkynyl, O—(C₂-C₆)alkenyl, O—(C₃-C₇)cycloalkyl, O-alkylcycloalkyl, (C₁-C₆)alkyl, (C₂-C₆)alkynyl, (C₂-C₆)alkenyl, (C₃-C₇)cycloalkyl, (C₀-C₆)alkylhalo or (C₀-C₆)alkyl-CN;
R₂ represent independently hydrogen, an optionally substituted (C₁-C₆)alkyl, (C₂-C₆)alkynyl, (C₂-C₆)alkenyl, (C₃-C₇)cycloalkyl, (C₄-C₁₀)alkylcycloalkyl, (C₁-C₆)heterocycloalkyl, (C₁-C₆)alkyl-heteroaryl, (C₁-C₆)alkyl-aryl or (C₁-C₆)alkyl-CN;
   R₁ and R₂ according to the above definitions can be combined to form a heterocycloalkyl ring;
R₄ is independently selected from group consisted of hydrogen, OH, (C₀-C₆)alkyl-CN, (C₁-C₆)alkyl, (C₀-C₆)alkylhalo, (C₃-C₆)cycloalkyl, (C₁-C₆)alkyl-(C₃-C₈)cycloalkyl, (C₂-C₆)alkenyl, (C₂-C₆)alkynyl, (C₀-C₆)alkyl-OR₉, (C₁-C₆)alkyl-NR₉R₁₀, (C₀-C₆)-alkyl-NR₉COR₁₀, (C₀-C₆)alkyl -NR₉SO₂R₁₀, (C₀-C₆)alkyl-NR₁₁CONR₁₀R₉, (C₀-C₆)alkyl-SR₉, (C₀-C₆)alkyl-S(═O)R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀)R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-)cycloalkyl) or N((C₀-C₆)alkyl)(aryl) substituents;
R₅, R₆ are each independently selected from group consisted of hydrogen, (C₀-C₆)alkyl-CN, (C₁-C₆)alkyl, (C₀-C₆)alkylhalo, (C₃-C₆)cycloalkyl, (C₁-C₆)alkyl-(C₃-C₈)cycloalkyl, (C₂-C₆)alkenyl, (C₂-C₆)alkynyl, (C₀-C₆)alkyl-OR₉, (C₁-C₆)alkyl-NR₉R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O)R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀)R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-)cycloalkyl) or N((C₀-C₆)alkyl)(aryl) substituents;
G¹ is independently selected from a group consisting of hydrogen, OH, (C₀-C₆)alkyl-CN, (C₁-C₆)alkyl, (C₀-C₆)alkylhalo, (C₃-C₆)cycloalkyl, (C₀-C₆)alkyl-(C₃-C₈)cycloalkyl, (C₂-C₆)alkenyl, (C₂-C₆)alkynyl, (C₀-C₆)alkyl-OR₉, (C₀-C₆)alkyl-NR₉R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O)R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀)R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-)cycloalkyl) or N((C₀-C₆)alkyl)(aryl) substituents, wherein optionally two substituents are combined to the intervening atoms to form a bicyclic heterocycloalkyl, aryl or heteroaryl ring; wherein each ring is optionally further substituted with 1-5 independent halogen, CN, (C₁-C₆)alkyl, O—(C₀-C₆)alkyl, O-alkylcycloalkyl, O(aryl), O(heteroaryl), O-arylalkyl, O-heteroarylalkyl, N((—C₀-C₆)alkyl)((C₀-C₃)arylalkyl) or N((C₀-C₆)alkyl)(heteroarylalkyl) groups;
   R₉, R₁₀, R₁₁ each independently is hydrogen, (C₁-C₆)alkyl, (C₃-C₆)cycloalkyl, (C₁-C₆)alkyl-(C₃-C₈)cycloalkyl, (C₂-C₆)alkenyl, (C₂-C₆)alkynyl, (C₁-C₆)alkylhalo, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), N(C₀-C₆-alkyl)₂, —N((C₀-C₆)alkyl)((C₃-C₇-)cycloalkyl) or N((C₀-C₆)alkyl)(aryl) substituents;
n is an integer from 1 to 4, provided that when n>1, the G¹ groups may be equal or different from each other;
R₇ and R₈ represent independently an optionally substituted (C₁-C₄)alkyl, (C₁-C₆)alkylhalo, (C₀-C₆)alkyl-aryl, (C₁-C₆)alkyl-O—(C₀-C₆)-alkyl, (C₀-C₆)alkyl-heteroaryl, (C₀-C₆)alkyl-heterocycloalkyl, (C₀-C₆)alkyl-(C₃-C₇)cycloalkyl or R⁷ and R⁸ can together form a (C₃-C₆)cycloalkyl or an heterocycloalkyl group of formula:

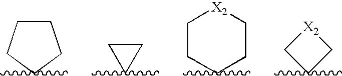

X₂ is independently selected from the group consisting of CH₂, O, S, SO₂;
M is independently selected from the group of consisting of a bond, an optionally substituted (C₁-C₆)alkyl, (C₂-C₆)alkynyl, (C₁-C₆)alkylhalo, (C₂-C₆)alkenyl, (C₁-C₆)alkyl-O—(C₀-C₆)alkyl, (C₁-C₆)-alkylhalo-O—(C₀-C₆)alkyl, (C₃-C₆)alkynyl-O—(C₀-C₆)alkyl, (C₃-C₆)alkenyl-O—(C₀-C₆)alkyl, (C₀-C₆)alkyl-S—(C₀-C₆)alkyl, (C₀-C₆)alkyl-S(═O)—(C₀-C₆)alkyl, (C₀-C₆)alkyl-S(═O)₂—(C₀-C₆)alkyl, (C₀-C₆)alkyl-NR₁₂—(C₀-C₆)alkyl, (C₀-C₆)alkyl-S(═O)₂NR₁₂—(C₀-C₆)alkyl, (C₀-C₆)alkyl-NR₁₂—S(═O)₂—(C₀-C₆)alkyl, (C₀-C₆)alkyl-C(═O)—NR₁₂—(C₀-C₆)alkyl, (C₀-C₆)alkyl-C(═O)—NR₁₂—(C₀-C₆)alkyl-O, (C₀-C₆)alkyl-C(═O)—NR₁₂—(C₀-C₆)alkyl-S, (C₀-C₆)alkyl-NR₁₂C(═O)—(C₀-C₆)alkyl, (C₀-C₆)alkyl-NR₁₂C(═O)—(C₂-C₆)alkyl-O, (C₀-C₆)alkyl-NR₁₂C(═O)—(C₂-C₆)alkyl-S, (C₀-C₆)alkyl-OC(═O)—(C₀-C₆)alkyl, (C₀-C₆)alkyl-C(═O)—O—(C₀-C₆)alkyl, (C₀-C₆)alkyl-C(═O)—(C₀-C₆)alkyl, (C₀-C₆)alkyl-NR₁₂—C(═O)—O—(C₀-C₆)alkyl, (C₀-C₆)alkyl-O—C(═O)—NR₁₂—(C₀-C₆)alkyl or (C₀-C₆)alkyl-NR₁₂—C(═O)—NR₁₃—(C₀-C₆)alkyl substituents;
   wherein optionally two substituents are combined to the intervening atoms to form a cycloalkyl, heterocycloalkyl or heteroaryl ring; wherein each ring is optionally further substituted with 1-5 independent hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₃-C₇)cycloalkyl, O—(C₁-C₆)alkyl-heteroaryl, O—(C₁-C₆)alkylaryl, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl;
   R₁₂ and R₁₃ are each independently selected from the group consisting of hydrogen, an optionally substituted (C₁-C₆)alkyl, (C₀-C₆)alkylhalo, (C₂-C₆)alkynyl, (C₂-C₆)alkenyl, (C₃-C₇)cycloalkyl, (C₁-C₆)alkyl-heteroaryl, (C₁-C₆)alkyl-aryl, aryl, heterocycloalkyl, heteroaryl ring; wherein each substitutable carbon atom in R₁₂, R₁₃ is optionally further substituted with hydrogen, OH, (C₁-C₆)alkyl, (C₀-C₄)alkyl-CN, (C₁-C₆)alkylhalo, OR₁₄, SR₁₄, NR₁₄R₁₅, NR₁₄C(═O)—R₁₅, C(═O)—NR₁₄R₁₅, S(═O)₂—NR₁₄R₁₅, NR₁₄S(═O)₂—R₁₅, C(═O)—OR₁₄; C(═NR₁₄)—NR₁₅, wherein R₁₄ and R₁₅ are each independently selected from H, (C₁-C₄)alkyl or (C₁-C₄)alkylhalo;
Q represent independently H, an optionally substituted (C₁-C₆)alkyl, (C₀-C₆)alkyl-CN, (C₁-C₆)alkylhalo, (C₃-C₇)cycloalkyl, (C₃-C₇) heterocycloalkyl or one of the following aryl or heteroaryl:

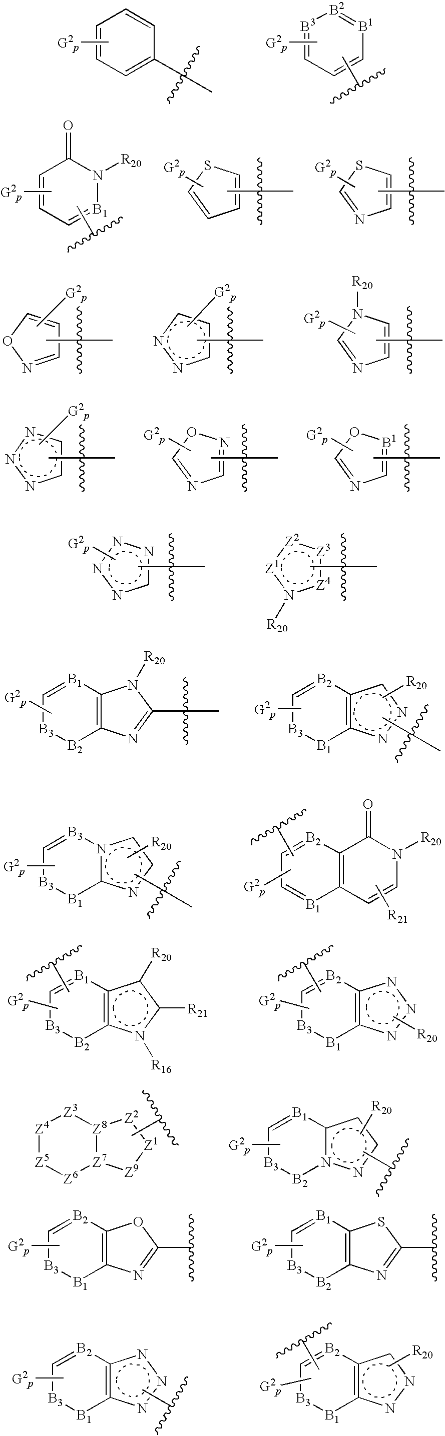

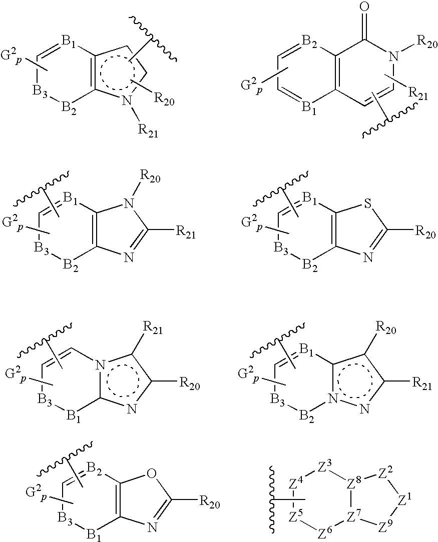

G² groups are each independently selected from the group consisting of hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₂-C₆)alkyl-OR₁₄, O—(C₃-C₇)cycloalkyl, O—(C₁-C₆)alkyl-heteroaryl, O—(C₁-C₆)alkyl-aryl, (C₀-C₆)alkyl-OR₁₄, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl, (C₁-C₆)alkyl-aryl, (C₁-C₆)alkylhalo-OR₁₇, (C₃-C₆)alkynyl-OR₁₇, (C₃-C₆)alkenyl-OR₁₇, (C₀-C₆)alkyl-S—R₁₇, O—(C₂-C₆)alkyl-S—R₁₇, (C₀-C₆)alkyl-S(═O)—R₁₇, O—(C₂-C₆)alkyl-S(═O)—R₁₇, (C₀-C₆)alkyl-S(═O)₂—R₁₇, O—(C₁-C₆)alkyl-S(═O)₂—R₁₇, (C₀-C₆)alkyl-NR₁₇R₁₈, O—(C₂-C₆)alkyl-NR₁₇R₁₈, (C₀-C₆)alkyl-S(═O)₂NR₁₇R₁₈, (C₀-C₆)alkyl-NR₁₇—S(═O)₂R₁₈, O—(C₁-C₆)alkyl-S(—O)₂NR₁₇R₁₈, O—(C₂-C₆)alkyl-NR₁₇—S(═O)₂R₁₈, (C₀-C₆)alkyl-C(═O)—NR₁₇R₁₈, (C₀-C₆)alkyl-NR₁₇C(═O)—R₁₈, O—(C₁-C₆)alkyl-C(═O)—NR₁₇R₁₈, O—(C₂-C₆)alkyl-NR₁₇C(═O)—R₁₈ , (C₀-C₆)alkyl-OC(═O)—R₁₇, (C₀-C₆)alkyl-C(═O)—OR₁₇, O—(C₂-C₆)alkyl-OC(═O)—R₁₇, O—(C₁-C₆)alkyl-C(═O)—OR₁₇, (C₀-C₆)alkyl-C(═O)—R₁₇, O—(C₁-C₆)alkyl-C(═O)—R₁₇, (C₀-C₆)alkyl-NR₁₇—C(═O)—OR₁₈, (C₀-C₆)alkyl-O—C(═O)—NR₁₇R₁₈ or (C₀-C₆)alkyl-NR₁₇—C(═O)—NR₁₈R₁₉ substituents;
wherein optionally two substituents are combined to the intervening atoms to form a bicyclic aryl, cycloalkyl, heterocycloalkyl or heteroaryl ring; wherein each ring is optionally further substituted with 1-5 independent hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₃-C₇)cycloalkyl, O—(C₁-C₆)alkyl-heteroaryl, O—(C₁-C₆)alkylaryl, (C₃-C₇)cyclo alkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl;
p is an integer that is selected from the group consisting of 1,2, 3, 4 and 5 provided that when p>1, the G² groups may be equal or different from each other;
R₁₆, R₁₇, R₁₈, R₁₉, R₂₀ and R₂₁ are each independently selected from the group consisting of hydrogen, an optionally substituted (C₁-C₆)alkyl, (C₁-C₆)alkylhalo, (C₁-C₆)alkyl-CN, (C₁-C₆)alkyl-O—(C₀-C₆)alkyl, (C₁-C₆)alkyl-N((C₀-C₆)alkyl)₂, (C₁-C₆)alkyl-C(═O)—O—(C₀-C₆)alkyl, (C₁-C₆)alkyl-heterocycloalkyl, (C₂-C₆)alkynyl, (C₂-C₆)alkenyl, (C₃-C₇)cycloalkyl, (C₃-C₇)cycloalkyl-(C₁-C₆)alkyl, heteroaryl, (C₁-C₆)alkyl-heteroaryl, aryl;
Z¹, Z², Z³, Z⁴, Z⁵, Z⁶, Z⁷, Z⁸ and Z⁹ are each independently selected from the group consisting of bond, —C=, —C═C—, —C(═O)—, —C(═S)—, —C—, —O—, —N═, —N— or —S— which may further be substituted by G²ₚ groups;
B¹, B² and B³ are each selected independently from —C—, —N—, —O— or —S— which may further be substituted by one G²ₚ group;
Any N or S bearing ring may be depicted in its N-oxide, S-oxide or S-dioxide form;

It being understood that:
If X, is O, then R₁ is represented by O—(C₁-C₆)alkyl, O—(C₂-C₆)alkynyl, O—(C₂-C₆)alkenyl, O—(C₃-C₇)cycloalkyl, O-alkylcycloalkyl;
X₁—R₂ and R₁ may not represent at the same time OH;
If R₇ and R₈ all represent CH₃ at the same time, then M-Q may not represent CH₃;
R₇ and R₈ may not represent at the same time (C₀-C₆)alkyl-aryl, (C₀-C₆)alkyl-heteroaryl;
If R₅ or R₆ are represented by (C₀-C₆)alkyl-OR₉, then R₉ may not represent an hydrogen;
G¹ₙ groups may not represent at the same time OH;
If R₇, R₈ and M represent at the same time an optionally substituted (C₁-C₄)alkyl, then Q can not be H;
If R₇, R₈ represent

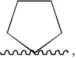

then compounds of the following list are excluded from the present invention:
3,4-dimethoxy-N-[4-[1-[[(4-methoxyphenyl)amino]carbonyl]cyclopentyl]phenyl]-benzamide
N-[4-(1-cyanocyclopentyl)phenyl]-3,4-dimethoxy-benzamide
When

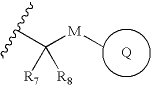

stands in the para-position of the phenyl ring with R₇ and R₈ are each independently selected from an optionally substituted (C₁-C₄)alkyl, or can together form a (C₃-C₆)cycloalkyl or an heterocycloalkyl group of formula:

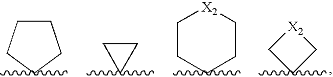

and G¹ₙ are hydrogens, then

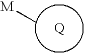

can not be

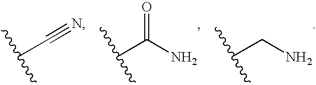

In one aspect, the compounds of the present invention are represented by formula I-A wherein R₁ and R₂ groups are specified as in the formula I-A1 depicted below

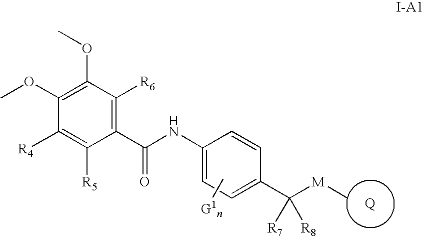

Or a pharmaceutically acceptable salt, hydrate or solvate of such compound

Wherein:
R₄, R₅, R₆ are each independently selected from group consisted of hydrogen, (C₀-C₆)alkyl-CN, (C₁-C₆)alkyl, (C₀-C₆)alkylhalo, (C₃-C₆)cycloalkyl, (C₁-C₆)alkyl-(C₃-C₈)cycloalkyl, (C₂-C₆)alkenyl, (C₂-C₆)alkynyl, (C₀-C₆)alkyl-OR₉, (C₁-C₆)alkyl-NR₉R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O)R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀)R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-)cycloalkyl) or N((C₀-C₆)alkyl)(aryl) substituents;
G¹ is independently selected from a group consisting of hydrogen, OH, (C₁-C₆)alkyl, (C₀-C₆)alkyl-CN, (C₀-C₆)alkylhalo, (C₃-C₆)cycloalkyl, (C₀-C₆)alkyl-(C₃-C₈)cycloalkyl, (C₂-C₆)alkenyl, (C₂-C₆)alkynyl, (C₀-C₆)alkyl-OR₉, (C₀-C₆)alkyl-NR₉R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O)R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀)R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-)cycloalkyl) or N((C₀-C₆)alkyl)(aryl) substituents, wherein optionally two substituents are combined to the intervening atoms to form a bicyclic heterocycloalkyl, aryl or heteroaryl ring; wherein each ring is optionally further substituted with 1-5 independent halogen, CN, (C₁-C₆)alkyl, O—(C₀-C₆)alkyl, O-alkylcycloalkyl, O(aryl), O(heteroaryl), O-arylalkyl, O-heteroarylalkyl, N((—C₀-C₆)alkyl)((C₀-C₃)arylalkyl) or N((C₀-C₆)alkyl)(heteroarylalkyl) groups;
   R₉, R₁₀, R₁₁ each independently is hydrogen, (C₁-C₆)alkyl, (C₃-C₆)cycloalkyl, (C₁-C₆)alkyl-(C₃-C₈)cycloalkyl, (C₂-C₆)alkenyl, (C₂-C₆)alkynyl, (C₁-C₆)alkylhalo, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), N(C₀-C₆-alkyl)₂, —N((C₀-C₆)alkyl)((C₃-C₇-)cycloalkyl) or N((C₀-C₆)alkyl)(aryl) substituents;
   n is an integer from 1 to 4, provided that when n>1, the G¹ groups may be equal or different from each other;
R₇ and R₈ are selected from group of formula:

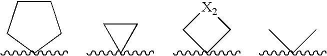

X₂ is independently selected from the group consisting of CH₂, O, S, SO₂;
M is independently selected from the group of consisting of a bond, an optionally substituted (C₁-C₆)alkyl, (C₂-C₆)alkynyl, (C₁-C₆)alkylhalo, (C₂-C₆)alkenyl, (C₁-C₆)alkyl-O—(C₀-C₆)alkyl, (C₁-C₆)-alkylhalo-O—(C₀-C₆)alkyl, (C₃-C₆)alkynyl-O—(C₀-C₆)alkyl, (C₃-C₆)alkenyl-O—(C₀-C₆)alkyl, (C₀-C₆)alkyl-S—(C₀-C₆)alkyl, (C₀-C₆)alkyl-S(═O)—(C₀-C₆)alkyl, (C₀-C₆)alkyl-S(═O)₂—(C₀-C₆)alkyl, (C₀-C₆)alkyl-NR₁₂—(C₀-C₆)alkyl, (C₀-C₆)alkyl-S(═O)₂NR₁₂—(C₀-C₆)alkyl, (C₀-C₆)alkyl-NR₁₂—S(═O)₂—(C₀-C₆)alkyl, (C₀-C₆)alkyl-C(═O)—NR₁₂—(C₀-C₆)alkyl, (C₀-C₆)alkyl-C(═O)—NR₁₂—(C₀-C₆)alkyl-O, (C₀-C₆)alkyl-C(═O)—NR₁₂—(C₀-C₆)alkyl-S , (C₀-C₆)alkyl-NR₁₂C(═O)—(C₀-C₆)alkyl, (C₀-C₆)alkyl-NR₁₂C(═O)—(C₂-C₆)alkyl-O, (C₀-C₆)alkyl-NR₁₂C(═O)—(C₂-C₆)alkyl-S, (C₀-C₆)alkyl-OC(═O)—(C₀-C₆)alkyl, (C₀-C₆)alkyl-C(═O)—O—(C₀-C₆)alkyl, (C₀-C₆)alkyl-C(═O)—(C₀-C₆)alkyl, (C₀-C₆)alkyl-NR₁₂—C(═O)—O—(C₀-C₆)alkyl, (C₀-C₆)alkyl-O—C(═O)—NR₁₂—(C₀-C₆)alkyl or (C₀-C₆)alkyl-NR₁₂—C(═O)—NR₁₃—(C₀-C₆)alkyl substituents;
   wherein optionally two substituents are combined to the intervening atoms to form a cycloalkyl, heterocycloalkyl or heteroaryl ring; wherein each ring is optionally further substituted with 1-5 independent hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₃-C₇)cycloalkyl, O—(C₁-C₆)alkyl-heteroaryl, O—(C₁-C₆)alkylaryl, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl;
   R₁₂ and R₁₃ are each independently selected from the group consisting of hydrogen, an optionally substituted (C₁-C₆)alkyl, (C₀-C₆)alkylhalo, (C₂-C₆)alkynyl, (C₂-C₆)alkenyl, (C₃-C₇)cycloalkyl, (C₁-C₆)alkyl-heteroaryl, (C₁-C₆)alkyl-aryl, aryl, heterocycloalkyl, heteroaryl ring; wherein each substitutable carbon atom in R₁₂, R₁₃ is optionally further substituted with hydrogen, OH, (C₁-C₆)alkyl, (C₀-C₄)alkyl-CN, (C₁-C₆)alkylhalo, OR₁₄, SR₁₄, NR₁₄R₁₅, NR₁₄C(═O)—R₁₅, C(═O)—NR₁₄R₁₅, S(═O)₂—NR₁₄R₁₅, NR₁₄S(═O)₂—R₁₅, C(═O)—OR₁₄; C(═NR₁₄)—NR₁₅, wherein R₁₄ and R₁₅ are each independently selected from H, (C₁-C₄)alkyl or (C₁-C₄)alkylhalo;
Q represent independently H, an optionally substituted (C₁-C₆)alkyl, (C₀-C₆)alkyl-CN, (C₁-C₆)alkylhalo, (C₃-C₇)cycloalkyl, (C₃-C₇)heterocycloalkyl or one of the following aryl or heteroaryl:

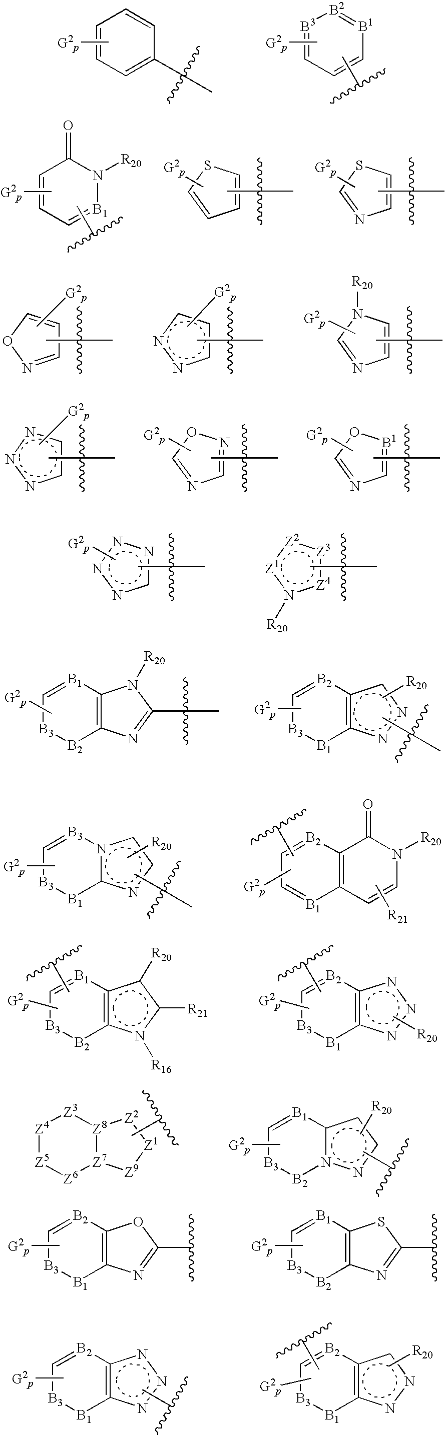

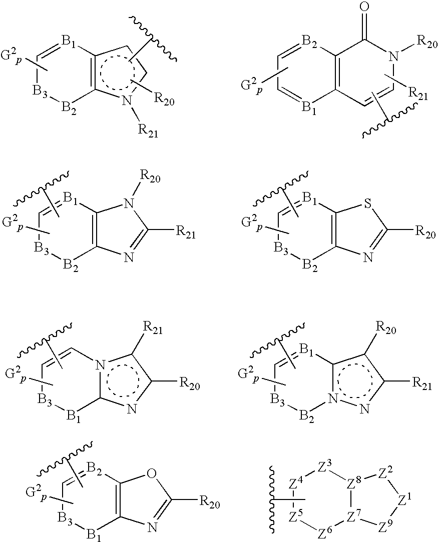

G² groups are each independently selected from the group consisting of hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₂-C₆)alkyl-OR₁₄, O—(C₃-C₇)cycloalkyl, O—(C₁-C₆)alkyl-heteroaryl, O—(C₁-C₆)alkyl-aryl, (C₀-C₆)alkyl-OR₁₄, (C₃-C₇)cyclo alkyl, (C₃-C₇)cycloalkyl-(C₁-C₆)alkyl , O—(C₃-C₇)cycloalkyl-(C₁-C₆)alkyl, O-heteroaryl, heteroaryl, (C₁-C₆)alkyl-heteroaryl, aryl, O-aryl, (C₁-C₆)alkyl-aryl, (C₁-C₆)alkylhalo-OR₁₇, (C₃-C₆)alkynyl-OR₁₇, (C₃-C₆)alkenyl-OR₁₇, (C₀-C₆)alkyl-S—R₁₇, O—(C₂-C₆)alkyl-S—R₁₇, (C₀-C₆)alkyl-S(═O)—R₁₇, O—(C₂-C₆)alkyl-S(═O)—R₁₇, (C₀-C₆)alkyl-S(═O)₂—R₁₇, O—(C₁-C₆)alkyl-S(═O)₂—R₁₇, (C₀-C₆)alkyl-NR₁₇R₁₈, O—(C₂-C₆)alkyl-NR₁₇R₁₈, (C₀-C₆)alkyl-S(═O)₂NR₁₇R₁₈, (C₀-C₆)alkyl-NR₁₇—S(═O)₂R₁₈, O—(C₁-C₆)alkyl-S(═O)₂NR₁₇R₁₈, O—(C₂-C₆)alkyl-NR₁₇—S(═O)₂R₁₈, (C₀-C₆)alkyl-C(═O)—NR₁₇R₁₈, (C₀-C₆)alkyl-NR₁₇C(═O)—R₁₈, O—(C₁-C₆)alkyl-C(═O)—NR₁₇R₁₈, O—(C₂-C₆)alkyl-NR₁₇C(═O)—R₁₈, (C₀-C₆)alkyl-OC(═O)—R₁₇, (C₀-C₆)alkyl-C(═O)—OR₁₇, O—(C₂-C₆)alkyl-OC(═O)—R₁₇, O—(C₁-C₆)alkyl-C(═O)—OR₁₇, (C₀-C₆)alkyl-C(═O)—R₁₇, O—(C₁-C₆)alkyl-C(═O)—R₁₇, (C₀-C₆)alkyl-NR₁₇—C(═O)—OR₁₈, (C₀-C₆)alkyl-O—C(═O)—NR₁₇R₁₈ or (C₀-C₆)alkyl-NR₁₇—C(═O)—NR₁₈R₁₉ substituents;
wherein optionally two substituents are combined to the intervening atoms to form a bicyclic aryl, cycloalkyl, heterocycloalkyl or heteroaryl ring; wherein each ring is optionally further substituted with 1-5 independent hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₃-C₇)cycloalkyl, O—(C₁-C₆)alkyl-heteroaryl, O—(C₁-C₆)alkylaryl, (C₃-C₇)cyclo alkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl;
   p is an integer that is selected from the group consisting of 1,2, 3, 4 and 5 provided that when p>1, the G² groups may be equal or different from each other;
   R₁₆, R₁₇, R₁₈, R₁₉, R₂₀ and R₂₁ are each independently selected from the group consisting of hydrogen, an optionally substituted (C₁-C₆)alkyl, (C₁-C₆)alkylhalo, (C₁-C₆)alkyl-CN, (C₁-C₆)alkyl-O—(C₀-C₆)alkyl, (C₁-C₆)alkyl-N((C₀-C₆)alkyl)₂, (C₁-C₆)alkyl-C(═O)—O—(C₀-C₆)alkyl, (C₁-C₆)alkyl-heterocycloalkyl, (C₂-C₆)alkynyl, (C₂-C₆)alkenyl, (C₃-C₇)cycloalkyl, (C₃-C₇)cycloalkyl-(C₁-C₆)alkyl, heteroaryl, (C₁-C₆)alkyl-heteroaryl, aryl;
   Z¹, Z², Z³, Z⁴, Z⁵, Z⁶, Z⁷, Z⁸ and Z⁹ are each independently selected from the group consisting of bond, —C═, —C═C—, —C(═O)—, —C(═S)—, —C—, —O—, —N═, —N— or —S— which may further be substituted by G²ₚ groups;
   B¹, B² and B³ are each selected independently from —C—, —N—, —O— or —S— which may further be substituted by one G²ₚ group;
   Any N or S bearing ring may be depicted in its N-oxide, S-oxide or S-dioxide form;

It being understood that:
When

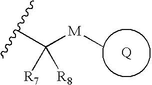

stands in the para-position of the phenyl ring with R₇ and R₈ are each independently selected from an optionally substituted (C₁-C₄)alkyl, or can together form a (C₃-C₆)cycloalkyl or an heterocycloalkyl group of formula:

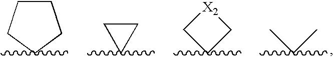

and G¹ₙ are hydrogens, then

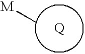

can not be

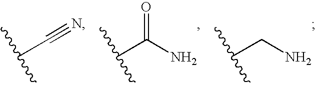

R₇ and R₈ may not represent at the same time (C₀-C₆)alkyl-aryl, (C₀-C₆)alkyl-heteroaryl;
If R₅ or R₆ are represented by (C₀-C₆)alkyl-OR₉, then R₉ may not represent an hydrogen;
G¹ₙ groups may not represent at the same time OH;
If R₇ and R₈ all represent CH₃ at the same time, then M-Q may not represent CH₃;
If R₇, R₈ and M represent at the same time an optionally substituted (C₁-C₄)alkyl, then Q can not be H;
If R₇, R₈ represent together

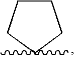

then compounds of the following list are excluded from the present invention:
3,4-dimethoxy-N-[4-[1-[[(4-methoxyphenyl)amino]carbonyl]cyclopentyl]phenyl]-benzamide
N-[4-(1-cyanocyclopentyl)phenyl]-3,4-dimethoxy-benzamide.

In a second aspect, the compounds of the present invention are represented by formula I-A1 wherein G¹ₙ groups are specified as in the formula I-A2 depicted below

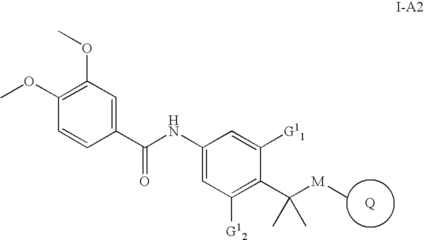

Or a pharmaceutically acceptable salt, hydrate or solvate of such compound

Wherein:
G¹₁ and G¹₂ are each independently selected from a group consisting of hydrogen, OH, (C₁-C₆)alkyl, (C₀-C₆)alkylhalo, (C₀-C₆)alkyl-CN, (C₃-C₆)cycloalkyl, (C₀-C₆)alkyl-(C₃-C₈)cycloalkyl, (C₂-C₆)alkenyl, (C₂-C₆)alkynyl, (C₀-C₆)alkyl-OR₉, (C₀-C₆)alkyl-NR₉R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O)R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀)R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-)cycloalkyl) or N((C₀-C₆)alkyl)(aryl) substituents, wherein optionally two substituents are combined to the intervening atoms to form a bicyclic heterocycloalkyl, aryl or heteroaryl ring; wherein each ring is optionally further substituted with 1-5 independent halogen, CN, (C₁-C₆)alkyl, O—(C₀-C₆)alkyl, O-alkylcycloalkyl, O(aryl), O(heteroaryl), O-arylalkyl, O-heteroarylalkyl, N((—C₀-C₆)alkyl)((C₀-C₃)arylalkyl) or N((C₀-C₆)alkyl)(heteroarylalkyl) groups;
   R₉, R₁₀, R₁₁ each independently is hydrogen, (C₁-C₆)alkyl, (C₃-C₆)cycloalkyl, (C₁-C₆)alkyl-(C₃-C₈)cycloalkyl, (C₂-C₆)alkenyl, (C₂-C₆)alkynyl, (C₁-C₆)alkylhalo, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), N(C₀-C₆-alkyl)₂, —N((C₀-C₆)alkyl)((C₃-C₇-)cycloalkyl) or N((C₀-C₆)alkyl)(aryl) substituents;
M is independently selected from the group of consisting of a bond, an optionally substituted (C₂-C₆)alkyl, (C₀-C₆)alkyl-CN, (C₁-C₆)alkyl-O—(C₀-C₆)alkyl, (C₀-C₆)alkyl-C(═O)—NR₁₂—(C₀-C₆)alkyl, (C₀-C₆)alkyl-C(═O)—NR₁₂—(C₀-C₆)alkyl-O, (C₀-C₆)alkyl-C(═O)—NR₁₂—(C₀-C₆)alkyl-S, (C₀-C₆)alkyl-NR₁₂C(═O)—(C₀-C₆)alkyl, (C₀-C₆)alkyl-NR₁₂C(═O)—(C₂-C₆)alkyl-O, (C₀-C₆)alkyl-NR₁₂C(═O)-(C₂-C₆)alkyl-S, (C₀-C₆)alkyl-OC(═O)—(C₀-C₆)alkyl, (C₀-C₆)alkyl-C(═O)—O—(C₀-C₆)alkyl, (C₀-C₆)alkyl-NR₁₂—C(═O)—O—(C₀-C₆)alkyl, (C₀-C₆)alkyl-O—C(═O)—NR₁₂—(C₀-C₆)alkyl or (C₀-C₆)alkyl-NR₁₂—C(═O)—NR₁₃—(C₀-C₆)alkyl substituents;
   wherein optionally two substituents are combined to the intervening atoms to form a cycloalkyl, heterocycloalkyl or heteroaryl ring; wherein each ring is optionally further substituted with 1-5 independent hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₃-C₇)cycloalkyl, O—(C₁-C₆)alkyl-heteroaryl, O—(C₁-C₆)alkylaryl, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl;
   R₁₂ and R₁₃ are each independently selected from the group consisting of hydrogen, an optionally substituted (C₁-C₆)alkyl, (C₀-C₆)alkylhalo, (C₂-C₆)alkynyl, (C₂-C₆)alkenyl, (C₃-C₇)cycloalkyl, (C₁-C₆)alkyl-heteroaryl, (C₁-C₆)alkyl-aryl, aryl, heterocycloalkyl, heteroaryl ring; wherein each substitutable carbon atom in R₁₂, R₁₃ is optionally further substituted with hydrogen, OH, (C₁-C₆)alkyl, (C₀-C₄)alkyl-CN, (C₁-C₆)alkylhalo, OR₁₄, SR₁₄, NR₁₄R₁₅, NR₁₄C(═O)—R₁₅, C(′O)—NR₁₄R₁₅, S(═O)₂—NR₁₄R₁₅, NR₁₄S(═O)₂—R₁₅, C(═O)—OR₁₄; C(═NR₁₄)—NR₁₅, wherein R₁₄ and R₁₅ are each independently selected from H, (C₁-C₄)alkyl or (C₁-C₄)alkylhalo;
Q represent independently H, an optionally substituted (C₁-C₆)alkyl, (C₀-C₆)alkyl-CN, (C₁-C₆)alkylhalo, (C₃-C₇)cycloalkyl, (C₃-C₇)heterocycloalkyl or one of the following aryl or heteroaryl:

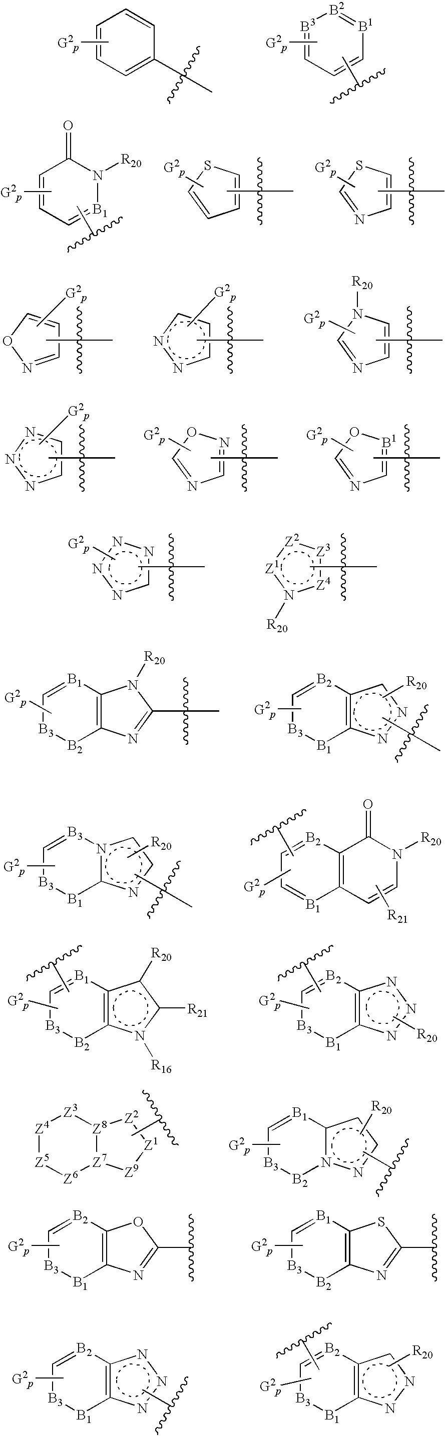

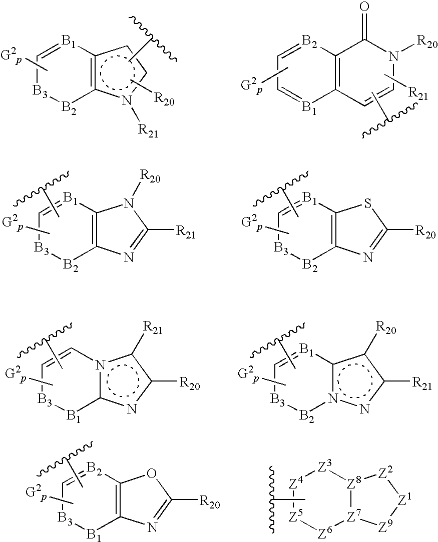

G² groups are each independently selected from the group consisting of hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₂-C₆)alkyl-OR₁₄, O—(C₃-C₇)cycloalkyl, O—(C₁-C₆)alkyl-heteroaryl, O—(C₁-C₆)alkyl-aryl, (C₀-C₆)alkyl-OR₁₄, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl, (C₁-C₆)alkyl-aryl, (C₁-C₆)alkylhalo-OR₁₇, (C₃-C₆)alkynyl-OR₁₇, (C₃-C₆)alkenyl-OR₁₇, (C₀-C₆)alkyl-S—R₁₇, O—(C₂-C₆)alkyl-S—R₁₇, (C₀-C₆)alkyl-S(═O)—R₁₇, O—(C₂-C₆)alkyl-S(═O)—R₁₇, (C₀-C₆)alkyl-S(═O)₂—R₁₇, O—(C₁-C₆)alkyl-S(═O)₂—R₁₇, (C₀-C₆)alkyl-NR₁₇R₁₈, O—(C₂-C₆)alkyl-NR₁₇R₁₈, (C₀-C₆)alkyl-S(═O)₂NR₁₇R₁₈, (C₀-C₆)alkyl-NR₁₇—S(═O)₂R₁₈, O—(C₁-C₆)alkyl-S(═O)₂NR₁₇R₁₈, O—(C₂-C₆)alkyl-NR₁₇—S(═O)₂R₁₈, (C₀-C₆)alkyl-C(═O)—NR₁₇R₁₈, (C₀-C₆)alkyl-NR₁₇C(═O)—R₁₈, O—(C₁-C₆)alkyl-C(═O)—NR₁₇R₁₈, O—(C₂-C₆)alkyl-NR₁₇C(═O)—R₁₈, (C₀-C₆)alkyl-OC(═O)—R₁₇, (C₀-C₆)alkyl-C(═O)—OR₁₇, O—(C₂-C₆)alkyl-OC(═O)—R₁₇, O—(C₁-C₆)alkyl-C(═O)—OR₁₇, (C₀-C₆)alkyl-C(═O)—R₁₇, O—(C₁-C₆)alkyl-C(═O)—R₁₇, (C₀-C₆)alkyl-NR₁₇—C(═O)—OR₁₈, (C₀-C₆)alkyl-O—C(═O)—NR₁₇R₁₈ or (C₀-C₆)alkyl-NR₁₇—C(═O)—NR₁₈R₁₉ substituents;
wherein optionally two substituents are combined to the intervening atoms to form a bicyclic aryl, cycloalkyl, heterocycloalkyl or heteroaryl ring; wherein each ring is optionally further substituted with 1-5 independent hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₃-C₇)cycloalkyl, O—(C₁-C₆)alkyl-heteroaryl, O—(C₁-C₆)alkylaryl, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl;
p is an integer that is selected from the group consisting of 1,2, 3, 4 and 5 provided that when p>1, the G² groups may be equal or different from each other;
R₁₆, R₁₇, R₁₈, R₁₉, R₂₀ and R₂₁ are each independently selected from the group consisting of hydrogen, an optionally substituted (C₁-C₆)alkyl, (C₁-C₆)alkylhalo, (C₁-C₆)alkyl-CN, (C₁-C₆)alkyl-O—(C₀-C₆)alkyl, (C₁-C₆)alkyl-N((C₀-C₆)alkyl)₂, (C₁-C₆)alkyl-C(═O)—O—(C₀-C₆)alkyl, (C₁-C₆)alkyl-heterocycloalkyl, (C₂-C₆)alkynyl, (C₂-C₆)alkenyl, (C₃-C₇)cyclo alkyl, (C₃-C₇)cycloalkyl-(C₁-C₆)alkyl, heteroaryl, (C₁-C₆)alkyl-heteroaryl, aryl;
Z¹, Z², Z³, Z⁴, Z⁵, Z⁶, Z⁷, Z⁸ and Z⁹ are each independently selected from the group consisting of bond, —C═, —C(═O)—, —C(═S)—, —C—, —O—, —N═, —N— or —S— which may further be substituted by G²ₚ groups;
B¹, B² and B³ are each selected independently from —C—, —N—, —O— or —S— which may further be substituted by one G²ₚ group;
Any N or S bearing ring may be depicted in its N-oxide, S-oxide or S-dioxide form;

It being understood that:
When G¹₁ and G¹₂ represent at the same time an hydrogen, then

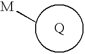

can not be

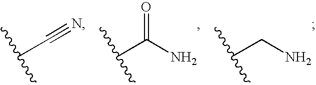

G¹₁ and G¹₂ groups may not represent at the same time OH;
If M represent an optionally substituted (C₁-C₄)alkyl, then Q can not be H.

Further preferred compounds of the present invention are compounds of formula I-A2-a

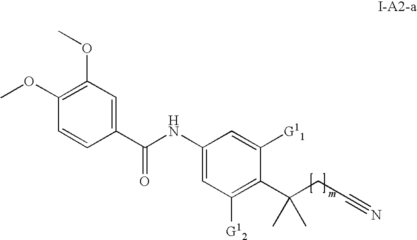

Or a pharmaceutically acceptable salt, hydrate or solvate of such compound

Wherein:
G¹₁ and G¹₂ are each independently selected from a group consisting of hydrogen, (C₁-C₆)alkyl, (C₀-C₆)alkylhalo, (C₀-C₆)alkyl-CN, (C₃-C₆)cycloalkyl, (C₀-C₆)alkyl-(C₃-C₈)cycloalkyl, (C₂-C₆)alkenyl, (C₂-C₆)alkynyl, (C₀-C₆)alkyl-OR₉, (C₀-C₆)alkyl-NR₉R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O)R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀)R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-)cycloalkyl) or N((C₀-C₆)alkyl)(aryl) substituents, wherein optionally two substituents are combined to the intervening atoms to form a bicyclic heterocycloalkyl, aryl or heteroaryl ring; wherein each ring is optionally further substituted with 1-5 independent halogen, CN, (C₁-C₆)alkyl, O—(C₀-C₆)alkyl, O-alkylcycloalkyl, O(aryl), O(heteroaryl), O-arylalkyl, O-heteroarylalkyl, N((—C₀-C₆)alkyl)((C₀-C₃)arylalkyl) or N((C₀-C₆)alkyl)(heteroarylalkyl) groups;
   R₉, R₁₀, R₁₁ each independently is hydrogen, (C₁-C₆)alkyl, (C₃-C₆)cycloalkyl, (C₁-C₆)alkyl-(C₃-C₈)cycloalkyl, (C₂-C₆)alkenyl, (C₂-C₆)alkynyl, (C₁-C₆)alkylhalo, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), N(C₀-C₆-alkyl)₂, —N((C₀-C₆)alkyl)((C₃-C₇-)cycloalkyl) or N((C₀-C₆)alkyl)(aryl) substituents;
m is an integer from 0 to 2;
   Any N or S bearing ring may be depicted in its N-oxide, S-oxide or S-dioxide form;

It being understood that:
When G¹₁ and G¹₂ represent at the same time an hydrogen, then m can not be equal to 0.

In a more preferred aspect of formula I-A2, the compounds of the present invention are represented by formula I-A2-b below

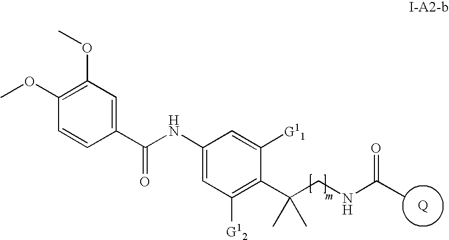

Or a pharmaceutically acceptable salt, hydrate or solvate of such compound

Wherein:
G¹₁ and G¹₂ are each independently selected from a group consisting of hydrogen, (C₁-C₆)alkyl, (C₀-C₆)alkylhalo, (C₀-C₆)alkyl-CN, (C₃-C₆)cycloalkyl, (C₀-C₆)alkyl-(C₃-C₈)cycloalkyl, (C₂-C₆)alkenyl, (C₂-C₆)alkynyl, (C₀-C₆)alkyl-OR₉, (C₀-C₆)alkyl-NR₉R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O)R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀)R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-)cycloalkyl) or N((C₀-C₆)alkyl)(aryl) substituents, wherein optionally two substituents are combined to the intervening atoms to form a bicyclic heterocycloalkyl, aryl or heteroaryl ring; wherein each ring is optionally further substituted with 1-5 independent halogen, CN, (C₁-C₆)alkyl, O—(C₀-C₆)alkyl, O-alkylcycloalkyl, O(aryl), O(heteroaryl), O-arylalkyl, O-heteroarylalkyl, N((—C₀-C₆)alkyl)((C₀-C₃)arylalkyl) or N((C₀-C₆)alkyl)(heteroarylalkyl) groups;
   R₉, R₁₀, R₁₁ each independently is hydrogen, (C₁-C₆)alkyl, (C₃-C₆)cycloalkyl, (C₁-C₆)alkyl-(C₃-C₈)cycloalkyl, (C₂-C₆)alkenyl, (C₂-C₆)alkynyl, (C₁-C₆)alkylhalo, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), N(C₀-C₆-alkyl)₂, —N((C₀-C₆)alkyl)((C₃-C₇-)cycloalkyl) or N((C₀-C₆)alkyl)(aryl) substituents;
m is an integer from 0 to 2;
Q represent independently H, an optionally substituted (C₁-C₆)alkyl, (C₀-C₆)alkyl-CN, (C₁-C₆)alkylhalo, (C₃-C₇)cycloalkyl, (C₃-C₇)heterocycloalkyl or one of the following aryl or heteroaryl:

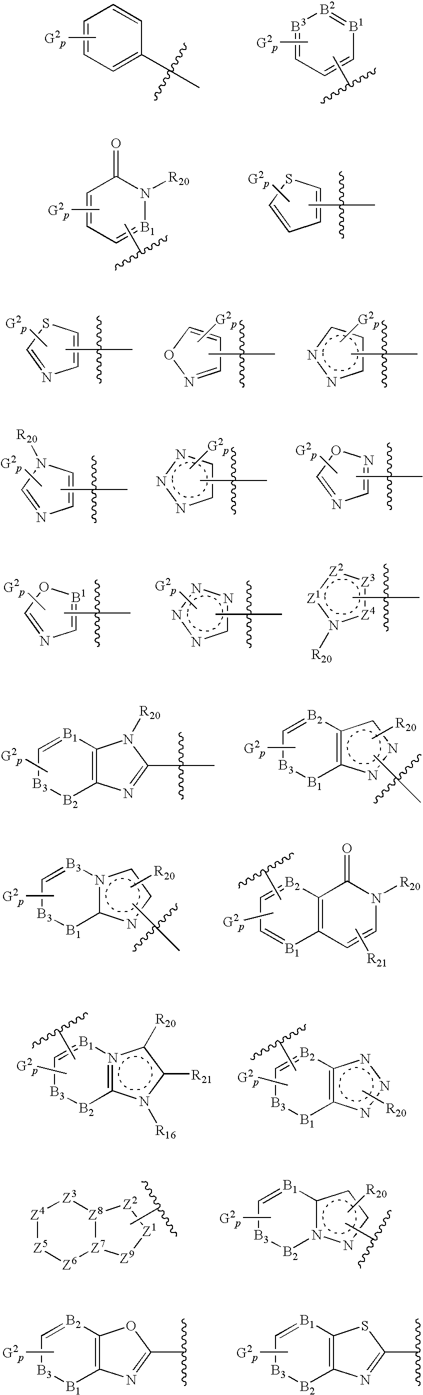

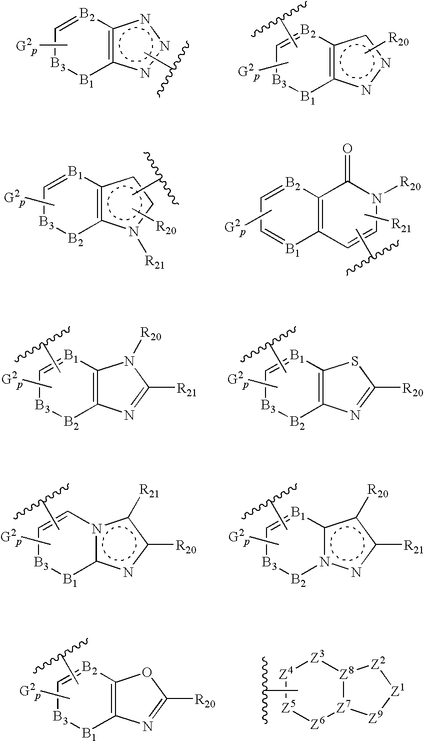

G² groups are each independently selected from the group consisting of hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₂-C₆)alkyl-OR₁₄, O—(C₃-C₇)cycloalkyl, O—(C₁-C₆)alkyl-heteroaryl, O—(C₁-C₆)alkyl-aryl, (C₀-C₆)alkyl-OR₁₄, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl, (C₁-C₆)alkyl-aryl, (C₁-C₆)alkylhalo-OR₁₇, (C₃-C₆)alkynyl-OR₁₇, (C₃-C₆)alkenyl-OR₁₇, (C₀-C₆)alkyl-S—R₁₇, O—(C₂-C₆)alkyl-S—R₁₇, (C₀-C₆)alkyl-S(═O)—R₁₇, O—(C₂-C₆)alkyl-S(═O)—R₁₇, (C₀C₆)alkyl-S(═O)₂—R₁₇, O—(C₁-C₆)alkyl-S(═O)₂—R₁₇, (C₀-C₆)alkyl-NR₁₇R₁₈, O—(C₂-C₆)alkyl-NR₁₇R₁₈, (C₀-C₆)alkyl-S(═O)₂NR₁₇R₁₈, (C₀-C₆)alkyl-NR₁₇—S(═O)₂R₁₈, O—(C₁-C₆)alkyl-S(═O)₂NR₁₇R₁₈, O—(C₂-C₆)alkyl-NR₁₇—S(═O)₂R₁₈, (C₀-C₆)alkyl-C(═O)—NR₁₇R₁₈, (C₀-C₆)alkyl-NR₁₇C(═O)—R₁₈, O—(C₁-C₆)alkyl-C(═O)—NR₁₇R₁₈, O—(C₂-C₆)alkyl-NR₁₇C(═O)—R₁₈, (C₀-C₆)alkyl-OC(═O)—R₁₇, (C₀-C₆)alkyl-C(═O)—OR₁₇, O—(C₂-C₆)alkyl-OC(═O—-R₁₇, O—(C₁-C₆)alkyl-C(═O)—OR₁₇, (C₀-C₆)alkyl-C(═O)—R₁₇, O—(C₁-C₆)alkyl-C(═O)—R₁₇, (C₀-C₆)alkyl-NR₁₇—C(═O)—OR₁₈, (C₀-C₆)alkyl-O—C(═O)—NR₁₇R₁₈ or (C₀-C₆)alkyl-NR₁₇—C(═O)—NR₁₈R₁₉ substituents;
wherein optionally two substituents are combined to the intervening atoms to form a bicyclic aryl, cycloalkyl, heterocycloalkyl or heteroaryl ring; wherein each ring is optionally further substituted with 1-5 independent hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₃-C₇)cycloalkyl, O—(C₁-C₆)alkyl-heteroaryl, O—(C₁-C₆)alkylaryl, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl;
p is an integer that is selected from the group consisting of 1,2, 3, 4 and 5 provided that when p>1, the G² groups may be equal or different from each other;
R₁₆, R₁₇, R₁₈, R₁₉, R₂₀ and R₂₁ are each independently selected from the group consisting of hydrogen, an optionally substituted (C₁-C₆)alkyl, (C₁-C₆)alkylhalo, (C₁-C₆)alkyl-CN, (C₁-C₆)alkyl-O—(C₀-C₆)alkyl, (C₁-C₆)alkyl-N((C₀-C₆)alkyl)₂, (C₁-C₆)alkyl-C(═O)—O—(C₀-C₆)alkyl, (C₁-C₆)alkyl-hetero cycloalkyl, (C₂-C₆)alkynyl, (C₂-C₆)alkenyl, (C₃-C₇)cycloalkyl, (C₃-C₇)cycloalkyl-(C₁-C₆)alkyl, heteroaryl, (C₁-C₆)alkyl-heteroaryl, aryl;
Z¹, Z², Z³, Z⁴, Z⁵, Z⁶, Z⁷, Z⁸ and Z⁹ are each independently selected from the group consisting of bond, —C═, —C═C—, —C(═O)—, —C(═S)—, —C—, —O—, —N═, —N— or —S— which may further be substituted by G²ₚ groups;
B¹, B² and B³ are each selected independently from —C—, —N—, —O— or —S— which may further be substituted by one G²ₚ group;
Any N or S bearing ring may be depicted in its N-oxide, S-oxide or S-dioxide form.

In one aspect, the compounds of the present invention are represented by formula I-A2-b wherein the heterocyclic ring system is specified as in the formula I-A2-b1 depicted below

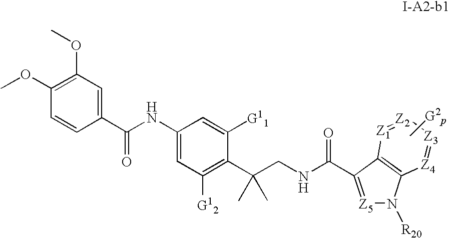

Or a pharmaceutically acceptable salt, hydrate or solvate of such compound

Wherein:
G¹₁ and G¹₂ are each independently selected from a group consisting of hydrogen, (C₀-C₆)alkyl-CN, (C₁-C₆)alkyl, (C₀-C₆)alkylhalo, (C₃-C₆)cycloalkyl, (C₀-C₆)alkyl-(C₃-C₈)cycloalkyl, (C₂-C₆)alkenyl, (C₂-C₆)alkynyl, (C₀-C₆)alkyl-OR₉, (C₀-C₆)alkyl-NR₉R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O)R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀)R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-)cycloalkyl) or N((C₀-C₆)alkyl)(aryl) substituents, wherein optionally two substituents are combined to the intervening atoms to form a bicyclic heterocycloalkyl, aryl or heteroaryl ring; wherein each ring is optionally further substituted with 1-5 independent halogen, CN, (C₁-C₆)alkyl, O—(C₀-C₆)alkyl, O-alkylcycloalkyl, O(aryl), O(heteroaryl), O-arylalkyl, O-heteroarylalkyl, N((—C₀-C₆)alkyl)((C₀-C₃)arylalkyl) or N((C₀-C₆)alkyl)(heteroarylalkyl) groups;
   R₉, R₁₀, R₁₁ each independently is hydrogen, (C₁-C₆)alkyl, (C₃-C₆)cycloalkyl, (C₁-C₆)alkyl-(C₃-C₈)cycloalkyl, (C₂-C₆)alkenyl, (C₂-C₆)alkynyl, (C₁-C₆)alkylhalo, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), N(C₀-C₆-alkyl)₂, —N((C₀-C₆)alkyl)((C₃-C₇-)cycloalkyl) or N((C₀-C₆)alkyl)(aryl) substituents;
G² groups are each independently selected from the group consisting of hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₂-C₆)alkyl-OR₁₄, O—(C₃-C₇)cycloalkyl, O—(C₁-C₆)alkyl-heteroaryl, O—(C₁-C₆)alkyl-aryl, (C₀-C₆)alkyl-OR₁₄, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl, (C₁-C₆)alkyl-aryl, (C₁-C₆)alkylhalo-OR₁₇, (C₃-C₆)alkynyl-OR₁₇, (C₃-C₆)alkenyl-OR₁₇, (C₀-C₆)alkyl-S—R₁₇, O—(C₂-C₆)alkyl-S—R₁₇, (C₀-C₆)alkyl-S(═O)—R₁₇, O—(C₂-C₆)alkyl-S(═O)—R₁₇, (C₀-C₆)alkyl-S(═O)₂—R₁₇, O—(C₁-C₆)alkyl-S(═O)₂—R₁₇, (C₀-C₆)alkyl-NR₁₇R₁₈, O—(C₂-C₆)alkyl-NR₁₇R₁₈, (C₀-C₆)alkyl-S(═O)₂NR₁₇R₁₈, (C₀-C₆)alkyl-NR₁₇—S(═O)₂R₁₈, O—(C₁-C₆)alkyl-S(═O)₂NR₁₇R₁₀, O—(C₂-C₆)alkyl-NR₁₇—S(═O)₂R₁₈, (C₀-C₆)alkyl-C(═O)—NR₁₇R₁₈, (C₀-C₆)alkyl-NR₁₇C(═O)—R₁₈, O—(C₁-C₆)alkyl-C(═O)—NR₁₇R₁₈, O—(C₂-C₆)alkyl-NR₁₇C(═O)—R₁₈, (C₀-C₆)alkyl-OC(═O)—R₁₇, (C₀-C₆)alkyl-C(═O)—OR₁₇, O—(C₂-C₆)alkyl-OC(═O)-R₁₇, O—(C₁-C₆)alkyl-C(═O)—OR₁₇, (C₀-C₆)alkyl-C(═O)—R₁₇, O—(C₁-C₆)alkyl-C(═O)—R₁₇, (C₀-C₆)alkyl-NR₁₇—C(═O)—OR₁₈, (C₀-C₆)alkyl-O—C(═O)—NR₁₇R₁₈ or (C₀-C₆)alkyl-NR₁₇—C(═O)—NR₁₈R₁₉ substituents;
   wherein optionally two substituents are combined to the intervening atoms to form a bicyclic aryl, cycloalkyl, heterocycloalkyl or heteroaryl ring; wherein each ring is optionally further substituted with 1-5 independent hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₃-C₇)cycloalkyl, O—(C₁-C₆)alkyl-heteroaryl, O—(C₁-C₆)alkylaryl, (C₃-C₇)cyclo alkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl;
   p is an integer that is selected from the group consisting of 1,2, 3, 4 and 5 provided that when p>1, the G² groups may be equal or different from each other;
   R₁₆, R₁₇, R₁₈, R₁₉ and R₂₀ are each independently selected from the group consisting of hydrogen, an optionally substituted (C₁-C₆)alkyl, (C₁-C₆)alkylhalo, (C₁-C₆)alkyl-CN, (C₁-C₆)alkyl-O—(C₀-C₆)alkyl, (C₁-C₆)alkyl-N((C₀-C₆)alkyl)₂, (C₁-C₆)alkyl-C(═O)—O—(C₀-C₆)alkyl, (C₁-C₆)alkyl-heterocycloalkyl, (C₂-C₆)alkynyl, (C₂-C₆)alkenyl, (C₃-C₇)cycloalkyl, (C₃-C₇)cycloalkyl-(C₁-C₆)alkyl, heteroaryl, (C₁-C₆)alkyl-hetero aryl, aryl;
   Z¹, Z², Z³ and Z⁴ are each independently selected from the group consisting of bond, —C═, —C═C—, —C(═O)—, —C(═S)—, —C—, —O—, —N═, —N— or —S— which may further be substituted by G²ₚ groups;
   Z⁵ is independently selected from —C— or —N— which may further be substituted by one G²ₚ group;
   Any N or S bearing ring may be depicted in its N-oxide, S-oxide or S-dioxide form.

Preferred compounds of the present invention are compounds of formula I-A2-b2 depicted below

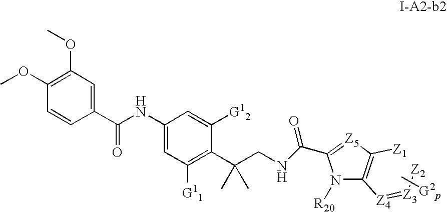

Or a pharmaceutically acceptable salt, hydrate or solvate of such compound

Wherein:

G¹₁ and G¹₂ are each independently selected from a group consisting of hydrogen, (C₀-C₆)alkyl-CN, (C₁-C₆)alkyl, (C₀-C₆)alkylhalo, (C₃-C₆)cycloalkyl, (C₀-C₆)alkyl-(C₃-C₈)cycloalkyl, (C₂-C₆)alkenyl, (C₂-C₆)alkynyl, (C₀-C₆)alkyl-OR₉, (C₀-C₆)alkyl-NR₉R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O)R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₁-C₆)alkyl-C(O)—O—R₉, (C₀-C₆)alkyl-C(═O)NR₁₀R₉, (C₀-C₆)alkyl-C(═NR₁₀)R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-)cycloalkyl) or N((C₀-C₆)alkyl)(aryl) substituents, wherein optionally two substituents are combined to the intervening atoms to form a bicyclic heterocycloalkyl, aryl or heteroaryl ring; wherein each ring is optionally further substituted with 1-5 independent halogen, CN, (C₁-C₆)alkyl, O—(C₀-C₆)alkyl, O-alkylcycloalkyl, O(aryl), O(heteroaryl), O-arylalkyl, O-heteroarylalkyl, N((—C₀-C₆)alkyl)((C₀-C₃)arylalkyl) or N((C₀-C₆)alkyl)(heteroarylalkyl) groups;
R₉, R₁₀, R₁₁ each independently is hydrogen, (C₁-C₆)alkyl, (C₃-C₆)cycloalkyl, (C₁-C₆)alkyl-(C₃-C₈)cycloalkyl, (C₂-C₆)alkenyl, (C₂-C₆)alkynyl, (C₁-C₆)alkylhalo, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), N(C₀-C₆-alkyl)₂, —N((C₀-C₆)alkyl)((C₃-C₇-)cycloalkyl) or N((C₀-C₆)alkyl)(aryl) substituents;

G² groups are each independently selected from the group consisting of hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₂-C₆)alkyl-OR₁₄, O—(C₃-C₇)cycloalkyl, O—(C₁-C₆)alkyl-heteroaryl, O—(C₁-C₆)alkyl-aryl, (C₀-C₆)alkyl-OR₁₄, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl, (C₁-C₆)alkyl-aryl, (C₁-C₆)alkylhalo-OR₁₇, (C₃-C₆)alkynyl-OR₁₇, (C₃-C₆)alkenyl-OR₁₇, (C₀-C₆)alkyl-S—R₁₇, O—(C₂-C₆)alkyl-S—R₁₇, (C₀-C₆)alkyl-S(═O)—R₁₇, O-(C₂-C₆)alkyl-S (═O)—R₁₇, (C₀-C₆)alkyl-S(═O)₂—R₁₇, O—(C₁-C₆)alkyl-S(═O)₂-R₁₇, (C₀-C₆)alkyl-NR₁₇R₁₈, O—(C₂-C₆)alkyl-NR₁₇R₁₈, (C₀-C₆)alkyl-S(═O)₂NR₁₇R₁₈, (C₀-C₆)alkyl-NR₁₇—S(═O)₂R₁₈, O—(C₁-C₆)alkyl-S(═O)₂NR₁₇R₁₈, O—(C₂-C₆)alkyl-NR₁₇—S(═O)₂R₁₈, (C₀-C₆)alkyl-C(═O)—NR₁₇R₁₈, (C₀-C₆)alkyl-NR₁₇C(═O)—R₁₈, O—(C₁-C₆)alkyl-C(═O)—NR₁₇R₁₈, O—(C₂-C₆)alkyl-NR₁₇C(═O)—R₁₈, (C₀-C₆)alkyl-OC(═O)—R₁₇, (C₀-C₆)alkyl-C(═O)—OR₁₇, O—(C₂-C₆)alkyl-OC(═O)—R₁₇, O—(C₁-C₆)alkyl-C(═O)—OR₁₇, (C₀-C₆)alkyl-C(═O)—R₁₇, O—(C₁-C₆)alkyl-C(═O)—R₁₇, (C₀-C₆)alkyl-NR₁₇—C(═O)—OR₁₈, (C₀-C₆)alkyl-O—C(═O)—NR₁₇R₁₈ or (C₀-C₆)alkyl-NR₁₇—C(═O)—NR₁₈R₁₉ substituents;
wherein optionally two substituents are combined to the intervening atoms to form a bicyclic aryl, cycloalkyl, heterocycloalkyl or heteroaryl ring; wherein each ring is optionally further substituted with 1-5 independent hydrogen, halogen, CN, OH, nitro; an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₃-C₇)cycloalkyl, O—(C₁-C₆)alkyl-heteroaryl, O—(C₁-C₆)alkylaryl, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl;
p is an integer that is selected from the group consisting of 1,2, 3, 4 and 5 provided that when p>1, the G² groups may be equal or different from each other;
R₁₆, R₁₇, R₁₈, R₁₉ and R₂₀ are each independently selected from the group consisting of hydrogen, an optionally substituted (C₁-C₆)alkyl, (C₁-C₆)alkylhalo, (C₁-C₆)alkyl-CN, (C₁-C₆)alkyl-O—(C₀-C₆)alkyl, (C₁-C₆)alkyl-N((C₀-C₆)alkyl)₂, (C₁-C₆)alkyl-C(═O)—O—(C₀-C₆)alkyl, (C₁-C₆)alkyl-hetero cycloalkyl, (C₂-C₆)alkynyl, (C₂-C₆)alkenyl, (C₃-C₇)cycloalkyl, (C₃-C₇)cycloalkyl-(C₁-C₆)alkyl, heteroaryl, (C₁-C₆)alkyl-heteroaryl, aryl;
Z¹, Z², Z³ and Z⁴ are each independently selected from the group consisting of bond, —C═, —C═C—, —C(═O)—, —C(═S)—, —C—, —O—, —N═, —N— or —S— which may further be substituted by G²ₚ groups;
Z⁵ is independently selected from —C— or —N— which may further be substituted by one G²ₚ group;
Any N or S bearing ring may be depicted in its N-oxide, S-oxide or S-dioxide form.

Further preferred compounds of the present invention are compounds of formula I-A2-b3

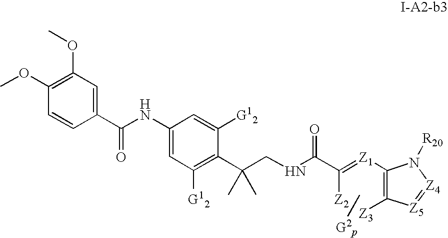

Or a pharmaceutically acceptable salt, hydrate or solvate of such compound

Wherein:

G¹₁ and G¹₂ are each independently selected from a group consisting of hydrogen, (C₀-C₆)alkyl-CN, (C₁-C₆)alkyl, (C₀-C₆)alkylhalo, (C₃-C₆)cycloalkyl, (C₀-C₆)alkyl-(C₃-C₈)cycloalkyl, (C₂-C₆)alkenyl, (C₂-C₆)alkynyl, (C₀-C₆)alkyl-OR₉, (C₀-C₆)alkyl-NR₉R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O)R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀)R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-)cycloalkyl) or N((C₀-C₆)alkyl)(aryl) substituents, wherein optionally two substituents are combined to the intervening atoms to form a bicyclic heterocycloalkyl, aryl or heteroaryl ring; wherein each ring is optionally further substituted with 1-5 independent halogen, CN, (C₁-C₆)alkyl, O—(C₀-C₆)alkyl, O-alkylcycloalkyl, O(aryl), O(heteroaryl), O-arylalkyl, O-heteroarylalkyl, N((-C₀-C₆)alkyl)((C₀-C₃)arylalkyl) or N((C₀-C₆)alkyl)(heteroarylalkyl) groups;
R₉, R₁₀, R₁₁ each independently is hydrogen, (C₁-C₆)alkyl, (C₃-C₆)cycloalkyl, (C₁-C₆)alkyl-(C₃-C₈)cycloalkyl, (C₂-C₆)alkenyl, (C₂-C₆)alkynyl, (C₁-C₆)alkylhalo, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), N(C₀-C₆-alkyl)₂, —N((C₀-C₆)alkyl)((C₃-C₇-)cycloalkyl) or N((C₀-C₆)alkyl)(aryl) substituents;

G² groups are each independently selected from the group consisting of hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₂-C₆)alkyl-OR₁₄, O—(C₃-C₇)cycloalkyl, O—(C₁-C₆)alkyl-heteroaryl, O—(C₁-C₆)alkyl-aryl, (C₀-C₆)alkyl-OR₁₄, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl, (C₁-C₆)alkyl-aryl, (C₁-C₆)alkylhalo-OR₁₇, (C₃-C₆)alkynyl-OR₁₇, (C₃-C₆)alkenyl-OR₁₇, (C₀-C₆)alkyl-S—R₁₇, O—(C₂-C₆)alkyl-S—R₁₇, (C₀-C₆)alkyl-S(═O)—R₁₇, O—(C₂-C₆)alkyl-S(═O)—R₁₇, (C₀-C₆)alkyl-S(═O)₂—R₁₇, O—(C₁-C₆)alkyl-S(═O)₂—R₁₇, (C₀-C₆)alkyl-NR₁₇R₁₈, O—(C₂-C₆)alkyl-NR₁₇R₁₈, (C₀-C₆)alkyl-S(═O)₂NR₁₇R₁₈, (C₀-C₆)alkyl-NR₁₇—S(═O)₂R₁₈, O—(C₁-C₆)alkyl-S(═O)₂NR₁₇R₁₈, O—(C₂-C₆)alkyl-NR₁₇—S(═O)₂R₁₈, (C₀-C₆)alkyl-C(═O)—NR₁₇R₁₈, (C₀-C₆)alkyl-NR₁₇C(═O)—R₁₈, O—(C₁-C₆)alkyl-C(═O)—NR₁₇R₁₈, O—(C₂-C₆)alkyl-NR₁₇C(═O)—R₁₈, (C₀-C₆)alkyl-OC(═O)—R₁₇, (C₀-C₆)alkyl-C(═O)—OR₁₇, O—(C₂-C₆)alkyl-OC(═O)—R₁₇, O—(C₁-C₆)alkyl-C(═O)—OR₁₇, (C₀-C₆)alkyl-C(═O)—R₁₇, O—(C₁-C₆)alkyl-C(═O)—R₁₇, (C₀-C₆)alkyl-NR₁₇—C(═O)—OR₁₈, (C₀-C₆)alkyl-O—C(═O)—NR₁₇R₁₈ or (C₀-C₆)alkyl-NR₁₇—C(═O)—NR₁₈R₁₉ substituents;
wherein optionally two substituents are combined to the intervening atoms to form a bicyclic aryl, cycloalkyl, heterocycloalkyl or heteroaryl ring; wherein each ring is optionally further substituted with 1-5 independent hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₃-C₇)cycloalkyl, O—(C₁-C₆)alkyl-heteroaryl, O—(C₁-C₆)alkylaryl, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl;
p is an integer that is selected from the group consisting of 1,2, 3, 4 and 5 provided that when p>1, the G² groups may be equal or different from each other;
R₁₆, R₁₇, R₁₈, R₁₉ and R₂₀ are each independently selected from the group consisting of hydrogen, an optionally substituted (C₁-C₆)alkyl, (C₁-C₆)alkylhalo, (C₁-C₆)alkyl-CN, (C₁-C₆)alkyl-O—(C₀-C₆)alkyl, (C₁-C₆)alkyl-N((C₀-C₆)alkyl)₂, (C₁-C₆)alkyl-C(═O)—O—(C₀-C₆)alkyl, (C₁-C₆)alkyl-heterocycloalkyl, (C₂-C₆)alkynyl, (C₂-C₆)alkenyl, (C₃-C₇)cycloalkyl, (C₃-C₇)cycloalkyl-(C₁-C₆)alkyl, heteroaryl, (C₁-C₆)alkyl-heteroaryl, aryl;
Z¹, Z² and Z³ are each independently selected from the group consisting of bond, —C═, —C═C—, —C(═O)—, —C(═S)—, —C—, —O—, —N═, —N— or —S— which may further be substituted by G²ₚ groups;
Z⁴ and Z⁵ are each independently selected from —C— or —N— which may further be substituted by one G²ₚ group;
Any N or S bearing ring may be depicted in its N-oxide, S-oxide or S-dioxide form.

Further specific embodiments of the present invention are compounds of formula I-A2-b4

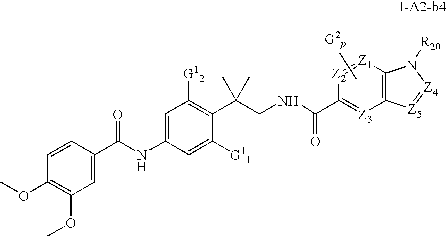

Or a pharmaceutically acceptable salt, hydrate or solvate of such compound

Wherein:

G¹₁ and G¹₂ are each independently selected from a group consisting of hydrogen, (C₀-C₆)alkyl-CN, (C₁-C₆)alkyl, (C₀-C₆)alkylhalo, (C₃-C₆)cycloalkyl, (C₀-C₆)alkyl-(C₃-C₈)cycloalkyl, (C₂-C₆)alkenyl, (C₂-C₆)alkynyl, (C₀-C₆)alkyl-OR₉, (C₀-C₆)alkyl-NR₉R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O)R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀)R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-)cycloalkyl) or N((C₀-C₆)alkyl)(aryl) substituents, wherein optionally two substituents are combined to the intervening atoms to form a bicyclic heterocycloalkyl, aryl or heteroaryl ring; wherein each ring is optionally further substituted with 1-5 independent halogen, CN, (C₁-C₆)alkyl, O—(C₀-C₆)alkyl, O-alkylcycloalkyl, O(aryl), O(heteroaryl), O-arylalkyl, O-heteroarylalkyl, N((—C₀-C₆)alkyl)((C₀-C₃)arylalkyl) or N((C₀-C₆)alkyl)(heteroarylalkyl) groups;
R₉, R₁₀, R₁₁ each independently is hydrogen, (C₁-C₆)alkyl, (C₃-C₆)cycloalkyl, (C₁-C₆)alkyl-(C₃-C₈)cycloalkyl, (C₂-C₆)alkenyl, (C₂-C₆)alkynyl, (C₁-C₆)alkylhalo, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), N(C₀-C₆-alkyl)₂, —N((C₀-C₆)alkyl)((C₃-C₇-)cycloalkyl) or N((C₀-C₆)alkyl)(aryl) substituents;

G² groups are each independently selected from the group consisting of hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₂-C₆)alkyl-OR₁₄, O—(C₃-C₇)cycloalkyl, O—(C₁-C₆)alkyl-heteroaryl, O—(C₁-C₆)alkyl-aryl, (C₀-C₆)alkyl-OR₁₄, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl, (C₁-C₆)alkyl-aryl, (C₁-C₆)alkylhalo-OR₁₇, (C₃-C₆)alkynyl-OR₁₇, (C₃-C₆)alkenyl-OR₁₇, (C₀-C₆)alkyl-S—R₁₇, O—(C₂-C₆)alkyl-S—R₁₇, (C₀-C₆)alkyl-S(═O)—R₁₇, O—(C₂-C₆)alkyl-S(═O)—R₁₇, (C₀-C₆)alkyl-S(═O)₂—R₁₇, O—(C₁-C₆)alkyl-S(═O)₂—R₁₇, (C₀-C₆)alkyl-NR₁₇R₁₈, O—(C₂-C₆)alkyl-NR₁₇R₁₈, (C₀-C₆)alkyl-S(═O)₂NR₁₇R₁₈, (C₀-C₆)alkyl-NR₁₇—S(═O)₂R₁₈, O—(C₁-C₆)alkyl-S(═O)₂NR₁₇R₁₈, O—(C₂-C₆)alkyl-NR₁₇—S(═O)₂R₁₈, (C₀-C₆)alkyl-C(═O)—NR₁₇R₁₈, (C₀-C₆)alkyl-NR₁₇C(═O)—R₁₈, O—(C₁-C₆)alkyl-C(═O)—NR₁₇R₁₈, O—(C₂-C₆)alkyl-NR₁₇C(═O)—R₁₈, (C₀-C₆)alkyl-OC(═O)—R₁₇, (C₀-C₆)alkyl-C(═O)—OR₁₇, O—(C₂-C₆)alkyl-OC(═O)—R₁₇, O—(C₁-C₆)alkyl-C(═O)—OR₁₇, (C₀-C₆)alkyl-C(═O)—R₁₇, O—(C₁-C₆)alkyl-C(═O)—R₁₇, (C₀-C₆)alkyl-NR₁₇—C(═O)—OR₁₈, (C₀-C₆)alkyl-O—C(═O)—NR₁₇R₁₈ or (C₀-C₆)alkyl-NR₁₇—C(═O)—NR₁₈R₁₉ substituents;
wherein optionally two substituents are combined to the intervening atoms to form a bicyclic aryl, cycloalkyl, heterocycloalkyl or heteroaryl ring; wherein each ring is optionally further substituted with 1-5 independent hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₃-C₇)cycloalkyl, O—(C₁-C₆)alkyl-heteroaryl, O—(C₁-C₆)alkylaryl, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl;
p is an integer that is selected from the group consisting of 1, 2, 3, 4 and 5 provided that when p>1, the G² groups may be equal or different from each other;
R₁₆, R₁₇, R₁₈, R₁₉ and R₂₀ are each independently selected from the group consisting of hydrogen, an optionally substituted (C₁-C₆)alkyl, (C₁-C₆)alkylhalo, (C₁-C₆)alkyl-CN, (C₁-C₆)alkyl-O—(C₀-C₆)alkyl, (C₁-C₆)alkyl-N((C₀-C₆)alkyl)₂, (C₁-C₆)alkyl-C(═O)—O—(C₀-C₆)alkyl, (C₁-C₆)alkyl-heterocycloalkyl, (C₂-C₆)alkynyl, (C₂-C₆)alkenyl, (C₃-C₇)cycloalkyl, (C₃-C₇)cycloalkyl-(C₁-C₆)alkyl, heteroaryl, (C₁-C₆)alkyl-heteroaryl, aryl;
Z¹, Z² and Z³ are each independently selected from the group consisting of bond, —C═, —C═C—, —C(═O)—, —C(═S)—, —C—, —O—, —N═, —N— or —S— which may further be substituted by G²ₚ groups;
Z⁴ and Z⁵ are each independently selected from —C— or —N— which may further be substituted by one G²ₚ group;
Any N or S bearing ring may be depicted in its N-oxide, S-oxide or S-dioxide form.

Particularly preferred compounds of the present invention are compounds of formula I-B

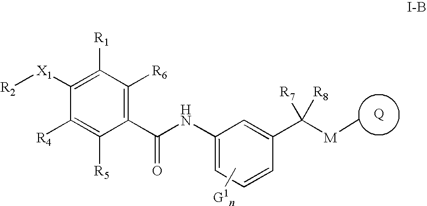

Or a pharmaceutically acceptable salt, hydrate or solvate of such compound

Wherein:

X₁ is selected from O, NR₃;

R₃ is independently selected from the group consisting of hydrogen, an optionally substituted (C₁-C₆)alkyl, (C₂-C₆)alkynyl, (C₂-C₆)alkenyl, (C₃-C₇)cycloalkyl, (C₂-C₆)alkylhalo, (C₁-C₆)alkyl-CN, (C₂-C₆)alkyl-O—(C₁-C₆)alkyl, (C₂-C₆)alkyl-O—(C₂-C₆)alkynyl, (C₂-C₆)alkyl-O—(C₂-C₆)alkenyl, (C₂-C₆)alkyl-O—(C₃-C₇)cycloalkyl or (C₂-C₆)alkyl-O-alkylcycloalkyl;

R₁ represent independently hydrogen, OH, an optionally substituted O—(C₀-C₆)alkyl, O—(C₂-C₆)alkynyl, O—(C₂-C₆)alkenyl, O—(C₃-C₇)cycloalkyl, O-alkylcycloalkyl, (C₁-C₆)alkyl, (C₂-C₆)alkynyl, (C₂-C₆)alkenyl, (C₃-C₇)cycloalkyl, (C₀-C₆)alkylhalo or (C₀-C₆)alkyl-CN;

R₂ represent independently hydrogen, an optionally substituted (C₁-C₆)alkyl, (C₂-C₆)alkynyl, (C₂-C₆)alkenyl, (C₃-C₇)cycloalkyl, (C₄-C₁₀)alkylcycloalkyl, (C₁-C₆)heterocycloalkyl, (C₁-C₆)alkyl-heteroaryl, (C₁-C₆)alkyl-aryl or (C₁-C₆)alkyl-CN;
R₁ and R₂ according to the above definitions can be combined to form a heterocycloalkyl ring;

R₄ is independently selected from group consisted of hydrogen, OH, (C₀-C₆)alkyl-CN, (C₁-C₆)alkyl , (C₀-C₆)alkylhalo, (C₃-C₆)cycloalkyl, (C₁-C₆)alkyl-(C₃-C₈)cycloalkyl, (C₂-C₆)alkenyl, (C₂-C₆)alkynyl, (C₀-C₆)alkyl-OR₉, (C₁-C₆)alkyl-NR₉R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O)R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR_{1O})R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-)cycloalkyl) or N((C₀-C₆)alkyl)(aryl) substituents;

R₅, R₆ are each independently selected from group consisted of hydrogen, (C₀-C₆)alkyl-CN, (C₁-C₆)alkyl, (C₀-C₆)alkylhalo, (C₃-C₆)cycloalkyl, (C₁-C₆)alkyl-(C₃-C₈)cycloalkyl, (C₂-C₆)alkenyl, (C₂-C₆)alkynyl, (C₀-C₆)alkyl-OR₉, (C₁-C₆)alkyl-NR₉R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O)R₉, (C_{O}-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀)R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-)cycloalkyl) or N((C₀-C₆)alkyl)(aryl) substituents;

G¹ is independently selected from a group consisting of hydrogen, OH, (C₀-C₆)alkyl-CN, (C₁-C₆)alkyl, (C₀-C₆)alkylhalo, (C₃-C₆)cycloalkyl, (C₀-C₆)alkyl-(C₃-C₈)cycloalkyl, (C₂-C₆)alkenyl, (C₂-C₆)alkynyl, (C₀-C₆)alkyl-OR₉, (C₀-C₆)alkyl-NR₉R₁₀, (C₀-C₆)-alkyl-NR₉COR₁₀, (C_{0-C}₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O)R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀)R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-)cycloalkyl) or N((C₀-C₆)alkyl)(aryl) substituents, wherein optionally two substituents are combined to the intervening atoms to form a bicyclic heterocycloalkyl, aryl or heteroaryl ring; wherein each ring is optionally further substituted with 1-5 independent halogen, CN, (C₁-C₆)alkyl, O—(C₀-C₆)alkyl, O-alkylcycloalkyl, O(aryl), O(heteroaryl), O-arylalkyl, O-heteroarylalkyl, N((—C₀-C₆)alkyl)((C₀-C₃)arylalkyl) or N((C₀-C₆)alkyl)(heteroarylalkyl) groups;
R₉, R₁₀, R₁₁ each independently is hydrogen, (C₁-C₆)alkyl, (C₃-C₆)cycloalkyl, (C₁-C₆)alkyl-(C₃-C₈)cycloalkyl, (C₂-C₆)alkenyl, (C₂-C₆)alkynyl, (C₁-C₆)alkylhalo, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), N(C₀-C₆-alkyl)₂, —N((C₀-C₆)alkyl)((C₃-C₇-)cycloalkyl) or N((C₀-C₆)alkyl)(aryl) substituents;

n is an integer from 1 to 4, provided that when n>1, the G¹ groups may be equal or different from each other;

R₇ and R₈ represent independently an optionally substituted (C₁-C₄)alkyl, (C₁-C₆)alkylhalo, (C₀-C₆)alkyl-aryl, (C₁-C₆)alkyl-O—(C₀-C₆)-alkyl, (C₀-C₆)alkyl-heteroaryl, (C₀-C₆)alkyl-heterocycloalkyl, (C₀-C₆)alkyl-(C₃-C₇)cycloalkyl or R⁷ and R⁸ can together form a (C₃-C₆)cycloalkyl or an heterocycloalkyl group of formula:

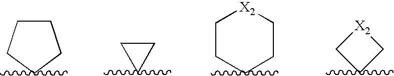

X₂ is independently selected from the group consisting of CH₂, O, S, SO₂;

M is independently selected from the group of consisting of a bond, an optionally substituted (C₁-C₆)alkyl, (C₂-C₆)alkynyl, (C₁-C₆)alkylhalo, (C₂-C₆)alkenyl, (C₁-C₆)alkyl-O—(C₀-C₆)alkyl, (C₁-C₆)-alkylhalo-O—(C₀-C₆)alkyl, (C₃-C₆)alkynyl-O—(C₀-C₆)alkyl, (C₃-C₆)alkenyl-O—(C₀-C₆)alkyl, (C₀-C₆)alkyl-S—(C₀-C₆)alkyl, (C₀-C₆)alkyl-S(═O)—(C₀-C₆)alkyl, (C₀-C₆)alkyl-S(═O)₂—(C₀-C₆)alkyl, (C₀-C₆)alkyl-NR₁₂—(C₀-C₆)alkyl, (C₀-C₆)alkyl-S(═O)₂NR₁₂—(C₀-C₆)alkyl, (C₀-C₆)alkyl-NR₁₂—S(═O)₂—(C₀-C₆)alkyl, (C₀-C₆)alkyl-C(═O)—NR₁₂—(C₀-C₆)alkyl, (C₀-C₆)alkyl-C(═O)—NR₁₂—(C₀-C₆)alkyl-O, (C₀-C₆)alkyl-C(═O)—NR₁₂—(C₀-C₆)alkyl-S, (C₀-C₆)alkyl-NR₁₂C(═O)—(C₀-C₆)alkyl, (C₀-C₆)alkyl-NR₁₂C(═O)—(C₂-C₆)alkyl-O, (C₀-C₆)alkyl-NR₁₂C(═O)—(C₂-C₆)alkyl-S, (C₀-C₆)alkyl-OC(═O)—(C₀-C₆)alkyl, (C₀-C₆)alkyl-C(═O)—O—(C₀-C₆)alkyl, (C₀-C₆)alkyl-C(═O)—(C₀-C₆)alkyl, (C₀-C₆)alkyl-NR₁₂—C(═O)—O—(C₀-C₆)alkyl, (C₀-C₆)alkyl-O—C(═O)—NR₁₂—(C₀-C₆)alkyl or (C₀-C₆)alkyl-NR₁₂—C(═O)—NR₁₃—(C₀-C₆)alkyl substituents;
wherein optionally two substituents are combined to the intervening atoms to form a cycloalkyl, heterocycloalkyl or heteroaryl ring; wherein each ring is optionally further substituted with 1-5 independent hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₃-C₇)cycloalkyl, O—(C₁-C₆)alkyl-heteroaryl, O—(C₁-C₆)alkylaryl, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl;
R₁₂ and R₁₃ are each independently selected from the group consisting of hydrogen, an optionally substituted (C₁-C₆)alkyl, (C₀-C₆)alkylhalo, (C₂-C₆)alkynyl, (C₂-C₆)alkenyl, (C₃-C₇)cycloalkyl, (C₁-C₆)alkyl-heteroaryl, (C₁-C₆)alkyl-aryl, aryl, heterocycloalkyl, heteroaryl ring; wherein each substitutable carbon atom in R₁₂, R₁₃ is optionally further substituted with hydrogen, OH, (C₁-C₆)alkyl, (C₀-C₄)alkyl-CN, (C₁-C₆)alkylhalo, OR₁₄, SR₁₄, NR₁₄R₁₅, NR₁₄C(═O)—R₁₅, C(═O)—NR₁₄R₁₅, S(═O)₂—NR₁₄R₁₅, NR₁₄S(═O)₂—R₁₅, C(═O)—OR₁₄; C(═NR₁₄)—NR₁₅, wherein R₁₄ and R₁₅ are each independently selected from H, (C₁-C₄)alkyl or (C₁-C₄)alkylhalo;

Q represent independently H, an optionally substituted (C₁-C₆)alkyl, (C₀-C₆)alkyl-CN, (C₁-C₆)alkylhalo, (C₃-C₇)cycloalkyl, (C₃-C₇)heterocycloalkyl or one of the following aryl or heteroaryl:

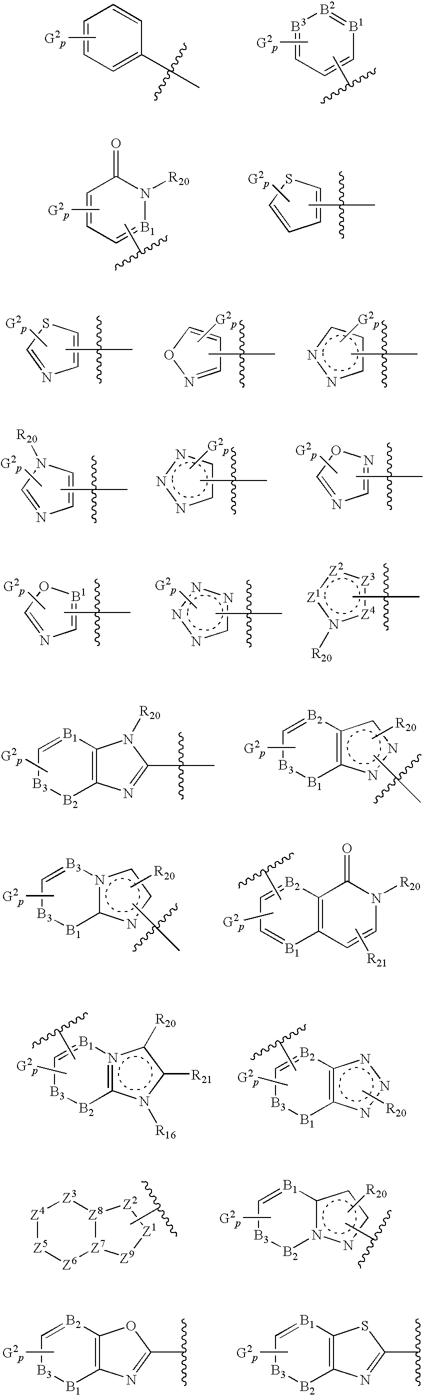

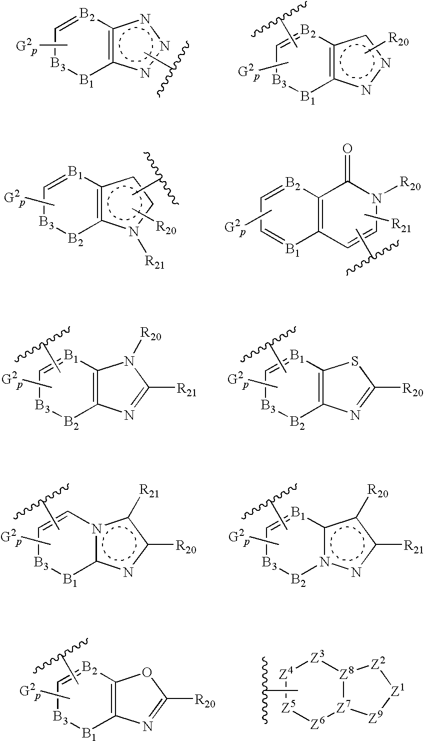

G² groups are each independently selected from the group consisting of hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₂-C₆)alkyl-OR₁₄, O—(C₃-C₇)cycloalkyl, O—(C₁-C₆)alkyl-heteroaryl, O—(C₁-C₆)alkyl-aryl, (C₀-C₆)alkyl-OR₁₄, (C₃-C₇)cycloalkyl, (C₃-C₇)cycloalkyl-(C₁-C₆)alkyl, O—(C₃-C₇)cycloalkyl-(C₁-C₆)alkyl, O—heteroaryl, heteroaryl, (C₁-C₆)alkyl-heteroaryl, aryl, O-aryl, (C₁-C₆)alkyl-aryl, (C₁-C₆)alkylhalo-OR₁₇, (C₃-C₆)alkynyl-OR₁₇, (C₃-C₆)alkenyl-OR₁₇, (C₀-C₆)alkyl-S—R₁₇, O—(C₂-C₆)alkyl-S—R₁₇, (C₀-C₆)alkyl-S(═O)—R₁₇, O—(C₂-C₆)alkyl-S(═O)—R₁₇, (C₀-C₆)alkyl-S(═O)₂—R₁₇, O—(C₁-C₆)alkyl-S(═O)₂—R₁₇, (C₀-C₆)alkyl-NR₁₇R₁₈, O—(C₂-C₆)alkyl-NR₁₇R₁₈, (C₀-C₆)alkyl-S(═O)₂NR₁₇R₁₈, (C₀-C₆)alkyl-N₁₇—S(═O)₂R₁₈, O—(C₁-C₆)alkyl-S(═O)₂NR₁₇R₁₈, O—(C₂-C₆)alkyl-NR₁₇—S(═O)₂R₁₈, (C₀-C₆)alkyl-C(═O)—NR₁₇R₁₈, (C₀-C₆)alkyl-NR₁₇C(═O)—R₁₈, O—(C₁-C₆)alkyl-C(═O)—NR₁₇R₁₈, O—(C₂-C₆)alkyl-NR₁₇C(═O)—R₁₈, (C₀-C₆)alkyl-OC(═O)—R₁₇, (C₀-C₆)alkyl-C(═O)—OR₁₇, O—(C₂-C₆)alkyl-OC(═O)—R₁₇, O—(C₁-C₆)alkyl-C(═O)—OR₁₇, (C₀-C₆)alkyl-C(═O)—R₁₇, O—(C₁-C₆)alkyl-C(═O)—R₁₇, (C₀-C₆)alkyl-NR₁₇—C(═O)—OR₁₈, (C₀-C₆)alkyl-O—C(═O)—NR₁₇R₁₈ or (C₀-C₆)alkyl-NR₁₇—C(═O)—NR₁₈R₁₉ substituents;
wherein optionally two substituents are combined to the intervening atoms to form a bicyclic aryl, cycloalkyl, heterocycloalkyl or heteroaryl ring; wherein each ring is optionally further substituted with 1-5 independent hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₃-C₇)cycloalkyl, O—(C₁-C₆)alkyl-heteroaryl, O—(C₁-C₆)alkylaryl, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl;
p is an integer that is selected from the group consisting of 1, 2, 3, 4 and 5 provided that when p>1, the G² groups may be equal or different from each other;
R₁₆, R₁₇, R₁₈, R₁₉, R₂₀ and R₂₁ are each independently selected from the group consisting of hydrogen, an optionally substituted (C₁-C₆)alkyl, (C₁-C₆)alkylhalo, (C₁-C₆)alkyl-CN, (C₁-C₆)alkyl-O—(C₀-C₆)alkyl, (C₁-C₆)alkyl-N((C₀-C₆)alkyl)₂, (C₁-C₆)alkyl-C(═O)—O—(C₀-C₆)alkyl, (C₁-C₆)alkyl-heterocycloalkyl, (C₂-C₆)alkynyl, (C₂-C₆)alkenyl, (C₃-C₇)cycloalkyl, (C₃-C₇)cycloalkyl-(C₁-C₆)alkyl, heteroaryl, (C₁-C₆)alkyl-heteroaryl, aryl;
Z¹, Z², Z³, Z⁴, Z⁵, Z⁶, Z⁷, Z⁸ and Z⁹ are each independently selected from the group consisting of bond, —C═, —C═C—, —C(═O)—, —C(═S)—, —C—, —O—, —N═, —N— or —S— which may further be substituted by G²ₚ groups;
B¹, B² and B³ are each selected independently from —C—, —N—, —O— or —S— which may further be substituted by one G²ₚ group;
Any N or S bearing ring may be depicted in its N-oxide, S-oxide or S-dioxide form;

It being understood that:
If X₁ is O, then R₁ is represented by O—(C₁-C₆)alkyl, O—(C₂-C₆)alkynyl, O—(C₂-C₆)alkenyl, O—(C₃-C₇)cycloalkyl, O-alkylcycloalkyl;
X₁—R₂ and R₁ may not represent at the same time OH;
If R₇ and R₈ all represent CH₃ at the same time, then M-Q may not represent CH₃;
If R₅ or R₆ are represented by (C₀-C₆)alkyl-OR₉, then R₉ may not represent an hydrogen;
R₇ and R₈ may not represent at the same time (C₀-C₆)alkyl-aryl, (C₀-C₆)alkyl-heteroaryl;
G¹ₙ groups may not represent at the same time OH;
If R₇, R₈ and M represent at the same time an optionally substituted (C₁-C₄)alkyl, then Q can not be H.
In one aspect, the compounds of the present invention are represented by formula I-B wherein R₁ and R₂ groups are specified as in the formula I-B1 depicted below

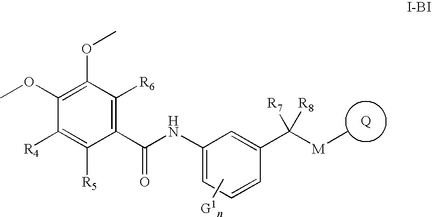

Or a pharmaceutically acceptable salt, hydrate or solvate of such compound

Wherein:

R₄, R₅, R₆ are each independently selected from group consisted of hydrogen, (C₀-C₆)alkyl-CN, (C₁-C₆)alkyl, (C₀-C₆)alkylhalo, (C₃-C₆)cycloalkyl, (C₁-C₆)alkyl-(C₃-C₈)cycloalkyl, (C₂-C₆)alkenyl, (C₂-C₆)alkynyl, (C₀-C₆)alkyl-OR₉, (C₁-C₆)alkyl-NR₉R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O)R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀)R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-)cycloalkyl) or N((C₀-C₆)alkyl)(aryl) substituents;

G¹ is independently selected from a group consisting of hydrogen, OH, (C₁-C₆)alkyl, (C₀-C₆)alkyl-CN, (C₀-C₆)alkylhalo, (C₃-C₆)cycloalkyl, (C₀-C₆)alkyl-(C₃-C₈)cycloalkyl, (C₂-C₆)alkenyl, (C₂-C₆)alkynyl, (C₀-C₆)alkyl-OR₉, (C₀-C₆)alkyl-NR₉R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O)R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀)R₉, or (C₀-C_{C}₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-)cycloalkyl) or N((C₀-C₆)alkyl)(aryl) substituents, wherein optionally two substituents are combined to the intervening atoms to form a bicyclic heterocycloalkyl, aryl or heteroaryl ring; wherein each ring is optionally further substituted with 1-5 independent halogen, CN, (C₁-C₆)alkyl, O—(C₀-C₆)alkyl, O-alkylcycloalkyl, O(aryl), O(heteroaryl), O-arylalkyl, O-heteroarylalkyl, N((—C₀-C₆)alkyl)((C₀-C₃)arylalkyl) or N((C₀-C₆)alkyl)(heteroarylalkyl) groups;

R₉, R₁₀, R₁₁ each independently is hydrogen, (C₁-C₆)alkyl, (C₃-C₆)cycloalkyl, (C₁-C₆)alkyl-(C₃-C₈)cycloalkyl, (C₂-C₆)alkenyl, (C₂-C₆)alkynyl, (C₁-C₆)alkylhalo, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), N(C₀-C₆-alkyl)₂, —N((C₀-C₆)alkyl)((C₃-C₇-)cycloalkyl) or N((C₀-C₆)alkyl)(aryl) substituents;

n is an integer from 1 to 4, provided that when n>1 , the G¹ groups may be equal or different from each other;

R₇ and R₈ are selected from group of formula:

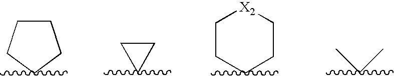

X₂ is independently selected from the group consisting of CH₂, O, S, SO₂;

M is independently selected from the group of consisting of a bond, an optionally substituted (C₁-C₆)alkyl, (C₂-C₆)alkynyl, (C₁-C₆)alkylhalo, (C₂-C₆)alkenyl, (C₁-C₆)alkyl-O—(C₀-C₆)alkyl, (C₁-C₆)-alkylhalo-O—(C₀-C₆)alkyl, (C₃-C₆)alkynyl-O—(C₀-C₆)alkyl, (C₃-C₆)alkenyl-O—(C₀-C₆)alkyl, (C₀-C₆)alkyl-S—(C₀-C₆)alkyl, (C₀-C₆)alkyl-S(═O)—(C₀-C₆)alkyl, (C₀-C₆)alkyl-S(═O)₂—(C₀-C₆)alkyl, (C₀-C₆)alkyl-NR₁₂—(C₀-C₆)alkyl, (C₀-C₆)alkyl-S(═O)₂NR₁₂—(C₀-C₆)alkyl, (C₀-C₆)alkyl-NR₁₂—S(═O)₂—(C₀-C₆)alkyl, (C₀-C₆)alkyl-C(═O)—NR₁₂—(C₀-C₆)alkyl, (C₀-C₆)alkyl-C(═O)—NR₁₂—(C₀-C₆)alkyl-O, (C₀-C₆)alkyl-C(═O)—NR₁₂—(C₀-C₆)alkyl-S, (C₀-C₆)alkyl-NR₁₂C(═O)—(C₀-C₆)alkyl, (C₀-C₆)alkyl-NR₁₂C(═O)—(C₂-C₆)alkyl-O, (C₀-C₆)alkyl-NR₁₂C(═O)—(C₂-C₆)alkyl-S, (C₀-C₆)alkyl-OC(═O)—(C₀-C₆)alkyl, (C₀-C₆)alkyl-C(═O)—O—(C₀-C₆)alkyl, (C₀-C₆)alkyl-C(═O)—(C₀-C₆)alkyl, (C₀-C₆)alkyl-NR₁₂—C(═O)—O—(C₀-C₆)alkyl, (C₀-C₆)alkyl-O—C(═O)—NR₁₂—(C₀-C₆)alkyl or (C₀-C₆)alkyl-NR₁₂—C(═O)—NR₁₃—(C₀-C₆)alkyl substituents;
wherein optionally two substituents are combined to the intervening atoms to form a cycloalkyl, heterocycloalkyl or heteroaryl ring;
wherein each ring is optionally further substituted with 1-5 independent hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₃-C₇)cycloalkyl, O—(C₁-C₆)alkyl-heteroaryl, O—(C₁-C₆)alkylaryl, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl;
R₁₂ and R₁₃ are each independently selected from the group consisting of hydrogen, an optionally substituted (C₁-C₆)alkyl, (C₀-C₆)alkylhalo, (C₂-C₆)alkynyl, (C₂-C₆)alkenyl, (C₃-C₇)cycloalkyl, (C₁-C₆)alkyl-heteroaryl, (C₁-C₆)alkyl-aryl, aryl, heterocycloalkyl, heteroaryl ring; wherein each substitutable carbon atom in R₁₂, R₁₃ is optionally further substituted with hydrogen, OH, (C₁-C₆)alkyl, (C₀-C₄)alkyl-CN, (C₁-C₆)alkylhalo, OR₁₄, SR₁₄, NR₁₄R₁₅, NR₁₄C(═O)—R₁₅, C(═O)—NR₁₄R₁₅, S(═O)₂—NR₁₄R₁₅, NR₁₄S(═O)₂—R₁₅, C(═O)—OR₁₄; C(═NR₁₄)—NR₁₅, wherein R₁₄ and R₁₅ are each independently selected from H, (C₁-C₄)alkyl or (C₁-C₄)alkylhalo;

Q represent independently H, an optionally substituted (C₁-C₆)alkyl, (C₀-C₆)alkyl-CN, (C₁-C₆)alkylhalo, (C₃-C₇)cycloalkyl, (C₃⁻C₇)heterocycloalkyl or one of the following aryl or heteroaryl:

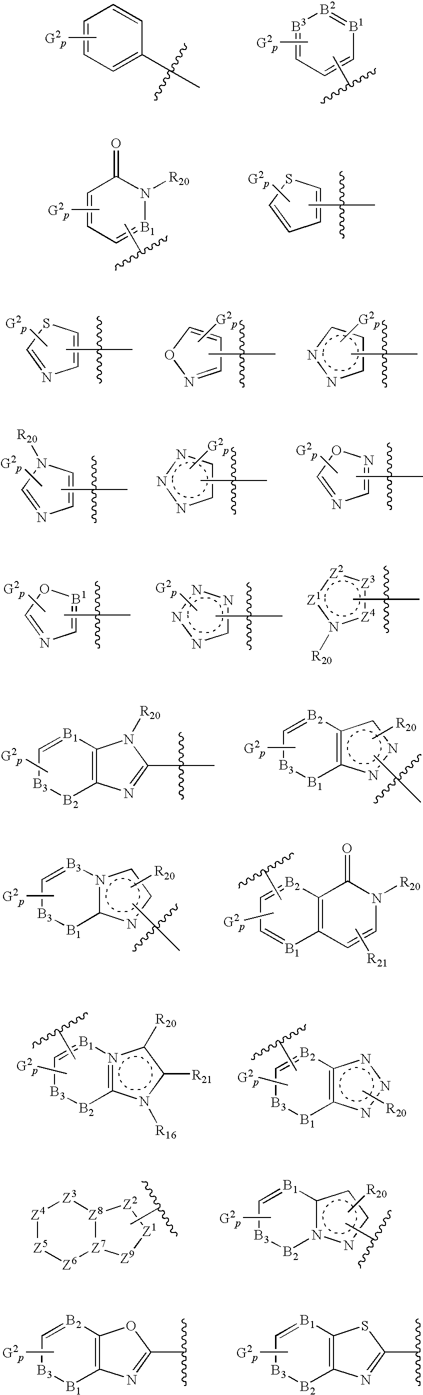

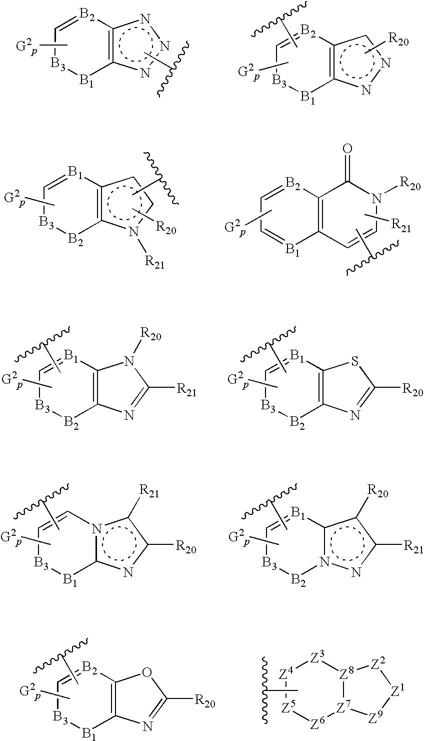

G² groups are each independently selected from the group consisting of hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₂-C₆)alkyl-OR₁₄, O—(C₃-C₇)cycloalkyl, O—(C₁-C₆)alkyl-heteroaryl, O—(C₁-C₆)alkyl-aryl, (C₀-C₆)alkyl-OR₁₄, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl, (C₁-C₆)alkyl-aryl, (C₁-C₆)alkylhalo-OR₁₇, (C₃-C₆)alkynyl-OR₁₇, (C₃-C₆)alkenyl-OR₁₇, (C₀-C₆)alkyl-S—R₁₇, O—(C₂-C₆)alkyl-S—R₁₇, (C₀-C₆)alkyl-S(═O)—R₁₇, O—(C₂-C₆)alkyl-S(═O)—R₁₇, (C₀-C₆)alkyl-S(═O)₂—R₁₇, O—(C₁-C₆)alkyl-S(═O)₂—R₁₇, (C₀-C₆)alkyl-NR₁₇R₁₈, O—(C₂-C₆)alkyl-NR₁₇R₁₈, (C₀-C₆)alkyl-S(═O)₂NR₁₇R₁₈, (C₀-C₆)alkyl-NR₁₇—S(═O)₂R₁₈, O—(C₁-C₆)alkyl-S(═O)₂NR₁₇R₁₈, O—(C₂-C₆)alkyl-NR₁₇—S(═O)₂R₁₈, (C₀-C₆)alkyl-C(═O)—NR₁₇R₁₈, (C₀-C₆)alkyl-NR₁₇C(═O)—R₁₈, O—(C₁-C₆)alkyl-C(═O)—NR₁₇R₁₈, O—(C₂-C₆)alkyl-NR₁₇C(═O)—R₁₈, (C₀-C₆)alkyl-OC(═O)—R₁₇, (C₀-C₆)alkyl-C(═O)—OR₁₇, O—(C₂-C₆)alkyl-OC(═O)—R₁₇, O—(C₁-C₆)alkyl-C(═O)—OR₁₇, (C₀-C₆)alkyl-C(═O)—R₁₇, O—(C₁-C₆)alkyl-C(═O)—R₁₇, (C₀-C₆)alkyl-NR₁₇—C(═O)—OR₁₈, (C₀-C₆)alkyl-O—C(═O)—NR₁₇R₁₈ or (C₀-C₆)alkyl-NR₁₇—C(═O)—NR₁₈R₁₉ substituents;
wherein optionally two substituents are combined to the intervening atoms to form a bicyclic aryl, cycloalkyl, heterocycloalkyl or heteroaryl ring; wherein each ring is optionally further substituted with 1-5 independent hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₃-C₇)cycloalkyl, O—(C₁-C₆)alkyl-heteroaryl, O—(C₁-C₆)alkylaryl, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl;
p is an integer that is selected from the group consisting of 1, 2, 3, 4 and 5 provided that when p>1 , the G² groups may be equal or different from each other;
R₁₆, R₁₇, R₁₈, R₁₉, R₂₀ and R₂₁ are each independently selected from the group consisting of hydrogen, an optionally substituted (C₁-C₆)alkyl, (C₁-C₆)alkylhalo, (C₁-C₆)alkyl-CN, (C₁-C₆)alkyl-O—(C₀-C₆)alkyl, (C₁-C₆)alkyl-N((C₀-C₆)alkyl)₂, (C₁-C₆)alkyl-C(═O)-O—(C₀-C₆)alkyl, (C₁-C₆)alkyl-heterocycloalkyl, (C₂-C₆)alkynyl, (C₂-C₆)alkenyl, (C₃-C₇)cycloalkyl, (C₃-C₇)cycloalkyl-(C₁-C₆)alkyl, heteroaryl, (C₁-C₆)alkyl-heteroaryl, aryl;
Z¹, Z², Z³, Z⁴, Z⁵, Z⁶, Z⁷, Z⁸ and Z⁹ are each independently selected from the group consisting of bond, —C═, —C═C—, —C(═O)—, —C(═S)—, —C—, —O—, —N═, —N— or —S— which may further be substituted by G²ₚ groups;
B¹, B² and B³ are each selected independently from —C—, —N—, —O— or —S— which may further be substituted by one G²ₚ group;
Any N or S bearing ring may be depicted in its N-oxide, S-oxide or S-dioxide form;

It being understood that:
R₇ and R₈ may not represent at the same time (C₀-C₆)alkyl-aryl, (C₀-C₆)alkyl-heteroaryl;
G¹ₙ groups may not represent at the same time OH;
If R₇ and R₈ all represent CH₃ at the same time, then M-Q may not represent CH₃;
If R₅ or R₆ are represented by (C₀-C₆)alkyl-OR₉, then R₉ may not represent an hydrogen;
If R₇, R₈ and M represent at the same time an optionally substituted (C₁-C₄)alkyl, then Q can not be H.
In a second aspect, the compounds of the present invention are represented by Formula I-B1 wherein G¹ₙ groups are specified as in the formula I-B2 depicted below

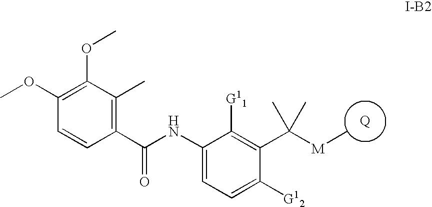

Or a pharmaceutically acceptable salt, hydrate or solvate of such compound

Wherein:

G¹₁ and G¹₂ are each independently selected from a group consisting of hydrogen, OH, (C₁-C₆)alkyl, (C₀-C₆)alkylhalo, (C₀-C₆)alkyl-CN, (C₃-C₆)cycloalkyl, (C₀-C₆)alkyl-(C₃-C₈)cycloalkyl, (C₂-C₆)alkenyl, (C₂-C₆)alkynyl, (C₀-C₆)alkyl-OR₉, (C₀-C₆)alkyl-NR₉R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O)R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀)R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-)cycloalkyl) or N((C₀-C₆)alkyl)(aryl) substituents, wherein optionally two substituents are combined to the intervening atoms to form a bicyclic heterocycloalkyl, aryl or heteroaryl ring; wherein each ring is optionally further substituted with 1-5 independent halogen, CN, (C₁-C₆)alkyl, O—(C₀-C₆)alkyl, O-alkylcycloalkyl, O(aryl), O(heteroaryl), O-arylalkyl, O-heteroarylalkyl, N((—C₀-C₆)alkyl)((C₀-C₃)arylalkyl) or N((C₀-C₆)alkyl)(heteroarylalkyl) groups;

R₉, R₁₀, R₁₁ each independently is hydrogen, (C₁-C₆)alkyl, (C₃-C₆)cycloalkyl, (C₁-C₆)alkyl-(C₃-C₈)cycloalkyl, (C₂-C₆)alkenyl, (C₂-C₆)alkynyl, (C₁-C₆)alkylhalo, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), N(C₀-C₆-alkyl)₂, —N((C₀-C₆)alkyl)((C₃-C₇-)cycloalkyl) or N((C₀-C₆)alkyl)(aryl) substituents;

M is independently selected from the group of consisting of a bond, an optionally substituted (C₂-C₆)alkyl, (C₀-C₆)alkyl-CN, (C₁-C₆)alkyl-O—(C₀-C₆)alkyl, (C₀-C₆)alkyl-C(═O)—NR₁₂—(C₀-C₆)alkyl, (C₀-C₆)alkyl-C(═O)—NR₁₂—(C₀-C₆)alkyl-O, (C₀-C₆)alkyl-C(═O)—NR₁₂—(C₀-C₆)alkyl-S, (C₀-C₆)alkyl-NR₁₂C(═O)—(C₀-C₆)alkyl, (C₀-C₆)alkyl-NR₁₂C(═O)—(C₂-C₆)alkyl-O, (C₀-C₆)alkyl-NR₁₂C(═O)—(C₂-C₆)alkyl-S, (C₀-C₆)alkyl-OC(═O)—(C₀-C₆)alkyl, (C₀-C₆)alkyl-C(═O)—O—(C₀-C₆)alkyl, (C₀-C₆)alkyl-NR₁₂—C(═O)—O—(C₀-C₆)alkyl, (C₀-C₆)alkyl-O—C(═O)—NR₁₂—(C₀-C₆)alkyl or (C₀-C₆)alkyl-NR₁₂—C(═O)—NR₁₃—(C₀-C₆)alkyl substituents;
R₁₂ and R₁₃ are each independently selected from the group consisting of hydrogen, an optionally substituted (C₁-C₆)alkyl, (C₀-C₆)alkylhalo, (C₂-C₆)alkynyl, (C₂-C₆)alkenyl, (C₃-C₇)cycloalkyl, (C₁-C₆)alkyl-hetero aryl, (C₁-C₆)alkyl-aryl, aryl, heterocycloalkyl, heteroaryl ring; wherein each substitutable carbon atom in R₁₂, R₁₃ is optionally further substituted with hydrogen, OH, (C₁-C₆)alkyl, (C₀-C₄)alkyl-CN, (C₁-C₆)alkylhalo, OR₁₄, SR₁₄, NR₁₄R₁₅, NR₁₄C(═O)—R₁₅, C(═O)—NR₁₄R₁₅, S(═O)₂—NR₁₄R₁₅, NR₁₄S(═O)₂—R₁₅, C(═O)—OR₁₄; C(═NR₁₄)—NR₁₅, wherein R₁₄ and R₁₅ are each independently selected from H, (C₁-C₄)alkyl or (C₁-C₄)alkylhalo;

Q represent independently H, an optionally substituted (C₁-C₆)alkyl, (C₀-C₆)alkyl-CN, (C₁-C₆)alkylhalo, (C₃-C₇)cycloalkyl, (C₃-C₇)heterocycloalkyl or one of the following aryl or heteroaryl:

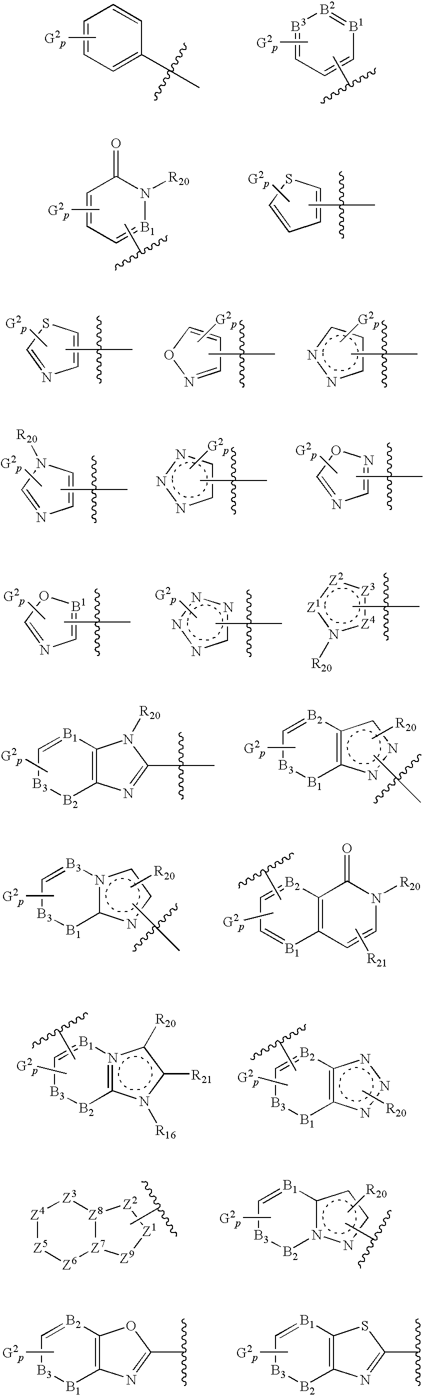

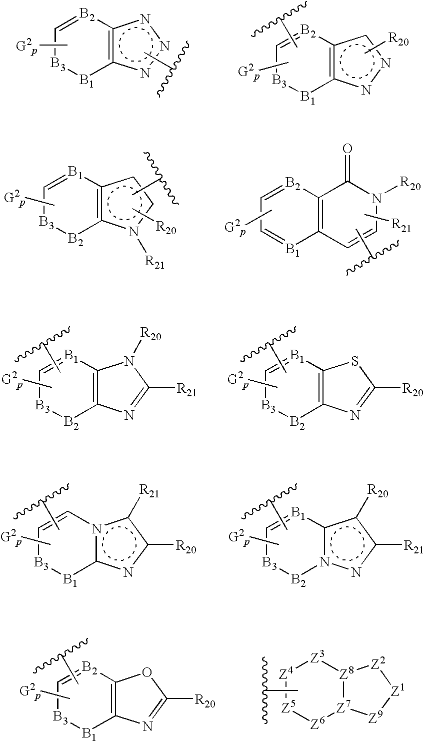

G² groups are each independently selected from the group consisting of hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₂-C₆)alkyl-OR₁₄, O—(C₃-C₇)cycloalkyl, O—(C₁-C₆)alkyl-heteroaryl, O—(C₁-C₆)alkyl-aryl, (C₀-C₆)alkyl-OR₁₄, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl, (C₁-C₆)alkyl-aryl, (C₁-C₆)alkylhalo-OR₁₇, (C₃-C₆)alkynyl-OR₁₇, (C₃-C₆)alkenyl-OR₁₇, (C₀-C₆)alkyl-S—R₁₇, O—(C₂-C₆)alkyl-S—R₁₇, (C₀-C₆)alkyl-S(═O)—R₁₇, O—(C₂-C₆)alkyl-S(═O)—R₁₇, (C₀-C₆)alkyl-S(═O)₂—R₁₇, O—(C₁-C₆)alkyl-S(═O)₂—R₁₇, (C₀-C₆)alkyl-NR₁₇R₁₈, O—(C₂-C₆)alkyl-NR₁₇R₁₈, (C₀-C₆)alkyl-S(═O)₂NR₁₇R₁₈, (C₀-C₆)alkyl-NR₁₇—S(═O)₂R₁₈, O—(C₁-C₆)alkyl-S(═O)₂NR₁₇R₁₈, O—(C₂-C₆)alkyl-NR₁₇—S(═O)₂R₁₈, (C₀-C₆)alkyl-C(═O)—NR₁₇R₁₈, (C₀-C₆)alkyl-NR₁₇C(═O)—R₁₈, O—(C₁-C₆)alkyl-C(═O)—NR₁₇R₁₈, O—(C₂-C₆)alkyl-NR₁₇C(═O)—R₁₈, (C₀-C₆)alkyl-OC(═O)—R₁₇, (C₀-C₆)alkyl-C(═O)—OR₁₇, O—(C₂-C₆)alkyl-OC(═O)—R₁₇, O—(C₁-C₆)alkyl-C(═O)—OR₁₇, (C₀-C₆)alkyl-C(═O)—R₁₇, O—(C₁-C₆)alkyl-C(═O)—R₁₇, (C₀-C₆)alkyl-NR₁₇—C(═O)—OR₁₈, (C₀-C₆)alkyl-O—C(═O)-NR₁₇R₁₈ or (C₀-C₆)alkyl-NR₁₇—C(═O)—NR₁₈R₁₉ substituents;
wherein optionally two substituents are combined to the intervening atoms to form a bicyclic aryl, cycloalkyl, heterocycloalkyl or heteroaryl ring; wherein each ring is optionally further substituted with 1-5 independent hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₃-C₇)cycloalkyl, O—(C₁-C₆)alkyl-heteroaryl, O—(C₃-C₆)alkylaryl, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl;
p is an integer that is selected from the group consisting of 1, 2, 3, 4 and 5 provided that when p>1, the G² groups may be equal or different from each other;
R₁₆, R₁₇, R₁₈, R₁₉, R₂₀ and R₂₁ are each independently selected from the group consisting of hydrogen, an optionally substituted (C₁-C₆)alkyl, (C₁-C₆)alkylhalo, (C₁-C₆)alkyl-CN, (C₁-C₆)alkyl-O—(C₀-C₆)alkyl, (C₁-C₆)alkyl-N((C₀-C₆)alkyl)₂, (C₁-C₆)alkyl-C(═O)—O—(C₀-C₆)alkyl, (C₁-C₆)alkyl-heterocycloalkyl, (C₂-C₆)alkynyl, (C₂-C₆)alkenyl, (C₃-C₇)cycloalkyl, (C₃-C₇)cycloalkyl-(C₁-C₆)alkyl, heteroaryl, (C₁-C₆)alkyl-heteroaryl, aryl;
Z¹, Z², Z³, Z⁴, Z⁵, Z⁶, Z⁷, Z⁸ and Z⁹ are each independently selected from the group consisting of bond, —C═, —C═C—, —C(═O)—, —C(═S)—, —C—, —O—, —N═, —N— or —S— which may further be substituted by G²ₚ groups;
B¹, B² and B³ are each selected independently from —C—, —N—, —O— or —S— which may further be substituted by one G²ₚ group;
Any N or S bearing ring may be depicted in its N-oxide, S-oxide or S-dioxide form;

It being understood that:
G¹₁ and G¹₂ groups may not represent at the same time OH;
If R₇, R₈ and M represent at the same time an optionally substituted (C₁-C₄)alkyl, then Q can not be H.
Further preferred compounds of the present invention are compounds of formula I-B2-a

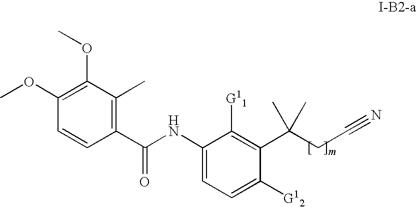

Or a pharmaceutically acceptable salt, hydrate or solvate of such compound

Wherein:

G¹₁ and G¹₂ are each independently selected from a group consisting of hydrogen, (C₁-C₆)alkyl, (C₀-C₆)alkylhalo, (C₀-C₆)alkyl-CN, (C₃-C₆)cycloalkyl, (C₀-C₆)alkyl-(C₃-C₈)cycloalkyl, (C₂-C₆)alkenyl, (C₂-C₆)alkynyl, (C₀-C₆)alkyl-OR₉, (C₀-C₆)alkyl-NR₉R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O)R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀)R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-)cycloalkyl) or N((C₀-C₆)alkyl)(aryl) substituents, wherein optionally two substituents are combined to the intervening atoms to form a bicyclic heterocycloalkyl, aryl or heteroaryl ring; wherein each ring is optionally further substituted with 1-5 independent halogen, CN, (C₁-C₆)alkyl, O—(C₀-C₆)alkyl, O-alkylcycloalkyl, O(aryl), O(heteroaryl), O-arylalkyl, O-heteroarylalkyl, N((-C₀-C₆)alkyl)((C₀-C₃)arylalkyl) or N((C₀-C₆)alkyl)(heteroarylalkyl) groups;
R₉, R₁₀, R₁₁ each independently is hydrogen, (C₁-C₆)alkyl, (C₃-C₆)cycloalkyl, (C₁-C₆)alkyl-(C₃-C₈)cycloalkyl, (C₂-C₆)alkenyl, (C₂-C₆)alkynyl, (C₁-C₆)alkylhalo, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), N(C₀-C₆-alkyl)₂, —N((C₀-C₆)alkyl)((C₃-C₇-)cycloalkyl) or N((C₀-C₆)alkyl)(aryl) substituents;

m is an integer from 0 to 2;
Any N or S bearing ring may be depicted in its N-oxide, S-oxide or S-dioxide form.
In a more preferred aspect of formula I-B2, the compounds of the present invention are represented by formula I-B2-b below

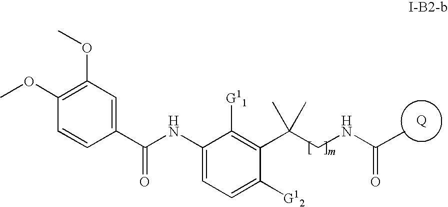

Or a pharmaceutically acceptable salt, hydrate or solvate of such compound

Wherein:

G¹₁ and G¹₂ are each independently selected from a group consisting of hydrogen, (C₁-C₆)alkyl, (C₀-C₆)alkylhalo, (C₀-C₆)alkyl-CN, (C₃-C₆)cycloalkyl, (C₀-C₆)alkyl-(C₃-C₈)cycloalkyl, (C₂-C₆)alkenyl, (C₂-C₆)alkynyl, (C₀-C₆)alkyl-OR₉, (C₀-C₆)alkyl-NR₉R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O)R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀)R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-)cycloalkyl) or N((C₀-C₆)alkyl)(aryl) substituents, wherein optionally two substituents are combined to the intervening atoms to form a bicyclic heterocycloalkyl, aryl or heteroaryl ring; wherein each ring is optionally further substituted with 1-5 independent halogen, CN, (C₁-C₆)alkyl, O—(C₀-C₆)alkyl, O-alkylcycloalkyl, O(aryl), O(heteroaryl), O-arylalkyl, O-heteroarylalkyl, N((—C₀-C₆)alkyl)((C₀-C₃)arylalkyl) or N((C₀-C₆)alkyl)(heteroarylalkyl) groups;

R₉, R₁₀, R₁₁ each independently is hydrogen, (C₁-C₆)alkyl, (C₃-C₆)cycloalkyl, (C₁-C₆)alkyl-(C₃-C₈)cycloalkyl, (C₂-C₆)alkenyl, (C₂-C₆)alkynyl, (C₁-C₆)alkylhalo, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), N(C₀-C₆-alkyl)₂, —N((C₀-C₆)alkyl)((C₃-C₇-)cycloalkyl) or N((C₀-C₆)alkyl)(aryl) substituents;

m is an integer from 0 to 2;

Q represent independently H, an optionally substituted (C₁-C₆)alkyl, (C₀-C₆)alkyl-CN, (C₁-C₆)alkylhalo, (C₃-C₇)cycloalkyl, (C₃-C₇)heterocycloalkyl or one of the following aryl or heteroaryl:

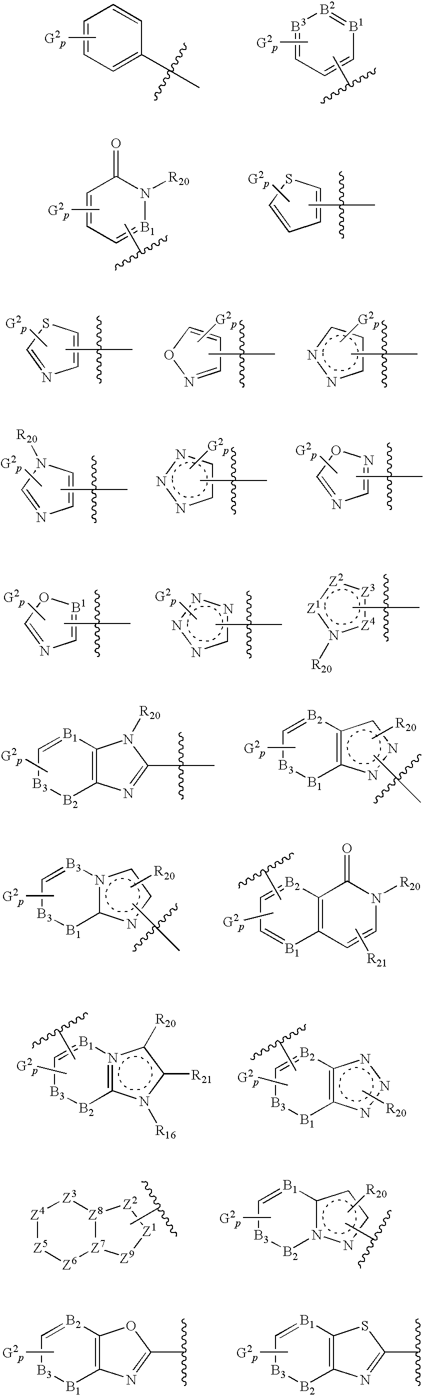

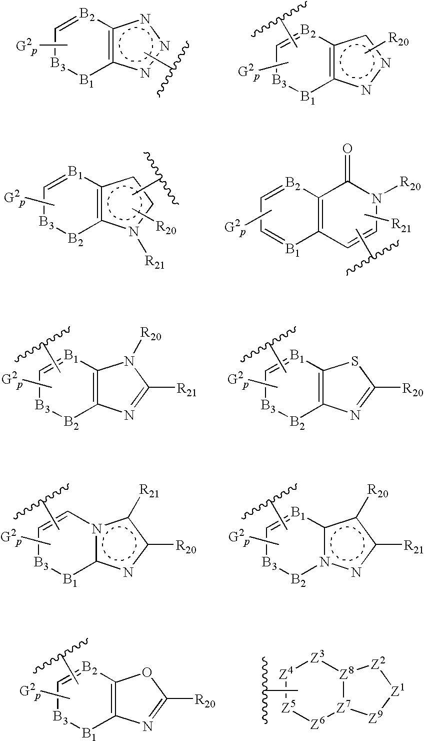

G² groups are each independently selected from the group consisting of hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₂-C₆)alkyl-OR₁₄, O—(C₃-C₇)cycloalkyl, O—(C₁-C₆)alkyl-heteroaryl, O—(C₁-C₆)alkyl-aryl, (C₀-C₆)alkyl-OR₁₄, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl, (C₁-C₆)alkyl-aryl, (C₁-C₆)alkylhalo-OR₁₇, (C₃-C₆)alkynyl-OR₁₇, (C₃-C₆)alkenyl-OR₁₇, (C₀-C₆)alkyl-S—R₁₇, O—(C₂-C₆)alkyl-S—R₁₇, (C₀-C₆)alkyl-S(═O)—R₁₇, O—(C₂-C₆)alkyl-S(═O)—R₁₇, (C₀-C₆)alkyl-S(═O)₂—R₁₇, O—(C₁-C₆)alkyl-S(═O)₂—R₁₇, (C₀-C₆)alkyl-NR₁₇R₁₈, O—(C₂-C₆)alkyl-NR₁₇R₁₈, (C₀-C₆)alkyl-S(═O)₂NR₁₇R₁₈, (C₀-C₆)alkyl-NR₁₇—S(═O)₂R₁₈, O—(C₁-C₆)alkyl-S(═O)₂NR₁₇R₁₈, O—(C₂-C₆)alkyl-NR₁₇—S(═O)₂R₁₈, (C₀-C₆)alkyl-C(═O)—NR₁₇R₁₈, (C₀-C₆)alkyl-NR₁₇C(═O)—R₁₈, O—(C₁-C₆)alkyl-C(═O)—NR₁₇R₁₈, O—(C₂-C₆)alkyl-NR₁₇C(═O)—R₁₈, (C₀-C₆)alkyl-OC(═O)—R₁₇, (C₀-C₆)alkyl-C(═O)—OR₁₇, O—(C₂-C₆)alkyl-OC(═O)—R₁₇, O—(C₁-C₆)alkyl-C(═O)—OR₁₇, (C₀-C₆)alkyl-C(═O)—R₁₇, O—(C₁-C₆)alkyl-C(═O)—R₁₇, (C₀-C₆)alkyl-NR₁₇—C(═O)—OR₁₈, (C₀-C₆)alkyl-O—C(═O)—NR₁₇R₁₈ or (C₀-C₆)alkyl-NR₁₇C(═O)—NR₁₈R₁₉ substituents;
wherein optionally two substituents are combined to the intervening atoms to form a bicyclic aryl, cycloalkyl, heterocycloalkyl or heteroaryl ring; wherein each ring is optionally further substituted with 1-5 independent hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₃-C₇)cycloalkyl, O—(C₁-C₆)alkyl-heteroaryl, O—(C₁-C₆)alkylaryl, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl;
p is an integer that is selected from the group consisting of 1, 2, 3, 4 and 5 provided that when p>1, the G² groups may be equal or different from each other;
R₁₆, R₁₇, R₁₈, R₁₉, R₂₀ and R₂₁ are each independently selected from the group consisting of hydrogen, an optionally substituted (C₁-C₆)alkyl, (C₁-C₆)alkylhalo, (C₁-C₆)alkyl-CN, (C₁-C₆)alkyl-O—(C₀-C₆)alkyl, (C₁-C₆)alkyl-N((C₀-C₆)alkyl)₂, (C₁-C₆)alkyl-C(═O)-O—(C₀-C₆)alkyl, (C₁-C₆)alkyl-hetero cycloalkyl, (C₂-C₆)alkynyl, (C₂-C₆)alkenyl, (C₃-C₇)cycloalkyl, (C₃-C₇)cycloalkyl-(C₁-C₆)alkyl, heteroaryl, (C₁-C₆)alkyl-heteroaryl, aryl;
Z¹, Z², Z³, Z⁴, Z⁵, Z⁶, Z⁷, Z⁸ and Z⁹ are each independently selected from the group consisting of bond, —C═, —C═C—, —C(═O)—, —C(═S)—, —C—, —O—, —N═, —N— or —S— which may further be substituted by G²ₚ groups;
B¹, B² and B³ are each selected independently from —C—, —N—, —O— or —S— which may further be substituted by one G²ₚ group;
Any N or S bearing ring may be depicted in its N-oxide, S-oxide or S-dioxide form.
In one aspect, the compounds of the present invention are represented by formula I-B2-b wherein the heterocyclic ring system is specified as in the formula I-B2-b1 depicted below

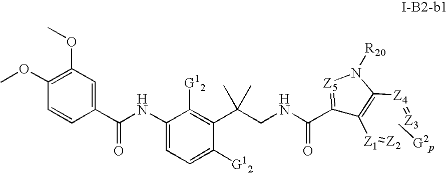

Or a pharmaceutically acceptable salt, hydrate or solvate of such compound

Wherein:

G¹₁ and G¹₂ are each independently selected from a group consisting of hydrogen, (C₀-C₆)alkyl-CN, (C₁-C₆)alkyl, (C₀-C₆)alkylhalo, (C₃-C₆)cycloalkyl, (C₀-C₆)alkyl-(C₃-C₈)cycloalkyl, (C₂-C₆)alkenyl, (C₂-C₆)alkynyl, (C₀-C₆)alkyl-OR₉, (C₀-C₆)alkyl-NR₉R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O)R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀)R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-)cycloalkyl) or N((C₀-C₆)alkyl)(aryl) substituents, wherein optionally two substituents are combined to the intervening atoms to form a bicyclic heterocycloalkyl, aryl or heteroaryl ring; wherein each ring is optionally further substituted with 1-5 independent halogen, CN, (C₁-C₆)alkyl, O—(C₀-C₆)alkyl, O-alkylcycloalkyl, O(aryl), O(heteroaryl), O-arylalkyl, O-heteroarylalkyl, N((-C₀-C₆)alkyl)((C₀-C₃)arylalkyl) or N((C₀-C₆)alkyl)(heteroarylalkyl) groups;
R₉, R₁₀, R₁₁ each independently is hydrogen, (C₁-C₆)alkyl, (C₃-C₆)cycloalkyl, (C₁-C₆)alkyl-(C₃-C₈)cycloalkyl, (C₂-C₆)alkenyl, (C₂-C₆)alkynyl, (C₁-C₆)alkylhalo, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), N(C₀-C₆-alkyl)₂, —N((C₀-C₆)alkyl)((C₃-C₇-)cycloalkyl) or N((C₀-C₆)alkyl)(aryl) substituents;
G² groups are each independently selected from the group consisting of hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₂-C₆)alkyl-OR₁₄, O—(C₃-C₇)cycloalkyl, O—(C₁-C₆)alkyl-heteroaryl, O—(C₁-C₆)alkyl-aryl, (C₀-C₆)alkyl-OR₁₄, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl, (C₁-C₆)alkyl-aryl, (C₁-C₆)alkylhalo-OR₁₇, (C₃-C₆)alkynyl-OR₁₇, (C₃-C₆)alkenyl-OR₁₇, (C₀-C₆)alkyl-S—R₁₇, O—(C₂-C₆)alkyl-S—R₁₇, (C₀-C₆)alkyl-S (═O)—R₁₇, O—(C₂-C₆)alkyl-S(═O)—R₁₇, (C₀-C₆)alkyl-S(═O)₂—R₁₇, O—(C₁-C₆)alkyl-S(═O)₂—R₁₇, (C₀-C₆)alkyl-NR₁₇R₁₈, O—(C₂-C₆)alkyl-NR₁₇R₁₈, (C₀-C₆)alkyl-S(═O)₂NR₁₇R₁₈, (C₀-C₆)alkyl-NR₁₇-S(═O)₂R₁₈, O—(C₁-C₆)alkyl-S(═O)₂NR₁₇R₁₈, O—(C₂-C₆)alkyl-NR₁₇—S(═O)₂R₁₈, (C₀-C₆)alkyl-C(═O)—NR₁₇R₁₈, (C₀-C₆)alkyl-NR₁₇C(═O)—R₁₈, O—(C₁-C₆)alkyl-C(═O)—NR₁₇R₁₈, O—(C₂-C₆)alkyl-NR₁₇C(═O)—R₁₈, (C₀-C₆)alkyl-OC(═O)—R₁₇, (C_{O}-C₆)alkyl-C(═O)—OR₁₇, O—(C₂-C₆)alkyl-OC(═O)-R₁₇, O—(C₁-C₆)alkyl-C(═O)—OR₁₇, (C₀-C₆)alkyl-C(═O)—R₁₇, O—(C₁-C₆)alkyl-C(═O)—R₁₇, (C₀-C₆)alkyl-NR₁₇-C(═O)—OR₁₈, (C₀-C_{C}₆)alkyl-O—C(═O)—NR₁₇R₁₈ or (C₀-C₆)alkyl-NR₁₇—C(═O)—NR₁₈R₁₉ substituents;
wherein optionally two substituents are combined to the intervening atoms to form a bicyclic aryl, cycloalkyl, heterocycloalkyl or heteroaryl ring; wherein each ring is optionally further substituted with 1-5 independent hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O-(C₃-C₆)alkenyl, O—(C₃-C₇)cycloalkyl, O—(C₁-C₆)alkyl-heteroaryl, O—(C₁-C₆)alkylaryl, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl;
p is an integer that is selected from the group consisting of 1, 2, 3, 4 and 5 provided that when p>1, the G² groups may be equal or different from each other;
R₁₆, R₁₇, R₁₈, R₁₉ and R₂₀ are each independently selected from the group consisting of hydrogen, an optionally substituted (C₁-C₆)alkyl, (C₁-C₆)alkylhalo, (C₁-C₆)alkyl-CN, (C₁-C₆)alkyl-O—(C₀-C₆)alkyl, (C₁-C₆)alkyl-N((C₀-C₆)alkyl)₂, (C₁-C₆)alkyl-C(═O)-O—(C₀-C₆)alkyl, (C₁-C₆)alkyl-heterocycloalkyl, (C₂-C₆)alkynyl, (C₂-C₆)alkenyl, (C₃-C₇)cycloalkyl, (C₃-C₇)cycloalkyl-(C₁-C₆)alkyl, heteroaryl, (C₁-C₆)alkyl-heteroaryl, aryl;
Z¹, Z², Z³ and Z⁴ are each independently selected from the group consisting of bond, —C═, —C═C—, —C(═O)—, —C(═S)—, —C—, —O—, —N═, —N— or —S— which may further be substituted by G²ₚ groups;
Z⁵ is independently selected from —C— or —N— which may further be substituted by one G² group;
Any N or S bearing ring may be depicted in its N-oxide, S-oxide or S-dioxide form.

Preferred compounds of the present invention are compounds of formula I-B2-b2 are depicted below

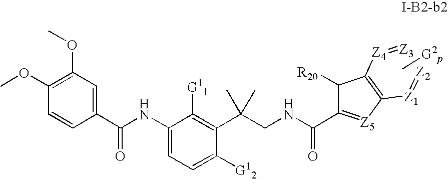

Or a pharmaceutically acceptable salt, hydrate or solvate of such compound

Wherein:

G¹₁ and G¹₂ are each independently selected from a group consisting of hydrogen, (C₀-C₆)alkyl-CN, (C₁-C₆)alkyl, (C₀-C₆)alkylhalo, (C₃-C₆)cycloalkyl, (C₀-C₆)alkyl-(C₃-C₈)cycloalkyl, (C₂-C₆)alkenyl, (C₂-C₆)alkynyl, (C₀-C₆)alkyl-OR₉, (C₀-C₆)alkyl-NR₉R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O)R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀)R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-)cycloalkyl) or N((C₀-C₆)alkyl)(aryl) substituents, wherein optionally two substituents are combined to the intervening atoms to form a bicyclic heterocycloalkyl, aryl or heteroaryl ring; wherein each ring is optionally further substituted with 1-5 independent halogen, CN, (C₁-C₆)alkyl, O—(C₀-C₆)alkyl, O-alkylcycloalkyl, O(aryl), O(heteroaryl), O-arylalkyl, O-heteroarylalkyl, N((-C₀-C₆)alkyl)((C₀-C₃)arylalkyl) or N((C₀-C₆)alkyl)(heteroarylalkyl) groups;
R₉, R₁₀, R₁₁ each independently is hydrogen, (C₁-C₆)alkyl, (C₃-C₆)cycloalkyl, (C₁-C₆)alkyl-(C₃-C₈)cycloalkyl, (C₂-C₆)alkenyl, (C₂-C₆)alkynyl, (C₁-C₆)alkylhalo, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), N(C₀-C₆-alkyl)₂, —N((C₀-C₆)alkyl)((C₃-C₇-)cycloalkyl) or N((C₀-C₆)alkyl)(aryl) substituents;

G² groups are each independently selected from the group consisting of hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₂-C₆)alkyl-OR₁₄, O—(C₃-C₇)cycloalkyl, O—(C₁-C₆)alkyl-heteroaryl, O—(C₁-C₆)alkyl-aryl, (C₀-C₆)alkyl-OR₁₄, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl, (C₁-C₆)alkyl-aryl, (C₁-C₆)alkylhalo-OR₁₇, (C₃-C₆)alkynyl-OR₁₇, (C₃-C₆)alkenyl-OR₁₇, (C₀-C₆)alkyl-S—R₁₇, O—(C₂-C₆)alkyl-S—R₁₇, (C₀-C₆)alkyl-S(═O)—R₁₇, O—(C₂-C₆)alkyl-S(═O)—R₁₇, (C₀-C₆)alkyl-S(═O)₂—R₁₇, O—(C₁-C₆)alkyl-S(═O)₂—R₁₇, (C₀-C₆)alkyl-NR₁₇R₁₈, O—(C₂-C₆)alkyl-NR₁₇R₁₈, (C₀-C₆)alkyl-S(═O)₂NR₁₇R₁₈, (C₀-C₆)alkyl-NR₁₇—S(═O)₂R₁₈, O—(C₁-C₆)alkyl-S(═O)₂NR ₇R₁₈, O—(C₂-C₆)alkyl-NR₁₇—S(═O)₂R₁₈, (C₀-C₆)alkyl-C(═O)—NR₁₇R₁₈, (C₀-C₆)alkyl-NR₁₇C(═O)—R₁₈, O—(C₁-C₆)alkyl-C(═O)—NR₁₇R₁₈, O—(C₂-C₆)alkyl-NR₁₇C(═O)—R₁₈, (C₀-C₆)alkyl-OC(═O)—R₁₇, (C₀-C₆)alkyl-C(═O)—OR₁₇, O—(C₂-C₆)alkyl-OC(═O)—R₁₇, O—(C₁-C₆)alkyl-C(═O)—OR₁₇, (C₀-C₆)alkyl-C(═O)—R₁₇, O—(C₁-C₆)alkyl-C(═O)—R₁₇, (C₀-C₆)alkyl-NR₁₇—C(═O)—OR₁₈, (C₀-C₆)alkyl-O—C(═O)—NR₁₇R₁₈ or (C₀-C₆)alkyl-NR₁₇—C(═O)—NR₁₈R₁₉ substituents;
wherein optionally two substituents are combined to the intervening atoms to form a bicyclic aryl, cycloalkyl, heterocycloalkyl or heteroaryl ring; wherein each ring is optionally further substituted with 1-5 independent hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₃-C₇)cycloalkyl, O—(C₁-C₆)alkyl-heteroaryl, O—(C₁-C₆)alkylaryl, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl;
p is an integer that is selected from the group consisting of 1, 2, 3, 4 and 5 provided that when p>1, the G² groups may be equal or different from each other;
R₁₆, R₁₇, R₁₈, R₁₉ and R₂₀ are each independently selected from the group consisting of hydrogen, an optionally substituted (C₁-C₆)alkyl, (C₁-C₆)alkylhalo, (C₁-C₆)alkyl-CN, (C₁-C₆)alkyl-O—(C₀-C₆)alkyl, (C₁-C₆)alkyl-N((C₀-C₆)alkyl)₂, (C₁-C₆)alkyl-C(═O)-O—(C₀-C₆)alkyl, (C₁-C₆)alkyl-heterocycloalkyl, (C₂-C₆)alkynyl, (C₂-C₆)alkenyl, (C₃-C₇)cycloalkyl, (C₃-C₇)cycloalkyl-(C₁-C₆)alkyl, heteroaryl, (C₁-C₆)alkyl-heteroaryl, aryl;
Z¹, Z², Z³ and Z⁴ are each independently selected from the group consisting of bond, —C═, —C═C—, —C(═O)—, —C(═S)—, —C—, —O—, —N═, —N— or —S— which may further be substituted by G²ₚ groups;
Z⁵ is independently selected from —C— or —N— which may further be substituted by one G²ₚ group;
Any N or S bearing ring may be depicted in its N-oxide, S-oxide or S-dioxide form.
Further preferred compounds of the present invention are compounds of formula I-B2-b3

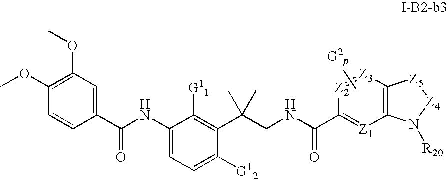

Or a pharmaceutically acceptable salt, hydrate or solvate of such compound

Wherein:

G¹₁ and G¹₂ are each independently selected from a group consisting of hydrogen, (C₀-C₆)alkyl-CN, (C₁-C₆)alkyl, (C₀-C₆)alkylhalo, (C₃-C₆)cycloalkyl, (C₀-C₆)alkyl-(C₃-C₈)cycloalkyl, (C₂-C₆)alkenyl, (C₂-C₆)alkynyl, (C₀-C₆)alkyl-OR₉, (C₀-C₆)alkyl-NR₉R₁₀, (C₀-C₆)-alkyl-NR₉COR₁₀, (C₀-C₆)alkyl-NR₉ SO₂R₁₀, (C₀-C₆)alkyl-NR₁₁CONR₁₀R₉, (C₀-C₆)alkyl-SR₉, (C₀-C₆)alkyl-S(═O)R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀)R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-)cycloalkyl) or N((C₀-C₆)alkyl)(aryl) substituents, wherein optionally two substituents are combined to the intervening atoms to form a bicyclic heterocycloalkyl, aryl or heteroaryl ring; wherein each ring is optionally further substituted with 1-5 independent halogen, CN, (C₁-C₆)alkyl, O—(C₀-C₆)alkyl, O-alkylcycloalkyl, O(aryl), O(heteroaryl), O-arylalkyl, O-heteroarylalkyl, N((—C₀-C₆)alkyl)((C₀-C₃)arylalkyl) or N((C₀-C₆)alkyl)(heteroarylalkyl) groups;
R₉, R₁₀, R₁₁ each independently is hydrogen, (C₁-C₆)alkyl, (C₃-C₆)cycloalkyl, (C -C₆)alkyl-(C₃-C₈)cycloalkyl, (C₂-C₆)alkenyl, (C₂-C₆)alkynyl, (C₁-C₆)alkylhalo, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), N(C₀-C₆-alkyl)₂, —N((C₀-C₆)alkyl)((C₃-C₇-)cycloalkyl) or N((C₀-C₆)alkyl)(aryl) substituents;

G² groups are each independently selected from the group consisting of hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₂-C₆)alkyl-OR₁₄, O—(C₃-C₇)cycloalkyl, O—(C₁-C₆)alkyl-heteroaryl, O—(C₁-C₆)alkyl-aryl, (C₀-C₆)alkyl-OR₁₄, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl, (C₁-C₆)alkyl-aryl, (C₁-C₆)alkylhalo-OR₁₇, (C₃-C₆)alkynyl-OR₁₇, (C₃-C₆)alkenyl-OR₁₇, (C₀C₆)alkyl-S—R₁₇, O—(C₂-C₆)alkyl-S—R₁₇, (C₀-C₆)alkyl-S(═O)—R₁₇, O—(C₂-C₆)alkyl-S(═O)—R₁₇, (C₀-C₆)alkyl-S(═O)₂—R₁₇, O—(C₁-C₆)alkyl-S(═O)₂-R₁₇, (C₀-C₆)alkyl-NR₁₇R₁₈, O—(C₂-C₆)alkyl-NR₁₇R₁₈, (C₀-C₆)alkyl-S(═O)₂NR₁₇R₁₈, (C₀-C₆)alkyl-NR₁₇—S(═O)₂R₁₈, O—(C₁-C₆)alkyl-S(═O)₂NR₁₇R₁₈, O—(C₂-C₆)alkyl-NR₁₇—S(═O)₂R₁₈, (C₀-C₆)alkyl-C(═O)—NR₁₇R₁₈, (C₀-C₆)alkyl-NR₁₇C(═O)—R₁₈, O—(C₁-C₆)alkyl-C(═O)—NR₁₇R₁₈, O—(C₂-C₆)alkyl-NR₁₇C(═O)—R₁₈, (C₀-C₆)alkyl-OC(═O)—R₁₇, (C₀-C₆)alkyl-C(═O)—OR₁₇, O—(C₂-C₆)alkyl-OC(═O)—R₁₇, O—(C₁-C₆)alkyl-C(═O)—OR₁₇, (C₀-C₆)alkyl-C(═O)—R₁₇, O—(C₁-C₆)alkyl-C(═O)—R₁₇, (C₀-C₆)alkyl-NR₁₇—C(═O)—OR₁₈, (C₀-C₆)alkyl-O—C(═O)—NR₁₇R₁₈ or (C₀-C₆)alkyl-NR₁₇—C(═O)—NR₁₈R₁₉ substituents;
wherein optionally two substituents are combined to the intervening atoms to form a bicyclic aryl, cycloalkyl, heterocycloalkyl or heteroaryl ring; wherein each ring is optionally further substituted with 1-5 independent hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₃-C₇)cycloalkyl, O—(C₁-C₆)alkyl-heteroaryl, O—(C₁-C₆)alkylaryl, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl;
p is an integer that is selected from the group consisting of 1, 2, 3, 4 and 5 provided that when p>1, the G² groups may be equal or different from each other;
R₁₆, R₁₇, R₁₈, R₁₉, and R₂₀ are each independently selected from the group consisting of hydrogen, an optionally substituted (C₁-C₆)alkyl, (C₁-C₆)alkylhalo, (C₁-C₆)alkyl-CN, (C₁-C₆)alkyl-O—(C₀-C₆)alkyl, (C₁-C₆)alkyl-N((C₀-C₆)alkyl)₂, (C₁-C₆)alkyl-C(═O)—O—(C₀-C₆)alkyl, (C₁-C₆)alkyl-hetero cycloalkyl, (C₂-C₆)alkynyl, (C₂-C₆)alkenyl, (C₃-C₇)cycloalkyl, (C₃-C₇)cycloalkyl-(C₁-C₆)alkyl, heteroaryl, (C₁-C₆)alkyl-hetero aryl, aryl;
Z¹, Z² and Z³ are each independently selected from the group consisting of bond, —C═, —C(═O)—, —C(═S)—, —C—, —O—, —N═, —N— or —S— which may further be substituted by G²ₚ groups;
Z⁴ and Z⁵ are each independently selected from —C— or —N— which may further be substituted by one G²ₚ group;
Any N or S bearing ring may be depicted in its N-oxide, S-oxide or S-dioxide form.

Further specific embodiments of the present invention are compounds of formula I-B2-b4

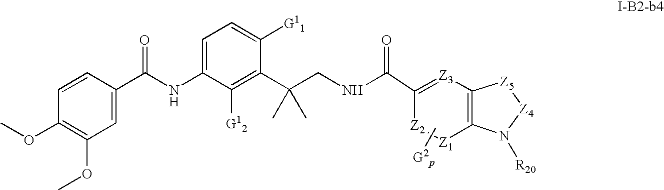

Or a pharmaceutically acceptable salt, hydrate or solvate of such compound

Wherein:

G¹₁ and G¹₂ are each independently selected from a group consisting of hydrogen, (C₀-C₆)alkyl-CN, (C₁-C₆)alkyl, (C₀-C₆)alkylhalo, (C₃-C₆)cycloalkyl, (C₀-C₆)alkyl-(C₃-C₈)cycloalkyl, (C₂-C₆)alkenyl, (C₂-C₆)alkynyl, (C₀-C₆)alkyl-OR₉, (C₀-C₆)alkyl-NR₉R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O)R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀)R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-)cycloalkyl) or N((C₀-C₆)alkyl)(aryl) substituents, wherein optionally two substituents are combined to the intervening atoms to form a bicyclic heterocycloalkyl, aryl or heteroaryl ring; wherein each ring is optionally further substituted with 1-5 independent halogen, CN, (C₁-C₆)alkyl, O—(C₀-C₆)alkyl, O-alkylcycloalkyl, O(aryl), O(heteroaryl), O-arylalkyl, O-heteroarylalkyl, N((-C₀-C₆)alkyl)((C₀-C₃)arylalkyl) or N((C₀-C₆)alkyl)(heteroarylalkyl) groups;
R₉, R₁₀, R₁₁ each independently is hydrogen, (C₁-C₆)alkyl, (C₃-C₆)cycloalkyl, (C₁-C₆)alkyl-(C₃-C₈)cycloalkyl, (C₂-C₆)alkenyl, (C₂-C₆)alkynyl, (C₁-C₆)alkylhalo, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), N(C₀-C₆-alkyl)₂, —N((C₀-C₆)alkyl)((C₃-C₇-)cycloalkyl) or N((C₀-C₆)alkyl)(aryl) substituents;

G² groups are each independently selected from the group consisting of hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₂-C₆)alkyl-OR₁₄, O—(C₃-C₇)cycloalkyl, O—(C₁-C₆)alkyl-heteroaryl, O—(C₁-C₆)alkyl-aryl, (C₀-C₆)alkyl-OR₁₄, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-hetero aryl, aryl, O-aryl, (C₁-C₆)alkyl-aryl, (C₁-C₆)alkylhalo-OR₁₇, (C₃-C₆)alkynyl-OR₁₇, (C₃-C₆)alkenyl-OR₁₇, (C₀-C₆)alkyl-S—R₁₇, O—(C₂-C₆)alkyl-S—R₁₇, (C₀-C₆)alkyl-S(═O)—R₁₇, O—(C₂-C₆)alkyl-S(═O)—R₁₇, (C₀-C₆)alkyl-S(═O)₂—R₁₇, O—(C₁-C₆)alkyl-S(═O)₂—R₁₇, (C₀-C₆)alkyl-NR₁₇R₁₈, O—(C₂-C₆)alkyl-NR₁₇R₁₈, (C₀-C₆)alkyl-S(═O)₂NR₁₇R₁₈, (C₀-C₆)alkyl-NR₁₇—S(═O)₂R₁₈, O—(C₁-C₆)alkyl-S(═O)₂NR₁₇R₁₈, O—(C₂-C₆)alkyl-NR₁₇—S(═O)₂R₁₈, (C₀-C₆)alkyl-C(═O)—NR₁₇R₁₈, (C₀-C₆)alkyl-NR₁₇C(═O)—R₁₈, O—(C₁-C₆)alkyl-C(═O)—NR₁₇R₁₈, O—(C₂-C₆)alkyl-NR₁₇C(═O)—R₁₈, (C₀-C₆)alkyl-OC(═O)—R₁₇, (C₀-C₆)alkyl-C(═O)—OR₁₇, O—(C₂-C₆)alkyl-OC(═O)—R₁₇, O—(C₁-C₆)alkyl-C(═O)—OR₁₇, (C₀-C₆)alkyl-C(═O)—R₁₇, O—(C₁-C₆)alkyl-C(═O)—R₁₇, (C₉-C₆)alkyl-NR₁₇—C(═O)—OR₁₈, (C₀-C₆)alkyl-O—C(═O)—NR₁₇R₁₈ or (C₀-C₆)alkyl-NR₁₇—C(═O)—NR₁₈R₁₉ substituents;
wherein optionally two substituents are combined to the intervening atoms to form a bicyclic aryl, cycloalkyl, heterocycloalkyl or heteroaryl ring; wherein each ring is optionally further substituted with 1-5 independent hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₃-C₇)cycloalkyl, O—(C₁-C₆)alkyl-heteroaryl, O—(C₁-C₆)alkylaryl, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl;
p is an integer that is selected from the group consisting of 1, 2, 3, 4 and 5 provided that when p>1, the G² groups may be equal or different from each other;
R₁₆, R₁₇, R₁₈, R₁₉ and R₂₀ are each independently selected from the group consisting of hydrogen, an optionally substituted (C₁-C₆)alkyl, (C₁-C₆)alkylhalo, (C₁-C₆)alkyl-CN, (C₁-C₆)alkyl-O—(C_{0-C}₆)alkyl, (C₁-C₆)alkyl-N((C₀-C₆)alkyl)₂, (C₁-C₆)alkyl-C(═O)-O—(C₉-C₆)alkyl, (C₁-C₆)alkyl-hetero cycloalkyl, (C₂-C₆)alkynyl, (C₂-C₆)alkenyl, (C₃-C₇)cycloalkyl, (C₃-C₇)cycloalkyl-(C₁-C₆)alkyl, heteroaryl, (C₁-C₆)alkyl-heteroaryl, aryl;
Z¹, Z² and Z³ are each independently selected from the group consisting of bond, —C═, —C═C—, —C(═O)—, —C(═S)—, —C—, —O—, —N═, —N— or —S— which may further be substituted by G²ₚ groups;
Z⁴ and Z⁵ are each independently selected from —C— or —N— which may further be substituted by one G²ₚ group;
Any N or S bearing ring may be depicted in its N-oxide, S-oxide or S-dioxide form.

Specifically preferred compounds are:

3,4-Dimethoxy-N-[4-(1-methyl-1-pyridin-4-yl-ethyl)-phenyl]-benzamide

1-[4-(3,4-Dimethoxy-benzoylamino)-phenyl]-cyclopentanecarboxylic acid methyl ester

3,4-Dimethoxy-N-[4-(1-methylcarbamoyl-cyclopentyl)-phenyl]-benzamide

N-[4-(1-Dimethylcarbamoyl-cyclopentyl)-phenyl]-3,4-dimethoxy-benzamide

3,4-Dimethoxy-N-{4-[1-(5-methyl-[1,2,4]oxadiazol-3-yl)-cyclopentyl]-phenyl}-benzamide

N-{4-[1-(Acetylamino-methyl)-cyclopentyl]-phenyl}-3,4-dimethoxy-benzamide

N-[3-(1-Cyano-cyclopentyl)-phenyl]-3,4-dimethoxy-benzamide

{1-[4-(3,4-Dimethoxy-benzoylamino)-phenyl]-cyclopentyl}-carbamic acid methyl ester

3,4-Dimethoxy-N-{4-[1-(morpholine-4-carbonyl)-cyclopentyl]-phenyl}-benzamide

N-[4-(1-Hydroxymethyl-cyclopentyl)-phenyl]-3,4-dimethoxy-benzamide

N-(4-{1-[(2,2-Dimethyl-propionylamino)-methyl]-cyclopentyl}-phenyl)-3,4-dimethoxy-benzamide

3,4-Dimethoxy-N-[4-(1-ureidomethyl-cyclopentyl)-phenyl]-benzamide

N-[4-(2-Acetylamino-1,1-dimethyl-ethyl)-phenyl]-3,4-dimethoxy-benzamide

N-[4-(1-Acetylamino-1-methyl-ethyl)-phenyl]-3,4-dimethoxy-benzamide

3,4-Dimethoxy-N-{4-[1-methyl-1-(5-methyl-[1,2,4]oxadiazol-3-yl)-ethyl]-phenyl}-benzamide

Thiazole-4-carboxylic acid {1-[4-(3,4-dimethoxy-benzoylamino)-phenyl]-cyclopentylmethyl}-amide

N-{4-[2-(Cyclopropanecarbonyl-amino)-1,1-dimethyl-ethyl]-phenyl}-3,4-dimethoxy-benzamide

N-[4-(2-Benzoylamino-1,1-dimethyl-ethyl)-phenyl]-3,4-dimethoxy-benzamide

Furan-2-carboxylic acid {2-[4-(3,4-dimethoxy-benzoylamino)-phenyl]-2-methyl-propyl}-amide

Benzothiazole-2-carboxylic acid {2-[4-(3,4-dimethoxy-benzoylamino)-phenyl]-2-methyl-propyl}-amide

N-{4-[1,1-Dimethyl-2-(3-phenyl-propionylamino)-ethyl]-phenyl}-3,4-dimethoxy-benzamide

N-{4-[2-(Cyclopentanecarbonyl-amino)-1,1-dimethyl-ethyl]-phenyl}-3,4-dimethoxy-benzamide

N-{2-[4-(3,4-Dimethoxy-benzoylamino)-phenyl]-2-methyl-propyl}-isonicotinamide

N-[4-(1,1-Dimethyl-2-propionylamino-ethyl)-phenyl]-3,4-dimethoxy-benzamide

3,4-Dimethoxy-N-{4-[2-(2-methoxy-acetylamino)-1,1-dimethyl-ethyl]-phenyl}-benzamide

3,4-Dimethoxy-N-(4-{1-[(2-methoxy-ethyl)-methyl-carbamoyl]-cyclopentyl}-phenyl)-benzamide

N-{4-[2-(4-Fluoro-benzoylamino)-1,1-dimethyl-ethyl]-phenyl}-3,4-dimethoxy-benzamide

Pyrazolo[1,5-a]pyridine-3-carboxylic acid {2-[4-(3,4-dimethoxy-benzoylamino)-phenyl]-2-methyl-propyl}-amide

N-{4-[2-(2-Cyclopentyl-acetylamino)N-{4-[2-(2-Cyclopentyl-acetylamino)methoxy-benzamide

3,4-Dimethoxy-N-{4-[1-methyl-1-(5-phenyl-[1,2,4]oxadiazol-3-yl)-ethyl]-phenyl}-benzamide

N-{4-[1,1-Dimethyl-2-(2,2,2-trifluoro-acetylamino)-ethyl]-phenyl}-3,4-dimethoxy-benzamide

N-{4-[2-(Acetyl-methyl-amino)-1,1-dimethyl-ethyl]-phenyl}-3,4-dimethoxy-benzamide

N-{4-[1,1-Dimethyl-2-(3-methyl-butyrylamino)-ethyl]-phenyl}-3,4-dimethoxy-benzamide

N-{4-[1,1-Dimethyl-2-(2-phenoxy-acetylamino)-ethyl]-phenyl}-3,4-dimethoxy-benzamide

5-Methyl-2-phenyl-2H-[1,2,3]triazole-4-carboxylic acid {2-[4-(3,4-dimethoxy-benzoylamino)-phenyl]-2-methyl-propyl}-amide

3,4-Dimethoxy-N-{4-[2-(2-methoxy-benzoylamino)-1,1-dimethyl-ethyl]-phenyl}-benzamide

N-(4-{2-[2-(2,5-Dimethyl-thiazol-4-yl)-acetylamino]-1,1-dimethyl-ethyl}-phenyl)-3,4-dimethoxy-benzamide

{2-[4-(3,4-Dimethoxy-benzoylamino)-phenyl]-2-methyl-propyl}-carbamic acid methyl ester

3,4-Dimethoxy-N-{4-[1-methyl-1-(5-phenoxymethyl-[1,2,4]oxadiazol-3-yl)-ethyl]-phenyl}-benzamide

N-{4-[1-(Acetylamino-methyl)-cyclopropyl]-phenyl}-3,4-dimethoxy-benzamide

5-Oxo-pyrrolidine-2-carboxylic acid {2-[4-(3,4-dimethoxy-benzoylamino)-phenyl]-2-methyl-propyl}-amide

Tetrahydro-pyran-4-carboxylic acid {2-[4-(3,4-dimethoxy-benzoylamino)-phenyl]-2-methyl-propyl}-amide

N-(4-{1,1-Dimethyl-2-[((1S,2S)-2-phenyl-cyclopropanecarbonyl)-amino]-ethyl}-phenyl)-3,4-dimethoxy-bezamide

5-Chloro-1H-indole-3-carboxylic acid {2-[4-(3,4-dimethoxy-benzoylamino)-phenyl]-2-methyl-propyl}-amide

1-Methyl-1H-indazole-3-carboxylic acid {2-[4-(3,4-dimethoxy-benzoylamino)-phenyl]-2-methyl-propyl}-amide

N-{4-[1,1-Dimethyl-2-(2-oxo-oxazolidin-3-yl)-ethyl]-phenyl}-3,4-dimethoxy-benzamide

1,3-Dimethyl-1H-thieno[2,3-c]pyrazole-5-carboxylic acid {2-[4-(3,4-dimethoxy-benzoylamino)-phenyl]-2-methyl-propyl}-amide

1-Methyl-1H-pyrrole-2-carboxylic acid {2-[4-(3,4-dimethoxy-benzoylamino)-phenyl]-2-methyl-propyl}-amide

2-Dimethylamino-thiazole-4-carboxylic acid {2-[4-(3,4-dimethoxy-benzoylamino)-phenyl]-2-methyl-propyl}-amide

2-Acetylamino-thiazole-4-carboxylic acid {2-[4-(3,4-dimethoxy-benzoylamino)-phenyl]-2-methyl-propyl}-amide

Thiazole-4-carboxylic acid {2-[4-(3,4-dimethoxy-benzoylamino)-phenyl]-2-methyl-propyl}-amide

1H-Imidazole-4-carboxylic acid {2-[4-(3,4-dimethoxy-benzoylamino)-phenyl]-2-methyl-propyl}-amide

N-{4-[1,1-Dimethyl-2-(2-phenoxy-propionylamino)-ethyl]-phenyl}-3,4-dimethoxy-benzamide

3,5-Dimethyl-isoxazole-4-carboxylic acid {2-[4-(3,4-dimethoxy-benzoylamino)-phenyl]-2-methyl-propyl}-amide

N-{2-[4-(3,4-Dimethoxy-benzoylamino)-phenyl]-2-methyl-propyl}-nicotinamide

Thiophene-2-carboxylic acid {2-[4-(3,4-dimethoxy-benzoylamino)-phenyl]-2-methyl-propyl}-amide

[1,2,3]Thiadiazole-4-carboxylic acid {2-[4-(3,4-dimethoxy-benzoylamino)-phenyl]-2-methyl-propyl}-amide

Thiophene-3-carboxylic acid {2-[4-(3,4-dimethoxy-benzoylamino)-phenyl]-2-methyl-propyl}-amide

1-Methyl-1H-imidazole-4-carboxylic acid 2-[4-(3,4-dimethoxy-benzoylamino)-phenyl]-2-methyl-propyl}-amide

Pyridine-2-carboxylic acid {2-[4(3,4-dimethoxy-benzoylamino)-phenyl]-2-methyl-propyl}-amide

N-[4-(Cyano-dimethyl-methyl)-2-methoxy-phenyl]-3,4-dimethoxy-benzamide

Thiazole-5-carboxylic acid {2-[4-(3,4-dimethoxy-benzoylamino)-phenyl]-2-methyl-propyl}-amide

3,4-Dimethoxy-N-{4-[1-methyl-1-(5-methyl-[1,3,4]oxadiazol-2-yl)-ethyl]-phenyl}-benzamide

N-[3-(Cyano-dimethyl-methyl)-phenyl]-3,4-dimethoxy-benzamide

3,4-Dimethoxy-N-[4-(2-methoxy-1,1-dimethyl-ethyl)-phenyl]-benzamide

1-Methyl-1H-indole-3-carboxylic acid {2-[4-(3,4-dimethoxy-benzoylamino)-phenyl]-2-methyl-propyl}-amide

Pyrazolo[1,5-a]pyrimidine-3-carboxylic acid {2-[4-(3,4-dimethoxy-benzoylamino)-phenyl]-2-methyl-propyl}-amide

1-Methyl-1H-pyrazole-3-carboxylic acid {2-[4-(3,4-dimethoxy-benzoylamino)-phenyl]-2-methyl-propyl}-amide

4-Bromo-1-methyl-1H-pyrazole-3-carboxylic acid {2-[4(3,4-dimethoxy-benzoylamino)-phenyl]-2-methyl-propyl}-amide

1,5-Dimethyl-1H-pyrazole-3-carboxylic acid {2-[4-(3,4-dimethoxy-benzoylamino)-phenyl]-2-methyl-propyl}-amide

2,5-Dimethyl-2H-pyrazole-3-carboxylic acid {2-[4-(3,4-dimethoxy-benzoylamino)-phenyl]-2-methyl-propyl}-amide

1H-Indazole-3-carboxylic acid {2-[4-(3,4-dimethoxy-benzoylamino)-phenyl]-2-methyl-propyl}-amide

N-[3-(2-Acetylamino-1,1-dimethyl-ethyl)-phenyl]-3,4-dimethoxy-benzamide

N-[2-Chloro-4-(cyano-dimethyl-methyl)-phenyl]-3,4-dimethoxy-benzamide

N-[3-(1-Cyano-cyclopropyl)-phenyl]-3,4-dimethoxy-benzamide

1-Methyl-1H-indazole-3-carboxylic acid {2-[3-(3,4-dimethoxy-benzoylamino)-phenyl]-2-methyl-propyl}-amide

1-Methyl-4-phenyl-1H-pyrazole-3-carboxylic acid {2-[4-(3,4-dimethoxy-benzoylamino)-phenyl]-2-methyl-propyl}-amide

N-[4-(Cyano-dimethyl-methyl)-2-methyl-phenyl]-3,4-dimethoxy-benzamide

N-[4-(Cyano-dimethyl-methyl)-3-methyl-phenyl]-3,4-dimethoxy-benzamide

N-[4-(Cyano-dimethyl-methyl)-3-fluoro-phenyl]-3,4-dimethoxy-benzamide

N-[4-(Cyano-methyl-phenyl-methyl)-phenyl]-3,4-dimethoxy-benzamide

N-[3-Chloro-4-(cyano-dimethyl-methyl)-phenyl]-3,4-dimethoxy-benzamide

N-[4-(Cyano-dimethyl-methyl)-3-trifluoromethyl-phenyl]-3,4-dimethoxy-benzamide

N-[4-(Cyano-dimethyl-methyl)-3-methoxy-phenyl]-3,4-dimethoxy-benzamide

1H-Indole-3-carboxylic acid 2-[4-(3,4-dimethoxy-benzoylamino)-phenyl]-2-methyl-propyl}-amide

1-Methyl-1H-indazole-3-carboxylic acid {1-[3-(3,4-dimethoxy-benzoylamino)-phenyl]-cyclopentylmethyl}-amide

1H-Indazole-3-carboxylic acid {1-[3-(3,4-dimethoxy-benzoylamino)-phenyl]-cyclopentylmethyl}-amide

1-Acetyl-1H-indole-3-carboxylic acid {2-[4-(3,4-dimethoxy-benzoylamino)-phenyl]-2-methyl-propyl}-amide

1-Isopropyl-1H-indole-3-carboxylic acid {2-[4-(3,4-dimethoxy-benzoylamino)-phenyl]-2-methyl-propyl}-amide

1H-Indole-2-carboxylic acid {2-[4-(3,4-dimethoxy-benzoylamino)-phenyl]-2-methyl-propyl}-amide

1H-Indazole-3-carboxylic acid {2-[3-(3,4-dimethoxy-benzoylamino)-phenyl]-2-methyl-propyl}-amide

1-Methyl-1H-indole-2-carboxylic acid {2-[4-(3,4-dimethoxy-benzoylamino)-phenyl]-2-methyl-propyl}-amide

1H-Indazole-3-carboxylic acid {2-[4-(3,4-dimethoxy-benzoylamino)-2-methyl-phenyl]-2-methyl-propyl}-amide

1-Methyl-1H-Indazole-3-carboxylic acid {2-[4-(3,4-dimethoxy-benzoylamino)-2-methyl-phenyl]-2-methyl-propyl}-amide

N-[3-Bromo-4-(cyano-dimethyl-methyl)-phenyl]-3,4-dimethoxy-benzamide

5-Methoxy-1H-indazole-3-carboxylic acid {2-[4-(3,4-dimethoxy-benzoylamino)-phenyl]-2-methyl-propyl}-amide

1H-Indazole-3-carboxylic acid {2-[2-chloro-4-(3,4-dimethoxy-benzoylamino)-phenyl]-2-methyl-propyl}-amide

N-[4-(2-Cyano-1,1-dimethyl-ethyl)-phenyl]-3,4-dimethoxy-benzamide

N-{4-[1,1-Dimethyl-2-(4-sulfamoyl-benzoylamino)-ethyl]-phenyl}-3,4-dimethoxy-benzamide

Benzo[b]thiophene-3-carboxylic acid {2-[4-(3,4-dimethoxy-benzoylamino)-phenyl]-2-methyl-propyl}-amide

6-Oxo-1,6-dihydro-pyridazine-3-carboxylic acid {2-[4-(3,4-dimethoxy-benzoylamino)-phenyl]-2-methyl-propyl}-amide

N-[6-(Cyano-dimethyl-methyl)-biphenyl-3-yl]-3,4-dimethoxy-benzamide

5-Fluoro-1H-indazole-3-carboxylic acid {2-[4-(3,4-dimethoxy-benzoylamino)-phenyl]-2-methyl-propyl}-amide

N-[3,5-Dichloro-4-(cyano-dimethyl-methyl)-phenyl]-3,4-dimethoxy-benzamide

1-Methyl-1H-indazole-3-carboxylic acid {2-[2-chloro-4-(3,4-dimethoxy-benzoylamino)-phenyl]-2-methyl-propyl}-amide

N-[4-(2-Acetylamino-1,1-dimethyl-ethyl)-3-chloro-phenyl]-3,4-dimethoxy-benzamide

1H-Indazole-6-carboxylic acid {2-[4-(3,4-dimethoxy-benzoylamino)-phenyl]-2-methyl-propyl}-amide

1-Oxo-1,2-dihydro-isoquinoline-3-carboxylic acid {2-[4-(3,4-dimethoxy-benzoylamino)-phenyl]-2-methyl-propyl}-amide

N-[4-Chloro-3-(cyano-dimethyl-methyl)-phenyl]-3,4-dimethoxy-benzamide

N-{4-[2-(2-1H-Indol-3-yl-acetylamino)-1,1-dimethyl-ethyl]-phenyl}-3,4-benzamide

5-Methyl-1H-pyrazole-3-carboxylic acid {2-[4-(3,4-dimethoxy-benzoylamino)-phenyl]-2-methyl-propyl}-amide

2-Oxo-1,2-dihydro-quinoline-3-carboxylic acid {2-[4-(3,4-dimethoxy-benzoylamino)-phenyl]-2-methyl-propyl}-amide

N-{4-[2-(2-Hydroxy-benzoylamino)-1,1-dimethyl-ethyl]-phenyl}-3,4-dimethoxy-benzamide

N-{4-[2-(4-Hydroxy-benzoylamino)-1,1-dimethyl-ethyl]-phenyl}-3,4-dimethoxy-benzamide

1-Methyl-1H-indazole-3-carboxylic acid {2-[2-chloro-5-(3,4-dimethoxy-benzoylamino)-phenyl]-2-methyl-propyl}-amide

N-[4-(Cyano-dimethyl-methyl)-3-pyridin-3-yl-phenyl]-3,4-dimethoxy-benzamide

N-[4-(Cyano-dimethyl-methyl)-3-ethyl-phenyl]-3,4-dimethoxy-benzamide

N-{4-[2-(3-Hydroxy-benzoylamino)-1,1-dimethyl-ethyl]-phenyl}-3,4-dimethoxy-benzamide

1-Isopropyl-1H-indazole-3-carboxylic acid {2-[4-(3,4-dimethoxy-benzoylamino)-phenyl]-2-methyl-propyl}-amide

1-Butyl-1H-indazole-3-carboxylic acid {2-[4-(3,4-dimethoxy-benzoylamino)-phenyl]-2-methyl-propyl}-amide

3,5-Dimethyl-1H-pyrazole-4-carboxylic acid {2-[4-(3,4-dimethoxy-benzoylamino)-phenyl]-2-methyl-propyl}-amide

5-Chloro-1H-indazole-3-carboxylic acid {2-[4-(3,4-dimethoxy-benzoylamino)-phenyl]-2-methyl-propyl}-amide

5-Fluoro-1H-indole-3-carboxylic acid {2-[4-(3,4-dimethoxy-benzoylamino)-phenyl]-2-methyl-propyl}-amide

5-Methoxy-1H-indole-3-carboxylic acid {2-[4-(3,4-dimethoxy-benzoylamino)-phenyl]-2-methyl-propyl}-amide

N-4-[2-(4-Methanesulfonylamino-benzoylamino)-1,1-dimethyl-ethyl]-phenyl}-3,4-dimethoxy-benzamide

1H-Indole-5-carboxylic acid {2-[4-(3,4-dimethoxy-benzoylamino)-phenyl]-2-methyl-propyl}-amide

6-Fluoro-1H-indazole-3-carboxylic acid {2-[4-(3,4-dimethoxy-benzoylamino)-phenyl]-2-methyl-propyl}-amide

5-Phenyl-1H-pyrazole-3-carboxylic acid {2-[4-(3,4-dimethoxy-benzoylamino)-phenyl]-2-methyl-propyl}-amide

3-Phenyl-1H-pyrazole-4-carboxylic acid {2-[4-(3,4-dimethoxy-benzoylamino)-phenyl]-2-methyl-propyl}-amide

3,5-Dimethyl-1H-indole-2-carboxylic acid {2-[4-(3,4-dimethoxy-benzoylamino)-phenyl]-2-methyl-propyl}-amide

1H-Indazole-5-carboxylic acid {2-[4-(3,4-dimethoxy-benzoylamino)-phenyl]-2-methyl-propyl}-amide

3-Methyl-1H-pyrazole-4-carboxylic acid {2-[4-(3,4-dimethoxy-benzoylamino)-phenyl]-2-methyl-propyl}-amide

1H-Indole-4-carboxylic acid {2-[4-(3,4-dimethoxy-benzoylamino)-phenyl]-2-methyl-propyl}-amide

7-Fluoro-1H-indazole-3-carboxylic acid {2-[4-(3,4-dimethoxy-benzoylamino)-phenyl]-2-methyl-propyl}-amide

N-[4-(2-Acetylamino-1,1-dimethyl-ethyl)-3-fluoro-phenyl]-3,4-dimethoxy-benzamide

N-[6-(Cyano-dimethyl-methyl)-4′-trifluoromethyl-biphenyl-3-yl]-3,4-dimethoxy-benzamide

N-[2′-Chloro-6-(cyano-dimethyl-methyl)-biphenyl-3-yl]-3,4-dimethoxy-benzamide

N-(3′-chloro-6-(2-cyanopropan-2-yl)biphenyl-3-yl)-3,4-dimethoxybenzamide

N-[4-(Cyano-dimethyl-methyl)-3-pyridin-4-yl-phenyl]-3,4-dimethoxy-benzamide

N-[4-(3-Acetyl amino-1,1-dimethyl-propyl)-phenyl]-3,4-dimethoxy-benzamide

Imidazo[1,2-a]pyridine-3-carboxylic acid {2-[4-(3,4-dimethoxy-benzoylamino)-phenyl]-2-methyl-propyl}-amide

1H-Benzoimidazole-2-carboxylic acid {2-[4-(3,4-dimethoxy-benzoylamino)-phenyl]-2-methyl-propyl}-amide

N-[4-(Cyano-dimethyl-methyl)-3-isopropenyl-phenyl]-3,4-dimethoxy-benzamide

N-[4-(Cyano-dimethyl-methyl)-3-hydroxy-phenyl]-3,4-dimethoxy-benzamide

N-[4-(Cyano-dimethyl-methyl)-3-cyclopropyl-phenyl]-3,4-dimethoxy-benzamide

N-[3-Cyano-4-(cyano-dimethyl-methyl)-phenyl]-3,4-dimethoxy-benzamide

N-[6-(Cyano-dimethyl-methyl)-4′-methyl-biphenyl-3-yl]-3,4-dimethoxy-benzamide

N-[6-(Cyano-dimethyl-methyl)-4′-methoxy-biphenyl-3-yl]-3,4-dimethoxy-benzamide

N-[4′-Chloro-6-(cyano-dimethyl-methyl)-biphenyl-3-yl]-3,4-dimethoxy-benzamide

N-[4-(Cyano-dimethyl-methyl)-3-thiophen-3-yl-phenyl]-3,4-dimethoxy-benzamide

5-Methyl-1H-indazole-3-carboxylic acid {2-[4-(3,4-dimethoxy-benzoylamino)-phenyl]-2-methyl-propyl}-amide

1H-Indole-6-carboxylic acid {2-[4-(3,4-dimethoxy-benzoylamino)-phenyl]-2-methyl-propyl}-amide

N-(4-{2-[2-(2-Methane sulfonylamino-phenyl)-acetylamino]-1,1-dimethyl-ethyl}-phenyl)-3,4-dimethoxy-benzamide

N-[4-(Cyano-dimethyl-methyl)-3-(6-methoxy-pyridin-3-yl)-phenyl]-3,4-dimethoxy-benzamide

6-Fluoro-1H-indole-3-carboxylic acid {2-[4-(3,4-dimethoxy-benzoylamino)-phenyl]-2-methyl-propyl}-amide

5-Fluoro-1H-indole-2-carboxylic acid {2-[4-(3,4-dimethoxy-benzoylamino)-phenyl]-2-methyl-propyl}-amide

1H-Pyrrolo[2,3-b]pyridine-3-carboxylic acid {2-[4-(3,4-dimethoxy-benzoylamino)-phenyl]-2-methyl-propyl}-amide

4-Fluoro-1H-indole-3-carboxylic acid {2-[4-(3,4-dimethoxy-benzoylamino)-phenyl]-2-methyl-propyl}-amide

7-Fluoro-1H-indole-3-carboxylic acid {2-[4-(3,4-dimethoxy-benzoylamino)-phenyl]-2-methyl-propyl}-amide

1H-Pyrrolo[3,2-b]pyridine-3-carboxylic acid {2-[4-(3,4-dimethoxy-benzoylamino)-phenyl]-2-methyl-propyl}-amide

1H-Pyrrolo[3,2-c]pyridine-3-carboxylic acid {2-[4-(3,4-dimethoxy-benzoylamino)-phenyl]-2-methyl-propyl}-amide

N-[4-(Cyano-dimethyl-methyl)-3-pyridin-2-yl-phenyl]-3,4-dimethoxy-benzamide

N-[4-(Cyano-dimethyl-methyl)-3-pyrimidin-5-yl-phenyl]-3,4-dimethoxy-benzamide

Imidazo[1,2-a]pyrimidine-3-carboxylic acid {2-[4-(3,4-dimethoxy-benzoylamino)-phenyl]-2-methyl-propyl}-amide

Imidazo[1,2-a]pyrimidine-2-carboxylic acid {2-[4-(3,4-dimethoxy-benzoylamino)-phenyl]-2-methyl-propyl}-amide

N-(4-{2-[2-(5-Fluoro-indol-1-yl)-acetylamino]-1,1-dimethyl-ethyl}-phenyl)-3,4-dimethoxy-benzamide

1H-Pyrrolo[2,3-b]pyridine-5-carboxylic acid {2-[4-(3,4-dimethoxy-benzoylamino)-phenyl]-2-methyl-propyl}-amide

Imidazo[1,2-a]pyridine-6-carboxylic acid {2-[4-(3,4-dimethoxy-benzoylamino)-phenyl]-2-methyl-propyl}-amide

5-Fluoro-1-(2-methoxy-ethyl)-1H-indole-3-carboxylic acid {2-[4-(3,4-dimethoxy-benzoylamino)-phenyl]-2-methyl-propyl}-amide

1-(3-Dimethylamino-propyl)-5-fluoro-1H-indole-3-carboxylic acid {2-[4-(3,4-dimethoxy-benzoylamino)-phenyl]-2-methyl-propyl}-amide

6-Fluoro-1H-benzoimidazole-2-carboxylic acid {2-[4-(3,4-dimethoxy-benzoylamino)-phenyl]-2-methyl-propyl}-amide

Imidazo[1,2-a]pyridine-3-carboxylic acid {3-[4-(3,4-dimethoxy-benzoylamino)-phenyl]-3-methyl-butyl}-amide

N-[4-(2-Acetylamino-1,1-dimethyl-ethyl)-3-pyridin-3-yl-phenyl]-3,4-dimethoxy-benzamide

3H-Imidazo[4,5-b]pyridine-2-carboxylic acid {3-[4-(3,4-dimethoxy-benzoylamino)-phenyl]-3-methyl-butyl}-amide

3H-Imidazo[4,5-b]pyridine-6-carboxylic acid {2-[4-(3,4-dimethoxy-benzoylamino)-phenyl]-2-methyl-propyl}-amide

3H-Imidazo[4,5-b]pyridine-6-carboxylic acid {2-[4-(3,4-dimethoxy-benzoylamino)-2-ethyl-phenyl]-2-methyl-propyl}-amide

3H-Imidazo[4,5-b]pyridine-6-carboxylic acid {3-[4-(3,4-dimethoxy-benzoylamino)-phenyl]-3-methyl-butyl}-amide

N-[4-(2-Acetylamino-1,1-dimethyl-ethyl)-3-ethyl-phenyl]-3,4-dimethoxy-benzamide

1H-Pyrrolo[2,3-b]pyridine-5-carboxylic acid {2-[4-(3,4-dimethoxy-benzoylamino)-phenyl]-2-methyl-propyl}-amide

1H-Pyrrolo[2,3-b]pyridine-5-carboxylic acid {2-[4-(3,4-dimethoxy-benzoylamino)-phenyl]-2-methyl-propyl}-amide

Imidazo[1,2-a]pyridine-3-carboxylic acid {2-[3-(3,4-dimethoxy-benzoylamino)-phenyl]-2-methyl-propyl}-amide

N-[4-(Cyano-dimethyl-methyl)-3-morpholin-4-yl-phenyl]-3,4-dimethoxy-benzamide

N-[4-(Cyano-dimethyl-methyl)-3-(1-methyl-1H-pyrazol-4-yl)-phenyl]-3,4-dimethoxy-benzamide

N-[4-(Cyano-dimethyl-methyl)-3-thiophen-2-yl-phenyl]-3,4-dimethoxy-benzamide

1H-Benzoimidazole-5-carboxylic acid {2-[4-(3,4-dimethoxy-benzoylamino)-phenyl]-2-methyl-propyl}-amide

2-Methyl-1H-benzoimidazole-5-carboxylic acid {2-[4-(3,4-dimethoxy-benzoylamino)-phenyl]-2-methyl-propyl}-amide

1,2-Dimethyl-1H-benzoimidazole-5-carboxylic acid {2-[4-(3,4-dimethoxy-benzoylamino)-phenyl]-2-methyl-propyl}-amide

1,3-Dimethyl-2-oxo-2,3-dihydro-1H-benzoimidazole-5-carboxylic acid {2-[4-(3,4-dimethoxy-benzoylamino)-phenyl]-2-methyl-propyl}-amide

6-Fluoro-imidazo[1,2-a]pyridine-3-carboxylic acid {2-[4-(3,4-dimethoxy-benzoylamino)-phenyl]-2-methyl-propyl}-amide

Imidazo[1,2-a]pyridine-3-carboxylic acid {2-[4-(3,4-dimethoxy-benzoylamino)-phenyl]-3-hydroxy-2-methyl-propyl}-amide

3H-Imidazo[4,5-b]pyridine-2-carboxylic acid {2-[4-(3,4-dimethoxy-benzoylamino)-phenyl]-2-methyl-propyl}-amide

3H-Imidazo[4,5-c]pyridine-2-carboxylic acid {2-[4-(3,4-dimethoxy-benzoylamino)-phenyl]-2-methyl-propyl}-amide

1H-Pyrrolo[2,3-c]pyridine-3-carboxylic acid {2-[4-(3,4-dimethoxy-benzoylamino)-phenyl]-2-methyl-propyl}-amide

5-Fluoro-1H-pyrrolo[2,3-b]pyridine-3-carboxylic acid {2-[4-(3,4-dimethoxy-benzoylamino)-phenyl]-2-methyl-propyl}-amide

7-Fluoro-1H-pyrrolo[2,3-c]pyridine-3-carboxylic acid {2-[4-(3,4-dimethoxy-benzoylamino)-phenyl]-2-methyl-propyl}-amide

5-Chloro-1H-pyrrolo[2,3-c]pyridine-3-carboxylic acid {2-[4-(3,4-dimethoxy-benzoylamino)-phenyl]-2-methyl-propyl}-amide

5-Fluoro-1H-pyrrolo[2,3-c]pyridine-3-carboxylic acid {2-[4-(3,4-dimethoxy-benzoylamino)-phenyl]-2-methyl-propyl}-amide

N-[4-(Cyano-dimethyl-methyl)-3-vinyl-phenyl]-3,4-dimethoxy-benzamide

N-(4-(4-acetamido-2-methylbutan-2-yl)-3-(pyridin-3-yl)-phenyl)-3,4-dimethoxybenzamide

The present invention includes both possible stereoisomers and includes not only racemic compounds but the individual enantiomers as well.

The present invention relates to the pharmaceutically acceptable acid addition salts of compounds of the formula (I) and compositions including such compounds with pharmaceutically acceptable carriers or excipients.

The present invention relates to a method of treating or preventing a condition in a mammal, including a human, the treatment or prevention of which is affected or facilitated by the modulation effect of FSH antagonists.

The present invention relates to a method useful for treating or preventing disorders selected from the group consisting of uterine fibroids, endometriosis, polycystic ovarian disease, dysfunctional uterine bleeding, osteoporosis, breast cancer and ovarian cancer; depletion of oocytes; spermatocyte depletion; or female and male contraception, in a subject in need thereof, comprising administering to the subject a therapeutically effective amount of the compound of claim 1.

The present invention relates to pharmaceutical compositions which provide from about 0.01 to 1000 mg of the active ingredient per unit dose. The compositions may be administered by any suitable route. For example orally in the form of capsules, etc. . . . , parenterally in the form of solutions for injection, topically in the form of onguents or lotions, ocularly in the form of eye-drops, rectally in the form of suppositories, intranasally or transcutaneously in the form of delivery system like patches.

The pharmaceutical formulations of the invention may be prepared by conventional methods in the art; the nature of the pharmaceutical composition employed will depend on the desired route of administration. The total daily dose usually ranges from about 0.05-2000 mg.

Methods of Synthesis

The compounds of this invention can be prepared according to standard chemical methodology described in the literature from either commercially available starting material, or starting material that can be prepared as described in the literature.

Compounds of general formula I may be prepared according to the following synthetic schemes. Unless otherwise noted, R₁,R₂,R₄,R₅,R₆, R₇, R₈, X₁, G¹ₙ, M and Q are defined above.

In all the schemes described below, it is well understood that protecting groups for sensitive or reactive groups are employed where necessary in accordance with general principles of chemistry. Protecting groups are manipulated according to standard methods of organic synthesis (Green T. W. and Wuts P. G. M. (1991) Protecting Groups in Organic Synthesis, John Wiley et Sons). These groups are removed at a convenient stage of the compound synthesis using methods that are readily apparent to those skilled in the art. The selection of process as well as the reaction conditions and order of their execution shall be consistent with the preparation of compounds of formula I.

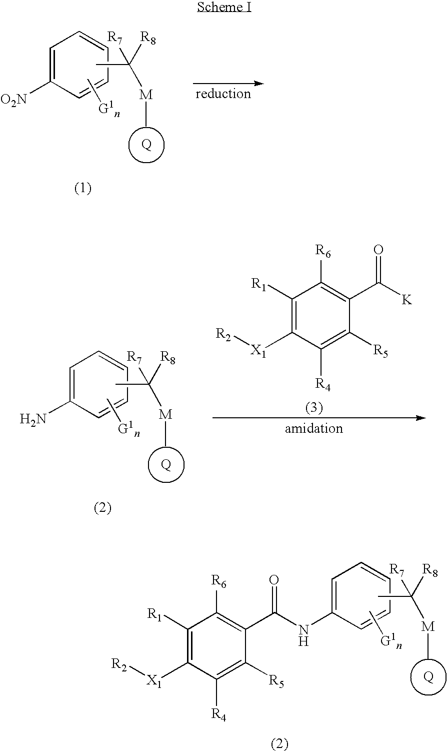

According to Scheme I, the nitro compounds of general formula (1) can be reduced to the corresponding aniline derivatives (2) under conditions readily apparent to those skilled in the art. The nitro group can be most conveniently reduced by catalytic hydrogenation, in presence of a suitable catalyst such as palladium or platinum catalyst. This reaction is typically carried out in lower alcohol (methanol, ethanol and the like), at about atmospheric pressure of hydrogen and at about room temperature.

Compounds of general formula (I) can be prepared by coupling anilines of formula (2) with compounds of formula (3), in which K can be either a hydroxyl group or a halide such as chlorine (a survey of the suitable reactions is given by Carey, F. A. and Sundeberg, R. J. Advanced Organic Chemistry, Third Edition (1990), Plenum Press, New York and London, pg 145). Compounds (3) are either commercially available, or are known in the art, or can be readily prepared using procedures which are analogue to those reported in the literature for known compounds. The coupling between anilines (2) and reagent (3) may be conducted in several ways. For instance, in the case where K is halogen such as chlorine, the aniline (2) is reacted with the suitable acyl halide (3), using methods that are readily apparent to those skilled in the art. The reaction may be promoted by a base such as triethylamine, pyridine, 4-dimethylaminopyridine and the like, either neat or in a suitable solvent (e.g. dichloromethane). This reaction is usually performed in a temperature range from 0° C. to 130° C. over a period of 1 hour up to 74 hours. The reaction may be conducted under conventional heating (using an oil bath) or under microwave heating. The reaction may be carried out in an open vessel or in a sealed tube. In some embodiments of the present invention, the needed acyl halide (3) can be readily prepared from the corresponding acid (3) (K═OH). This activation can be effected according to one of the standard procedures broadly reported in the literature. For instance, treatment of acid (3) (K═OH) with one or more equivalents of oxalyl chloride in the presence of a catalytic amount of DMF in a halocarbon solvent, such as dichlormethane, at temperature ranging form 0° C. to 35° C., affords the required acyl chloride (3) (K═Cl).

In another process for the preparation of compound (I) of the present invention, the aniline (2) can be activated by treatment with a strong base (e.g. sodium hydride) in an aprotic solvent such as acetonitrile, at about room temperature. Subsequent reaction of the activated intermediates (salt of the aniline (2)) with the appropriately substituted acyl halide (3), in which for example K is chlorine, leads to the desired compounds of formula (I).

Alternatively, compounds (I) can be efficiently prepared by the condensation between anilines (2) and acid (3) (K═OH) under standard amidation and peptide coupling conditions. For instance, treatment of the acid (3) (K=OH) with one or more equivalent of a commercially available condensation agents such as a carbodiimide (e.g. 1-(3-dimethylamino)propyl)-3-ethylcarbodiimide hydrochloride (EDC)), in the presence of N-hydroxybenzotriazole (HOBt) (or commercially available analogues) followed by reaction of the activated intermediate with anilines (2), results in the formation of compounds (I). An organic base such as triethylamine and the like may be also present in the reaction mixture. The activated intermediate can be either isolated, or pre-formed or generated in situ. Suitable solvents for the coupling include, but are not limited to, halocarbon solvent (e.g. dichloromethane), dioxane and acetonitrile. The reaction typically proceeds at temperature range from 0° C. up to 170° C., for a time in the range of about 1 hour up to 72 hours. The reaction may be carried out under conventional heating (using an oil bath) or under microwave irradiation. The reaction may be conducted either in an open vessel or in a sealed tube.

In another process for the preparation of the compounds of the present invention, acid (3) (K═OH) can be activated with other commercially available activating agents such as bromotripyrrolidinophosphoniuni hexafluorophosphate (PyBrOP), in the suitable aprotic solvent (e.g. dichlorometahane), at about room temperature. Subsequent reaction of the activated intermediate with anilines (2) provides the desired compound of formula (I). The reaction may also require the use of an organic base such as diisopropylethylamine and the like and usually proceeds at about room temperature.

Alternatively, acylation of anilines (2) to give compounds of general formula (I) can be accomplished using procedures which convert in situ the acid (3) (K═OH) into the corresponding acyl halides. For example, anilines (2) are reacted with acids (3) (K═OH) in presence of triphenylphosphine and a halocarbon solvent such as carbon tetrachloride or dichloromethane, at about room temperature, in a maximum period of time of 16 hours (Lee, J. B. J. Am. Chem. Soc., 1966, 88, 3440).

The method of choice for the preparation of compounds of formula (I) from anilines (2) and compounds (3) is ultimately chosen on the bases the reactivity of the anilines (2), the commercial availability of reagents such as (3) and the compatibility with the sensitive groups present in both the starting materials.

The required appropriately substituted nitro compounds of formula (1) of Scheme I in which M is a bond and Q is a nitrile such as in compounds of general formula (1-a), can be either commercially available or conveniently prepared as shown in Scheme II.

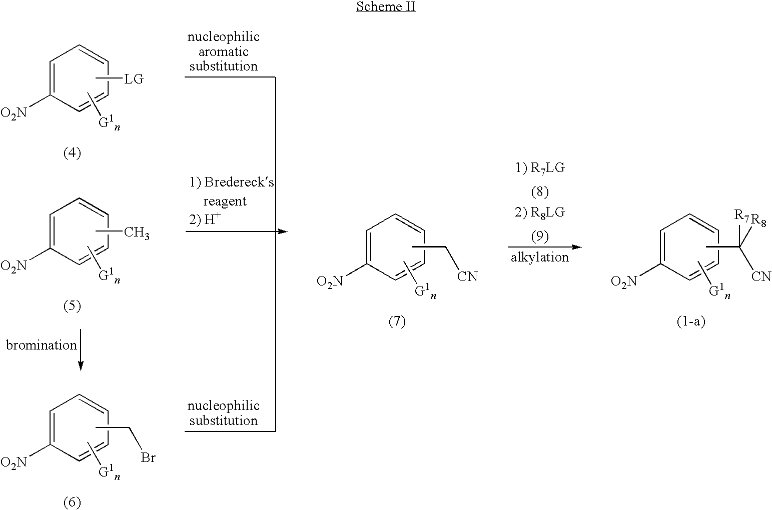

The nitro-phenyl-acetonitriles of formula (7), required as precursors for the preparation of compounds of general formula (1-a) according to Scheme II, are commercially available or may be conveniently prepared starting from compounds (4), in which LG is a suitable leaving group such as an halide (e.g chlorine or fluorine, most preferably fluorine), by means of nucleophilic aromatic substitution. In a typical experiment, this reaction may be carried out treating compound (4) (e.g. 1-chloro-4-nitro-trifluormethyl-benzene) with an acetonitrile derivative (preferably ethyl-cyanoacetate and the like), in the presence of a base such as an alkali metal carbonate, hydroxide or hydride (e.g. potassium carbonate, potassium hydroxide, sodium hydride) in a suitable solvent such as DMSO, DMF, dioxane and the like. Potassium iodine may be also present in the reaction mixture. The reaction typically proceeds at temperature ranging from 0° C. to 100° C. over a period ranging from 2 hours up to 72 hours. Subsequent hydrolysis of the ester moiety and decarboxylation of the resulting carboxylic acid provides the desired intermediates of general formula (7). This reaction may be conducted by treating the ester intermediate with a strong acid, such as hydrochloric or acetic acid, either neat or in a suitable solvent (e.g. dimethyl sulfoxide (DMSO), dioxane, water and the like), at a temperature ranging from 80° C. to 165° C. over a period ranging from 30 minutes up to 30 hours. Salts such as lithium chloride may be also employed in this process (Ahlenius et al, Eur. J. Med. (1996), 31, 133-142).

Alternatively, the compounds of general formula (7) may be prepared starting from the corresponding methyl-nitro-benzene (5) (e.g. (2-chloro-1-methyl-4-nitro-benzene), by first reacting them with Bredereck's reagent (tert-butoxy bis(dimethylamino)methane), followed by reaction of the resulting enamine intermediates with an acid, such us hydroxylamine-O-sulfonic acid (Brederck, H. et al. Chem. Ber. 1968, 101,12, 4048-4056).

In another process for the synthesis of compounds (7), the starting material (5) (e.g. 1-chloro-2-methyl-4-nitro-benzene) can be brominated on the benzylic carbon to prepare intermediates (6) (Lisitsyn, V. N. and Lugovskaya, E. K., JOC USSR (1974), 10, 92-95). Typically, this is most conveniently done using N-bromosuccinimide (NBS) and a catalytic amount of benzoyl peroxide in an inert solvent such as carbon tetrachloride and the like, at temperature ranging from room temperature to the reflux temperature of the solvent over a period of time ranging from 30 minutes to 8 hours. The bromine (6) can be treated with potassium cyanide in a water-ethanol mixture, at temperature about the reflux temperature of the solvent over a period of time ranging from 30 min to 16 hours, to afford intermediate (7).

According to Scheme II, compounds of the general formula (1-a), in which R₇, R₈ and G¹ₙ have the above-given meanings, may be conveniently prepared by alkylation of a nitro-phenyl-acetonitriles of formula (7), for example (4-nitro-phenyl)-acetonitrile, with the alkylating agents of general formula (8) and (9) in which R₇ and R₈ are defined herein-above, and LG is a leaving group such as an halogen atom (preferably a bromine or iodine atom). Different examples of this kind of reactions can be found in literature, for example: Ackerley, N. and Brewster, A. G. J. Med. Chem., 1985 38,10, 1608-1628; Gross et al. JOC, 1976, 41,7, 1187-1191; Cerenini, G. et al. Farmaco, 1973, 28, 265-277. R₇ in formula (8) and R₈ in formula (9) may be the same or different. R₇ and R₈ may be also connected one to the other. In this case, the alkylation reaction (for example performed using as the alkylating agent an alkyl dihalide such as 1,4-dibromo-butane) provides a compound of general formula (1-a) in which R₇ and R₈, together with the carbon atom to which they are attached, form a cyclic ring. If R₇ and R₈ are the same or are connected one to the other, the alkylation is carried out treating compounds of formula (4) with the suitable alkylating agents in presence of a base, for example sodium hydride, sodium hydroxide and the like, in a suitable inert solvent (e.g. dimethylformamide (DMF), dimethyl sulfoxide (DMSO), diethylether, toluene, tetrahydrofuran and the like). Water may be used as a co-solvent in the process. Typically, if the reaction is carried out under phase transfer conditions, the reaction is most conveniently carried out in presence of phase transfer catalyst such as tetrabuthylammonium bromide or benzyltriethylammonium chloride.

Typically the reaction is performed at temperature ranging from 0° C. to room temperature, over a period ranging from 1 hour up to 24 hours.

When R₇ and R₈ are different, the alkylation must be carried out in a stepwise manner. Thus, the compounds (7) are reacted with one or slightly more than one equivalent of a strong base (e.g. sodium hydride), in a suitable solvent such us N,N-dimethylformamide (DMF) and the like, followed by reaction with an alkylating agent of formula (8). Treatment of resulting intermediate with a second equivalent of strong base, followed by reaction with an alkylating agent of formula (9), provides the desired compounds of formula (1-a).

In another process for the preparation of the compounds of the present invention, the required appropriately substituted nitro compounds of formula (1) of Scheme I, in which M is a bond and Q is a nitrile such as in compounds of general formula (1-aa), can be conveniently prepared as shown in Scheme III.

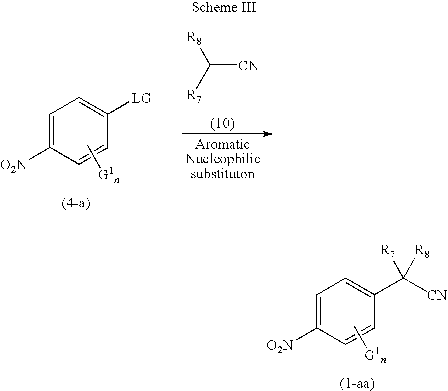

Thus, compound of formula (4-a), in which LG is a suitable leaving group such as chlorine or fluorine, can be submitted to a nucleophilic aromatic substitution to achieve desired compounds (1-aa). For instance, compound (4-a) can be reacted with a suitable reagent (10) (e.g. 2-phenyl-propionitrile) in presence of a base such as sodium hydroxide, in a suitable solvent such as acetonitrile, water and the like, at a temperature ranging from room temperature to 50° C. over a maximum period of 3 hours. When a mixture of acetonitrile and water is used as the solvent, the reaction is typically carried out using also a phase transfer catalyst, such as triethylbenzylammonium chloride or alternative commercially available analogues (Makosza, M. et al. Tetrahedron (1974), 30, 3723-3735).

As an alternative, the required appropriately substituted para-nitro compounds of general formula (1-b), which can be employed as the starting material of compounds of general formula (I) as described in Scheme I, can be conveniently prepared in Scheme IV.

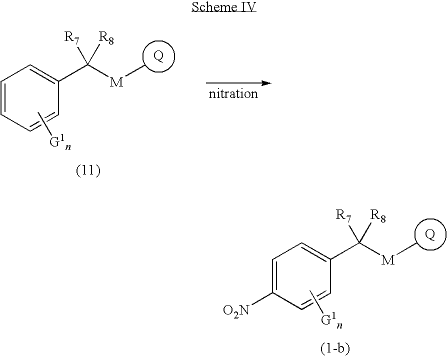

Thus, compounds of general formula (11) (e.g. 1-phenyl-cyclopropanecarbonitrile, (2-chloro-1,1-dimethyl-ethyl)-benzene and N-(1-methyl-1-phenyl-ethyl)-acetamide) can be submitted to standard nitration conditions, which includes, but are not limited to, the use of sulphuric acid in a mixture with potassium nitrate or nitric acid (Eckert, T. S., Rominger, R. L. JOC (1987), 52, 24, 5474-5475;Harvey, L. et al., Tetrahedron (1969) 25, 5019-5026). The reaction is generally performed at a temperature ranging from −7° C. to room temperature, in a period of time ranging from 1 hour to 2 hours. When (2-chloro-1,1-dimethyl-ethyl)-benzene is used as the substrate of the nitration reaction, the resulting product can be further converted to another compound of formula (1-b), in which M is a methylene group and Q is nitrile, by reaction with a cyanide donor (e.g. trimethylsilyl cyanide) in the suitable solvent (e.g. acetonitrile), heating at high temperature (up to 150° C.) for a maximum period of 6 hours. Typically the reaction is performed in presence of quaternary salts such as tetrabuthyl ammonium fluoride (Soli, E. D. et al., JOC (1999), 64, 9, 3171-3177).

Compounds (11) are either commercially available, or are known in the art, or can be readily prepared using procedures which are analogue to those reported in the literature for known compounds. For instance, in the case where M is a methylene group, Q is a carboxylic acid and G¹ₙ is hydrogen, compound (11) can be conveniently prepared by means of Lewis acid-catalyzed electrophilic aromatic substitution such as Friedel-Crafts reaction, for instance following a procedure similar to that described by Smith and Spillane in JACS, 1943, 65, 202-208 or by Hillery and Cohen in JACS, 1983, 105, 2760-2770. Thus a suitable arene such as benzene is reacted with the opportune alkene (e.g. 3-methylbut-2-enoic acid) in presence of a Lewis acid, preferably anhydrous aluminum chloride or similar. This reaction is typically conducted at a temperature ranging from 5° C. to room temperature, in a period of time ranging from 1 hour to 16 hours.

According to Scheme V, the compounds of general formula (1-a) may be converted into others compounds of general formula (1), such us (1-c), (1-d), (1-e) or (1-f), which can be used as starting materials for the synthesis of compounds of general formula (I), following the procedure reported in Scheme I.

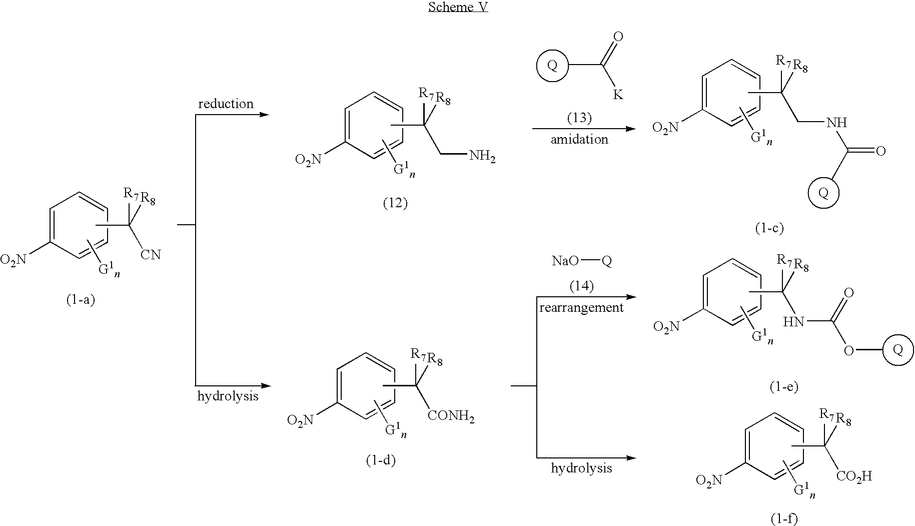

According to the scheme, the nitrile derivatives of formula (1-a) are converted into the corresponding primary amine derivatives (12), following a procedure similar to that described by Weinstock, J. et al. in J. Med. Chem., 1987,30, 7, 1166-1176. Thus, intermediates (1-a) are reacted with a reducing reagent such as borane, preferably borane-tetrahydrofuran complex, in an aprotic solvent such as tetrahydrofuran. The reaction typically proceeds by heating the reaction from ambient temperature up to the reflux temperature of the solvent, for a time of about one hour. Subsequent coupling of the resulting compounds (12) with a suitable reagent (13) affords the compounds (1-c). In the reagent (13) in Scheme V, Q has the above-given meanings and K can be halogen or —OH. For instance, in the case where K is halogen, the amines (12) are reacted with an acyl halide, preferably and acyl chloride, using methods that are readily apparent to those skilled in the art. The reaction may be promoted by a base such as triethylamine, in a suitable solvent (e.g. dichloromethane) at temperatures ranging from 0° C. to room temperature. In the case where K is —OH, the amines of formula (12) are reacted with the carboxylic acids (13), promoting the coupling with an activating agent such as 1-ethyl-3-(3-dimethylamino-propyl) carbodiimide hydrochloride or other analogues known in the art, and in the presence of 1-hydrobenzotriazole. In some embodiments of this process, the coupling is performed in presence of an organic base such as triethylamine. The reaction is typically performed in an aprotic solvent (e.g. dichloromethane), at room temperature.

In another process for preparing compounds of the present invention, the nitrile moiety of compounds (1-a) may be converted to the corresponding primary amide, such as in compounds (1-d). For instance this reaction may be conveniently performed by treating compound (1-a) with an aqueous base, such as potassium hydroxide, in a suitable solvent (e.g. ethanol), at temperature about 110° C. Typically, this reaction is most conveniently performed heating with a microwave oven.

Alternatively, treatment of compound (1-a) with an oxidizing reagent such as hydrogen peroxide, in presence of an aqueous base such as potassium carbonate, in a protic solvent such as ethanol and the like, as described for example by Erdelmeier, I. et al. in JOC, 2000, 65, 24, 8152-8157, provides the desired compounds of formula (1-d).

The resulting primary amides (1-d) may be used as a starting material for the synthesis of compounds of general formula (I), according to the procedures reported in Scheme (I). Otherwise, they can be converted either to compounds (1-e) or to compounds (14), as shown in Scheme V.

Compounds (1-e) can be achieved by means of a rearrangement reaction, such as Hofmann reaction. In a typical experiment, amides (1-d) are reacted with a hypobromide ion, which is most conveniently generated in situ by treatment of bromine with a base such as sodium alkoxyde of general formula (14) (e.g. Q-ONa is sodium methoxyde), in the corresponding alcoholic solvent (i.e. if (14) is sodium methoxyde, the solvent is methanol). The reaction temperature range is generally comprised between 0° C. and 50° C. (Timberlake, J. W. et al., JOC (1995), 60, 16, 5295-5298).

Compounds (1-f) can be prepared by the hydrolysis of the amide moiety of compounds (1-d), according to one of the standard procedures extensively reported in literature. These standard procedures include, but are not limited to, the treatment of amide (1-d) with an acid, such as hydrochloric acid, in a suitable solvent such as tetrahydrofuran and water, at the temperature of reflux of the solvent, for a period of time of about 20 hours.

Resulting compounds (1-f) may be either used as the substrate for the synthesis of compounds of general formula (I) in Scheme I, or converted to other compounds of general formula (1), such as compounds (1-g) and (1-h) in Scheme VI.

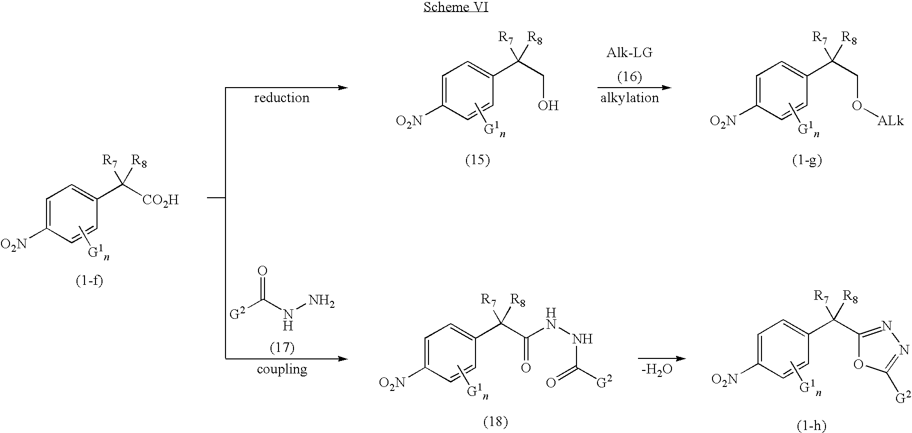

Thus, acids (1-f) can be reduced to the corresponding primary alcohol (15), under conditions well known to those skilled in the art. For example, one possible process for the synthesis of compounds (15) consists of treating acid of formula (1-f) with an activating agent, such as a chloroformate (e.g. n-butyl chloroformate), in presence of a base (e.g. N-methylmorpholine and the like) in an inert solvent such as 1,2-dimethoxyethane at low temperature (between −10 and 0° C.), for short period of time (about 10-20 minutes). Subsequent reduction of the resulting mixed anhydride with a suitable reducing reagent affords desired alcohol (15). For instance, this reduction can be conveniently performed using sodium borohydride in an alcoholic solvent (preferably ethanol). Alkylation reactions of alcohols are well known in the art. For instance, a solution of compound of formula (15) in a suitable solvent, such as tetrahydrofuran and the like, is treated with a base (e.g. sodium hydride) and the appropriate alkylating reagent of general formula (16), in which ALK is an alkyl group and LG a leaving group (e.g. an halide such as iodine). The temperature range is typically comprised between 0° C. and 35° C. (J. Chem. Soc. Perkin Trans. (1992), 1, 17, 2203-2214).

Alternatively, the acids (1-f) can be used as the starting materials for the synthesis of the [1,3,4]oxadiazole derivatives (1-h), using procedures readily apparent to those skilled in the art (a survey of the suitable reactions is given by Katritzky, A. R. and Rees, C. W., Comprehensive Heterocyclic Chemistry, First edition (1984), Pergamon Press, Oxford, volume 6, pg 440). For instance, acids (1-f) are converted to an acyl halide (most preferably an acyl chloride) by reaction with oxalyl chloride or similar reagents known in the art, in presence of a catalytic amount of N,N-dimethylformamide, in an aprotic solvent such as dichlormethane and the like, at temperatures ranging from 0° C. to room temperature. Subsequent coupling of the resulting acyl halide with an appropriately substituted hydrazide of general formula (17), which are commercially available or readily prepared following procedure described in literature, in an inert solvent such as dichloromethane and the like, at about room temperature for a maximum period of time of 16 hours, provides compounds of general formula (18). The intramolecular cyclization can be accomplished using different reaction conditions reported in literature, most notably using phosphorus oxychloride. In a typical procedure, this reaction is conducted in acetonitrile, at the temperature of reflux of the solvent, for a period of time of about 2 hours (Peet, N. P. and Sunder, S., J. Heterocycl. Chem. (1983), 20, 1355-1357).

In some processes for the preparation of the compounds of the present invention, anilines of general formula (2) in the Scheme I, can be converted to others anilines of general formula (2) before proceeding with the coupling with compounds (3). For instance, anilines (2) in which M is a bond and Q a nitrile, such as in anilines (2-a) of Scheme VII, may be conveniently converted to compounds (2-b).

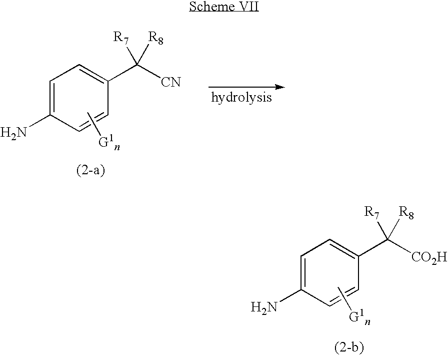

This conversion can be performed using conditions readily apparent to those skilled in the art. One notable procedure consists of treating compounds of formula (2-a) with an aqueous base, such as potassium hydroxide and the like, in an a suitable solvent such as ethanol, water and the like, at a temperature above 80° C., preferably about 100° C., for a period of time no less than 1 hour, generally about 9 hours.

When G¹ₙ in intermediate (2-a) is hydrogen, such as in compounds (2-aa) in Scheme VIII, these anilines may be converted into other anilines of formula (2-c) by means of a halogenation reaction. This is most conveniently accomplished using a halide donor, such as an N-halogen succinimide (e.g. N-chlorosuccinimide) or others analogues known in the art, in a suitable solvent, generally an alcoholic solvent (e.g. iso-propanol), at a temperature ranging from ambient to reflux temperature of the solvent over a period of about 1 hour.

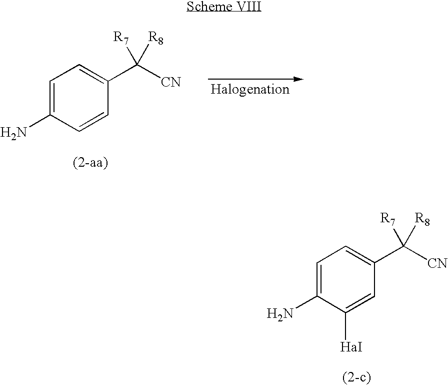

When G¹ₙ in intermediate (2-a) is an halogen (most preferably a bromine), such as in compounds (2-ab) in Scheme IX, this compound can be converted into other compounds of general formula (2-d), in which G¹ₙ can be an optionally substituted alkyl, alkenyl, aryl or heteroaryl group.

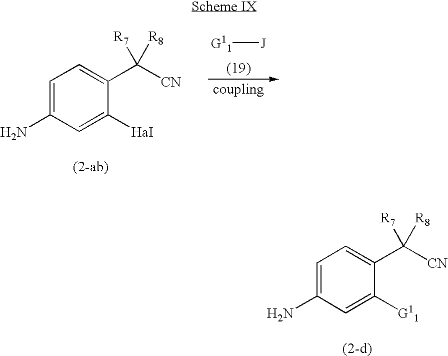

This kind of conversion can be carried out using different cross-coupling conditions, which include, but are not limited to, the Suzuki-cross coupling conditions (Suzuki, Pure & Appl. Chem., 1994, 66, 213-222; Suzuki, A. and Miyaura, N., Chem. Rev. (1995), 95, 2457-2483). In this case, compounds (2-ab) can be reacted with a suitable boron derivative (19) such as a boronic acid (J=B(OH)₂) or a borane (J=B(G¹₁)₂), in the presence of a palladium catalyst such as tetrakis(triphenylphosphine) palladium (0), and a base such us potassium carbonate, in a mixture of solvents such as 1,2-dimethoxyethane-water, dioxane-water and the like, at a temperature ranging from ambient to 110° C., over a period of time ranging from one to 20 hours. The reaction may be carried out under conventional heating (using an oil bath) or under microwave irradiation. The reaction may be conducted either in an open vessel or in a sealed tube.

In another process for preparing compounds of the present invention, compounds of general formula (2-ab) can be converted into compounds of general formula (2-d) in which G¹ₙ is a nitrile group. For instance, this conversion can be effected by means of a Stille reaction. In a typical procedure compound (2-ab) is treated with tributyltin cyanide (i.e. J=SnBu₃) in presence of a palladium catalyst (e.g. bis(triphenylphosphine)palladium (II) chloride) and a suitable base (e.g. potassium carbonate). Tetrabuthylammonium bromide or other commercially available analogues can be also present in the reaction mixture. This reaction is usually performed in DMF at a temperature about 100° C. over a period of about 2 hours.

In some embodiments of present invention, the same aforementioned cross-coupling reactions can be most conveniently performed using compounds of general formula (1) in Scheme I in which G^{l}ₙ is an opportune halogen (e.g. bromine) instead of the corresponding compounds of general formula (2) in Scheme I (e.g. compounds (2a-b) in Scheme IX).

Compounds of general formula (I) in Scheme I, in which M is a bond and Q a nitrile as in compounds of general formula (I-1) in Scheme X, can also be converted into other compounds of general formula (I) such as (I-2), (I-3) and (I-5) as shown in the scheme.

Compounds of general formula (I) in Scheme I, in which M is a bond and Q a nitrile as in compounds of general formula (I-1) in Scheme X, can also be converted into other compounds of general formula (I) such as (I-2), (I-3) and (I-5) as shown in the scheme.

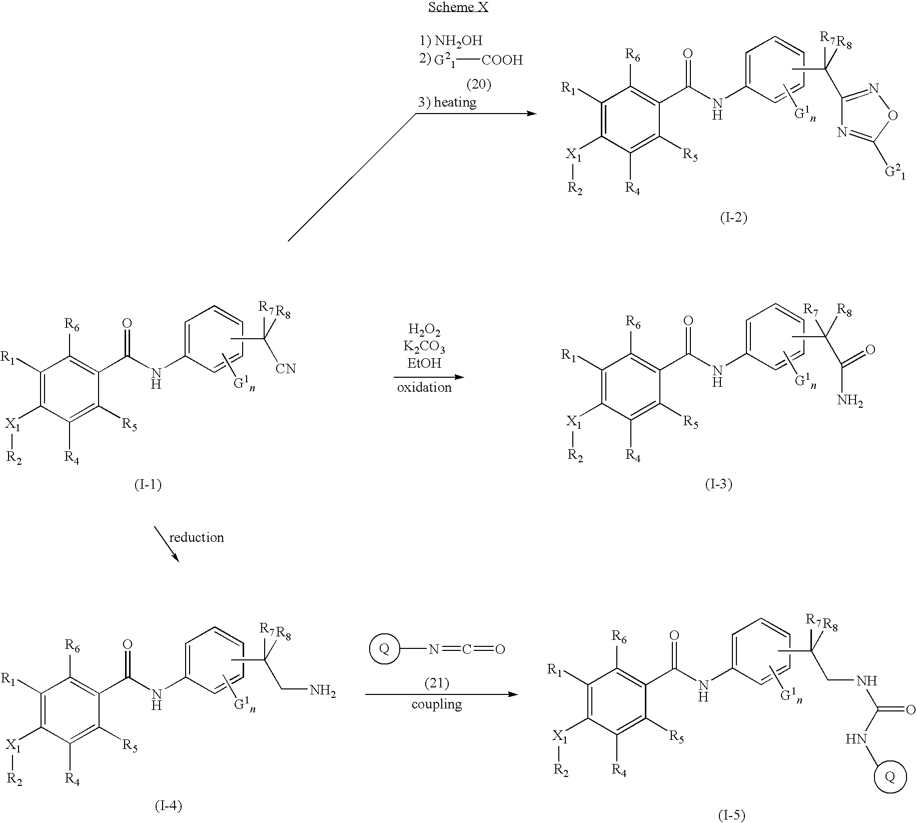

According to Scheme X, the compounds of general formula (I-1) may be converted into compounds of general formula (I-2). Thus, the cyano derivatives (I-1) (e.g. N-[4-(1-cyano-cyclopentyl)-phenyl]-3,4-dimethoxy-benzamide) are treated with aqueous hydroxylamine in an organic protic solvent such as ethanol and the like, at a temperature ranging from ambient to the reflux temperature of the solvent, followed by reaction of the resulting N-hydroxy-amidine intermediates with an appropriately substituted carboxylic acid of formula (20), in which G²₁ is defined hereinbefore. The coupling may be promoted by coupling agents known in the art such us 1-ethyl-3-(3-dimethylamino-propyl) carbodiimide hydrochloride, in the presence of a co-catalyst such as 1-hydroxybenzotriazole, in a suitable solvent (e.g. dioxane). Typically, an organic base such as triethylamine is also present in the reaction mixture. The reaction normally proceeds at ambient temperature for a time ranging from about 2 hours to 16 hours. Finally, the intermolecular cyclization can be accomplished by heating the reaction mixture at the reflux temperature of the solvent for about 8 hours.

The desired compounds of formula (I-3) of Scheme X can be prepared by treating compounds of formula (I-1) (e.g. N-[4-(cyano-dimethyl-methyl)-phenyl]-3,4-dimethoxy-benzamide) with an oxidizing reagent such as hydrogen peroxide, in a presence of an aqueous base such as potassium carbonate, in a protic solvent such as ethanol and the like, as described for example by Erdelmeier, I. et al. in JOC, 2000, 65, 24, 8152-8157. Typically the reaction proceeds by allowing the temperature to warm slowly from ambient temperature to 60° C.

The desired compounds of formula (I-5) can be obtained following the pathway reported in Scheme X. Thus, the cyano moiety of the compounds of formula (I-1) can be reduced to a primary amine, achieving compounds of general formula (I-4). The reduction is carried out by a catalytic hydrogenation using a catalyst such as palladium on charcoal, in a solvent such as methanol and the like. Typically, a Bronsted acid such as hydrochloric acid is also present in the reaction mixture. Subsequent coupling of the primary amine (I-4) with an appropriate substituted isocyanate of formula (21), in an inert solvent such as tetrahydrofuran, at temperatures ranging from 0° C. to ambient, yields the desired compounds of formula (I-5). Particularly, a notable protocol to prepare compounds (I-5) in which Q is an hydrogen, consists of treating intermediates (I-4) with trimethylsilyl-isocianate, in an inert solvent such as tetrahydrofuran, at about room temperature for a period of time of about one day, followed by cleavage of the trimethylsilyl moiety using standards conditions, which include, but are not limited to, the use of an aqueous base such as sodium bicarbonate, at about 35° C. for about 20-40 minutes.

Alternatively, compounds (I-4) can be employed as shown in Scheme XI in order to prepare other derivatives such as compounds (I-7), (I-8) and (I-10).

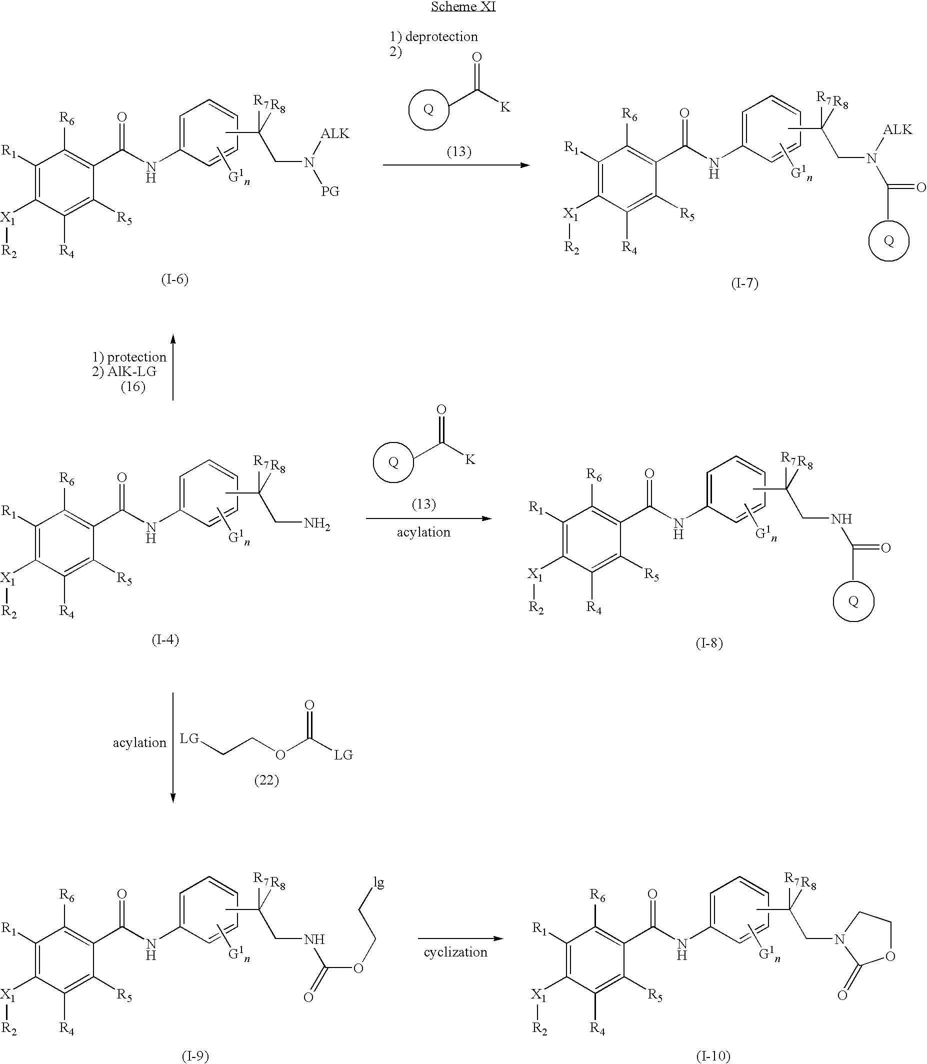

Tertiary amides of general formula (I-7) can be prepared using different synthetic approaches, which are well known to those skilled in the art. For instance, this may be conveniently done according to the procedure shown in Scheme XI. Thus, compounds of general formula (I-4) can be protected with a suitable N-protecting group (PG) using standard methodologies. For instance, PG can be a benzyl group, in this case the protection can be most conveniently done by heating amine (I-4) and benzaldehyde in a suitable solvent such as toluene, in the presence of an agent removing water (e.g molecular sieves) at a temperature higher than 100° C., usually about 110° C. Subsequent reduction of the imine intermediate, for example using a reducing agent such as sodium borohydride and the like, in a suitable solvent (e.g. ethanol), provides the N-protected intermediate. Alkyaltion of this intermediate under standard conditions affords intermediate such as (I-6). Standard conditions include, but are not limited to, the use of an alkylating agent of general formula (16) (e.g. iodomethane), in which ALK is an optionally substituted (C₁-C₆)alkyl group and LG a suitable leaving group such as an halide (most preferably iodine), in presence of a base, such as sodium bicarbonate and the like, in an inert solvent (e.g. acetonitrile). This reaction is typically conducted at a temperature ranging from ambient to 40° C., in a period of time ranging from 1 hour to 10 hours. The protecting group can be removed using standard conditions. For instance, when PG is a benzyl group, it can efficiently removed by catalytic hydrogenation, most notably using palladium on charcoal, in presence of an acid such as hydrochloric acid, in alcoholic solvent such as methanol. Subsequent coupling with a reagent of general formula (13), in which K can be a halide such as chlorine, affords the desired tertiary amides of general formula (I-7). This reaction is typically conducted using a suitable acylating reagent (13) (e.g. acetyl chloride), in presence of an organic base such as triethylamine and the like. The reaction is generally performed in an inert solvent such as dichloromethane, at room temperature.

Alternatively, amines (I-4) can be directly coupled with compounds of general formula (13) to achieve compounds of general formula (I-8). Compounds (13) are either commercially available, or are known in the art, or can be readily prepared using procedure analogues to those reported in the literature for known compounds. For instance, in the case where K is halogen, the amines (I-4) are reacted either with an acyl halide or with an alkyl haloformate (most preferably acyl chloride and alkyl chloroformate), using methods that are readily apparent to those skilled in the art. The reaction may be promoted by a base such as triethylamine, in a suitable solvent (e.g. dichloromethane) at room temperature.

In the case where K is —OH, the amines of formula (I-4) are reacted with the carboxylic acids (13), promoting the coupling with an activating agent such as 1-ethyl-3-(3-dimethylamino-propyl) carbodiimide hydrochloride or other analogues known in the art, and in the presence of 1-hydrobenzotriazole. In some embodiments of this process, the coupling is performed in the presence of an organic base such as triethylamine, N-methylmorpholine and the like, and in an aprotic solvent (dichloromethane, dioxane and the like).

When the acylating species (13) are anhydrides (K=QCOO—), the coupling is typically conducted in the presence of a base (e.g. triethylamine), in an inert solvent such as dichloromethane, at a temperature ranging from 0° C. to 35° C.

The method of choice for the preparation of compounds of formula (I-8) from amines (I-4) and compounds (13) is ultimately chosen on the bases of the reactivity of the amines (I-4), the commercial availability of reagents such as (13) and the compatibility with the sensitive groups present in both the starting materials.

In another process for the synthesis of the compounds of the present invention, compounds of general formula (I-4) may be converted to compounds of formula (I-10) of Scheme XI. The oxazolidin-2-one moiety can be prepared starting from primary amides following different synthetic approaches, which are well described in literature, most notably using one or slightly more than one equivalents of a reagent of general formula 22 (e.g. 2-chloroethyl chloroformate), in the presence of a base such as triethylamine and the like, in an inert solvent such as dichloromethane, to provide intermediate of formula (I-9). Subsequent treatment of this intermediate with a strong base such as sodium hydride, in a suitable solvent (e.g. N,N-dimethylformamide), yields desired compounds of formula (I-10). This reaction is most conveniently done in the presence of a catalytic amount of potassium iodine, at a temperature ranging from 35° C. to 70° C. over a time period of about 2 hours.

Compounds of general formula (I) in Scheme I, in which M is a carbonyl and Q an hydroxy group as in compounds of general formula (I-11) in Scheme XII, can also be converted into other compounds of general formula (I) such as (I-12) and (I-13) as shown in the scheme.

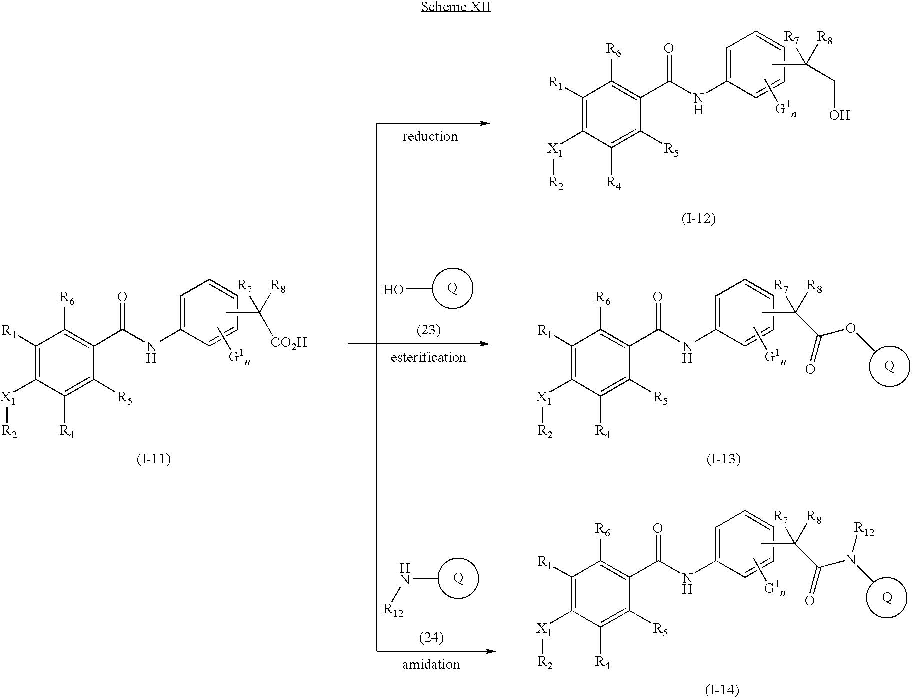

Thus, the acid (I-11) may be reduced to alcohol of general formula (I-12), using procedures well known to those skilled in the art. These procedures include, but are not limited to, treatment of acid of formula (I-11) with an activating agent, such as a chloroformate (e.g. n-butyl chloroformate), in the presence of a base (e.g. N-methylmorpholine and the like) in an inert solvent such as 1,2-dimethoxyethane at low temperature (between −10 and 0° C.), for a short period of time (about 10-20 minutes).

In another process for preparing compounds of the present invention, compounds of general formula (I-11) can be converted to the corresponding ester of general formula (I-13), according to standard procedures extensively reported in literature. For example, the condensation between acids (I-11) and the appropriately substituted alcohol of general formula (23) can be conducted under Fischer esterification conditions, using a suitable acid such as a sulfonic acid as the catalyst. This reaction is most conveniently done using an acidic resin (e.g. Amberlyst® 15 hydrogen form) as the acidic catalyst, alcohol (23) as the reaction solvent and heating at a temperature higher than 100° C., generally at about 120° C.

Alternatively, acids (I-11) can be reacted with amines of general formula (24), in which R₁₂ has the above-given meanings, to produce amide compounds (I-14). This condensation can be carried out under different conditions, which are readily apparent to those skilled in the art. For instance, treatment of acid (I-11) (e.g. 1-[4-(3,4-dimethoxy-benzoylamino)-phenyl]-cyclopentanecarboxylic acid) with commercially available carbodiimide such as 1-(3-dimethylamino)propyl)-3-ethylcarbodiimide hydrochloride (EDC), and subsequent reaction with the opportunely substituted amines (24) (e.g. morpholine) results in the formation of compounds (I-14). The reaction is most conveniently performed with one or slightly more than one equivalents of commercially available additive N-hydroxybenzotriazole (HOBt), or alternative analogues known in the art, in the presence of an organic base such as triethylamine. Solvents generally useful include halocarbon solvents such as dichloromethane.

Compounds of general formula I in Scheme (I), in which Q is a heteroaryl as described above featuring an NH inside the ring (e.g indole, indazole, benzoimidazole and the like in which R₂₀ and/or R₂₁ are H), can be further reacted to prepare other compounds of general formula (I) in which the nitrogen bears groups different from hydrogen (e.g. an acyl group such as an acetyl group). In a typical procedure compounds of general formula (I), such as. 1H-indole-3-carboxylic acid {2-[4-(3,4-dimethoxy-benzoylamino)-phenyl]-2-methyl-propyl}-amide, can be reacted with one or slightly more than one equivalents of acyl halide (e.g. acethyl chloride) in the presence of a base such as 4-dimethylamino pyridine to provide the corresponding N-amide derivatives (e.g. 1-acetyl-1H-indole-3-carboxylic acid {2-[4-(3,4-dimethoxy-benzoylamino)-phenyl]-2-methyl-propyl}-amide). The reaction typically proceeds in an inert solvent such as dichloromethane and a temperature higher than 70° C.

Compounds of general formula I in Scheme (I), in which Q is an aryl or heteroaryl and G²ₚ is a halogen, can be further reacted to prepare other compounds of general formula (I) in which G²ₚ is an aryl or heteroaryl, in accordance with the G²ₚ definition previously reported. This is most conveniently done by means of a Suzuki cross-coupling reaction (Suzuki, Pure & Appl. Chem., 1994, 66, 213-222; Suzuki, A. and Miyaura, N., Chem. Rev. (1995), 95, 2457-2483). In a typical procedure, compounds of general formula (I), such as 4-bromo-1-methyl-1H-pyrazole-3-carboxylic acid {2-[4-(3,4-dimethoxy-benzoylamino)-phenyl]-2-methyl-propyl}-amide, is reacted with a boron derivative such as a boronic acid (e.g phenylboronic acid), in the presence of a palladium catalyst such as palladium (II) acetate and the like, and a base such as potassium fluoride, in a suitable solvent such as methanol. In some embodiments, water may be a co-solvent in the process. The reaction is typically performed at a temperature ranging from room temperature to 100° C., over a period of about 2 hours.

In another process for the preparation of the compounds of the present invention, compounds of general formula (I) of Scheme I, in which either R₁ is hydroxy group such as in compounds of formula (I-15) in Scheme XIII and R₂ hydrogen such as in compounds of formula (I-16) in Scheme XIII can be converted to compounds of general formula (I).

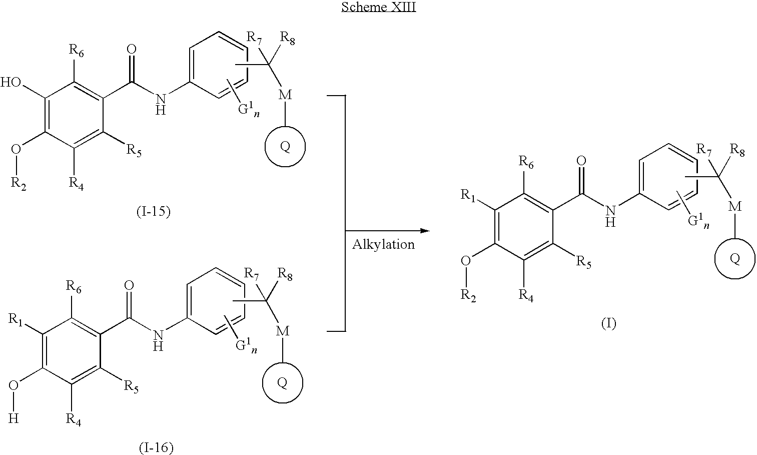

The selective O-alkylation of compounds of general formula (I-15) or (I-16) with the suitable alkyl halides (e.g 2-iodo-propane) can be carried out using one or slightly more than one equivalent of a base such as potassium carbonate, in a polar solvent such as N,N-dimethylformamide (DMF) and the like. Typically, the reaction proceeds at room temperature, over a period ranging from 16 hours up to 40 hours (Osborn, N. J. and Robinson, J. A., Tetrahedron (1993), 49, 14, 2873-2884).

In another process for preparing compounds of the present invention, compounds of general formula (I) in Scheme I in which G¹ₙ is a bromine, M is a bond and Q a nitrile such as in compounds of general formula (I-17), may be converted into other compounds of formula (I), such as (I-18), as illustrated in Scheme XIV. This kind of conversion can be carried out using different cross-coupling conditions, which include, but are not limited to, the Suzuki-cross coupling conditions (Suzuki, Pure & Appl. Chem., 1994, 66, 213-222; Suzuki, A. and Miyaura, N., Chem. Rev. (1995), 95, 2457-2483). In this case, compounds (I-17) can be reacted with a suitable boron derivative (19) such as a boronic acid (J=B(OH)₂) or a borane (J=B(G¹₁)₂), in the presence of a palladium catalyst such as tetrakis(triphenylphosphine) palladium (0), and a base such us potassium carbonate or potassium fluoride, in a solvents such as 1,2-dimethoxyethane, methanol, or xylene at a temperature about 110° C.-140° C. over a period of 1 or 2 hours. Typically, this reaction is most conveniently performed heating with a microwave oven.

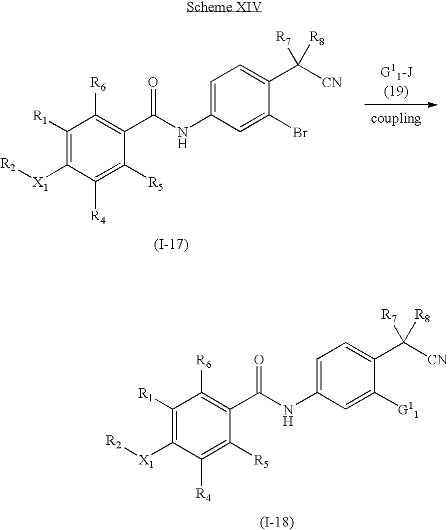

In other process of the present invention, compounds of general formula (I-18) may be obtained following the synthetic route described in scheme XV.

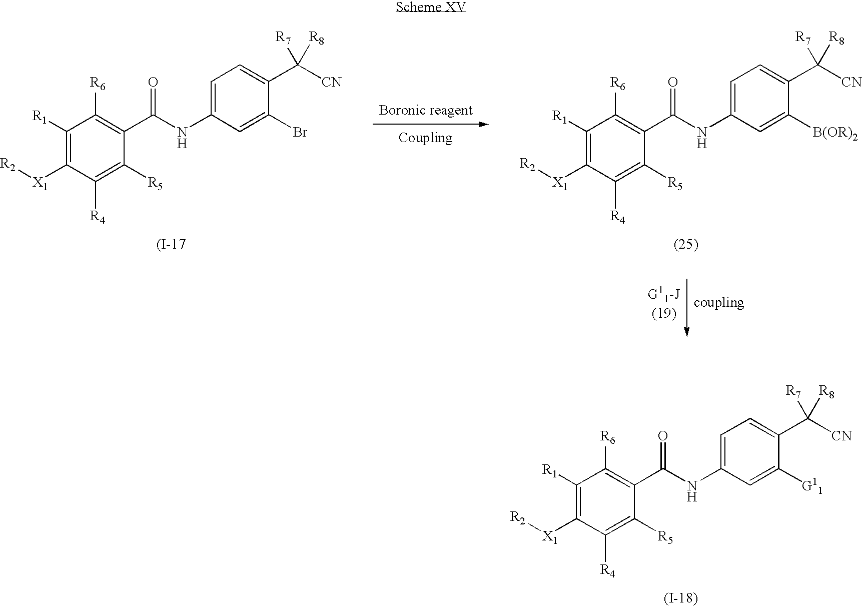

In this case, compounds (I-17) can be converted into the corresponding boronic derivative by reaction with a suitable boronic reagent such as bis(pinacolato)diboron or an dialkyl-boronate, in the presence of a palladium catalyst such as 1,1′-bis(diphenylphosphino)ferrocenedichloropalladium (II), and a base such as potassium carbonate, in a aprotic polar solvent such as DMSO and the like, at a temperature of about 95° C., during a period of 1-2 hours.

The subsequent Suzuki coupling of the intermediate (25) with a suitable halogen derivative (19) (e.g. J=Br), following the experimental conditions described in Scheme IX, affords the desired compound of general formula (I-18).

According to Scheme XVI, the intermediate compound 25 can be also used as the starting material for the synthesis of phenol derivatives of general formula (I-19).

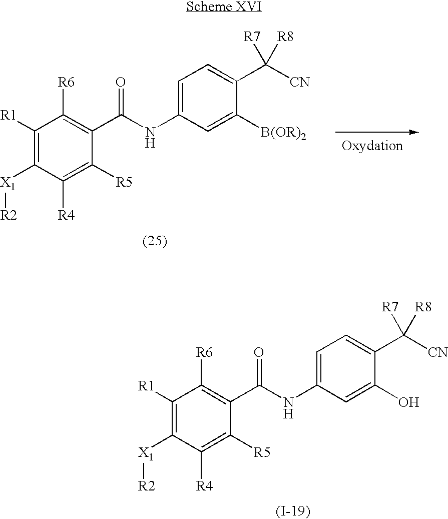

Thus, compound 25 can be treated with an oxidizing reagent such as hydrogen peroxide in a solvent such as dioxane and the like. This reaction is most conveniently done at a temperature about 40° C. and over a time period ranging from 2 to 4 hours.

Compounds of general formula (I) in Scheme I, in which M is a —(CH₂)₂—NH—CO— as in compounds of general formula (I-20) in Scheme XVII, can be prepared according the synthetic route illustrated below.

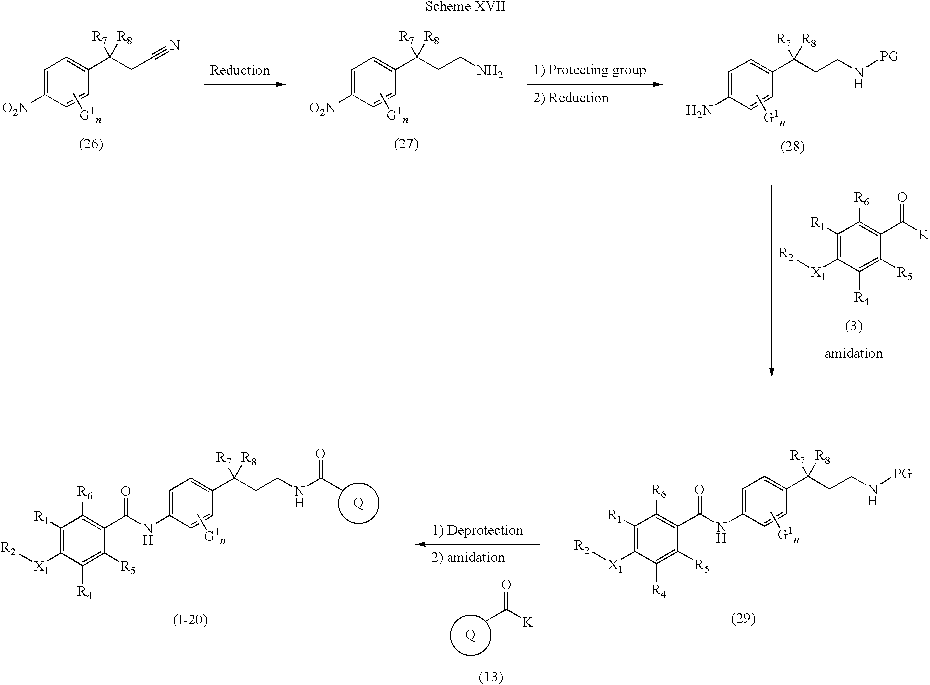

According to the scheme, the nitrile derivatives of formula (26), prepared as described in Scheme IV, are converted into the corresponding primary amine derivatives (27), following a procedure similar to that described by Weinstock, J. et al. in J. Med. Chem., 1987,30, 7, 1166-1176. Thus, intermediates (26) are reacted with a reducing reagent such as borane, preferably borane-tetrahydrofuran complex, in an aprotic solvent such as tetrahydrofuran. The reaction typically proceeds by heating the reaction from ambient temperature up to the reflux temperature of the solvent, for a time of about one hour. The primary amine in the derivative (27) can be protected with a suitable N-protecting group (PG), such as tButyloxycarbonyl, Benzyloxycarbonyl, Ethoxycarbonyl, Benzyl and the like, using standard methodologies. The nitro group of the N-protected intermediate can be reduced to produce (28), most conveniently by catalytic hydrogenation in presence of a suitable catalyst such as palladium or platinum catalyst. This reaction is typically carried out in lower alcohol (methanol, ethanol and the like), at about atmospheric pressure of hydrogen and at about room temperature. Subsequent coupling of the resulting compounds (28) with a suitable reagent (3) according to the reaction conditions described in the Scheme I affords the compounds (29). The PG protecting group is removed under conditions readily apparent to those skilled in the art to produce the primary amine which can be further converted into the corresponding amide (I-20) with a suitable reagent (13) following reaction conditions described in the scheme V.

In some embodiments of the present invention, compounds of general formula (I-ac) in Scheme XVII can be most conveniently prepared according to the synthetic route reported in Scheme XVIII.

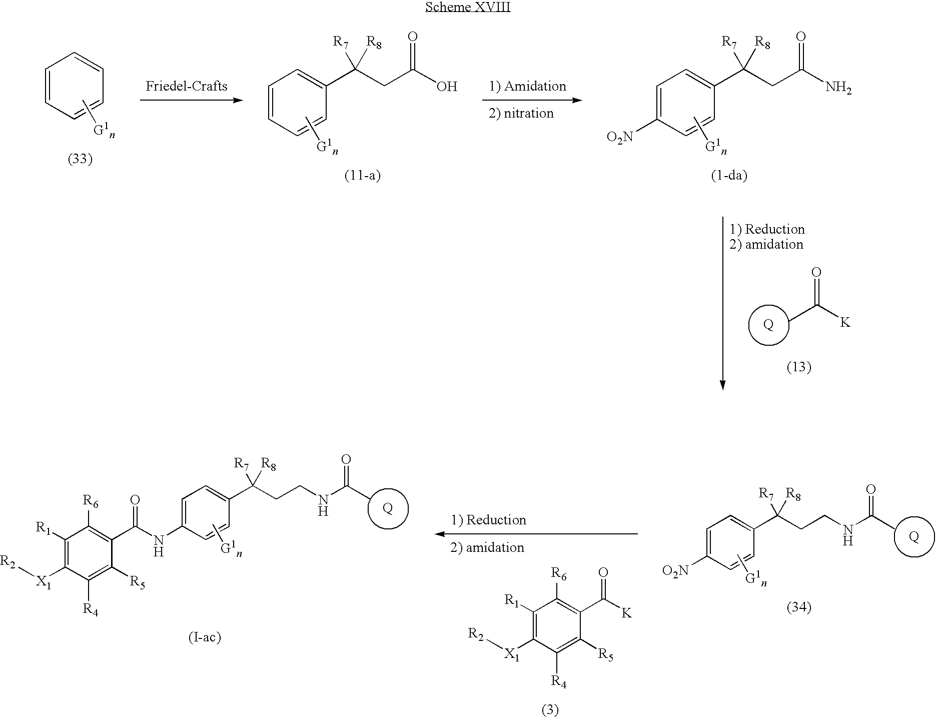

According to the scheme, the arenes of formula (33), are converted into the corresponding acid derivatives (11-a), for instance by means of Lewis acid-catalyzed electrophilic aromatic substitution such as Friedel-Crafts reaction (Smith and Spillane in JACS, 1943, 65, 202-208). Thus a suitable arene such as benzene is reacted with the opportune alkene (e.g. 3-methylbut-2-enoic acid) in presence of a Lewis acid, preferably anhydrous aluminum chloride or similar. This reaction is typically conducted at a temperature ranging from 5° C. to room temperature, in a period of time ranging from 1 hour to 16 hours. The acid group of (11-a) can be converted into a primary amide moiety using one of the methods that are readily apparent to those skilled in the art. For instance, treatment of acids (11-a) with one or more equivalents of oxalyl chloride in the presence of a catalytic amount of DMF in a halocarbon solvent, such as dichlormethane, at temperature ranging form 0° C. to 35° C., affords the corresponding acyl chlorides, which can be reacted with ammonia (gas, liquid or aqueous solution) in the suitable solvent (e.g. DCM or DMF) to give the corresponding amide intermediate. The resulting compounds are then transformed into compounds (1-da) by means of nitration reaction, using a protocol similar to that described in Scheme IV. Reduction of amide moiety according to the conditions described in Scheme V, followed by coupling of resulting amine with a suitable reagent (13) under similar condition to those described in Scheme V, provides desired compounds (34). The nitro group can be reduced to produce the corresponding aniline, most conveniently by catalytic hydrogenation in presence of a suitable catalyst such as palladium or platinum catalyst. This reaction is typically carried out in lower alcohol (methanol, ethanol and the like), at about atmospheric pressure of hydrogen and at about room temperature. Subsequent coupling of the resulting compounds with a suitable reagent (3) according to the reaction conditions described in the Scheme I affords the compounds (I-ac).

The intermediates in Scheme XVIII can be directly treated as reported in the scheme or converted into other intermediates, which can then undergo the same reaction reported in Scheme XVIII.

For instance, when G¹ₙ in intermediate (1-da) is hydrogen, such as in compounds (1-db) in Scheme XIX, it can be converted in compounds of formula (1-dc) by means of a halogenation reaction. This is most conveniently accomplished using a halide donor, such as an halide (e.g. bromine) or other analogues known in the art, most conveniently in presence of an activating species such as silver trifluoromethansulfonate (or equivalent) in strong acid (e.g. H₂SO₄). In a typical procedure, the reaction is carried out at temperature about room temperature over a period of about 3 hours.

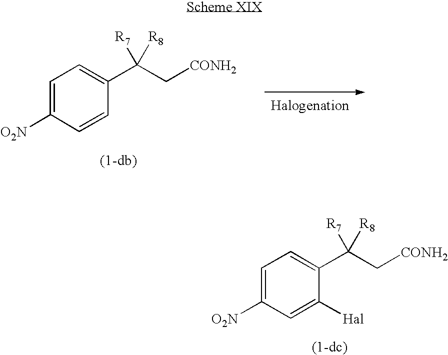

Compounds (1-dc) can then be used as compounds (1-da) in Scheme XVIII.

In another example, when G¹ₙ in intermediate (34) of Scheme XVIII is an halogen such as in compounds (34-a) in Scheme XX, it can be converted into other compounds of formula (34-b) in which G¹ₙ can be an optionally substituted alkyl, alkenyl, aryl or heteroaryl group.

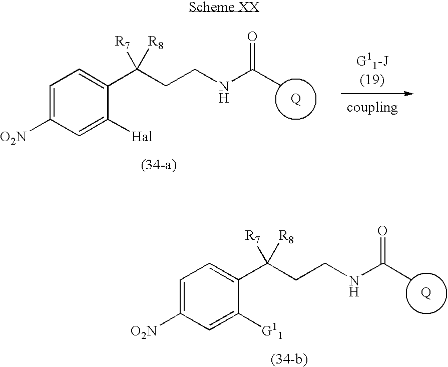

This kind of conversion can be carried out using different cross-coupling conditions, which include, but are not limited to, the Suzuki-cross coupling conditions (Suzuki, Pure & Appl. Chem., 1994, 66, 213-222; Suzuki, A. and Miyaura, N., Chem. Rev. (1995), 95, 2457-2483). In this case, compounds (34-a) can be reacted with a suitable boron derivative (19) such as a boronic acid (J=B(OH)₂) or a borane (J=B(G¹₁)₂), in the presence of a palladium catalyst such as tetrakis(triphenylphosphine) palladium (0), and a base such us potassium carbonate, in a mixture of solvents such as 1,2-dimethoxyethane-water, dioxane-water and the like, at a temperature ranging from ambient to 110° C., over a period of time ranging from one to 20 hours. The reaction may be carried out under conventional heating (using an oil bath) or under microwave irradiation. The reaction may be conducted either in an open vessel or in a sealed tube. Compounds (34-b) can then be used as compounds (34) in Scheme XVIII.

In another process for preparing compounds of the present invention, the intermediate compounds of general formula 15 (Scheme VI) can be most conveniently used as the starting materials for the compounds of general formula (34), for instance according to the synthetic route shown in Scheme XXI.

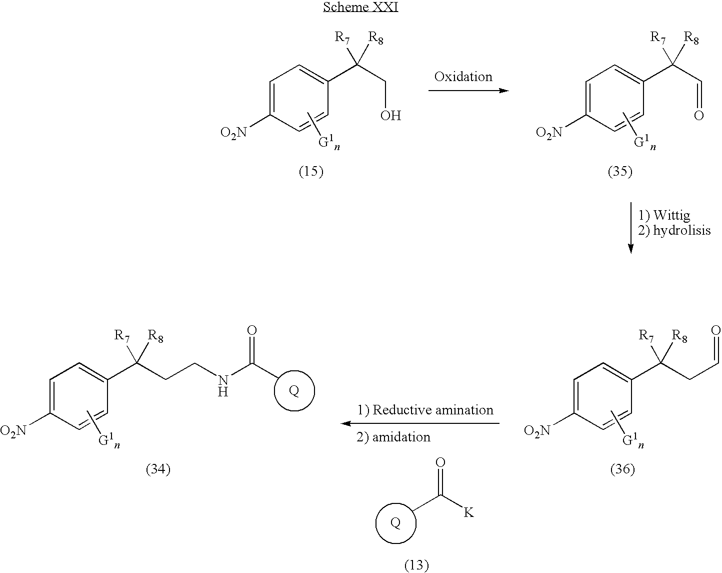

Thus, alcohols (15) can be oxidized into the corresponding aldehydes (35) using procedures well known to those skilled in the art (a survey of the suitable reactions is given by Larock, R. C. Comprehensive Organic Transformations, Second Edition (1999), Wiley-VCH, New York and London, pg 1234). These procedures include, but are not limited to, treatment of alcohols of formula (15) with an oxidizing reagent such us Dess-Martin periodinane in a suitable solvent such as dichloromethane, at about room temperature. The resulting aldehydes (35) can be then transformed into aldehydes (36) by means of one of the standard protocols broadly reported in the literature. For instance, this conversion can be efficiently effected using a Wittig-type reaction and related. In a typical procedure, aldehydes (35) are reacted with an ylide generated from an opportune phosphonium salt to give the insertion of an additional carbon atom. When (methoxymethyl)triphenylphosphonium chloride is used as the phosphonium salt, the resulting compounds can be efficiently hydrolyzed, typically using protic acid (e.g. trifluoroacetic acid) in aqueous environment at about room temperature, to obtain the aldehydes (36). These aldehydes may be transformed into compounds of general formula (34) by one of the methods know to those skilled in the art. For instance, this can be obtained transforming the aldehydes (36) into the corresponding primary amines, for instance by a reductive amination reaction. One, but not the only, procedure consists of treating compounds (36) with ammonia, most conveniently generated in situ from an opportune ammonium salt (e.g. ammonium acetate), in presence of a reducing species such as sodium cyanoborohydride, in solvents such as methanol and the like and at about room temperature. Coupling of resulting amines with suitable reagents (13) under similar condition to those described in Scheme V, provides desired compounds (34).

Pharmacology

The compounds provided in this invention are negative allosteric modulators of GPCR; in particular they are negative allosteric modulators of FSH receptors. They are expected to exert their effect at FSH receptors by virtue of their ability to decrease the response of such receptors to FSH or FSH agonists, inhibiting the response of the receptor. Hence, the present invention relates to a compound for use as a medicine, as well as to the use of a compound according to the invention or a pharmaceutical composition according to the invention for the manufacture of a medicament for treating or preventing a condition in a mammal, including a human, the treatment or prevention of which is affected or facilitated by the modulatory effect of FSH allosteric modulators, in particular negative FSH allosteric modulators.

Also, the present invention relates to the use of a compound according to the invention or a pharmaceutical composition according to the invention for the manufacture of a medicament for treating, or preventing, ameliorating, controlling or reducing the risk of various disorders associated with FSH receptor dysfunction as well as contraception in a mammal, including a human, the treatment or prevention of which is affected or facilitated by the modulatory effect of FSH negative allosteric modulators.

Where the invention is said to relate to the use of a compound or composition according to the invention for the manufacture of a medicament for e.g. the treatment of a mammal, it is understood that such use is to be interpreted in certain jurisdictions as a method of e.g. treatment of a mammal, comprising administering to a mammal in need of such a treatment, an effective amount of a compound or composition according to the invention.

In particular, the diverse disorders associated with FSH receptor dysfunction, include one or more of the following conditions or diseases: estrogen-related disorders such as uterine fibroids, endometriosis, polycystic ovarian disease, dysfunctional uterine bleeding, breast cancer and ovarian cancer. Others include the depletion of oocytes or spermatocyte a common side effect observed during chemotherapies and osteoporosis.

Because negative allosteric modulators of FSH receptors, including compounds of Formula I, inhibit the response of FSH receptors to FSH and FSH agonists, it is understood that the present invention extends to the treatment of disorders associated with FSH dysfunction and/or contraception by administering an effective amount of a negative allosteric modulator of FSH receptors, including compounds of Formula I, in combination with agent that affect the viability or motility or fertilizability of sperm or with others known contraceptives.

The compounds of the present invention may be utilized in combination with one or more other drugs in the treatment, prevention, control, amelioration, or reduction of risk of diseases or conditions for which compounds of Formula (I) or the other drugs may have utility, where the combination of the drugs together are safer or more effective than either drug alone.

Reasonable variations are not to be regarded as a departure from the scope of the invention. It will be obvious that the thus described invention may be varied in many ways by those skilled in the art.

The compounds provided in the present invention are negative allosteric modulators of FSH receptors. As such, these compounds do not activate the FSH receptors by themselves. Compounds of Formula (I) are expected to have their effect at FSH receptors by virtue of their ability to antagonize the function of the receptor upon FSH or a FSH receptors agonist activation. The behavior of negative allosteric modulators, such as the ones described in Formula I, at FSH receptors is shown in the following paragraph, describing a biological assay which is suitable for the identification of such compounds.

Intracellular cAMP Measurement Assay

The Intracellular cAMP measurement assay is a functional cell-based assay used to study GPCR function. This method relies on a Time-Resolved Fluorescence (HTRF) assay to measure the cAMP accumulation upon receptor-mediated Gs protein activation in cells expressing recombinant GPCR or in cells from native tissues. In brief, this method is a competitive immunoassay between native cAMP produced by cells and the cAMP labeled with a fluorescent tag. The endogenously produced cAMP competes with exogenous added d2-labelled cAMP for the cAMP binding site on a Eu3+ cryptate labelled anti-cAMP antibody. The d2-labelled cAMP and the Eu³⁺ labelled antibody produces a basal fluroresence via HTRF and therefore an intracellular increase in unlabelled cAMP is detected as decrease in this signal. cAMP is one of the most important intracellular mediators. Its concentration in cells can be increased upon binding of many hormones to their receptors. The most studied pathway consists in the release of α-subunit GTP-binding proteins following ligand-receptor interaction, which in turn activates or inhibits the ATP/cAMP conversion function of adenylate cyclase enzyme. cAMP is then involved in many complex regulatory processes such as protein kinase activation or ion channel gating. This method is widely used to study receptor activation of G protein in cells over-expressing GPCRs or in native cells, including FSH receptors expressing cells (Gabriel et al., Assay Drug Dev. Technol., 1, 291-303, 2003).

Transfection and cell culture: The cDNA encoding the rat follicle stimulating hormone receptor (rFSHR), (accession number NM_{—}199237, NCBI Nucleotide database browser) was subcloned into an expression vector containing also the hygromycin resistance gene. Transfection of this vector into HEK293 cells with PolyFect reagent (Qiagen) according to supplier's protocol, and hygromycin treatment allowed selection of antibiotic resistant cells which had integrated stably one or more copies of the plasmid. Positive cellular clones expressing rFSHR were identified in a functional assay measuring cAMP production in cells following stimulation by addition of purified human follicle stimulating hormone (hFSH).

HTRF cAMP assay: On the day of the experiment, cells were detached from the Petri dish and distributed in a low volume, black-walled 384-well plate at a density of 5,000 cells/well in an assay buffer containing 1 mM IBMX to prevent cAMP degradation by cytoplasmic phosphodiesterases. The determination of the cAMP accumulation was performed using an HTRF assay (Trinquet et al., Anal. Biochem., 358, 126-135, 2006). Briefly, cells were incubated for 3 min in the presence of increasing concentrations of negative allosteric modulators (from 1 nM to 60 μM) and then 30 min in the presence of 1 ng/ml of hFSH, an agonist of rat FSH receptor, that has been determined in previous experiments to correspond to the EC₇₀, a concentration that gives 70% of the maximal response of the agonist, and is in accordance with published data (Fox et al., Mol. Endocrin., 15, 378-389, 2001). Likewise, 10-point concentration-response curves of FSH receptor-specific agonist such as hFSH, were tested in the absence or in the presence of increasing concentrations of negative allosteric modulator in order to detect a rightward-shift of the concentration-response curve of the agonist (revealed by a increase in the EC₅₀) and a decrease of its maximal efficacy (characteristic of negative allosteric modulation). Cells were then lysed by adding the HTRF assay components, the europium cryptate-labeled anti-cAMP antibody, and the XL665-labeled cAMP analog, previously diluted in a HEPES buffer (50 mM, pH 7.0) containing 0.8 M potassium fluoride, 0.2% (w/v) BSA, and 1% (v/v) Triton X-100, a percentage of detergent that ensure complete cell lysis. Assay was then incubated for 1 hr at room temperature, and HTRF signal was measured after excitation at 337 nm, and dual emission at 620 and 665 nm, using a RubyStar fluorimeter (BMG Labtechnologies). Moreover, the fluorescence ratio of an appropriate range of known concentrations of cAMP standards was also included on each assay plate to produce a standard cAMP curve. Providing that the fluorescence ratio of the cAMP inhibited by the compound falls in the linear part of the cAMP standard curve (i.e. where a change in fluorescence ratio is proportional to change in cAMP concentration) this allows the exact concentration of cAMP inhibited by the compounds to be calculated. The assay signal was therefore expressed as the percentage of signal inhibition.

Data analysis: The concentration-response curves of representative compounds of the present invention in the presence of EC₇₀ of FSH receptor agonist were generated using the Prism Graph-Pad program (Graph Pad Software Inc, San Diego, USA). The curves were fitted to a four-parameter logistic equation (Y=Bottom+(Top−Bottom)/(1+10̂((LogIC₅₀−X)*Hill Slope) allowing determination of IC₅₀ values. Each curve was performed using duplicate sample per data point and 10 concentrations. The concentration-response curves of a selective FSH receptor agonist in the absence or in the presence of representative compounds of the present invention were also generated using Prism Graph-Pad program (Graph Pad Software Inc, San Diego, USA). The curves were fitted to a four-parameter logistic equation (Y=Bottom+(Top−Bottom)/(1+10̂((LogEC₅₀−X)*Hill Slope) allowing determination of EC₅₀ values of the selective FSH receptor agonist. Each curve was performed using duplicate sample per data point and 10 concentrations.

Table 1 shows representative compounds of the present invention that were clustered into four classes according to their ability (IC₅₀) to antagonise an EC₇₀ of FSH receptors agonist such as hFSH. Class A: IC₅₀<150 nM; Class B: 150 nM≦IC₅₀≦400 nM; Class C: 400 nM≦IC₅₀≦1000 nM; Class D: IC₅₀≧1000 nM.

TABLE 1
Summary of activity-data

| Example | Class | Example | Class |
|---|---|---|---|
| 31 | B | 109 | B |
| 32 | B | 110 | B |
| 34 | B | 113 | B |
| 41 | A | 116 | A |
| 59 | B | 120 | B |
| 72 | B | 121 | B |
| 74 | B | 125 | D |
| 87 | B | 126 | B |
| 95 | B | 127 | C |
| 99 | B | 132 | B |
| 101 | B | 133 | A |
| 102 | D | 139 | A |
| 104 | C | 142 | B |
| 108 | C | 183 | D |
| 184 | D | 203 | B |
| 185 | C | 204 | D |
| 186 | D | 205 | C |
| 187 | A | 206 | A |
| 189 | D | 207 | B |
| 190 | D | 208 | C |
| 191 | B | 209 | D |
| 192 | D | 210 | C |
| 193 | A | 211 | A |
| 194 | B | 212 | D |
| 195 | D | 213 | C |
| 196 | B | 214 | B |
| 198 | A | 215 | C |
| 199 | B | 216 | B |
| 200 | A | 217 | B |
| 201 | B | 218 | B |
| 202 | A | 219 | A |
| 221 | C | | |

The following non-limiting examples are intending to illustrate the invention. The physical data given for the compounds exemplified is consistent with the assigned structure of those compounds.

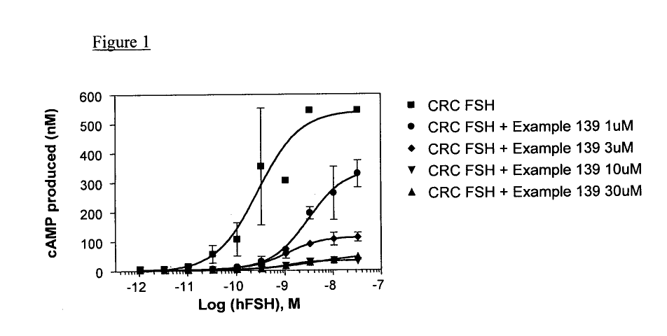

FIG. 1 shows 10-point concentration-response curves (crc) of FSH receptor-specific agonist (hFSH), tested in the absence or in the presence of increasing concentrations of negative allosteric modulator in order to detect a rightward-shift of the concentration-response curve of the agonist (revealed by a increase in the EC₅₀) and a decrease of its maximal efficacy (characteristic of negative allosteric modulation).

### Examples

Unless otherwise noted, all starting materials were obtained from commercial suppliers and used without any further purification.

Specifically, the following abbreviation may be used in the examples and throughout the specification.

| | |
|---|---|
| g (grams) | H₂O (water) |
| mg (milligrams) | DMF (N,N-dimethylformamide) |
| mL (millilitres) | DCM (dichloromethane) |
| uL (microliters) | CH₃CN (acetonitrile) |
| um (micrometers) | MeOH (methanol) |
| mmol (millimoles) | EtOH (ethanol) |
| M (molar) | EtOAc (ethyl acetate) |
| N (normal) | THF (tetrahydrofurane) |
| Å (Angstrom) | iPr₂O (isopropyl ether) |
| sat. (saturated aqueous solution) | Et₂O (diethyl ether) |
| % (percent) | DMSO (dimethyl sulfoxide) |
| h (hour) | K₂CO₃ (potassium carbonate) |
| min (minutes) | NaHCO₃ (sodium bicarbonate) |
| rt (room temperature) | HCl (hydrochloric acid) |
| RT (Retention Time) | TFA (trifluoroacetic acid) |
| MP (melting point) | AcOH (acetic acid) |
| LC-MS (Liquid Chromatography Mass Spectrum) | H₂SO₄ (sulfuric acid) |
| HPLC (High Performance Liquid Chromatography) | NaH (sodium hydride) |
| NMR (Nuclear Magnetic Resonance) | TEA (triethyl-amine) |
| 1H (proton) | NH₄OH (ammonium hydoxide) |
| Hz (Hertz) | KOH (potassium hydroxide) |
| MHz (megahertz) | NaOH (sodium hydroxide) |
| CDCl₃ (deuterated chloroform) | LiCl (lithoium chloride) |
| DMSO-d6 (deuterated dimethyl sulfoxide) | NH₄Cl (ammonium chloride) |
| MeOD (deuterated methanol) | Na₂SO₄ (sodium sulphate) |
| PS (polimer supported) | KNO₃ (potassium nitrate) |
| SCX (strong cationic exchange) | EDC (1-3(Dimethylaminopropyl)-3- |
| | ethylcarbodiimide, hydrochloride) |
| MW (microwave) | HOBt (1-hydroxybenzotriazole) |
| PtO₂ (platinum dioxide) | |
| 10% Pd/C (10% of palladium on activated charcoal) | |

All references to brine refer to a saturated aqueous solution of NaCl. Unless otherwise indicated, all temperatures are expressed in ° C. (degrees Centigrade). All reactions are conducted under an inert atmosphere at room temperature unless otherwise noted.

¹H NMR spectra were recorded on a Bruker ARX300 Spectrometer at 300.13 MHz (1H) using deuterated solvents such as DMSO (d6) or CDCl3 or MeOD. The instrument was equipped with a multinuclear inverse probe and temperature controller. Chemical shifts are expressed in parts of million (ppm, δ units). Coupling constants are in units of hertz (Hz) Splitting patterns describe apparent multiplicities and are designated as s (singlet), d (doublet), t (triplet), q (quadruplet), q (quintuplet), m (multiplet).

LCMS were Recorded Under the Following Conditions:

Method A) Waters Alliance 2795 HT Micromass ZQ. Column Waters XTerra MS C18 (50×4.6 mm, 2.5 um). Flow rate 1 ml/min Mobile phase: A phase=water/CH₃CN 95/5+0.05% TFA, B phase=water/CH₃CN=5/95+0.05% TFA. 0-1 min (A: 95%, B: 5%), 1-4 min (A: 0%, B: 100%), 4-6 min (A: 0%, B: 100%), 6-6.1 min (A: 95%, B: 5%). T=35° C.; UV detection: Waters Photodiode array 996, 200-400 nm.

Method B) Waters Alliance 2795 HT Micromass ZQ. Column Waters Symmetry C18 (75×4.6 mm, 3.5 um). Flow rate 1.5 ml/min. Mobile phase: A phase=water/CH₃CN 95/5+0.05% TFA, B phase=water/CH₃CN=5/95+0.05% TFA. 0-0.5 min (A: 95%, B: 5%), 0.5-7 min (A: 0%, B: 100%), 7-8 min (A: 0%, B: 100%), 8-8.1 min (A: 95%, B: 5%). T=35° C.; UV detection: Waters Photodiode array 996, 200-400 nm.

Method C) Waters Alliance 2795 HT Micromass ZQ. Column Waters Symmetry C18 (75×4.6 mm, 3.5 um). Flow rate 1.0 ml/min. Mobile phase: A phase=water/CH₃CN 95/5+0.05% TFA, B phase=water/CH₃CN=5/95+0.05% TFA. 0-1.0 min (A: 95%, B: 5%), 1.0-11.0 min (A: 0%, B: 100%), 11.0-12.0 min (A: 0%, B: 100%), 12.0-12.1 min (A: 95%, B: 5%). T=35° C.; UV detection: Waters Photodiode array 996, 200-400 nm.

Method D) UPLC system Waters Acquity, Micromass ZQ2000 Single quadrupole (Waters). Column 2.1×50 mm stainless steel packed with 1.7 um Acquity UPLC-BEH; flow rate 0.50 ml/min; mobile phase: A phase=water/acetonitrile 95/5+0.05% TFA, B phase=water/acetonitrile 5/95+0.05% TFA. 0-0.1 min (A: 95%, B: 5%), 1.6 min (A: 0%, B: 100%), 1.6-1.9 min (A: 0%, B: 100%), 2.4min (A: 95%, B: 5%).

Method E) UPLC system Waters Acquity, Micromass ZQ2000 Single quadrupole (Waters). Column 2.1×50 mm stainless steel packed with 1.7 um Acquity UPLC-BEH; flow rate 0.50 ml/min; mobile phase: A phase=water/acetonitrile 95/5+0.05% TFA, B phase=water/acetonitrile 5/95+0.05% TFA. 0-0.3 min (A: 95%, B: 5%), 3.3 min (A: 0%, B: 100%), 3.3-3.9 min (A: 0%, B: 100%), 4.4 min (A: 95%, B: 5%).

Method F) Waters Alliance 2795 HT Micromass ZQ. Column Waters Symmetry C18 (7533 4.6 mm, 3.5 um). Flow rate 1.5 ml/min. Mobile phase: A phase=water/CH₃CN 95/5+0.05% TFA, B phase=water/CH₃CN=5/95+0.05% TFA. 0-0.1 min (A: 95%, B: 5%), 6 min (A: 0%, B: 100%), 6-8 min (A: 0%, B: 100%), 8.1 min (A: 95%, B: 5%). T=35° C.; UV detection: Waters Photodiode array 996, 200-400 nm.

Method G) Instrument: ZQ2000 (Waters) coupled with UPLC and Sample Organizer and UV detector MS detector: Waters ZQ2000. Column: Acquity UPLC-BEH C18 50×2.1 mm×1.7 um; flow rate 0.6 ml/min; mobile phase: A phase=water/acetonitrile 95/5+0.1% TFA, B phase=water/acetonitrile 5/95+0.1% TFA. 0-0.25 min (A: 98%, B: 2%), 3.3 min (A: 0%, B: 100%), 3.3-4.00 min (A: 0%, B: 100%), 4.1 min (A: 98%, B: 2%), 4.10-5.00 min (A: 98%, B: 2%); Mass spectrometer conditions: Capillary 3.25 kV, cone 20V, source temperature 115° C. desolvation T 350° C.

Method H) Instrument: ZQ2000 (Waters) coupled with UPLC and Sample Organizer and UV detector MS detector: Waters ZQ2000. Column: Acquity UPLC-BEH C18 50×2.1 mm×1.7 um; flow rate 0.4 ml/min; mobile phase: A phase=water/acetonitrile 95/5+0.1% TFA, B phase=water/acetonitrile 5/95+0.1% TFA. 0-0.25 min (A: 98%, B: 2%), 4.00 min (A: 0%, B: 100%), 4.00-5.00 min (A: 0%, B: 100%), 5.10 min (A: 98%, B: 2%), 5.10-6.00 min (A: 98%, B: 2%); Mass spectrometer conditions: Capillary 3.25 kV, cone 20V, source temperature 115° C., desolvation T 350° C.

Method I) Instrument: ZQ2000 (Waters) coupled with UPLC and Sample Organizer and UV detector MS detector: Waters ZQ2000. Column: Acquity UPLC-BEH C18 50×2.1 mm×1.7 um; flow rate 0.6 ml/min; mobile phase: A phase=water/acetonitrile 95/5+0.1% TFA, B phase=water/acetonitrile 5/95+0.1% TFA. 0-0.50 min (A: 98%, B: 2%), 6.00 min (A: 0%, B: 100%), 6.00-7.00 min (A: 0%, B: 100%), 7.1 min (A: 98%, B: 2%), 7.1-8.50 min (A: 98%, B: 2%); Mass spectrometer conditions: Capillary 3.25 kV, cone 20V, source temperature 115° C. desolvation T 350° C.

Method L) Instrument: ZQ2000 (Waters) coupled with UPLC and Sample Organizer and UV detector MS detector: Waters ZQ2000. Column: Acquity UPLC-BEH C18 50×2.1 mm×1.7 um; flow rate 0.4 ml/min; mobile phase: A phase=water/acetonitrile 95/5+0.1% TFA, B phase=water/acetonitrile 5/95+0.1% TFA. 0-0.50 min (A: 98%, B: 2%), 7.0 min (A: 0%, B: 100%), 7.0-8.0 min (A: 0%, B: 100%), 9.10 min (A: 98%, B: 2%), 9.10-10.00 min (A: 98%, B: 2%); Mass spectrometer conditions: Capillary 3.25 kV, cone 20V, source temperature 115° C. desolvation T 350° C. All mass spectra were taken under electrospray ionisation (ESI) methods.

Method M) Instrument: ZQ2000 (Waters) coupled with UPLC and Sample Organizer and UV detector. MS detector: Waters ZQ2000. Column: Acquity UPLC-BEH C18 50×2.1 mm×1.7 um; flow rate 0.6 ml/min; mobile phase: A phase=water/acetonitrile 95/5+0.1% TFA, B phase=water/acetonitrile 5/95+0.1% TFA. 0-0.25 min (A: 95%, B: 5%), 3.3 min (A: 0%, B: 100%), 3.3-4.00 min (A: 0%, B: 100%), 4.1 min (A: 95%, B: 5%), 4.10-5.00 min (A: 95%, B: 5%); Mass spectrometer conditions: Capillary 3.25 kV, cone 20V, source temperature 115° C. desolvation T 350° C.

Method N) Instrument: ZQ2000 (Waters) coupled with UPLC and Sample Organizer and UV detector. MS detector: Waters ZQ2000. Column: Acquity UPLC-BEH C18 50×2.1 mm×1.7 um; flow rate 0.6 ml/min; mobile phase: A phase=water/acetonitrile 95/5+0.1% TFA, B phase=water/acetonitrile 5/95+0.1% TFA. 0-0.50 min (A: 95%, B: 5%), 6.00 min (A: 0%, B: 100%), 6.00-7.00 min (A: 0%, B: 100%), 7.1 min (A: 95%, B: 5%), 7.1-8.50 min (A: 95%, B: 5%); Mass spectrometer conditions: Capillary 3.25 kV, cone 20V, source temperature 115° C. desolvation T 350° C.

Method O) Instrument: ZQ2000 (Waters) coupled with 2777 Sample manager and 2996 Photodiode Array Detector. MS detector: Waters ZQ2000. Column: Synergi 20×2.0 mm 2.5 um; flow rate 0.7 ml/min; mobile phase: A phase=water/acetonitrile 95/5+0.1% TFA, B phase=water/acetonitrile 5/95+0.1% TFA. 0-0.25 min (A: 95%, B: 5%), 3.50 min (A: 0%, B: 100%), 3.50-4.50 min (A: 0%, B: 100%), 4.60 min (A: 95%, B: 5%), 4.60-6.00 min (A: 95%, B: 5%); Mass spectrometer conditions: Capillary 3.25 kV, cone 20V, source temperature 115° C. desolvation T 350° C.

Method P) Instrument: ZQ2000 (Waters) coupled with 2777 Sample manager and 2996 Photodiode Array Detector. MS detector: Waters ZQ2000. Column: Synergi 20×2.0 mm 2.5 um; flow rate 0.7 ml/min; mobile phase: A phase=water/acetonitrile 95/5+0.1% TFA, B phase=water/acetonitrile 5/95+0.1% TFA. 0-0.5 min (A: 95%, B: 5%), 1.50 min (A: 85%, B: 15%), 6.50 min (A: 70%, B: 30%), 7.50 min (A: 0%, B: 100%), 7.50-8.50 min (A: 0%, B: 100%), 8.60 min (A: 95%, B: 5%), 8.60-9.50 min (A: 95%, B: 5%); Mass spectrometer conditions: Capillary 3.25 kV, cone 20V, source temperature 115° C. desolvation T 350° C.

Preparative HPLC Purifications were Performed Under the Following Conditions:

Method Q) Instrument: Shimadzu (LC/8A and SCL/10A) coupled with UV spectrophotometric dector (SPD/6A). Column: Waters SymmetryPrep C18 19×30 mm×7 um; flow rate: 20ml/min; mobile phase: A phase=water/acetonitrile 9/1+0.5% TFA, B phase=water/acetonitrile 5/95+0.5% TFA using a 30 min gradient of 5-100% solvent B.

Method R) Instrument: HPLC-MS preparative system Waters (2767 and 2525) coupled with photodiode array detector and Micromass ZQ. Column: Waters XTerra MS C18 (19×300 mm, 10 um). Flow rate 20 ml/min. Mobile phase: A phase=water+0.1% TFA, B phase=acetonitrile+0.1% TFA. 0-3.0 min (A: 90%, B: 10%), 3.0 min (A: 90%, B: 10%), 3.0-26.0 min (A: 5%, B: 95%), 26.0 min (A: 5%, B: 95%), 26.0-30.0 min (A: 5%, B: 95%), 30.0 min (A: 5%, B: 95%), 30.0-30.5 min (A: 90%, B: 10%), 30.5 min (A: 90%, B: 10%), 30.5-31.5 min (A: 90%, B: 10%).

Method S) Instrument: HPLC-MS preparative system Waters (2767 and 2525) coupled with photodiode array detector and Micromass ZQ. Column: Waters XTerra MS C18 (19×300 mm, 10 um). Flow rate 20 ml/min. Mobile phase: A phase=water+0.1% TFA, B phase=acetonitrile +0.1% TFA. 0-1.0 min (A: 90%, B: 10%), 1.0 min (A: 90%, B: 10%), 1.0-13.0 min (A: 5%, B: 95%), 13.0 min (A: 5%, B: 95%), 13.0-15.0 min (A: 5%, B: 95%), 15.0 min (A: 5%, B: 95%), 15.0-15.5 min (A: 90%, B: 10%), 15.5 min (A: 90%, B: 10%), 15.5-16.5 min (A: 90%, B: 10%).

Method T) Instrument Parallel Flex Biotage. Column: Waters Symmetry Prep C18 (19×300 mm, 10 um). Flow rate 20 ml/min. Mobile phase: A phase=water+0.1% TFA, B phase=acetonitrile+0.1% TFA. 0-5.0 min (A: 95%, B: 5%), 5.0 min (A: 95%, B: 5%), 5.0-20.0 min (A: 5%, B: 95%), 20.0 min (A: 5%, B: 95%), 22.0 min (A: 5%, B: 95%), 22.0-23.0 min (A: 95%, B: 5%).

The fractions containing the pure material were pooled and neutralized with NaHCO₃. The acetonitrile was removed under reduced pressure and the residue was portioned between DCM and water. The organic phase was dried over Na₂SO₄, filtered and evaporated to dryness.

Most of the reactions were monitored by thin-layer chromatography on 0.25 mm Macherey-Nagel silica gel plates (60E-2254), visualized with UV light. Flash column chromatography was performed on silica gel (220-440 mesh, Fluka). Melting point determination was performed on a Buchi B-540 apparatus.

The examples are presented to illustrate the scope of this invention and the scope is defined in the attached claims.

In the following examples, the compounds of examples 5 and 21 are excluded from the claimed invention. Examples 1-4, 9, 10, 15, 17, 19, 20, 25, 48, 77, 153-155, 160 and 177 are excluded from the claimed invention and are intermediates in the synthesis of claimed compounds.

### Example 1

### N-[4-(Cyano-dimethyl-methyl)-phenyl]-3,4-dimethoxy-benzamide.

### 1(A) 2-Methyl-2-(4-nitro-phenyl)-propionitrile

To a solution of (4-nitro-phenyl)-acetonitrile (5.00 g; 30.9 mmol) in dry DMF (30 mL) cooled at 0° C., under N₂ atmosphere, was added NaH (60% dispersion in mineral oil; 1.23 g; 30.9 mmol) portionwise and the mixture was stirred for 15 min at 0° C. Then iodomethane (1.92 mL; 30.9 mmol) was added and the mixture was stirred at room temperature for 1.5 hour. The reaction was re-cooled at 0° C. and NaH (60% dispersion in mineral oil; 1.23 g; 30.9 mmol) was added again portionwise. After stirring at 0° C. for 15 min, iodomethane (1.92 mL; 30.9 mmol) was added and the reaction was stirred at room temperature for 16 hours. The solvent was evaporated under vacuum and the residue was taken up with EtOAc, washed with brine, dried over Na₂SO₄, filtered and concentrated under vacuum. The crude was purified by chromatography [SiO₂, Petroleum ether/EtOAc (95/5 to 8/2)] to give the title compound as a yellow solid (3.50 g, 60% yield).

LCMS (RT): 1.42 min (Method A); MS (ES+) gave m/z: 191.1 (MH+).

### 1(B) 2-(4-Amino-phenyl)-2-methyl-propionitrile

10% Pd/C (300 mg) was added to a solution of 2-methyl-2-(4-nitro-phenyl)-propionitrile (3.00 g; 15.8 mmol), prepared as in 1(A), in MeOH (65 mL). The mixture was hydrogenated at 1 bar at room temperature for 2.5 hours, the catalyst was filtered off and the filtrate was concentrated under reduced pressure to give the title compound as a yellow oil (2.40 g; 80% yield).

LCMS (RT): 0.78 min (Method A); MS (ES+) gave m/z: 161.1 (MH+).

### 1(C) N-[4-(Cyano-dimethyl-methyl)-phenyl]-3,4-dimethoxy-benzamide

3,4-Dimethoxy-benzoyl chloride (2.77 g; 13.8 mmol) was added portionwise to a solution of 2-(4-amino-phenyl)-2-methyl-propionitrile (1.85 g; 11.5 mmol), prepared as in 1(B), in triethylamine (3.20 mL; 23.0 mmol) and dry DCM (30 mL). The reaction was stirred at room temperature for 16 hours and then diluted with DCM, washed sequentially with 2M K₂CO₃, 1N HCl and brine. The organic layer was dried over sodium sulphate, filtered and evaporated under reduced pressure. The crude compound was purified by flash chromatography [SiO₂, Petroleum ether/EtOAc (95/5 to 8/2)] to afford the title compound as a white powder (2.68 g; 72% yield).

¹H NMR (300 MHz, CDCl3) δ(ppm): 7.78 (br. s., 1H), 7.62-7.73 (m, 2H), 7.51 (d, 1H), 7.46-7.51 (m, 2H), 7.40 (dd, 1H), 6.93 (d, 1H), 3.98 (s, 3H), 3.96 (s, 3H), 1.74 (s, 6H)

LCMS (RT): 2.10 min (Method B); MS (ES+) gave m/z: 325.19 (MH+).

MP: 139-141° C.

### Example 2

### N-[4-(1-Cyano-cyclopropyl)-phenyl]-3,4-dimethoxy-benzamide

### 2(A) 1-(4-Nitro-phenyl)-cyclopropanecarbonitrile

A solution of KNO₃ (1.10 g; 10.8 mmol) in conc. H₂SO₄ (9 mL) was added dropwise to a solution of 1-phenyl-cyclopropanecarbonitrile (1.50 g; 10.8 mmol) in conc. H₂SO₄ (9 mL), cooling with ice-acetone bath. The reaction was allowed to stir at ambient temperature for 1.5 hour and then was poured onto ice. The precipitate was filtered, dissolved in EtOAc and washed with water and then brine. The organic phase was dried over Na₂SO₄, filtered and evaporated under vacuum to give the title compound as a yellow solid (1.30 g). The compound was used in the next step without any further purification.

### 2(B) 1-(4-Amino-phenyl)-cyclopropanecarbonitrile

Prepared according to Example 1(B) starting from 1-(4-nitro-phenyl)-cyclopropanecarbonitrile (1.30 g; 6.91 mmol), prepared as in Example 2(A), and using 10% Pd/C (20 mg) in MeOH (30 mL). The catalyst was filtered off and the filtrate was evaporated under vacuum to give the title compound as dark oil (1.02 g). The compound was used in the next step without any further purification.

### 2(C) N-[4-(1-Cyano-cyclopropyl)-phenyl]-3,4-dimethoxy-benzamide

Prepared according to Example 1(C) starting from 1-(4-amino-phenyl)-cyclopropanecarbonitrile (158 mg; 1.00 mmol), prepared as in 2(B), and using 3,4-dimethoxy-benzoyl chloride (220 mg; 1.10 mmol), and triethylamine (167 uL; 1.20 mmol) in dry DCM (4.5 mL). Crystallization from isopropyl ether-DCM (1/1) afforded the title compound as a pale yellow solid (234 mg; 48% yield over three steps).

¹H NMR (300 MHz, CDCl3) δ(ppm): 7.78 (s, 1H), 7.56-7.68 (m, 2H), 7.49 (d, 1H), 7.39 (dd, 1H), 7.27-7.33 (m, 2H), 6.91 (d, 1H), 3.95 (s, 3H), 3.95 (s, 3H), 1.65-1.77 (m, 2H), 1.32-1.44 (m, 2H)

LCMS (RT): 5,67 min (Method G); MS (ES+) gave m/z: 323.2 (MH+). MP: 204-207° C.

### Example 3

### 2,3-Dihydro-benzo[1,4]dioxine-6-carboxylic acid [4-(1-cyano-cyclopentyp-phenyl]-amide

### 3(A) 1-(4-Nitro-phenyl)-cyclopentanecarbonitrile

A solution of (4-nitro-phenyl)-acetonitrile (6.00 g; 37.0 mmol) and 1,4-dibromo-butane (4.42 ml; 37.0 mmol) in DMSO/Et₂O (20 mL/20 mL) was added dropwise to a suspension of Nail (60% dispersion in mineral oil; 1.80 g; 81.4 mmol) in DMSO (20 mL), keeping the temperature below 30° C. After stirring at room temperature for one day, the reaction was quenched by adding isopropyl alcohol (5 mL) and then H₂O (5 mL). The mixture was concentrated under vacuum and the resulting aqueous solution was treated with 2N hydrochloric acid and extracted three times with Et₂O. The organic layers were pooled, washed with brine, dried over Na₂SO₄, filtered and concentrated under vacuum to dryness. The crude compound was purified by chromatography [SiO₂, Petroleum ether/EtOAc (9/1 to 8/2)] to yield the title compound as an orange solid (3.94 g; 50% yield).

LCMS (RT): 8.5 min (Method C); MS (ES+) gave m/z: 217.29 (MH+).

### 3(B) 1-(4-Amino-phenyl)-cyclopentanecarbonitrile

Prepared according to Example 1(B) starting from 1-(4-nitro-phenyl)-cyclopentanecarbonitrile (3.00 g; 13 9 mmol), prepared as in 3(A), and using 10% Pd/C (0.30 g) in MeOH (40 mL). The catalyst was filtered off and the filtrate was concentrated under reduced pressure to give the title compound as a white solid (2.38 g; 92% yield).

LCMS (RT): 2.67 min (Method B); MS (ES+) gave m/z: 187.33 (MH+).

### 3(C) 2,3-Dihydro-benzo[1,4]dioxin-6-carboxylic acid [4-(1-cyano-cyclopentyl)-phenyl]-amide

2,3-Dihydro-benzo[1,4]dioxine-6-carbonyl chloride (213 mg; 1.07 mmol) was added portionwise to a solution of 1-(4-amino-phenyl)-cyclopentanecarbonitrile (200 mg; 1.07 mmol), prepared as in 3(B), and triethylamine (180 uL; 1.29 mmol) in dry DCM (10 mL). The reaction was stirred at room temperature for 16 hours. Then 4-dimethylaminopyridine (131 mg, 1.07 mmol) was added and the resulting solution was heated to 130° C. under microwave irradiation for 3 hours. The solvent was evaporated under vacuum and the crude was partially purified by flash chromatography [SiO₂, Hexane/EtOAc (95/5 to 6/4)]. The resulting compound was further purified by preparative HPLC (Method R) to afford the title compound as a light yellow solid (0.12 mg; 3.2% yield).

¹H NMR (300 MHz, CDCl3) δ(ppm): 7.71 (br. s., 1H), 7.64 (m, 2H), 7.42-7.48 (m, 3H), 7.38 (dd, 1H), 6.96 (d, 1H), 4.28-4.37 (m, 4H), 2.38-2.59 (m, 2H), 1.89-2.15 (m, 6 H)

LCMS (RT): 6.41 min (Method H); MS (ES+) gave m/z: 349.3 (MH+).

MP: 164-167° C.

### Example 4

### N-[4-(1-Cyano-cyclopentyl)-phenyl]-4-dimethylamino-benzamide

To a solution of bromotripyrrolidinophosphonium hexafluorophosphate (192 mg; 0.41 mmol) in dry DCM (5 mL), was added 4-dimethylamino-benzoic acid (62.1 mg; 0.38 mmol) followed by a solution of 1-(4-amino-phenyl)-cyclopentanecarbonitrile (70.0 mg; 0.38 mmol), prepared as in 3(B), and ethyl-diisopropyl-amine (70 uL; 0.41 mmol) in dry DCM (3 mL). The reaction was stirred at room temperature for 72 hours, and then it was diluted with DCM and washed with water. The organic phase was dried (Na₂SO₄), filtered and evaporated to dryness. The crude product was purified by preparative HPLC (Method S) and then by chromatography [SiO₂, Hexane/EtOAc (9/1 to 6/4)]. The title compound was collected as a light yellow amorphous solid (6.00 mg; 5% yield).

¹H NMR (300 MHz, CDCl3) δ(ppm): 7.85 (m, 2H), 7.81 (br. s., 1H), 7.67 (m, 2H), 7.45 (m, 2H), 6.96 (m, 2H), 3.09 (s, 6H), 2.40-2.61 (m, 2H), 1.83-2.15 (m, 6H)

LCMS (RT): 2.94 min (Method H); MS (ES+) gave m/z: 334.2 (MH+).

### Example 5

### N-[4-(1-Carbamoyl-cyclopentyl)-phenyl]-3,4-dimethoxy-benzamide

### 5(A) 1-(4-Nitro-phenyl)-cyclopentanecarboxylic acid amide

A solution of 1-(4-nitro-phenyl)-cyclopentanecarbonitrile (225 mg; 1.04 mmol), prepared as in 3(A), and NaOH (125 mg; 3.12 mmol) in EtOH (3 mL) and H₂O (1 mL) was heated under microwave irradiation at 115° C. for 1.5 hour. The solvent was evaporated under reduced pressure and the aqueous phase was extracted twice with EtOAc. The combined organic layers were dried (Na₂SO₄) and evaporated under vacuum. The crude was purified by chromatography [SiO₂, Hexane/EtOAc (7/3)] to give the title compound as a yellow solid (50.0 mg; 20% yield).

LCMS (RT): 4.06 min (Method B); MS (ES+) gave m/z: 235.3 (MH+).

### 5(B) 1-(4-Amino-phenyl)-cyclopentanecarboxylic acid amide

Prepared according to Example 1(B) starting from 1-(4-nitro-phenyl)-cyclopentanecarboxylic acid amide (40.0 mg; 0.17 mmol), prepared as in 5(A), and using 10% Pd/C (5 mg) in MeOH (5 mL). The catalyst was filtered off and the filtrate was evaporated under vacuum to give the title compound as a white powder (31.0 mg; 89% yield).

LCMS (RT): 1.18 min (Method B); MS (ES+) gave m/z: 205.33 (MH+).

### 5(C) N-[4-(1-Carbamoyl-cyclopentyl)-phenyl]-3,4-dimethoxy-benzamide

The titled compound was prepared using the procedure of Example 1(C), starting from 1-(4-amino-phenyl)-cyclopentanecarboxylic acid amide (30.0 mg; 0.15 mmol), prepared as in 5(B), and using 3,4-dimethoxy-benzoyl chloride (30.0 mg; 0.15 mmol) and TEA (26 uL; 0.19 mmol). After stirring at room temperature for 40 hours, the reaction was diluted with DCM and washed twice with H₂O. The organic phase was dried over Na₂SO₄, filtered and evaporated to dryness by rotary evaporator. Purification by trituration with MeOH gave the title compound as a white powder (30.0 mg; 55%).

¹H NMR (300 MHz, DMSO-d6) δ(ppm): 9.99 (s, 1H), 7.46-7.73 (m, 4H), 7.32 (m, 2H), 7.00-7.11 (m, 1H), 6.90 (br. s., 1H), 6.72 (br. s., 1H), 3.84 (s, 3H), 3.84 (s, 3H), 3.36-3.50 (m, 2H), 1.54-1.87 (m, 6H).

LCMS (RT): 5.34 min (Method H); MS (ES+) gave m/z: 369.3 (MH+).

### Example 6

### 3,4-Dimethoxy-N-[4-(1-methyl-1-pyridin-4-yl-ethyl)-phenyl]-benzamide

### 6(A) 4-[1-Methyl-1-(4-nitro-phenyl)-ethyl]-pyridine

To a cold (0° C.) suspension of 4-(4-nitro-benzyl)-pyridine (1.00 g; 4.71 mmol) in dry DMF (30 mL), was added NaH (60% dispersion in mineral oil; 0.20 mg; 4.71 mmol) portionwise, followed by iodomethane (215 uL; 4.71 mmol). The cooling bath was removed and the solution was stirred at room temperature for 3 hours. After this time, a second portion of NaH (60% dispersion in mineral oil; 0.20 mg; 4.71 mmol) was added and the suspension was stirred at room temperature for 10 min and then at 90° C. for 1 hour. Iodomethane (215 uL; 4.71 mmol) was added again and the heating was maintained for additional 16 hours. The reaction was quenched with H₂O and concentrated under vacuum. The residue was portioned between EtOAc and H₂O. The organic phase was dried (Na₂SO₄), filtered and evaporated to dryness. Chromatography purification of the crude [SiO₂, Hexane/EtOAc (7/3) to EtOAc] provided the title compound as a yellow oil (0.11 g; 10% yield).

LCMS (RT): 3.23 min (Method A); MS (ES+) gave m/z: 242.38 (MH+).

### 6(B) 4-(1-Methyl-1-pyridin-4-yl-ethyl)-phenylamine

To a solution of 4-[1-methyl-1-(4-nitro-phenyl)-ethyl]-pyridine (110 mg; 0.45 mmol), obtained as described in 6(A), in MeOH (10 mL) and EtOAc (5 mL), was added 10% Pd/C (10 mg) and the resulting mixture was stirred at room temperature for 2 hours under hydrogen atmosphere (about 1 bar) using a Parr apparatus. The catalyst was removed by filtration through Celite® and the cake washed with EtOAc. The filtrate was concentrated under vacuum to afford the title compound as pale yellow oil (91.0 mg; quantitative yield).

LCMS (RT): 0.68 min (Method A); MS (ES+) gave m/z: 212.24 (MH+).

### 6(C) 3,4-Dimethoxy-N-[4-(1-methyl-1-pyridin-4-yl-ethyl)-phenyl]-benzamide

3,4-Dimethoxy-benzoyl chloride (94.0 mg; 0.47 mmol) was added portionwise to a solution of 4-(1-methyl-1-pyridin-4-yl-ethyl)-phenylamine. (91.0 mg; 0.43 mmol), prepared as in 6(B), and triethylamine (90 uL; 0.6 mmol) in dry DCM (6 mL), cooled at 0° C. The reaction was stirred at room temperature for 16 hours and then at 50° C. for 3 hours. The reaction was diluted with DCM, which was washed with water, dried over Na₂SO₄, filtered and concentrated under vacuum. The crude compound was purified by preparative HPLC (Method Q) to afford the title compound as a yellow oil (4.2 mg; 3% yield).

¹H NMR (300 MHz, DMSO-d6) δ(ppm): 10.01 (s, 1H), 8.52 (m, 2H), 7.46-7.75 (m, 4H), 7.25-7.44 (m, 3H), 7.21 (m, 1H), 7.07 (m, 1H), 3.84 (s, 6H), 1.67 (s, 3H), 1.24 (s, 3H).

LCMS (RT): 2.54 min (Method H); MS (ES+) gave m/z: 377.2 (MH+).

### Example 7

### 1-[4-(3,4-Dimethoxy-benzoylamino)-phenyl]-cyclopentanecarboxylic acid methyl ester

### 7(A) 1-(4-Amino-phenyl)-cyclopentanecarboxylic acid

1-(4-Amino-phenyl)-cyclopentanecarbonitrile (100 mg; 0.54 mmol), prepared as in 3(B), was dissolved in 50% KOH (2 mL) and EtOH (2 mL). The solution was heated to 105° C. under microwave irradiation for 9 hours. The solvent was evaporated under reduced pressure, the residue was taken up with water and the solution was acidified with 2N HCl. The resulting white precipitate was filtered and dried to provide title compound as hydrochloride salt (130 mg; quantitative yield).

LCMS (RT): 2.35 min (Method B); MS (ES+) gave m/z: 206.27 (MH+).

### 7(B) 1-[4-(3,4-Dimethoxy-benzoylamino)-phenyl]-cyclopentanecarboxylic acid

A mixture of 1-(4-amino-phenyl)-cyclopentanecarboxylic acid (hydrochloride salt; 130 mg; 0.54 mmol), prepared as in 7(A), 3,4-dimethoxy-benzoyl chloride (118 mg; 0.54 mmol) and TEA (223 uL; 1.61 mmol) in DCM (15 mL) was stirred at room temperature for 16 hours. The reaction was washed with water, dried over Na₂SO₄, filtered and concentrated under vacuum. Purification of the resulting crude compound by chromatography [SiO₂, DCM/MeOH/TFA (98.5/1.5/0.5)] provided the title compound as a white solid (75 mg; 38% yield).

¹H NMR (300 MHz, DMSO-d6) δ(ppm): 14.03 (br. s., 1H), 10.01 (br. s., 1H), 7.69 (m, 2H), 7.61 (dd, 1H), 7.53 (d, 1H), 7.31 (m, 2H), 7.08 (d, 1H), 3.84 (s, 3H), 3.84 (s, 3H), 2.52-2.58 (m, 2H), 1.58-1.87 (m, 6H).

LCMS (RT): 4.67 min (Method B); MS (ES+) gave m/z: 370.32 (MH+).

### 7(C) 1-[4-(3,4-Dimethoxy-benzoylamino)-phenyl]-cyclopentanecarboxylic acid methyl ester

To a suspension of Amberlyst®-15 hydrogen form (Fluka, 1.00 g; 3.52 mmol) in MeOH (6 mL), was added 1-[4-(3,4-dimethoxy-benzoylamino)-phenyl]-cyclopentanecarboxylic acid (0.13 g; 0.35 mmol), prepared as in 7(B), and the reaction was heated at 120° C. under microwave irradiation for 7 hours. The Amberlyst®-15 was collected by filtration and washed with MeOH. The combined filtrates were concentrated under vacuum and the residue was dissolved in DCM and washed with sat. NaHCO₃. The organic phase was dried over Na₂SO₄, filtered and concentrated to dryness. The title compound was isolated by chromatography [SiO₂, Petroleum ether/EtOAc (7/3)] as a pale yellow amorphous solid (0.03 g; 22% yield).

¹H NMR (300 MHz, CDCl3) δ(ppm): 7.70 (s, 1H), 7.53-7.59 (m, 2H), 7.49 (m, 1H), 7.33-7.40 (m, 3H), 6.86-6.96 (m, 1H), 3.96 (s, 3H), 3.95 (s, 3H), 3.61 (s, 3H), 2.55-2.74 (m, 2H), 1.83-2.06 (m, 2H), 1.45-1.78 (m, 2H), 1.06-1.34 (m, 1H), 0.79-0.93 (m, 1H).

LCMS (RT): 3.10 min (Method H); MS (ES+) gave m/z: 384.1 (MH+).

### Example 8

### 3,4-Dimethoxy-N-[4-(1-methylcarbamoyl-cyclopentyl)-phenyl]-benzamide

A solution of 1-[4-(3,4-dimethoxy-benzoylamino)-phenyl]-cyclopentanecarboxylic acid (20.0 mg; 0.05 mmol), prepared as in 7(B), HOBt (11.0 mg; 0.08 mmol), EDC (16.0 mg; 0.08 mmol), TEA (25 uL; 0.16 mmol) and methyl-amine (8M solution in EtOH; 1 mL; 8.00 mmol) in DCM (5 mL) was stirred at room temperature for 16 hours. After this time, the reaction was diluted with DCM, washed with 5% NaHCO₃ and then with water. The organic phase was dried (Na₂SO₄), filtered and evaporated to dryness. The residue was purified by trituration with MeOH. The white solid was collected and dried (7.0 mg; 34%).

¹H NMR (300 MHz, CDCl3) δ(ppm): 7.72 (br. s., 1H), 7.60-7.67 (m, 1H), 7.56-7.60 (m, 1H), 7.46-7.53 (m, 1H), 7.32-7.43 (m, 3H), 6.92 (m, 1H), 5.14 (q, 1H), 3.96 (s, 3H), 3.95 (s, 3H), 2.70 (d, 3H), 2.39-2.57 (m, 2H), 1.94-2.12 (m, 2H), 1.61-1.90 (m, 4H).

LCMS (RT): 2.43 min (Method H); MS (ES+) gave m/z: 383.1 (MH+).

### Example 9

### N-[4-(1-Cyano-cyclopentyl)-phenyl]-4-hydroxy-3-methoxy-benzamide

To a solution of 4-hydroxy-3-methoxy-benzoic acid (90.0 mg; 0.54 mmol) in DCM (1.8 mL) were added few drops of DMF. Oxalyl chloride (180 uL; 2.12 mmol) was added dropwise to this solution, which then was stirred at room temperature for 16 hours. The solvent was evaporated under vacuum and the resulting yellow oil was dissolved in DCM (5.52 mL). This solution was added dropwise to a stirred mixture of 1-(4-amino-phenyl)-cyclopentanecarbonitrile (100 mg; 0.54 mmol), prepared as in 3(B), and triethylamine (150 uL; 1.07 mmol) in DCM (4.6 mL) After stirring at room temperature for 3 hours, the solvent was evaporated under vacuum. The product was purified by preparative HPLC (Method Q), to yield the titled compound as a yellow oil (20.0 mg; 11% yield).

¹H NMR (300 MHz, CDCl3) δ(ppm): 7.83 (br. s., 1H), 7.64 (m, 2H), 7.53 (d, 1H), 7.43-7.50 (m, 2H), 7.34 (dd, 1H), 7.00 (d, 1H), 3.99 (s, 3H), 2.40-2.56 (m, 2H), 1.84-2.17 (m, 6H).

LCMS (RT): 2.76 min (Method H); MS (ES+) gave m/z: 337.1 (MH+).

### Example 10

### N-[4-(1-Cyano-cyclopentyl)-phenyl]-3-hydroxy-4-methoxy-benzamide

Prepared according to Example 9 starting from 3-hydroxy-4-methoxy-benzoic acid (90.0 mg; 0.54 mmol), and using few drops of DMF, oxalyl chloride (180 uL; 2.12 mmol) and then 1-(4-amino-phenyl)-cyclopentanecarbonitrile (100 mg; 0.54 mmol), prepared as in 3(B), and triethylamine (150 uL; 1.07 mmol). The crude product was purified by preparative HPLC (Method Q) and then by crystallization from MeOH to give the title compound as a white powder (5.0 mg, 3% yield).

¹H NMR (300 MHz, CDCl3) δ(ppm): 7.71 (br. s., 1H), 7.65 (m, 3H), 7.39-7.50 (m, 4H), 6.95 (dd, 2H), 5.70 (s, 1H), 2.40-2.57 (m, 2H), 1.88-2.18 (m, 6H).

LCMS (RT): 2.77 min (Method H); MS (ES+) gave m/z: 337.1 (MH+).

### Example 11

### N-[4-(1-Dimethylcarbamoyl-cyclopentyl)-phenyl]-3,4-dimethoxy-benzamide

Prepared according to Example 8 starting from 1-[4-(3,4-dimethoxy-benzoylamino)-phenyl]-cyclopentanecarboxylic acid (10.0 mg; 0.03 mmol), prepared as in 7(B), HOBt (6.0 mg; 0.04 mmol), EDC (8.0 mg; 0.04 mmol), TEA (10 uL; 0,05 mmol) and dimethyl-amine (2M solution in THF; 5 mL; 10.0 mmol). The crude product was purified by preparative HPLC (Method Q) to give the title compound as a white powder (5.0 mg, 46% yield).

¹H NMR (300 MHz, CDCl3) δ(ppm): 7.65 (br. s., 1H), 7.54-7.61 (m, 2H), 7.51 (d, 1H), 7.39 (dd, 1H), 7.21-7.26 (m, 2H), 6.94 (d, 1H), 3.97 (s, 3H), 3.95 (s, 3H), 2.79 (s, 6H), 2.42-2.53 (m, 2H), 1.98-2.09 (m, 2H), 1.72-1.82 (m, 4H).

LCMS (RT): 2.74 min (Method H); MS (ES+) gave m/z: 397.2 (MH+).

### Example 12

### 3,4-Dimethoxy-N-{4-[1-(5-methyl-[1,2,4]oxadiazol-3-yl)-cyclopentyl]-phenyl}-benzamide

### 12(A) N-[4-(1-Cyano-cyclopentyl)-phenyl]-3,4-dimethoxy-benzamide

Prepared according to Example 1(C) starting from 1-(4-amino-phenyl)-cyclopentanecarbonitrile (150 mg; 0.81 mmol), prepared as in 3(B), and using 3,4-dimethoxy-benzoyl chloride (162 mg; 0.81 mmol), and triethylamine (134 uL; 0.99 mmol) in dry DCM (5 mL). Crystallization from MeOH provided the title compound as a pale white solid (164 mg; 58% yield).

¹H NMR (300 MHz, CDCl3) δ(ppm): 7.78 (br. s., 1H), 7.66 (m, 2H), 7.51 (d, 1H), 7.46 (m, 2H), 7.40 (dd, 1H), 6.93 (d, 1H), 3.97 (s, 3H), 3.96 (s, 3H), 2.49 (br. s., 2H), 2.03 (br. s., 6H).

LCMS (RT): 5.11 min (Method B); MS (ES+) gave m/z: 351.33 (MH+).

### 12(B) 3,4-Dimethoxy-N-{4-[1-(5-methyl-[1,2,4]oxadiazol-3-yl)-cyclopentyl]-phenyl}-benzamide

To a solution of N-[4-(1-cyano-cyclopentyl)-phenyl]-3,4-dimethoxy-benzamide (100 mg; 0.29 mmol), prepared as in 12(A), in EtOH (5 mL), was added hydroxylamine (50% solution in water; 100 uL; 1.14 mmol) and the reaction was refluxed for 24 hours. The solvent was evaporated and the resulting white solid was dried under vacuum overnight. Then it was dissolved in dioxane (10 mL) and HOBt (50 mg; 0.37 mmol), EDC (71 mg; 0.37 mmol), TEA (80 uL; 0.58 mmol) and glacial acetic acid (16 uL; 0.29) were added. The resulting solution was stirred at room temperature for 16 hours and then heated to reflux for additional 8 hours. The solvent was removed under reduced pressure; the residue was dissolved in DCM and washed with 2M K₂CO₃ and water. The DCM phase was dried over Na₂SO₄, filtered and evaporated to dryness. Purification by preparative HPLC (Method Q) provided the title compound as a white solid (30 mg; 26% yield).

¹H NMR (600 MHz, CDCl3) δ(ppm): 7.71 (s, 1H), 7.57 (m, 2H), 7.49 (d, 1H), 7.41 (m, 2H), 7.37 (dd, 1H), 6.92 (d, 1H), 3.96 (s, 3H), 3.96 (s, 3H), 2.72-2.76 (m, 2H), 2.51 (s, 3H), 2.13-2.22 (m, 2H), 1.70-1.86 (m, 4H).

LCMS (RT): 4.16 min (Method H); MS (ES+) gave m/z: 408.2 (MH+).

### Example 13

### N-{4-[1-(Acetylamino-methyl)-cyclopentyl]-phenyl}-3,4-dimethoxy-benzamide

### 13(A) N-[4-(1-Aminomethyl-cyclopentyl)-phenyl]-3,4-dimethoxy-benzamide

To a solution of N-[4-(1-cyano-cyclopentyl)-phenyl]-3,4-dimethoxy-benzamide (500 mg; 1.42 mmol), prepared as in 12(A), in ethanol (40 mL) with few drops of 37% HCl, was added 10% Pd/C (100 mg) and the resulting suspension was hydrogenated at abut 3.3 bar at room temperature for 36 hours. The catalyst was filtered off and the filtrate was concentrated under reduced pressure. The crude was dissolved in DCM and loaded onto an ion-exchange (SCX) cartridge. The un-reacted starting material was recovered by eluting with DCM/MeOH (1/1) (100 mg), and then the title compound was recovered by eluting with MeOH/NH₄OH (9/1). N-[4-(1-Aminomethyl-cyclopentyl)-phenyl]-3,4-dimethoxy-benzamide was obtained as a light yellow solid (187 mg; 37% yield).

LCMS (RT): 1.59 min (Method E); MS (ES+) gave m/z: 355.1 (MH+).

### 13(B) N-4-[1-(Acetylamino-methyl)-cyclopentyl]-phenyl}-3,4-dimethoxy-benzamide

To a solution of N-[4-(1-aminomethyl-cyclopentyl)-phenyl]-3,4-dimethoxy-benzamide (26.0 mg; 0.07 mmol), prepared as described in 13(A), and TEA (12.0 uL; 0.09 mmol) in DCM (5 mL) at 0° C. under nitrogen, was added acetyl chloride (6.0 uL; 0.08 mmol). The cooling bath was removed and the solution was stirred at room temperature for 16 hours. The reaction was diluted with DCM and washed sequentially with sat. K₂CO₃, 10% HCl and brine. The organic phase was dried over Na₂SO₄, filtered and evaporated to dryness. The residue was purified by chromatography [SiO₂, Petroleum ether/EtOAc (9/1 to 2/8)] to furnish the title compound as a pale yellow amorphous solid (20.0 mg; 72% yield).

¹H NMR (300 MHz, CDCl3) δ(ppm): 7.77 (br. s., 1H), 7.61 (m, 2H), 7.53 (d, 1H), 7.41 (dd, 1H), 7.29-7.36 (m, 2H), 6.95 (d, 1H), 5.09 (br. s., 1H), 3.99 (s, 3H), 3.98 (s, 3H), 3.43 (d, 2H), 1.90 (s, 3H), 1.68-1.99 (m, 8H).

LCMS (RT): 3.27 min (Method L); MS (ES+) gave m/z: 397.1 (MH+).

### Example 14

### N-[3-(1-Cyano-cyclopentyl)-phenyl]-3,4-dimethoxy-benzamide

### 14(A) 1-(3-Nitro-phenyl)-cyclopentanecarbonitrile

Prepared according to Example 3(A) starting from (3-nitro-phenyl)-acetonitrile (4.00 g; 24.7 mmol), and using 1,4-dibromo-butane (2.95 mL; 24.7 mmol), NaH (60% dispersion in mineral oil; 1.97 g; 49.3 mmol). The crude product was purified by chromatography [SiO₂, Petroleum ether/EtOAc (9/1 to 8/2)] to give the title compound as a light orange solid (3.54 g, 66% yield).

LCMS (RT): 1.59 min (Method E); MS (ES+) gave m/z: 355.1 (MH+).

### 14(B) 1-(3-Amino-phenyl)-cyclopentanecarbonitrile

Prepared according to Example 1(B) starting from 1-(3-nitro-phenyl)-cyclopentanecarbonitrile (2.60 g; 12.1 mmol), prepared as in 14(A), and using 10% Pd/C (286 mg) in MeOH (50 mL) The catalyst was filtered off and the filtrate was concentrated under reduced pressure to give the title compound as a white solid (2.30 g; 97% yield).

LCMS (RT): 0.9 min (Method D); MS (ES+) gave m/z: 187.1 (MH+).

### 14(C) N-[3-(1-Cyano-cyclopentyl)-phenyl]-3,4-dimethoxy-benzamide

Prepared according to Example 1(C) starting from 1-(3-amino-phenyl)-cyclopentanecarbonitrile (350 mg; 1.88 mmol), prepared as in 14(B), and using 3,4-dimethoxy-benzoyl chloride (377 mg; 1.88 mmol), and triethylamine (313 uL; 2.26 mmol). The crude product was purified by preparative HPLC (Method Q) to yield the title compound as a white powder (290 mg; 44% yield).

¹H NMR (300 MHz, CDCl3) δ(ppm): 7.85 (s, 1H), 7.80 (t, 1H), 7.57-7.63 (m, 1H), 7.52 (d, 1H), 7.40-7.45 (m, 1H), 7.39 (t, 1H), 7.23-7.26 (m, 1H), 6.94 (d, 1H), 3.97 (s, 3H), 3.97 (s, 3H), 2.42-2.57 (m, 2H), 1.90-2.23 (m, 6H).

LCMS (RT): 2.97 min (Method H); MS (ES+) gave m/z: 351.1 (MH+).

MP: 59-61° C.

### Example 15

### N-[4-(1-Cyano-cyclopentyl)-phenyl]-4-isopropoxy-3-methoxy-benzamide

N-[4-(1-Cyano-cyclopentyl)-phenyl]-4-hydroxy-3-methoxy-benzamide (64.0 mg; 0.19 mmol), prepared as described in Example 9, and K₂CO₃ (26.0 mg; 0.19 mmol) were dissolved in dry DMF under nitrogen atmosphere. 2-Iodo-propane (18 uL; 0.19 mmol) was added and the reaction was stirred at room temperature for 16 hours. After this period, K₂CO₃ (13 mg; 0.08 mmol) and 2-iodo-propane (9 uL; 0.08 mmol) were added again and the reaction was stirred for additional 24 hours at room temperature. Then the solvent was removed under vacuum; the residue was taken up with DCM and washed with 1N NaOH. The organic phase was separated, dried (Na₂SO₄), filtered and evaporated to dryness. The crude product was purified by chromatography [SiO₂, Petroleum ether/EtOAc (8/2 to 6/4)] to afford the title compound as a white amorphous solid (18.0 mg; 25% yield).

¹H NMR (300 MHz, CDCl3) δ(ppm): 7.76 (br. s., 1H), 7.65 (m, 2H), 7.51 (d, 1H), 7.43-7.49 (m, 2H), 7.33-7.40 (m, 1H), 6.94 (d, 1H), 4.59-4.72 (m, 1H), 3.95 (s, 3H), 2.41-2.57 (m, 2H), 1.87-2.18 (m, 6H), 1.43 (d, 6H).

LCMS (RT): 3.25 min (Method H); MS (ES+) gave m/z: 379.3 (MH+).

### Example 16

### {1-[4-(3,4-Dimethoxy-benzoylamino)-phenyl]-cyclopentyl}-carbamic acid methyl ester

### 16(A) [1-(4-Nitro-phenyl)-cyclopentyl]-carbamic acid methyl ester

Na (16.5 mg; 0.72 mmol) was dissolved in MeOH (5 mL) at 0° C. under nitrogen atmosphere. To this solution, was added a solution of 1-(4-nitro-phenyl)-cyclopentanecarboxylic acid amide (84.0 mg; 0.36 mmol), prepared as in 5(A), in MeOH (5 mL). Then Bromine (37 uL; 0.72 mmol) was added and the resulting reaction was heated at 50° C. for 10 min. The reaction was cooled and water (10 mL) was added. The precipitate was collected by suction filtration, washed with cold water and dried under vacuum at 50° C. for 16 hours to afford the title compound as a white powder (54 mg; 57% yield).

LCMS (RT): 1.42min (Method D); MS (ES+) gave m/z: 265.21 (MH+).

### 16(B) [1-(4-Amino-phenyl)-cyclopentyl]-carbamic acid methyl ester

Prepared according to Example 1(B) starting from [1-(4-nitro-phenyl)-cyclopentyl]-carbamic acid methyl ester (54.0 mg; 0.20 mmol), prepared as in 16(A), and using 10% Pd/C (5 mg) in MeOH (10 mL). The catalyst was filtered off and the filtrate was concentrated under reduced pressure to yield the title compound as a white solid (48 g; quantitative yield).

LCMS (RT): 2.92 min (Method A); MS (ES+) gave m/z: 235.12 (MH+).

### 16(C) {1-[4-(3,4-Dimethoxy-benzoylamino)-phenyl]-cyclopentyl}-carbamic acid methyl ester

Prepared according to Example 1(C) starting from [1-(4-amino-phenyl)-cyclopentyl]-carbamic acid methyl ester (48 mg; 0.2 mmol), prepared as in 16(B), and using 3,4-dimethoxy-benzoyl chloride (41 mg; 0.2 mmol), and triethylamine (28 uL; 0.2 mmol). The crude product was purified by chromatography [SiO₂, Petroleum ether/EtOAc (8/2)] to yield the title compound as a white powder (61 mg; 74% yield).

¹H NMR (300 MHz, CDCl3) δ(ppm): 7.73 (br. s., 1H), 7.55-7.65 (m, 2H), 7.51 (d, 1H), 7.44 (m, 2H), 7.39 (dd, 1H), 6.94 (d, 1H), 5.02 (s, 1H), 3.98 (s, 3H), 3.97 (s, 3H), 3.58 (s, 3H), 2.20-2.46 (m, 2H), 1.98-2.18 (m, 2H), 1.74-1.95 (m, 4H).

LCMS (RT): 2.69 min (Method H); MS (ES+) gave m/z: 399.3 (MH+).

### Example 17

### 4-Methyl-3,4-dihydro-2H-benzo[1,4]oxazine-7-carboxylic acid [4-(1-cyano-cyclopentyl)-phenyl]-amide

A solution of 1-(4-amino-phenyl)-cyclopentanecarbonitrile (70.0 mg; 0.38 mmol), prepared as in 3(B), HOBt (76.0 mg; 0.56 mmol), EDC (108 mg; 0,56 mmol), TEA (130 uL; 0,94 mmol) and 4-methyl-3,4-dihydro-2H-1,4-benzoxazine-7-carboxylic acid (80 mg; 0.41 mmol) in DCM (10 mL) was stirred at room temperature for 72 hours. After this time, the reaction was diluted with DCM, washed with 5% NaHCO₃ and then with water. The organic phase was dried (Na2SO₄), filtered and evaporated to dryness. The residue was purified by preparative HPLC (Method Q) to afford the title compound as a dark oil (15.0 mg; 11%).

¹H NMR (300 MHz, CDCl3) δ(ppm): 7.66 (br. s., 1H), 7.58-7.63 (m, 2H), 7.36-7.44 (m, 3H), 7.27 (d, 1H), 6.65 (d, 1H), 4.25-4.30 (m, 2H), 3.32-3.42 (m, 2H), 2.97 (s, 3H), 2.36-2.48 (m, 2H), 1.82-2.15 (m, 6H).

LCMS (RT): 3.09 min (Method H); MS (ES+) gave m/z: 362.2 (MH+).

### Example 18

### 3,4-Dimethoxy-N-{4-[1-(morpholine-4-carbonyl)-cyclopentyl]-phenyl}-benzamide

Prepared according to Example 8 starting from 1-[4-(3,4-dimethoxy-benzoylamino)-phenyl]-cyclopentanecarboxylic acid (60.0 mg; 0.16 mmol), prepared as in 7(B), HOBt (33.0 mg; 0.24 mmol), EDC (47.0 mg; 0.24 mmol), TEA (50 uL; 0.35 mmol) and morpholine (14 uL; 0.16 mmol). The crude product was purified by chromatography [SiO₂, Petroleum ether/EtOAc (98/2 to 8/2)] to give the title compound as a white amorphous solid (40.0 mg, 57% yield).

¹H NMR (300 MHz, CDCl3) δ(ppm): 7.76 (s, 1H), 7.58-7.67 (m, 2H), 7.52 (d, 1H), 7.40 (dd, 1H), 7.18-7.28 (m, 2H), 6.94 (d, 1H), 3.98 (s, 3H), 3.97 (s, 3H), 3.73 (br. s., 8H), 2.35-2.55 (m, 2H), 1.92-2.10 (m, 2H), 1.66-1.88 (m, 4H).

LCMS (RT): 2.59 min (Method H); MS (ES+) gave m/z: 439.3 (MH+).

### Example 19

### Benzo[1,3]dioxole-5-carboxylic acid [4-(1-cyano-cyclopentyl)-phenyl]amide

To a suspension of PS-triphenylphosphine resin (Argonaut Technologies™; 358 mg; 0.86 mmol; loading:1.0-1.8 mmol/g) in DCM (20 mL), were added 1,3-benzodioxole-5-carboxylic acid (78.0 mg; 0.47 mmol), carbon tetrachloride (82 uL; 0.86 mmol) and then 1-(4-amino-phenyl)-cyclopentanecarbonitrile (80.0 mg; 0.43 mmol), prepared as in 3(B). The reaction was shake at room temperature for 16 hours, after that the resin was filtered off and the filtrate was concentrated under reduced pressure. Chromatography purification of the residue [SiO₂, Petroleum ether/EtOAc (8/2)] afforded the title compound as a white solid (61.0 mg; 43% yield).

¹H NMR (300 MHz, CDCl3) δ(ppm): 7.69-7.74 (m, 1H), 7.60-7.68 (m, 2H), 7.45-7.50 (m, 2H), 7.42 (dd, 1H), 7.39 (d, 1H), 6.91 (d, 1H), 6.08 (s, 2H), 2.39-2.57 (m, 2H), 1.86-2.17 (m, 6H).

LCMS (RT): 2.54 min (Method H); MS (ES+) gave m/z: 335.3 (MH+).

### Example 20

### N-[4-(1-Cyano-cyclohexyl)-phenyl]-3,4-dimethoxy-benzamide

### 20(A) 1-(4-Nitro-phenyl)-cyclohexanecarbonitrile

Prepared according to Example 3(A) starting from (4-nitro-phenyl)-acetonitrile (1.00 g; 6.17 mmol), and using 1,5-dibromo-pentane (0.83 mL; 6.17 mmol), NaH (60% dispersion in mineral oil; 0.31 g; 13.6 mmol). The crude product was purified by column chromatography [SiO₂, Petroleum ether/EtOAc (9/1)] to give the title compound as a yellow solid (0.51 g, 36% yield). The comound was used as such in the next step.

### 20(B) 1-(4-Amino-phenyl)-cyclohexanecarbonitrile

Prepared according to Example 1(B) starting from 1-(4-nitro-phenyl)-cyclohexanecarbonitrile (510 mg; 2,22 mmol), prepared as in 20(A), and using 10% Pd/C (50 mg) in MeOH (40 mL) The catalyst was filtered off and the filtrate was concentrated under reduced pressure to give the title compound, which was used in the next step without any further purification.

LCMS (RT): 3.1 min (Method B); MS (ES+) gave m/z: 201.07 (MH+).

### 20(C) N-[4-(1-Cyano-cyclohexyl)-phenyl]-3,4-dimethoxy-benzamide

Prepared according to Example 1(C) starting from 1-(4-amino-phenyl)-cyclohexanecarbonitrile (444 mg; 2.22 mmol), prepared as in 20(B), and using 3,4-dimethoxy-benzoyl chloride (445 mg; 2.22 mmol), and triethylamine (370 uL; 2.66 mmol) in dry DCM (15 mL). Purification by trituration with MeOH gave the title compound as a white powder (130 mg; 16% over three steps).

¹H NMR (300 MHz, CDCl3) δ(ppm): 7.83 (s, 1H), 7.61-7.72 (m, 2H), 7.46-7.52 (m, 3H), 7.41 (dd, 1H), 6.92 (d, 1H), 3.96 (s, 3H), 3.94-3.96 (m, 3H), 2.08-2.25 (m, 2H), 1.64-1.96 (m, 7 H), 1.16-1.39 (m, 1H).

LCMS (RT): 3.08 min (Method H); MS (ES+) gave m/z: 365.3 (MH+).

### Example 21

### N-[4-(1-Carbamoyl-1-methyl-ethyl)-phenyl]-3,4-dimethoxy-benzamide

To a solution of N-[4-(cyano-dimethyl-methyl)-phenyl]-3,4-dimethoxy-benzamide (93.0 mg; 0.29 mmol), prepared as described in 1(C), in EtOH (3 mL), were added 35% hydrogen peroxide (2.2 mL) and sat. K₂CO₃ (1 mL) The resulting solution was stirred at room temperature for 1 hour and then heated to 60° C. under microwave irradiation for 2 hours. The solvent was evaporated under vacuum, the residue dissolved in DCM (100 mL) and washed with water. The organic phase was dried (Na₂SO₄), filtered and evaporated under reduced pressure. Crystallization of the resulting residue from DCM afforded the title compound as a white solid (47 mg; 47% yield).

¹H NMR (300 MHz, CDCl3) δ(ppm): 8.16 (s, 1H), 7.66 (m, 2H), 7.52 (d, 1H), 7.44 (dd, 1H), 7.40 (m, 2H), 6.92 (d, 1H), 5.24 (br. s., 2H), 3.96 (s, 3H), 3.95 (s, 3H), 1.59 (s, 6H).

LCMS (RT): 2.03 min (Method G); MS (ES+) gave m/z: 343.3 (MH+).

MP:193-195° C.

### Example 22

### Butyl chloroformate (10 uL; 0.09 mmol) was added to a solution of 1-[4-(3,4-dimethoxy-benzoylamino)-phenyl]-cyclopentanecarboxylic acid (30.0 mg; 0.08 mmol), prepared according to 7(B), and N-methylmorpholine (8 uL; 0.08 mmol) in 1,2-dimethoxyethane (5 mL), at 0° C. After stirring at the same temperature for 20 min, the precipitate was removed by suction filtration. To the filtrate, was added a solution of sodium borohydride (6.0 mg; 0.16 mmol) in EtOH/H₂O (0.5 mL/0.5 mL) and the stirring was maintained for 2 hours at room temperature. The solvent was removed by rotary evaporator; the residue was taken up with EtOAc and washed in sequence with 1N NaOH (twice), 2M K₂CO₃ (twice), water and finally brine. The organic phase was dried (Na₂SO₄), filtered and concentrated under vacuum. The crude compound was purified by preparative HPLC (Method S) to provide the title compound as a pale yellow solid (7.0 mg; 25% yield).

¹H NMR (300 MHz, CDCl3) δ(ppm): 7.73 (s, 1H), 7.56-7.64 (m, 2H), 7.51 (d, 1H), 7.40 (dd, 1H), 7.31-7.37 (m, 2H), 6.93 (d, 1H), 3.97 (s, 3H), 3.96 (s, 3H), 3.56 (d, 2H), 3.50 (d, 1H), 1.84-2.08 (m, 4H), 1.67-1.82 (m, 4H).

LCMS (RT): 2.62 min (Method G); MS (ES+) gave m/z: 356.2 (MH+).

MP: 182-184° C.

### Example 23

### N-(4-{1-[(2,2-Dimethyl-propionylamino)-methyl]-cyclopentyl}-phenyl)-3,4-dimethoxy-benzamide

A mixture of 2,2-dimethyl-propionic acid (90.0 mg; 0.25 mmol), HOBt (43.0 mg; 0.32 mmol), EDC (49.0 mg; 0.25 mmol) and N-methylmorpholine (47 uL; 0.42 mmol) in DMF was stirred at room temperature for 10 min. Then, N-[4-(1-aminomethyl-cyclopentyl)-phenyl]-3,4-dimethoxy-benzamide (90.0 mg; 0.25 mmol), prepared as described in 13(A), was added and the stirring was maintained for additional 16 hours. The solvent was evaporated under reduced pressure. The residue was dissolved in DCM, washed sequentially with 2M K₂CO₃, 1N HCl and brine. The organic phase was dried over Na₂SO₄, filtered and evaporated to dryness. The crude compound was purified by preparative HPLC (Method S) to give the title compound as a white solid (54.0 mg; 58% yield).

¹H NMR (300 MHz, CDCl3) δ(ppm): 7.75 (s, 1H), 7.62 (m, 2H), 7.52 (d, 1H), 7.40 (dd, 1H), 7.30 (m, 2H), 6.93 (d, 1H), 5.30 (br. s., 1H), 3.98 (s, 3H), 3.97 (s, 3H), 3.39 (d, 2H), 1.67-1.99 (m, 8H), 1.10 (s, 9 H).

LCMS (RT): 3.01 min (Method G); MS (ES+) gave m/z: 439.4 (MH+).

MP: 165-167° C.

### Example 24

### 3,4-Dimethoxy-N-[4-(1-ureidomethyl-cyclopentyl)-phenyl]-benzamide

To a solution of N-[4-(1-aminomethyl-cyclopentyl)-phenyl]-3,4-dimethoxy-benzamide (40.0 mg; 0.12 mmol), prepared as described in 13(A), in dry THF (3 mL) under nitrogen atmosphere, was added trimethylsilyl-isocianate (18 uL; 0.14 mmol). After stirring at room temperature for 36 hours, the solvent was removed under vacuum; the residue was dissolved in sat. NaHCO₃ and the solution was stirred at room temperature for 30 min. The aqueous solution was extracted with DCM, which was collected, dried over Na₂SO₄, filtered and evaporated to dryness. The residue was triturated with acetonitrile and the title compound was collected by filtration as a white solid (16.0 mg; 36% yield).

¹H NMR (300 MHz, CDCl3) δ(ppm): 8.42 (s, 1H), 7.55 (m, 2H), 7.51 (d, 1H), 7.46 (dd, 1H), 7.25 (m, 2H), 6.89 (d, 1H), 4.67 (br. s., 1H), 4.49 (s, 2H), 3.94 (s, 3H), 3.92 (s, 3H), 3.30 (d, 2H), 1.62-1.95 (m, 8H).

LCMS (RT): 2.36 min (Method G); MS (ES+) gave m/z: 398.4 (MH+).

### Example 25

### N-[4-(1-Cyano-cyclopentyl)-phenyl]-3-isopropoxy-4-methoxy-benzamide

N-[4-(1-Cyano-cyclopentyl)-phenyl]-3-isopropoxy-4-methoxy-benzamide was prepared following the same procedure described in Example 15, starting from N-[4-(1-cyano-cyclopentyl)-phenyl]-3-hydroxy-4-methoxy-benzamide (100 mg; 0.30 mmol), prepared as in Example 10, and using K₂CO₃ (82.0 mg; 0.60 mmol) and 2-iodo-propane (29 uL; 0.30 mmol). The title compound was obtained as a white amorphous solid (23 mg: 20% yield).

¹H NMR (300 MHz, DMSO-d6) δ(ppm): 10.10 (s, 1H), 8.30 (s, 1H), 7.80 (m, 2H), 7.63 (dd, 0H), 7.54 (d, 1H), 7.46 (m, 2H), 7.09 (d, 1H), 4.56-4.69 (m, 1H), 3.84 (s, 3H), 2.33-2.46 (m, 2H), 1.98-2.17 (m, 2H), 1.80-1.98 (m, 4H), 1.29 (d, 6H).

LCMS (RT): 3.22 min (Method G); MS (ES+) gave m/z: 379.3 (MH+).

### Example 26

### N-[4-(2-Acetylamino-1,1-dimethyl-ethyl)-phenyl]-3,4-dimethoxy-benzamide

### 26(A) N-[4-(2-Amino-1,1-dimethyl-ethyl)-phenyl]-3,4-dimethoxy-benzamide

To a solution of N-[4-(cyano-dimethyl-methyl)-phenyl]-3,4-dimethoxy-benzamide (2.27 g; 7.00 mmol), prepared as in 1(C), in ethanol (90 mL), 10% Pd/C (455 mg) was added and the resulting suspension was hydrogenated at about 3.3 bar at room temperature for 15 hours. The catalyst was filtered off and the filtrate was concentrated under reduced pressure. The crude was dissolved in DCM and loaded onto an ion-exchange (SCX) cartridge. The un-reacted starting material was recovered by eluting with DCM/MeOH (1/1) (1.05 g), and then the title compound was recovered by eluting with MeOH/NH₄OH (9/1). N-[4-(2-Amino-1,1-dimethyl-ethyl)-phenyl]-3,4-dimethoxy-benzamide was obtained as a light yellow solid (1.20 g; 53% yield).

LCMS (RT): 0.97 min (Method D); MS (ES+) gave m/z: 329.1 (MH+).

### 26(B) N-[4-(2-Acetylamino-1,1-dimethyl-ethyl)-phenyl]-3,4-dimethoxy-benzamide

Triethylamine (48.0 ul; 0.27 mmol) was added to a solution of N-[4-(2-amino-1,1-dimethyl-ethyl)-phenyl]-3,4-dimethoxy-benzamide (75.0 mg; 0.23 mmol), prepared as in 26(A), in dry DCM (4 mL), at 0° C. After 5 min, a solution of acetyl chloride (20 ul; 0.27 mmol) in dry DCM (2 mL) was added dropwise and the resulting mixture was stirred at room temperature for 16 hours. The reaction was diluted with DCM and washed with sat. NaHCO₃ and then with 2N HCl. The organic layer was dried (Na₂SO₄), filtered and the solvent was evaporated under reduced pressure. The crude was purified by crystallization from DCM, affording the title compound as a white solid (43.0 mg; 51% yield).

¹H NMR (300 MHz, DMSO-d6) δ(ppm): 9.99 (s, 1H), 7.68 (m, 2H), 7.62 (dd, 1H), 7.56 (t, 1H), 7.53 (d, 1H), 7.33 (m, 2H), 7.08 (d, 1H), 3.85 (s, 6H), 3.25 (d, 2H), 1.79 (s, 3H), 1.23 (s, 6H).

LCMS (RT): 2.22 min (Method G); MS (ES+) gave m/z: 371.4 (MH+).

MP: 195-197° C.

### Example 27

### N-[4-(1-Acetylamino-1-methyl-ethyl)-phenyl]-3,4-dimethoxy-benzamide

### 27(A) N-(1-Methyl-1-phenyl-ethyl)-acetamide

A mixture of 1-methyl-1-phenyl-ethylamine (100 mg; 0.74 mmol), glacial acetic acid (42 uL; 0.74 mmol), HOBt (150 mg; 1.11 mmol), EDC (210 mg; 1.11 mmol) and TEA (230 uL; 1.63 mmol) in DCM (15 mL) was stirred at room temperature for 72 hours. The reaction was washed sequentially with H₂O, 2M K₂CO₃, 1N HCl and brine. The organic phase was dried over Na₂SO₄, filtered and evaporated to dryness to give the title compound as a white solid (130 mg), which was used in the next step without any further purification.

### 27(B) N-[1-Methyl-1-(4-nitro-phenyl)-ethyl]acetamide

A solution of KNO₃ (750 mg; 7.40 mmol) in conc. H₂SO₄ (4.5 mL) was added dropwise to a solution of N-(1-methyl-1-phenyl-ethyl)-acetamide (131 mg; 0.74 mmol), prepared as described in 27(A), in conc. H₂SO₄ (4 mL), at −7° C. (ice-NaCl cooling bath) under nitrogen atmosphere. The temperature was held below −5° C. during the dropping and then allowed to warm to room temperature. After stirring for 2 hours, the mixture was poured onto ice and the resulting aqueous phase was extracted with EtOAc (twice). The combined organic layers were washed with water and then with brine, dried over Na₂SO₄, filtered and evaporated under reduced pressure to afford 150 mg of the title compound as a yellow oil. This compound was used in the nest step without any further purification.

LCMS (RT): 3.69 min (Method B); MS (ES+) gave m/z: 223.1 (MH+).

### 27(C) N-[1-(4-Amino-phenyl)-1-methyl-ethyl]-acetamide

Prepared according to Example 1(B) starting from N-[1-methyl-1-(4-nitro-phenyl)-ethyl]-acetamide (150 mg; 0.68 mmol), prepared as in 27(B), and using 10% Pd/C (20 mg) in MeOH (15 mL). The catalyst was filtered off and the filtrate was concentrated under reduced pressure to give 103 mg of title compound, which was used in the next step without any further purification.

### 27(D) N-[4-(1-Acetylamino-1-methyl-ethyl)-phenyl]-3,4-dimethoxy-benzamide

Prepared according to Example 1(C) starting from N-[1-(4-amino-phenyl)-1-methyl-ethyl]-acetamide (103 mg; 0.54 mmol), prepared as in 27(C), and using 3,4-dimethoxy-benzoyl chloride (108 mg; 0.54 mmol), and triethylamine (112 uL; 0.81 mmol) in dry DCM (10 mL). After stirring for 2 hours at room temperature, the reaction was washed with water, dried over Na₂SO₄, filtered and concentrated under vacuum. The crude compound was purified by preparative HPLC (Method Q) to yield the title compound as a pink solid (15 mg; 6% yield over four steps).

¹H NMR (300 MHz, CDCl3) δ(ppm): 7.75 (s, 1H), 7.55-7.63 (m, 2H), 7.51 (d, 1H), 7.35-7.45 (m, 3H), 6.93 (d, 1H), 5.71 (s, 1H), 3.97 (d, 6H), 1.99 (s, 3H), 1.72 (s, 6H).

LCMS (RT): 2.15 min (Method G); MS (ES+) gave m/z: 357.4 (MH+).

### Example 28

### 3,4-Dimethoxy-N-{4-[1-methyl-1-(5-methyl-[1,2,4]oxadiazol-3-yl)-ethyl]-phenyl}-benzamide

Prepared according to Example 12(B), starting from N-[4-(cyano-dimethyl-methyl)-phenyl]-3,4-dimethoxy-benzamide (200 mg; 0.62 mmol), prepared as described in Example 1(C), and using hydroxylamine (50% solution in water; 210 uL; 2.48 mmol) and then HOBt (108 mg; 0.81 mmol), EDC (155 mg; 0.81 mmol), TEA (172 uL; 1.24 mmol) and glacial acetic acid (37 uL; 0.62 mmol). Purification by preparative HPLC (Method R) provided the title compound as a white amorphous solid (25.0 mg; 11% yield).

¹H NMR (300 MHz, CDCl3) δ(ppm): 7.71 (s, 1H), 7.58 (m, 2H), 7.50 (d, 1H), 7.39 (d, 1H), 7.35 (m, 2H), 6.92 (d, 1H), 3.97 (s, 3H), 3.96 (s, 3H), 2.54 (s, 3H), 1.78 (s, 6H).

LCMS (RT): 2.72 min (Method G); MS (ES+) gave m/z: 382.4 (MH+).

### Example 29

### Thiazole-4-carboxylic acid {1-[4-(3,4-dimethoxy-benzoylamino)-phenyl]-cyclopentylmethyl}-amide

Prepared according to Example 23, starting from N44-(1-aminomethyl-cyclopentyl)-phenyl]-3,4-dimethoxy-benzamide (85.0 mg; 0.24 mmol), prepared as described in 13(A), and using thiazole-4-carboxylic acid (26.0 mg; 0.20 mmol), HOBt (40.0 mg; 0.30 mmol), EDC (46.0 mg; 0.24 mmol), N-methylmorpholine (44 uL; 0.40 mmol). The crude compound was purified by preparative HPLC (Method S) to give the title compound as a white solid (50.0 mg; 54% yield).

¹H NMR (300 MHz, CDCl3) δ(ppm): 8.68 (d, 1H), 8.12 (d, 1H), 7.75 (s, 1H), 7.62 (m, 2H), 7.52 (d, 1H), 7.40 (d, 1H), 7.35 (m, 2H), 7.20 (br. s., 1H), 6.93 (d, 1H), 3.98 (s, 3H), 3.96 (s, 3H), 3.61 (d, 2H), 1.69-2.11 (m, 8H).

LCMS (RT): 2.87 min (Method G); MS (ES+) gave m/z: 466.4 (MH+).

MP: 101-104° C.

### Example 39

### 3,4-Dimethoxy-N-(4-{1-[(2-methoxy-ethyl)-methyl-carbamoyl]-cyclopentyl}-phenyl)-benzamide

Prepared according to Example 8 starting from 1-[4-(3,4-dimethoxy-benzoylamino)-phenyl]-cyclopentanecarboxylic acid (100 mg; 0.27 mmol), prepared as in 7(B), and using HOBt (44.0 mg; 0.35 mmol), EDC (72.0 mg; 0.38 mmol), TEA (76 uL; 0.54 mmol) and (2-methoxy-ethyl)-methyl-amine (47 uL; 0.54 mmol). The crude product was purified by chromatography [SiO₂, Petroleum ether/EtOAc (8/2 to 1/1)] to afford the title compound as a white amorphous powder (42 mg, 35% yield).

¹H NMR (300 MHz, DMSO-d6) δ(ppm): 10.03 (s, 1H), 7.70 (m, 2H), 7.61 (dd, 1H), 7.52 (d, 1H), 7.15 (m, 2H), 7.08 (d, 1H), 3.84 (s, 3H), 3.83 (s, 3H), 3.44 (br. s., 3H), 3.24 (s, 3H), 2.57 (br. s., 2H), 2.54-2.57 (m, 2H), 2.18-2.39 (m, 2H), 1.81-2.03 (m, 2H), 1.48-1.79 (m, 4H).

LCMS (RT): 2.17 min (Method G); MS (ES+) gave m/z: 441.2 (MH+).

### Example 43

### 3,4-Dimethoxy-N-{4-[1-methyl-1-(5-phenyl-[1,2,4]oxadiazol-3-yl)-ethyl]-phenyl}-benzamide

Prepared according to Example 12(B), starting from N-[4-(cyano-dimethyl-methyl)-phenyl]-3,4-dimethoxy-benzamide (100 mg; 0.31 mmol), prepared as described in 1(C), and using hydroxylamine (50% solution in water; 100 uL; 1.24 mmol) and then HOBt (50 mg; 0.36 mmol), EDC (69.0 mg; 0.36 mmol), TEA (51 uL; 0.73 mmol) and benzoic acid (34 mg; 0.28 mmol). Purification by preparative HPLC (Method Q) provided the title compound as a white powder (42 mg; 39% yield).

¹H NMR (300 MHz, CDCl3) δ(ppm): 8.06-8.16 (m, 2H), 7.45-7.64 (m, 6H), 7.33-7.45 (m, 4H), 6.92 (d, 1H), 3.96 (s, 3H), 3.95 (s, 3H), 1.85 (s, 6H).

LCMS (RT): 2.75 min (Method G); MS (ES+) gave m/z: 444.1 (MH+).

### Example 44

### N-{4-[1,1-Dimethyl-2-(2,2,2-trifluoro-acetylamino)-ethyl]-phenyl}-3,4-dimethoxy-benzamide

To a solution of N-[4-(2-amino-1,1-dimethyl-ethyl)-phenyl]-3,4-dimethoxy-benzamide (80.0 mg; 0.15 mmol), prepared as described in 26(A), and TEA (28 uL; 0.20 mmol) in dry DCM (3mL), trifluoroacetic anhydride (26 uL; 0.18 mmol) was added dropwise at 0° C. The reaction was warmed at room temperature and stirred for 16 hours then it was diluted with DCM, washed sequentially with 2M K₂CO₃, 1N HCl and brine. The organic phase was dried over Na₂SO₄, filtered and evaporated to dryness. The crude compound was purified by preparative HPLC (Method S) to give the title compound as a white amorphous solid (19.2 mg; 30% yield).

¹H NMR (300 MHz, DMSO-d6) δ(ppm): 10.01 (s, 1H), 9.23 (br. s., 1H), 7.69 (m, 2H), 7.62 (dd, 1H), 7.53 (d, 1H), 7.34 (m, 2H), 7.07 (d, 1H), 3.84 (s, 3H), 3.84 (s, 3H), 3.36 (s, 2H), 1.27 (s, 6H).

LCMS (RT): 2.26 min (Method G); MS (ES+) gave m/z: 425.2 (MH+).

### Example 45

### N-{4-[2-(Acetyl-methyl-amino)-1,1-dimethyl-ethyl]-phenyl}-3,4-dimethoxy-benzamide

### 45(A) N-[4-(2-Benzylamino-1,1-dimethyl-ethyl)-phenyl]-3,4-dimethoxy-benzamide

N-[4-(2-amino-1,1-dimethyl-ethyl)-phenyl]-3,4-dimethoxy-benzamide (100 mg; 0.30 mmol), prepared as described in 26(A), and benzaldehyde (31 uL; 0.30 mmol) were dissolved in dry toluene (15 mL) and heated to 110° C. for 5 hours under inert atmosphere and in presence of 4A molecular sieves. After this time, the molecular sieves were removed by filtration and the filtrate was concentrated under vacuum. The residue was dissolved in EtOH (15 mL) and treated with sodium borohydride (17.0 mg; 0.45 mmol). The reaction was stirred at room temperature for 72 hours, then quenched with water and concentrated under vacuum. The residue was taken up with water and extracted (twice) with EtOAc. The organic layers were combined, dried (Na₂SO₄), filtered and evaporated to dryness to give the title compound, which was used as such in the next step.

LCMS (RT): 1.16 min (Method D); MS (ES+) gave m/z: 419.0 (MH+).

### 45(B) N-{4-[2-(Benzyl-methyl-amino)-1,1-dimethyl-ethyl]-phenyl}-3,4-dimethoxy-benzamide

Iodomethane (18 uL; 0.30 mmol) was added to a solution of N-[4-(2-benzylamino-1,1-dimethyl-ethyl)-phenyl]-3,4-dimethoxy-benzamide (95 mg; 0.30 mmol), prepared as described in 45(A), and NaHCO₃ (28.0 mg; 0.33 mmol) in acetonitrile (10 mL). The reaction was stirred at room temperature for 5 hours and then heated to 40° C. for 5 hours. After that, the solvent was evaporated under vacuum, the resulting residue was taken up with DCM and washed with water. The organic layer was dried (Na₂SO₄), filtered and evaporated to dryness. The crude compound was dissolved in DCM (30 mL) and treated with PS-isocyanate resin (Argonaut Technologies™; 100 mg) in order to remove the un-reacted starting material. The resin was filtered off and washed first with DCM, and then with Et₂O. The filtrate was concentrated under vacuum to afford the title compound as a white solid (90.0 mg; 69% yield over two steps).

LCMS (RT): 1.17 min (Method D); MS (ES+) gave m/z: 433.0 (MH+).

### 45(C) N-{4-[2-(Acetyl-methyl-amino)-1,1-dimethyl-ethyl]-phenyl}-3,4-dimethoxy-benzamide

10% Pd/C (10 mg) was added to a solution of N-{4-[2-(benzyl-methyl-amino)-1,1-dimethyl-ethyl]-phenyl}-3,4-dimethoxy-benzamide (66.0 mg; 0.15 mmol), prepared as described in 45(B), and 37% HCl (few drops) in MeOH (15 mL). The mixture was hydrogenated at 2 bar at room temperature for 1 hour, then the catalyst was filtered off and the filtrate was evaporated to dryness. To a solution of the resulting residue in dry DCM (3 mL), were added sequentially TEA (63 uL; 0.45 mmol) and acetyl chloride (21 uL; 0.30 mmol). The reaction was stirred at room temperature for 30 min under inert atmosphere, then water was added and the phases were separated. The organic layer was dried (Na₂SO₄), filtered and concentrated by rotary evaporator. The crude compound was purified by chromatography [SiO₂, EtOAc/MeOH (98/2)] to yield the title compound as a pale yellow amorphous solid (36.0 mg; 63% yield).

¹H NMR (300 MHz, DMSO-d6, 373 K) δ(ppm): 9.63 (br. s., 1H), 7.68 (m, 2H), 7.61 (dd, 1H), 7.56-7.58 (m, 1H), 7.36 (m, 2H), 7.06 (d, 1H), 3.87 (s, 3H), 3.86 (s, 3H), 3.51 (s, 2H), 2.65 (s, 3H), 1.88 (br. s., 3H), 1.33 (s, 6H).

LCMS (RT): 1.93 min (Method G); MS (ES+) gave m/z: 385.2 (MH+).

### Example 48

### N-[4-(1-Cyano-cyclopentyl)-phenyl]-3-methoxy-4-(pyridin-4-ylmethoxy)-benzamide

### 48(A) 3-Methoxy-4-(pyridin-4-ylmethoxy)-benzoic acid methyl ester

To a solution of methyl vanillate (182 mg; 1.00 mmol) in THF (10 mL), were added 4-pyridinemethanol (141 mg; 1.30 mmol) and triphenylphosphine (341 mg; 1.30 mmol). After the solution was cooled at 0° C., diethyl azodicarboxylate (206 uL; 1.30 mmol) was added dropwise. Then the cooling bath was removed and the reaction was allowed to warm to room temperature and stirred for 1 hour. The solvent was removed under vacuum. The crude mixture was partially purified by ion-exchange chromatography [SCX, DCM/MeOH (1/1) to MeOH/NH₄OH (9/1)]. The resulting compound was further purified by column chromatography [SiO₂, Petroleum ether/EtOAc (8/2 to 6/4)] to furnish the title compound (220 mg; 80% yield).

LCMS (RT): 0.89 min (Method D); MS (ES+) gave m/z: 274.0 (MH+).

### 48(B) 3-Methoxy-4-(pyridin-4-ylmethoxy)-benzoic acid

A solution of 3-methoxy-4-(pyridin-4-ylmethoxy)-benzoic acid methyl ester (220 mg; 0.80 mmol), prepared as described in 48(A), and KOH (79.0 mg; 1.47 mmol) in MeOH (15 mL) was heated to reflux for 36 hours. Then the solution was treated with a large excess of Et₂O*HCl (saturated solution) and evaporated to dryness by rotary evaporator to give the title compound, which was used in the next step without any further purification.

LCMS (RT): 0.7 min (Method D); MS (ES+) gave m/z: 259.9 (MH+).

### 48(C) N-[4-(1-Cyano-cyclopentyl)-phenyl]-3-methoxy-4-(pyridin-4-ylmethoxy)-benzamide

Prepared according to Example 17, starting from 3-methoxy-4-(pyridin-4-ylmethoxy)-benzoic acid (207 mg; 0.80 mmol), prepared as in 48(B), and 1-(4-amino-phenyl)-cyclopentanecarbonitrile (149 mg; 0.80 mmol), prepared as in 3(B), and using HOBt (140 mg; 1.04 mmol), EDC (199 mg; 1.04 mmol), TEA (359 uL; 2.56 mmol) in DCM (5 mL) The residue was purified by crystallization from MeOH to afford the title compound as a pale yellow amorphous solid (100 mg; 29% yield).

¹H NMR (300 MHz, CDCl3) δ(ppm): 8.85 (s, 1H), 8.57 (d, 2H), 7.68 (d, 2H), 7.55 (d, 1H), 7.30-7.46 (m, 5H), 6.83 (d, 1H), 5.19 (s, 2H), 3.95 (s, 3H), 2.31-2.50 (m, 2H), 1.77-2.07 (m, 6H).

LCMS (RT): 1.94 min (Method G); MS (ES+) gave m/z: 428.1 (MH+).

### Example 50

### 3,4-Dimethoxy-N-{4-[2-(2-methoxy-benzoylamino)-1,1-dimethyl-ethyl]-phenyl}-benzamide

A mixture of 2-methoxy-benzoic acid (32.0 mg; 0.22 mmol), HOBt (37.0 mg; 0.29 mmol), EDC (53.0 mg; 0.29 mmol), TEA (66 uL; 0.47 mmol) in DCM (5 mL) was stirred at room temperature for 10 min. Then, N-[4-(2-amino-1,1-dimethyl-ethyl)-phenyl]-3,4-dimethoxy-benzamide (70.0 mg; 0.21 mmol), prepared as described in 26(A), was added and the stirring was maintained for additional 16 hours. The reaction was diluted with DCM, washed sequentially with 2M K₂CO₃, 1N HCl and brine. The organic phase was dried over Na₂SO₄, filtered and evaporated to dryness. The crude compound was partially purified by trituration with Et₂O/iPr₂O (1/1). The resulting compound was purified again by preparative HPLC (Method S) to give the title compound as a white solid (35.0 mg; 35% yield).

¹H NMR (300 MHz, CDCl3) δ(ppm): 8.18 (dd, 1H), 7.79 (s, 2H), 7.60-7.69 (m, 2H), 7.52 (d, 1H), 7.43-7.48 (m, 2H), 7.36-7.45 (m, 2H), 7.01-7.09 (m, 1H), 6.93 (d, 1H), 6.84-6.90 (m, 1H), 3.98 (s, 3H), 3.96 (s, 3H), 3.73 (d, 2H), 3.68 (s, 3H), 1.42 (s, 6H).

LCMS (RT): 2.29 min (Method G); MS (ES+) gave m/z: 463.2 (MH+).

### Example 52

### {2-[4-(3,4-Dimethoxy-benzoylamino)-phenyl]-2-methyl-propyl}-carbamic acid methyl ester

To a solution of N-[4-(2-amino-1,1-dimethyl-ethyl)-phenyl]-3,4-dimethoxy-benzamide (100 mg; 0.30 mmol), prepared as described in 26(A), and TEA (60 uL; 0.42 mmol) in DCM (10 mL), was added methyl chloroformate (28 uL; 0.36 mmol) and the resulting reaction was stirred at room temperature for 16 hours. The reaction was diluted with DCM and washed with 1N HCl. The organic layer was separated, dried over Na₂SO₄, filtered and evaporated to dryness. The crude compound was purified by chromatography [SiO₂, Petroleum ether/EtOAc (8/2 to 1/1)] to give the title compound as a pale white solid (103 mg; 88% yield).

¹H NMR (300 MHz, CDCl3) δ(ppm): 8.50 (s, 1H), 7.57-7.68 (m, 2H), 7.50 (d, 1H), 7.46 (dd, 1H), 7.31 (m, 2H), 6.89 (d, 1H), 4.50 (br. s., 1H), 3.93 (s, 3H), 3.91 (s, 3H), 3.58 (s, 3H), 3.33 (d, 2H), 1.29 (s, 6H).

LCMS (RT): 2.02 min (Method G); MS (ES+) gave m/z: 387.2 (MH+).

MP: 67-69° C.

### Example 53

### 3,4-Dimethoxy-N-{4-[1-methyl-1-(5-phenoxymethyl-[1,2,4]oxadiazol-3-yl)-ethyl]-phenyl}-benzamide

Prepared according to Example 12(B), starting from N-[4-(cyano-dimethyl-methyl)-phenyl]-3,4-dimethoxy-benzamide (100 mg; 0.31 mmol), prepared as described in 1(C), and using hydroxylamine (50% solution in water; 100 uL; 1.24 mmol) and then HOBt (50 mg; 0.36 mmol), EDC (69.0 mg; 0.36 mmol), TEA (51 uL; 0.73 mmol) and phenoxy-acetic acid (42 mg; 0.28 mmol). Purification by preparative chromatography [SiO₂, Petroleum ether/EtOAc (8/2 to 1/1)] afforded the title compound as a pale orange solid (31 mg; 23% yield).

¹H NMR (300 MHz, CDCl3) δ(ppm): 7.70 (s, 1H), 7.54-7.63 (m, 2H), 7.50 (d, 1H), 7.36-7.40 (m, 1H), 7.32-7.36 (m, 2H), 7.28-7.32 (m, 2H), 6.88-7.08 (m, 4H), 5.23 (s, 2H), 3.97 (s, 3H), 3.96 (s, 3H), 1.81 (s, 6H).

LCMS (RT): 2.64 min (Method G); MS (ES+) gave m/z: 474.2 (MH+).

MP: 165-167° C.

### Example 54

### N-{4-[1-(Acetylamino-methyl)-cyclopropyl]-phenyl}-3,4-dimethoxy-benzamide

### 54(A) C-[1-(4-Nitro-phenyl)-cyclopropyl]-methylamine

To a solution of 1-(4-nitro-phenyl)-cyclopropanecarbonitrile (180 mg; 0.96 mmol), prepared as described in 2(A), in dry THF (10 mL), borane-THF complex (1M solution in THF; 4.78 mL) was added dropwise over 15 min while stirring under nitrogen atmosphere. The resulting solution was refluxed for 1 hour, cooled at room temperature and quenched by adding methanol dropwise. The solvent was removed under vacuum, the residue was taken up with THF (10 mL). Few drops of 50% NaOH were added and the reaction was heated to 50° C. for 30 min. The reaction was concentrated under vacuum, the resulting residue was dissolved in DCM and washed with water, dried over Na₂SO₄, filtered and evaporated to dryness. The crude compound was partially purified by ion-exchange chromatography [SCX, DCM/MeOH (1/1) to MeOH/NH₄OH (9/1)] and used in the next step without any further purification.

LCMS (RT): 2.3 min (Method A); MS (ES+) gave m/z: 193.08 (MH+).

### 54(B) N-[1-(4-Nitro-phenyl)-cyclopropylmethyl]-acetamide

To a solution of C-[1-(4-nitro-phenyl)-cyclopropyl]-methylamine (184 mg; 0.96 mmol), prepared as in 54(A), and TEA (161 uL; 1.19 mmol) in dry DCM (10 mL), acetyl chloride (74 uL; 1.05 mmol) was added dropwise while stirring at 0° C. under nitrogen atmosphere. The reaction was allowed to rise to room temperature and stirred for additional 16 hours. The reaction was diluted with DCM, washed with water, 1N HCl and brine. The organic layer was separated, dried (Na₂SO₄), filtered and evaporated to dryness. The crude compound was triturated with MeOH, filtered and dried under vacuum, obtaining the title compound as a yellow powder (183 mg; 81% yield over two steps).

LCMS (RT): 1.14 min (Method D); MS (ES+) gave m/z: 235.0 (MH+).

### 54(C) N-[1-(4-Amino-phenyl)-cyclopropylmethyl]-acetamide

Prepared according to Example 1(B) starting from N-[1-(4-nitro-phenyl)-cyclopropylmethyl]-acetamide (183 mg; 0.78 mmol), prepared as in 54(C), and using 10% Pd/C (10 mg) in MeOH (10 mL). The catalyst was filtered off and the filtrate was concentrated under reduced pressure to give the title compound as a white solid (134 mg; 84% yield).

LCMS (RT): 0.56 min (Method D); MS (ES+) gave m/z: 205.1 (MH+).

### 54(D) N-{4-[1-(Acetylamino-methyl)-cyclopropyl]-phenyl}-3,4-dimethoxy-benzamide

Prepared according to Example 1(C) starting from N-[1-(4-amino-phenyl)-cyclopropylmethyl]-acetamide (134 mg; 0.65 mmol), prepared as in 54(C), and using 3,4-dimethoxy-benzoyl chloride (144 mg; 0.72 mmol), and triethylamine (110 uL; 0.78 mmol) in dry DCM (10 mL). The crude compound was purified by preparative HPLC (Method Q) to yield the title compound as a white powder (28 mg; 11% yield).

¹H NMR (300 MHz, CDCl3) δ(ppm): 7.73 (s, 1H), 7.54-7.62 (m, 2H), 7.51 (d, 1H), 7.39 (dd, 1H), 7.28-7.36 (m, 2H), 6.93 (d, 1H), 5.44 (br. s., 1H), 3.98 (s, 3H), 3.96 (s, 3H), 3.45 (d, 2H), 1.94 (s, 3H), 0.87-0.93 (m, 4H).

LCMS (RT): 1.71 min (Method G); MS (ES+) gave m/z: 369.1 (MH+).

MP: 204-206° C.

### Example 60

### N-{4-[1,1-Dimethyl-2-(2-oxo-oxazolidin-3-yl)-ethyl]-phenyl}-3,4-dimethoxy-benzamide

To a solution of N-[4-(2-amino-1,1-dimethyl-ethyl)-phenyl]-3,4-dimethoxy-benzamide (188 mg; 0.57 mmol), prepared as described in 26(A), and TEA (153 uL; 1.14 mmol) in DCM (5 mL), was added 2-chloroethyl chloroformate (88 uL; 0.85 mmol) and the resulting reaction was stirred at room temperature under nitrogen atmosphere for 24 hours. The reaction was quenched with water, the phases were separated and the aqueous one was extracted with DCM. The combined organic layers were dried over Na₂SO₄, filtered and evaporated to dryness. The residue was suspended in DMF (200 mL) and a catalytic amount of potassium iodine was added, followed by NaH (60% dispersion in mineral oil; 34 mg; 0.85 mmol). The resulting mixture was heated at 70° C. for 2 hours, then water was added and the solvent were removed under reduced pressure. The crude product was purified by chromatography [SiO₂, DCM/MeOH (97/3)]. The resulting compound was further purified by preparative HPLC (Method T) to give the title compound as a pale white solid (75 mg; 33% yield).

¹H NMR (300 MHz, CDCl3) δ(ppm): 7.79 (s, 1H), 7.56-7.69 (m, 2H), 7.51 (d, 1H), 7.35-7.46 (m, 3H), 6.93 (d, 1H), 4.04-4.12 (m, 2H), 3.97 (s, 3H), 3.96 (s, 3H), 3.43 (s, 2H), 2.86-2.93 (m, 2H), 1.41 (s, 6H).

LCMS (RT): 1.95 min (Method G); MS (ES+) gave m/z: 399.18 (MH+).

MP: 190-191° C.

### Example 75

### N-[4-(Cyano-dimethyl-methyl)-2-methoxy-phenyl]-3,4-dimethoxy-benzamide

### 75(A) (3-Methoxy-4-nitro-phenyl)-acetonitrile

2-Methoxy-4-methyl-1-nitro-benzene (0.50 g; 2.99 mmol) was dissolved in tert-butoxy-bis(dimethylamino)-methane (1.25 mL; 5.74 mmol) and the mixture was heated at 100° C. for 4 hours. The reaction was concentrated under reduced pressure to afford a dark-brown oil, which was taken up with water (20 mL) and treated with hydroxylamine-O-sulfonic acid (1.01 g; 8.97 mmol) at room temperature for 2 hours. The precipitate was filtered off, washed with cold water and dried under vacuum to give title compound as a yellow solid (0.17 g; 30% yield).

LCMS (RT): 3.44 min (Method B).

### 75(B) 2-(3-Methoxy-4-nitro-phenyl)-2-methyl-propionitrile

To a solution of (3-methoxy-4-nitro-phenyl)-acetonitrile (175 mg; 0.91 mmol), prepared as described in 75(A), and tetrabuthylammonium bromide (0.50 g; 0.15 mmol) in toluene (4 mL), was added a solution of NaOH (0.36 mg; 9.11 mmol) in water (4 mL), directly followed by iodomethane (285 uL; 4.56 mmol). The resulting reaction was vigorously stirred at room temperature for 4 hours then diluted with EtOAc, washed in sequence with 5%. NaHCO₃, 1N hydrochloric acid, and finally with brine. The organic phase was collected, dried over Na₂SO₄, filtered and evaporated to dryness. Flash chromatography of the residue [SiO₂, Petroleum ether/EtOAc (8/2)] afforded the title compound as a yellow solid (145 mg; 72% yield).

LCMS (RT): 4.96 min (Method B); MS (ES+) gave m/z: 221.05 (MH+).

### 75(C) 2-(4-Amino-3-methoxy-phenyl)-2-methyl-propionitrile

Prepared according to Example 1(B), starting from 2-(3-methoxy-4-nitro-phenyl)-2-methyl-propionitrile (145 mg; 0.66 mmol), prepared as in 75(B), and using 10% Pd/C (20 mg) in MeOH (20 mL). The catalyst was filtered off and the filtrate was evaporated under vacuum to give the title compound as a white solid (114 mg; 90% yield).

LCMS (RT): 2.47 min (Method B); MS (ES+) gave m/z: 191.09 (MH+).

### 75(D) N-[4-(Cyano-dimethyl-methyl)-2-methoxy-phenyl]-3,4-dimethoxy-benzamide

Prepared according to Example 1(C), starting from 2-(4-amino-3-methoxy-phenyl)-2-methyl-propionitrile (114 mg; 0.60 mmol), prepared as in 75(C), and using 3,4-dimethoxy-benzoyl chloride (120 mg; 0.60 mmol), and triethylamine (250 uL; 1.80 mmol) in dry DCM (5 mL). Crystallization from isopropyl ether/DCM (1/1) afforded the title compound as a white solid (199 mg; 93% yield).

¹H NMR (300 MHz, CDCl3) δ(ppm): 8.52 (d, 1H), 8.48 (s, 1H), 7.54 (d, 1H), 7.42 (dd, 1H), 7.00-7.14 (m, 2H), 6.94 (d, 1H), 3.99 (s, 3H), 3.98 (s, 3H), 3.96 (s, 3H), 1.75 (s, 6H).

LCMS (RT): 2.26 min (Method G); MS (ES+) gave m/z: 355.12 (MH+).

MP: 110-112° C.

### Example 77

### N-[4-(1-Cyano-1-ethyl-propyl)-phenyl]-3,4-dimethoxy-benzamide

### 77(A) 2-Ethyl-2-(4-nitro-phenyl)-butyronitrile

Prepared according to Example 75(B) starting from (4-nitro-phenyl)-acetonitrile (1.00 g; 6.17 mmol), and using tetrabuthylammonium bromide (0.34 g; 1.06 mmol), 50% NaOH (2.47 mL; 61.73 mmol) and iodo-ethane (2.00 mL; 24.7 mmol). The crude product was purified by chromatography [SiO₂, Petroleum ether/EtOAc (97/3 to 9/1)] to give the title compound as a yellow solid (0.07 g, 6% yield).

LCMS (RT): 1.12 min (Method D).

### 77(B) 2-(4-Amino-phenyl)-2-ethyl-butyronitrile

Prepared according to Example 1(B), starting from 2-ethyl-2-(4-nitro-phenyl)-butyronitrile (75.0 mg; 0.34 mmol), prepared as in 77(A), and using 10% Pd/C (10 mg) in MeOH (5 mL). The catalyst was filtered off and the filtrate was evaporated under vacuum. The crude mixture was purified by ion-exchange chromatography [SCX, DCM/MeOH (1/1) to MeOH/NH₄OH (9/1)] to give the title compound as a white solid (45.0 mg; 70% yield).

LCMS (RT): 0.96 min (Method D); MS (ES+) gave m/z: 188.1 (MH+).

### 77(C) N-[4-(1-Cyano-1-ethyl-propyl)-phenyl]-3,4-dimethoxy-benzamide

Prepared according to Example 1(C), starting from 2-(4-amino-phenyl)-2-ethyl-butyronitrile (45.0 mg; 0.24 mmol), prepared as in 77(B), and using 3,4-dimethoxy-benzoyl chloride (48.0 mg; 0.24 mmol), and triethylamine (40 uL; 0.28 mmol) in dry DCM (3 mL). The crude compound was purified first by chromatography [SiO₂, DCM/MeOH (9/1 to 7/3)], and then by preparative HPLC (Method S), to afford the title compound as a colourless amorphous solid (8.6 mg; 11% yield).

¹H NMR (300 MHz, CDCl3) δ(ppm): 7.78 (s, 1H), 7.68 (m, 2H), 7.51 (d, 1H), 7.37-7.43 (m, 3H), 6.94 (d, 1H), 3.98 (s, 3H), 3.97 (s, 3H), 1.86-2.13 (m, 4H), 0.94 (t, 6H).

LCMS (RT): 2.40 min (Method G); MS (ES+) gave m/z: 353.21 (MH+).

### Example 78

### 3,4-Dimethoxy-N-{4-[1-methyl-1-(5-methyl-[1,3,4]oxadiazol-2-yl)-ethyl]-phenyl}-benzamide

### 78(A) 2-(4-Nitro-phenyl)-isobutyramide

2-Methyl-2-(4-nitro-phenyl)-propionitrile (500 mg; 2.63 mmol), prepared as described in 1(A), was suspended in a mixture of 35% hydrogen peroxide (6 mL), sat. K₂CO₃ (3 mL) and EtOH (3 mL). After stirring at room temperature for 16 hours, the volatiles were evaporated under vacuum, the residue was taken up with DCM and washed with water. The organic phase was separated, dried over Na₂SO₄, filtered and evaporated under vacuum to give the title compound as a yellow solid (538 mg; 98% yield).

LCMS (RT): 1.05 min (Method D); MS (ES+) gave m/z: 209.0 (MH+).

### 78(B) 2-Methyl-2-(4-nitro-phenyl)-propionic acid

To a solution of 2-(4-nitro-phenyl)-isobutyramide (538 mg; 2.59 mmol), prepared as in 78(A), in THF (25 mL), was added 37% HCl (5 mL) and the resulting reaction was refluxed for 20 hours. Then, the solution was concentrated under vacuum and the residue was portioned between DCM and water. The organic layer was dried (Na₂SO₄), filtered and evaporate under vacuum to give the title compound as a white solid (512 mg; quantitative yield).

LCMS (RT): 3.1 min (Method A); MS (ES+) gave m/z: 210.00 (MH+).

### 78(C) Acetic acid N′-[2-methyl-2-(4-nitro-phenyl)-propionyl]-hydrazide

To a solution of 2-methyl-2-(4-nitro-phenyl)-propionic acid (200 mg; 0.96 mmol), prepared as described in 78(B), in DCM (10 mL) and in presence of few drops of DMF, was added oxalyl chloride (361 uL; 2.87 mmol) at 0° C. under nitrogen atmosphere. After stirring at room temperature for 3 hours, the solvent was evaporated under vacuum. The resulting acyl chloride was taken up with dry DCM (5 mL) and added dropwise to a cold solution of acetyl hydrazine (77.9 mg; 1.05 mmol) in DCM (5 mL). The reaction was stirred at room temperature for 16 hours, then it was diluted with DCM, washed with 1N NaOH and finally water. The organic phase was dried over Na₂SO₄, filtered and evaporated to dryness, to afford the title compound as a white solid (223 mg; 88% yield).

LCMS (RT): 2.5 min (Method A); MS (ES+) gave m/z: 266.07 (MH+).

### 78(D) 2-Methyl-5-[1-methyl-1-(4-nitro-phenyl)-ethyl]-[1,3,4]oxadiazole

Phosphorus oxychloride (86 uL; 0.92 mmol) was added dropwise to a solution of acetic acid N′-[2-methyl-2-(4-nitro-phenyl)-propionyl]-hydrazide (223 mg; 0.84 mmol) in acetonitrile (10 mL) and the resulting solution was heated at reflux for 2 hours. After this period, the reaction was concentrated under vacuum, quenched with water and the pH was adjusted to about 7 by adding NaHCO₃. The aqueous solution was extracted twice with DCM and the combined organic layers were dried (Na₂SO₄), filtered and evaporated under vacuum to yield a deep yellow gum. Chromatography purification [SiO₂, Petroleum ether/EtOAc (9/1 to 1/1)] afforded the title compound as a white solid (62 mg; 30% yield).

LCMS (RT): 1.28 min (Method D); MS (ES+) gave m/z: 248.0 (MH+).

### 78(E) 4-[1-Methyl-1-(5-methyl-[1,3,4]oxadiazol-2-yl)-ethyl]-phenylamine

Prepared according to Example 1(B), starting from 2-methyl-5-[1-methyl-1-(4-nitro-phenyl)-ethyl]-[1,3,4]oxadiazole (62.0 mg; 0.25 mmol), prepared as in 78(D), and using 10% Pd/C (10 mg) in MeOH (5 mL) The catalyst was filtered off and the filtrate was evaporated under vacuum to give the title compound as a pale yellow solid (53.0 mg; quantitative yield).

LCMS (RT): 1.6 min (Method A); MS (ES+) gave m/z: 218.12 (MH+).

### 78(F) 3,4-Dimethoxy-N-{4-[1-methyl-1-(5-methyl-[1,3,4]oxadiazol-2-yl)-ethyl]-phenyl}-benzamide

Prepared according to Example 1(C), starting from 4-[1-methyl-1-(5-methyl-[1,3,4]oxadiazol-2-yl)-ethyl]-phenylamine (53.0 mg; 0.24 mmol), prepared as in 78(E), and using 3,4-dimethoxy-benzoyl chloride (58.0 mg; 0.29 mmol), and triethylamine (51 uL; 0.37 mmol) in dry DCM (10 mL) The crude compound was purified first by chromatography [SiO₂, DCM/MeOH (8/2 to 1/1)], and then by preparative HPLC (Method Q), to afford the title compound as a white amorphous solid (9.0 mg; 10% yield).

¹H NMR (300 MHz, CDCl3) δ(ppm): 7.75 (s, 1H), 7.59 (m, 2H), 7.50 (d, 1H), 7.39 (dd, 1H), 7.31 (m, 2H), 6.92 (d, 1H), 3.97 (s, 3 H), 3.96 (s, 3 H), 2.47 (s, 3H), 1.82 (s, 6H).

LCMS (RT): 1.93 min (Method G); MS (ES+) gave m/z: 382.08 (MH+).

### Example 79

### N-[3-(Cyano-dimethyl-methyl)-phenyl]-3,4-dimethoxy-benzamide

### 79(A) 2-Methyl-2-(3-nitro-phenyl)-propionitrile

Prepared according to Example 75(B), starting from (3-nitro-phenyl)-acetonitrile (2.00 g; 12.3 mmol), and using tetrabuthylammonium bromide (0.79 g; 2.46 mmol), 50% NaOH (4.92 mL; 123 mmol) and iodomethane (3.05 mL; 49.4 mmol). The crude product was purified by chromatography [SiO₂, Petroleum ether/EtOAc (95/5 to 8/2)] to give the title compound as a white solid (1.20 g; 51% yield).

LCMS (RT): 1.55 min (Method D).

### 79(B) 2-(3-Amino-phenyl)-2-methyl-propionitrile

Prepared according to Example 1(B), starting from 2-methyl-2-(3-nitro-phenyl)-propionitrile (900 mg; 4.74 mmol), prepared as in 79(A), and using 10% Pd/C (20 mg) in MeOH (20 mL) The catalyst was filtered off and the filtrate was evaporated under vacuum to give the title compound as a pale yellow solid (748 mg; quantitative yield).

LCMS (RT): 1.9 min (Method A); MS (ES+) gave m/z: 161.06 (MH+).

### 79(C) N-[3-(Cyano-dimethyl-methyl)-phenyl]-3,4-dimethoxy-benzamide

Prepared according to Example 1(C), starting from 2-(3-amino-phenyl)-2-methyl-propionitrile (700 mg; 4.37 mmol), prepared as in 79(B), and using 3,4-dimethoxy-benzoyl chloride (962 mg; 4.81 mmol), and triethylamine (736 uL; 5.25 mmol) in dry DCM (50 mL). The crude compound was purified by chromatography [SiO₂, Petroleum ether/EtOAc (9/1 to 6/4)], to afford the title compound as a white powder (878 mg; 62% yield).

¹H NMR (300 MHz, DMSO-d6) δ(ppm): 10.17 (s, 1H), 7.90-8.03 (m, 1H), 7.78 (ddd, 1H), 7.64 (dd, 1H), 7.55 (d, 1H), 7.40 (t, 1H), 7.23 (ddd, 1H), 7.09 (d, 1H), 3.85 (s, 3H), 3.84 (s, 3H), 1.70 (s, 6H).

LCMS (RT): 2.2 min (Method G); MS (ES+) gave m/z: 325.2 (MH+).

### Example 80

### 3,4-Dimethoxy-N-[4-(2-methoxy-1,1-dimethyl-ethyl)-phenyl]-benzamide

### 80(A) 2-Methyl-2-(4-nitro-phenyl)-propan-1-ol

Butyl chloroformate (124 uL; 0.96 mmol) was added to a chilled solution (−15° C.) of 2-methyl-2-(4-nitro-phenyl)-propionic acid (200 mg; 0.96 mmol), prepared as in 78(B), and N-methylmorpholine (97 uL; 0.96 mmol) in 1,2-dimethoxyethane (15 mL). The reaction was stirred at the same temperature for 20 min, and then the precipitate was removed quickly by suction filtration. To the collected solution, was added a solution of sodium borohydride (73.0 mg; 1.91 mmol) in EtOH (5 mL) and the resulting reaction was stirred at room temperature for 1 hour. The solvent was removed under reduced pressure; the residue was taken up with DCM and washed with 2M K₂CO₃ (twice) and then water. The organic phase was dried (Na₂SO₄), filtered and evaporated to dryness to afford the title compound as a colourless oil (158 mg; 84% yield).

LCMS (RT): 1.28 min (Method D); MS (ES+) gave m/z: 196.0 (MH+).

### 80(B) 1-(2-Methoxy-1,1-dimethyl-ethyl)-4-nitro-benzene

NaH (60% dispersion in mineral oil; 46.0 mg; 0.97 mmol) was added in portions to a stirred solution of 2-methyl-2-(4-nitro-phenyl)-propan-1-ol (158 mg; 0.81 mmol), obtained as described in 80(A), in dry THF (15 mL) at 0° C. under nitrogen atmosphere. After 30 min, iodomethane was added and the mixture was warmed to room temperature and stirred for 16 hours. Then the mixture was dried under vacuum and the residue portioned between EtOAc and water. The organic phase was collected, dried over Na₂SO₄, filtered and evaporated to dryness to give the title compound as a yellow oil (163 mg), which was used in the next step without any further purification.

LCMS (RT): 1.63 min (Method D); MS (ES+) gave m/z: 210.1 (MH+).

### 80(C) 4-(2-Methoxy-1,1-dimethyl-ethyl)-phenylamine

Prepared according to Example 1(B), starting from 1-(2-methoxy-1,1-dimethyl-ethyl)-4-nitro-benzene (163 mg; 0.78 mmol), prepared as in 80(B), and using 10% Pd/C (10 mg) in MeOH (20 mL) The catalyst was filtered off and the filtrate was evaporated under vacuum to give the title compound as a pale yellow solid (134 mg). The compound was used as such in the following step.

LCMS (RT): 0.89 min (Method D); MS (ES+) gave m/z: 180.0 (MH+).

### 80(D) 3,4-Dimethoxy-N-[4-(2-methoxy-1,1-dimethyl-ethyl)-phenyl]-benzamide

Prepared according to Example 1(C), starting from 4-(2-methoxy-1,1-dimethyl-ethyl)-phenylamine (134 mg; 0.75 mmol), prepared as in 80(C), and using 3,4-dimethoxy-benzoyl chloride (179 mg; 0.90 mmol), and triethylamine (157 uL; 1.12 mmol) in dry DCM (10 mL). The crude compound was purified by chromatography [SiO₂, Petroleum ether/EtOAc (8/2 to 6/4)], to afford the title compound as a white powder (15.3 mg; 6% yield over three steps).

¹H NMR (300 MHz, DMSO-d6+TFA) δ(ppm): 9.98 (br. s., 1H), 7.65 (m, 2H), 7.61 (dd, 0H), 7.53 (d, 1H), 7.33 (m, 2H), 7.07 (d, 1H), 3.84 (s, 3H), 3.83 (s, 3H), 3.35 (s, 2H), 3.22 (s, 3H), 1.25 (s, 6H).

LCMS (RT): 2.34 min (Method G); MS (ES+) gave m/z: 344.06 (MH+).

### Example 84

### 4-Bromo-1-methyl-1H-pyrazole-3-carboxylic acid{2-[4-(3,4-dimethoxy-benzoylamino)-phenyl]-2-methyl-propyl}-amide

Prepared according to Example 50, starting from N44-(2-amino-1,1-dimethyl-ethyl)-phenyl]-3,4-dimethoxy-benzamide (100 mg; 0.30 mmol), prepared as described in 26(A), and using 4-bromo-1-methyl-1H-pyrazole-3-carboxylic acid (62.0 mg; 0.30 mmol), HOBt (49.0 mg; 0.36 mmol) and EDC (87.0 mg; 0.46 mmol). The crude compound was purified by chromatography [SiO₂, DCM to DCM/MeOH (98/2)], to yield the title compound as a white powder (103 mg; 64% yield).

¹H NMR (300 MHz, DMSO-d6) δ(ppm): 10.01 (s, 1H), 8.00 (s, 1H), 7.71 (m, 2H), 7.62 (dd, 1H), 7.53 (d, 1H), 7.40-7.46 (m, 1H), 7.38 (m, 2H), 7.08 (d, 1H), 3.85 (s, 6H), 3.84 (s, 3H), 3.44 (d, 2H), 1.28 (s, 6H).

LCMS (RT): 2.13 min (Method G); MS (ES+) gave m/z: 515.25; 517.25 (M; M+2).

MP: 112-113° C.

### Example 88

### N-[3-(2-Acetylamino-1,1-dimethyl-ethyl)-phenyl]-3,4-dimethoxy-benzamide

### 88(A) N-[3-(2-Amino-1,1-dimethyl-ethyl)-phenyl]-3,4-dimethoxy-benzamide

Prepared according to Example 26(A), starting from N43-(cyano-dimethyl-methyl)-phenyl]-3,4-dimethoxy-benzamide (800 mg; 2.47 mmol), prepared as in 79(C), and using 10% Pd/C (20 mg) and 37% HCl (1 mL). The crude mixture was purified by ion-exchange chromatography [SCX, DCM/MeOH (1/1) to MeOH/NH₄OH (9/1)] to give the title compound as a white powder (774 mg; 96% yield).

LCMS (RT): 1.04 min (Method D); MS (ES+) gave m/z: 329.1 (MH+).

### 88(B) N-[3-(2-Acetylamino-1,1-dimethyl-ethyl)-phenyl]-3,4-dimethoxy-benzamide

Prepared according to Example 26(B), starting from N-[3-(2-amino-1,1-dimethyl-ethyl)-phenyl]-3,4-dimethoxy-benzamide (50.0 mg; 0.14 mmol), prepared as in 88(A), and using acetyl chloride (11 uL; 0.15 mmol) and pyridine (31 uL; 0.15 mmol) in DCM (10 mL) The crude compound was purified by chromatography [SiO₂, DCM to DCM/MeOH (98/2)], to afford the title compound as a white powder (31 mg; 61% yield).

¹H NMR (300 MHz, DMSO-d6) δ(ppm): 10.00 (br. s., 1H), 7.74 (t, 1H), 7.66-7.73 (m, 1H), 7.57-7.66 (m, 2H), 7.54 (d, 1H), 7.28 (dd, 1H), 7.08 (d, 0H), 7.09 (ddd, 1H), 3.85 (s, 3H), 3.84 (s, 3H), 3.25 (d, 2H), 1.81 (s, 3H), 1.23 (s, 6H).

LCMS (RT): 1.81 min (Method G); MS (ES+) gave m/z: 371.30 (MH+).

MP: 132-134° C.

### Example 89

### N-[2-Chloro-4-(cyano-dimethyl-methyl)-phenyl]-3,4-dimethoxy-benzamide

### 89(A) 2-(4-Amino-3-chloro-phenyl)-2-methyl-propionitrile

N-Chloro-succinimide (91.0 mg; 0.68 mmol) was added to a solution of 2-(4-amino-phenyl)-2-methyl-propionitrile (100 mg; 0.62 mmol), prepared as described in 1(B), in isopropanol (3 mL). The resulting solution was stirred at reflux for 1 h. Then the solvent was evaporated under vacuum and the crude was portioned between EtOAc and H₂O. The layers were separated and the organic phase was washed with brine, dried over Na₂SO₄, filtrated and concentrated under reduced pressure. The crude was purified by chromatography [SiO₂, DCM] to afford the title compound as an orange oil (58.0 mg; 48% yield).

¹H NMR (300 MHz, CDCl3) δ(ppm): 7.34 (d, 1H), 7.18 (dd, 1H), 6.78 (d, 1H), 3.73 (br. s., 2H), 1.68 (s, 6H).

LCMS (RT): 4.63 min (Method B); MS (ES+) gave m/z: 195.05 (MH+).

### 89(B) N-[2-Chloro-4-(cyano-dimethyl-methyl)-phenyl]-3,4-dimethoxy-benzamide

A solution of 3,4-dimethoxy-benzoic acid (54.0 mg; 0.30 mmol), HOBt (60.0 mg; 0.45 mmol), EDC (86.0 mg; 0.45 mmol) and TEA (125 uL; 0.90 mmol) in DCM (5 mL) was stirred at room temperature for 16 hours. After this period, the reaction was diluted with DCM, washed with water, dried over Na₂SO₄, filtered and evaporated under vacuum to afford the activated ester as a white solid (70 mg; 78% yield). This intermediate was dissolved in acetonitrile (5 mL) and 2-(4-amino-3-chloro-phenyl)-2-methyl-propionitrile (58.0 mg; 0.30 mmol), prepared as described in 89(A), was added. The reaction was heated under microwave irradiation at 170° C. for 7 hours. The crude was partially purified by filtration through a ion-exchange (SCX) cartridge [DCM/MeOH (1/1)]. The resulting compound was purified by preparative HPLC (Method Q) to afford the title compound as an off white solid (11,5 mg; 11% yield).

¹H NMR (300 MHz, CDCl3) δ(ppm): 8.60 (d, 1H), 8.40 (s, 1H), 7.55 (dd, 2H), 7.44 (ddd, 2H), 6.97 (d, 1H), 3.99 (s, 3H), 3.98 (s, 3H), 1.75 (s, 6H).

LCMS (RT): 2.31 min (Method G); MS (ES+) gave m/z: 359.12 (MH+).

### Example 90

### N-[3-(1-Cyano-cyclopropyl)-phenyl]-3,4-dimethoxy-benzamide

### 90(A) 1-(3-Nitro-phenyl)-cyclopropanecarbonitrile

Prepared according to Example 3(A), starting from (3-nitro-phenyl)-acetonitrile (0.70 g; 4.32 mmol), and using 1,2-dibromo-ethane (0.37 mL; 4.32 mmol) and NaH (60% dispersion in mineral oil; 0.38 mg; 9.50 mmol). The crude product was purified by column chromatography [SiO₂, Petroleum ether-EtOAc (9:1 to 8:2)] to give the title compound as a yellow solid (0.51 g, 63% yield).

LCMS (RT): 1.37 min (Method D); MS (ES+) gave m/z: 189.1 (MH+)

### 90(B) 1-(3-Amino-phenyl)-cyclopropanecarbonitrile

Prepared according to Example 1(B), starting from 1-(3-nitro-phenyl)-cyclopropanecarbonitrile (200 mg; 1.06 mmol), prepared as in 90(A), and using 10% Pd/C (25 mg) in MeOH (15 mL). The catalyst was filtered off and the filtrate was evaporated under vacuum to give the title compound (148 mg; 88% yield).

LCMS (RT): 0.77 min (Method D); MS (ES+) gave m/z: 159.1 (MH+).

### 90(C) N-[3-(1-Cyano-cyclopropyl)-phenyl]-3,4-dimethoxy-benzamide

Prepared according to Example 1(C), starting from 1-(3-amino-phenyl)-cyclopropanecarbonitrile (148 mg; 0.94 mmol), prepared as in 90(B), and using 3,4-dimethoxy-benzoyl chloride (225 mg; 1.12 mmol), and triethylamine (226 uL; 1.22 mmol) in dry DCM (50 mL). The crude compound was purified by preparative HPLC (Method R), to afford the title compound as a pale yellow amorphous solid (104 mg; 34% yield).

¹H NMR (300 MHz, CDCl3) δ(ppm): 7.81 (s, 1H), 7.64 (t, 1H), 7.57 (ddd, 1H), 7.51 (d, 1H), 7.41 (dd, 1H), 7.36 (t, 1H), 7.11 (ddd, 1H), 6.94 (d, 1H), 3.98 (s, 3H), 3.97 (s, 3H), 1.71-1.81 (m, 2H), 1.43-1.53 (m, 2H).

LCMS (RT): 2.07 min (Method G); MS (ES+) gave m/z: 323.17 (MH+).

### Example 91

### 1-Methyl-1H-indazole-3-carboxylic acid{2-[3-(3,4-dimethoxy-benzoylamino)-phenyl]-2-methyl-propyl}-amide

Prepared according to Example 26(B), starting from N-[3-(2-amino-1,1-dimethyl-ethyl)-phenyl]-3,4-dimethoxy-benzamide (50.0 mg; 0.14 mmol), prepared as in 88(A), and using 1-methyl-1H-indazole-3-carbonyl chloride (30.0 mg; 0.15 mmol) and pyridine (31 uL; 0.15 mmol) in DCM (10 mL). The crude compound was purified by chromatography [SiO₂, DCM to DCM/MeOH (98/2)], to afford the title compound as a white powder (29 mg; 44% yield).

¹H NMR (300 MHz, DMSO-d6) δ(ppm): 10.04 (br. s., 1H), 8.15 (dt, 1H), 7.83 (t, 1H), 7.66-7.78 (m, 3H), 7.64 (dd, 1H), 7.55 (d, 1H), 7.42-7.50 (m, 1H), 7.33 (dd, 1H), 7.23-7.30 (m, 1H), 7.15-7.23 (m, 1H), 7.08 (d, 1H), 4.08 (s, 3H), 3.85 (s, 3H), 3.84 (s, 3H), 3.56 (d, 2H), 1.33 (s, 6H).

LCMS (RT): 2.44 min (Method G); MS (ES+) gave m/z: 487.31 (MH+).

### Example 92

### 1-Methyl-4-phenyl-1H-pyrazole-3-carboxylic acid{2-[4-(3,4-dimethoxy-benzoylamino)-phenyl]-2-methyl-propyl}-amide

A mixture of 4-bromo-1-methyl-1H-pyrazole-3-carboxylic acid{2-[4-(3,4-dimethoxy-benzoylamino)-phenyl]-2-methyl-propyl}-amide (50 mg; 0.10 mmol), prepared as in 84, phenylboronic acid (17.0 mg; 0.14 mmol), potassium fluoride (13.0 mg; 0.19 mmol) and palladium (II) acetate (3.0 mg; 0.01 mmol) in MeOH (3 mL), was heated at 100° C. for 2 hours by a microwave oven. The solvent was removed under vacuum, the residue was taken up with DCM and washed twice with water. The organic phase was dried (Na₂SO₄), filtered and evaporated to dryness. The crude compound was purified by chromatography [SiO₂, DCM to DCM/MeOH (98/2)], to afford the title compound as a white solid (24 mg; 48% yield).

¹H NMR (300 MHz, DMSO-d6) δ(ppm): 10.01 (br. s., 1H), 7.97 (s, 1H), 7.71 (m, 2H), 7.62 (dd, 1H), 7.45-7.56 (m, 4H), 7.38 (m, 2H), 7.27-7.35 (m, 2H), 7.17-7.27 (m, 1H), 7.08 (d, 1H), 3.87 (s, 3H), 3.85 (s, 3H), 3.84 (s, 3H), 3.46 (d, 2H), 1.28 (s, 6H).

LCMS (RT): 2.38 min (Method G); MS (ES+) gave m/z: 513.2 (MH+).

### Example 93

### N-[4-(Cyano-dimethyl-methyl)-2-methyl-phenyl]-3,4-dimethoxy-benzamide

### 93(A) (3-Methyl-4-nitro-phenyl)-acetonitrile

A mixture of ethyl-cyanoacetate (0.72 mL; 6.71 mmol) and KOH (0.38 g; 6.71 mmol) in DMSO was stirred at room temperature for 1 hour. 4-Fluoro-2-methyl-1-nitro-benzene (0.80 g; 5.16 mmol) was added and the stirring was maintained at the same temperature for 16 hours. After this period, the reaction was acidified with 37% HCl till pH was about 2, then AcOH (1.5 mL) was added and the solution was refluxed for 4 hours. The reaction was portioned between water and ethyl ether, the organic phase was separated, dried (Na₂SO₄), filtered and evaporated to dryness. Purification by chromatography [SiO₂, Petroleum ether/EtOAc (95/5 to 8/2)] afforded the title compound as a yellow solid (0.27 g; 29% yield).

LCMS (RT): 1.3 min (Method D); MS (ES+) gave m/z: 177.1 (MH+).

### 93(B) 2-Methyl-2-(3-methyl-4-nitro-phenyl)-propionitrile

Prepared according to Example 75(B), starting from (3-methyl-4-nitro-phenyl)-acetonitrile (268 mg; 1.52 mmol), prepared ad in 93(A), and using tetrabuthylammonium bromide (9.80 g; 0.30 mmol), NaOH (608 mg; 15.2 mmol) and iodomethane (376 uL; 6.09 mmol). The crude product was purified by chromatography [SiO₂, Petroleum ether/EtOAc (95/5 to 8/2)] to give the title compound as a pale yellow solid (158 mg, 51% yield).

LCMS (RT): 1.55 min (Method D).

### 93(C) 2-(4-Amino-3-methyl-phenyl)-2-methyl-propionitrile

Prepared according to Example 1(B), starting from 2-methyl-2-(3-methyl-4-nitro-phenyl)-propionitrile (158 mg; 0.77 mmol), prepared as in 93(B), and using 10% Pd/C (10 mg) in MeOH (20 mL). The catalyst was filtered off and the filtrate was evaporated under vacuum to give the title compound as a white solid (131 mg; quantitative yield).

LCMS (RT): 175.16 min (Method A); MS (ES+) gave m/z: 175.16 (MH+).

### 93(D) N-[4-(Cyano-dimethyl-methyl)-2-methyl-phenyl]-3,4-dimethoxy-benzamide

Prepared according to Example 1(C), starting from 2-(4-amino-3-methyl-phenyl)-2-methyl-propionitrile (131 mg; 0.75 mmol), prepared as in 93(C), and using 3,4-dimethoxy-benzoyl chloride (165 mg; 0.83 mmol), and triethylamine (126 uL; 0.90 mmol) in dry DCM (15 mL). The crude was purified by preparative HPLC (Method Q), affording the title compound as an off-white solid (82 mg; 33% yield).

¹H NMR (300 MHz, DMSO-d6) δ(ppm): 9.73 (s, 1H), 7.63 (dd, 1H), 7.55 (d, 1H), 7.25-7.47 (m, 3H), 7.08 (d, 1H), 3.84 (s, 6H), 2.26 (s, 3H), 1.70 (s, 6H).

LCMS (RT): 3.24 min (Method t12); MS (ES+) gave m/z: 339.26 (MH+).

### Example 95

### N-[4-(Cyano-dimethyl-methyl)-3-methyl-phenyl]-3,4-dimethoxy-benzamide

### 95(A) (2-Methyl-4-nitro-phenyl)-acetonitrile

A solution of ethyl-cyanoacetate (4.52 mL; 42.6 mmol) in dry DMF (15 mL) was added dropwise to a suspension of NaH (60% dispersion in mineral oil; 1.70 g; 42.6 mmol) in dry DMF (15 mL), cooled at 0° C. and under inert atmosphere. The reaction was allowed to warm slowly to room temperature and then 1-fluoro-2-methyl-4-nitro-benzene (2.20 g; 14.2 mmol) was added and the stirring was maintained for additional 16 hours at the same temperature. After this period, the solvent was removed under vacuum, the residue was taken up with EtOAc and washed twice with 2N HCl. The organic phase was dried (Na₂SO₄), filtered and concentrated by rotary evaporator. The crude was dissolved in dioxane (10 mL), glacial acetic acid (5 mL) and 37% HCl (2 mL) and the resulting solution was refluxed overnight. The solvent was evaporated under vacuum; the residue was dissolved in EtOAc and washed with sat. NaHCO₃ and brine. The organic phase was dried over Na₂SO₄, filtered and evaporated to dryness. Recrystallization from MeOH provided the title compound as a yellow solid (1.88 g; 75% yield).

LCMS (RT): 1.29 min (Method D); MS (ES+) gave m/z: 177.1 (MH+).

### 95(B) 2-Methyl-2-(2-methyl-4-nitro-phenyl)-propionitrile

Prepared according to Example 1(A), starting from (2-methyl-4-nitro-phenyl)-acetonitrile (1.88 g; 10.7 mmol), prepared as in 95(A), and using NaH (60% dispersion in mineral oil; 0.86 mg; 21.4 mmol) and iodomethane (1.32 mL; 21.4 mmol) in DMF (10 mL). The crude product was purified by column chromatography [SiO₂, Petroleum ether/EtOAc (95/5 to 8/2)] to give the title compound (0.95 g, 43% yield).

LCMS (RT): 1.48 min (Method D); MS (ES+) gave m/z: 205.1 (MH+).

### 95(C) 2-(4-Amino-2-methyl-phenyl)-2-methyl-propionitrile

Prepared according to Example 1(B), starting from 2-methyl-2-(2-methyl-4-nitro-phenyl)-propionitrile (140 mg; 0.69 mmol), prepared as in 95(B), and using 10% Pd/C (14 mg) in MeOH (15 mL). The catalyst was filtered off and the filtrate was evaporated under vacuum to give the title compound, which was used in the next step without any further purification.

LCMS (RT): 0.82 min (Method D); MS (ES+) gave m/z: 175.1 (MH+).

### 95(D) N-[4-(Cyano-dimethyl-methyl)-3-methyl-phenyl]-3,4-dimethoxy-benzamide

A solution of 3,4-dimethoxy-benzoyl chloride (1.38 g; 6.86 mmol) in dry DCM (20 mL) was added dropwise to a solution of 2-(4-amino-2-methyl-phenyl)-2-methyl-propionitrile (0.80 g; 4.57 mmol), prepared as in 95(C), and triethylamine (0.95 mL; 6.86 mmol) in dry DCM (20 mL). The reaction was stirred at room temperature for 72 hours and then diluted with DCM and washed with NaHCO₃. The organic layer was dried over sodium sulphate, filtered and evaporated under reduced pressure. The residue was dissolved in DCM (10 mL) and treated with trifluoroacetic acid (1 mL). After stirring at room temperature for 1.5 hour, the reaction was diluted with DCM and washed with 2M K₂CO₃. The organic phase was dried (Na₂SO₄), filtered and evaporated to dryness. The crude compound was triturated with DCM/isopropanol (1/1) and the resulting white powder was filtered and dried under vacuum to yield the title compound (0.73 g; 47% yield).

¹H NMR (300 MHz, DMSO-d6) δ(ppm): 10.05 (s, 1H), 7.58-7.73 (m, 3H), 7.54 (d, 1H), 7.33 (d, 1H), 7.08 (d, 1H), 3.85 (s, 3H), 3.84 (s, 3H), 2.57 (s, 3H), 1.73 (s, 6H).

LCMS (RT): 2.22 min (Method G); MS (ES+) gave m/z: 314.22 (MH+).

### Example 96

### N-[4-(Cyano-dimethyl-methyl)-3-fluoro-phenyl]-3,4-dimethoxy-benzamide

### 96(A) (2-Fluoro-4-nitro-phenyl)-acetonitrile

A mixture of 1,2-difluoro-4-nitro-benzene (0.50 g; 3.14 mmol), K₂CO₃ (0.61 mg; 4.40 mmol), KI (0.005 g; 0.031 mmol) and ethyl-cyanoacetate (0.37 mL; 3.46 mmol) in DMF (5 mL) was stirred at room temperature for 16 hours and then heated at 100° C. for 2 hours. The reaction was quenched with 10% citric acid and extracted twice with EtOAc. The combined organic layers were washed with brine, dried over Na₂SO₄, filtered and evaporated under vacuum. The resulting crude compound was dissolved in water/acetic acid (2.5 mL/1 mL) and then 37% HCl (0.35 mL) was added. The reaction was heated at 100° C. for 8 hours and then quenched with 10% K₂CO₃ and extracted three times with diethyl ether. The combined organic layers were washed with brine, dried over Na₂SO₄, filtered and concentrated under reduced pressure. The crude was purified by chromatography [SiO₂, Petroleum ether to petroleum ether/EtOAc (9/1)] to give the title compound as an orange oil (0,35 g, 45% yield).

¹H NMR (300 MHz, DMSO-d6) δ(ppm): 8.06-8.29 (m, 2H), 7.72-7.83 (m, 1H), 4.26 (s, 2H).

LCMS (RT): 4.25 min (Method B).

### 96(B) 2-(2-Fluoro-4-nitro-phenyl)-2-methyl-propionitrile

To a solution of (2-fluoro-4-nitro-phenyl)-acetonitrile (260 mg; 1.44 mmol), prepared as described in 96(A), and tetrabuthylammonium bromide (80.0 mg; 0.25 mmol) in toluene (5 mL), was added a solution of NaOH (580 mg; 14.4 mmol) in watere (5 mL), directly followed by iodomethane (450 uL; 7.22 mmol). The resulting reaction was vigorously stirred at room temperature for 20 hours then diluted with EtOAc, washed in sequence with 5% NaHCO₃, 1N hydrochloric acid, and finally with brine. The organic phase was dried over Na₂SO₄, filtered and evaporated to dryness. Flash chromatography of the residue [SiO₂, Petroleum ether/EtOAc (9/1)] afforded the title compound as a yellow solid (61 mg; 20% yield).

¹H NMR (300 MHz, CDCl3) δ(ppm): 8.09 (ddd, 1H), 8.01 (dd, 1H), 7.76 (dd, 1H), 1.87 (s, 3H), 1.86 (s, 3H).

LCMS (RT): 5.09 min (Method B).

### 96(C) 2-(4-Amino-2-fluoro-phenyl)-2-methyl-propionitrile

Prepared according to Example 1(B), starting from 2-(2-fluoro-4-nitro-phenyl)-2-methyl-propionitrile (60.0 mg; 0.29 mmol), prepared as in 96(B), and using 10% Pd/C (10 mg) in MeOH (15 mL). The catalyst was filtered off and the filtrate was evaporated under vacuum to give the title compound as a yellow oil (47.0 mg; 91% yield).

LCMS (RT): 3.47 min (Method B); MS (ES+) gave m/z: 179.11 (MH+).

### 96(D) N-[4-(Cyano-dimethyl-methyl)-3-fluoro-phenyl]-3,4-dimethoxy-benzamide

Prepared according to Example 1(C), starting from 2-(4-amino-2-fluoro-phenyl)-2-methyl-propionitrile (47.8 mg; 0.27 mmol), prepared as in 96(C), and using 3,4-dimethoxy-benzoyl chloride (55.0 mg; 0.27 mmol), and triethylamine (45 uL; 0.32 mmol) in dry DCM (3 mL). The crude mixture was partially purified by ion-exchange chromatography [SCX, DCM/MeOH (1/1) to MeOH/NH₄OH (9/1)]. Then, the resulting compound was further purified by column chromatography [SiO₂, Petroleum ether to petroleum ether/EtOAc (6/4)] to furnish the title compound as a white solid (49.0 mg; 53% yield).

¹H NMR (300 MHz, CDCl3) δ(ppm): 7.83 (br. s., 1H), 7.73 (dd, 1H), 7.51 (d, 1H), 7.47 (d, 1H), 7.40 (dd, 1H), 7.23-7.30 (m, 1H), 6.94 (d, 1H), 3.99 (s, 3H), 3.98 (s, 3H), 1.83 (s, 3H), 1.82 (s, 3H).

LCMS (RT): 2.24 min (Method G); MS (ES+) gave m/z: 343.23 (MH+).

### Example 97

### N-[4-(Cyano-methyl-phenyl-methyl)-phenyl]-3,4-dimethoxy-benzamide

### 97(A) 2-(4-Nitro-phenyl)-2-phenyl-propionitrile

1-Chloro-4-nitro-benzene (1.00 g; 6.37 mmol), 2-phenyl-propionitrile (0.84 mL; 6.37 mmol) and triethylbenzylammonium chloride (0.03 g; 0.13 mmol) were put in a three necked flask equipped with thermometer. Acetonitrile (30 mL) was added and, after short stirring period, 50% NaOH (10 mL; 250 mmol) was added and the reaction was cooled if necessary. The mixture was maintained at 50° C. for 3 hours with vigorous stirring, and then portioned between water and toluene. The organic phase was collected, dried over Na₂SO₄, filtered and evaporated to yield a dark solid which was purified by crystallization from MeOH, affording the title compound as a yellow solid (1.31 g; 82% yield).

LCMS (RT): 1.63 min (Method D).

### 97(B) 2-(4-Amino-phenyl)-2-phenyl-propionitrile

Prepared according to Example 1(B), starting from 2-(4-nitro-phenyl)-2-phenyl-propionitrile (1.31 g; 5.20 mmol), prepared as in 97(A), and using 10% Pd/C (20 mg) in MeOH (30 mL). The catalyst was filtered off and the filtrate was evaporated under vacuum to give the title compound as a yellow oil (1.01 g; quantitative yield).

LCMS (RT): 0.91 min (Method D); MS (ES+) gave m/z: 223.1 (MH+).

### 97(C) N-[4-(Cyano-methyl-phenyl-methyl)-phenyl]-3,4-dimethoxy-benzamide

Prepared according to Example 1(C), starting from 2-(4-amino-phenyl)-2-phenyl-propionitrile (200 mg; 0.90 mmol), prepared as in 97(B), and using 3,4-dimethoxy-benzoyl chloride (198 mg; 0.99 mmol), and triethylamine (151 uL; 1.08 mmol) in dry DCM (20 mL). The crude mixture was purified by preparative HPLC (Method Q) to furnish the title compound as a white powder (103 mg; 30% yield).

¹H NMR (300 MHz, DMSO-d6) δ(ppm): 10.14 (s, 1H), 7.80 (m, 2H), 7.62 (dd, 1H), 7.52 (d, 1H), 7.28-7.48 (m, 7H), 7.08 (d, 1H), 3.84 (s, 6H), 2.09 (s, 3H).

LCMS (RT): 2.49 min (Method G); MS (ES+) gave m/z: 387.19 (MH+).

### Example 98

### N-[3-Chloro-4-(cyano-dimethyl-methyl)-phenyl]-3,4-dimethoxy-benzamide

### 98(A) (2-Chloro-4-nitro-phenyl)-acetonitrile

2-Chloro-1-methyl-4-nitro-benzene (1.00 g; 5.83 mmol) was dissolved in tert-butoxy-bis(dimethylamino)-methane (6.00 mL; 39.1 mmol) and the mixture was heated at 100° C. for 3 hours. The reaction was concentrated under reduced pressure to afford a dark-red residue, which was taken up with water (20 mL) and treated with hydroxylamine-O-sulfonic acid (1.98 g; 17 5 mmol) at room temperature for 7 hours. The precipitate was filtered off, washed with cold water and dried under vacuum to give title compound (1.20 g; quantitative yield).

LCMS (RT): 2.05 min (Method E).

### 98(B) 2-(2-Chloro-4-nitro-phenyl)-2-methyl-propionitrile

Prepared according to Example 1(A), starting from (2-chloro-4-nitro-phenyl)-acetonitrile (1.20 g; 6.12 mmol), prepared as described in 98(A), and using NaH (60% dispersion in mineral oil; 0.47g; 12.2 mmol) and iodomethane (0.76 mL; 12.2 mmol). The crude product was purified by chromatography [SiO₂, Petroleum ether/EtOAc (98/2 to 9/1)] to give the title compound as a white solid (0.91 g, 66% yield).

LCMS (RT): 5.19 min (Method B); MS (ES+) gave m/z: 225.07 (MH+).

### 98(C) 2-(4-Amino-2-chloro-phenyl)-2-methyl-propionitrile

PtO₂ (90 mg) was added to a solution of 2-(2-chloro-4-nitro-phenyl)-2-methyl-propionitrile (0.91 g; 4.06 mmol), prepared as in 98(B), in MeOH (25 mL). The mixture was hydrogenated at 1 bar at room temperature for 1 hour, then the catalyst was removed by filtration and the filtrate was concentrated under reduced pressure to give the title compound as a yellow oil (0.72 g; 91% yield).

LCMS (RT): 3.77 min (Method B); MS (ES+) gave m/z: 195.1 (MH+).

### 98(D) N-[3-Chloro-4-(cyano-dimethyl-methyl)-phenyl]-3,4-dimethoxy-benzamide

3,4-Dimethoxy-benzoyl chloride (1.11 g; 5.57 mmol) was added portionwise to a solution of 2-(4-amino-2-chloro-phenyl)-2-methyl-propionitrile (0.72 g; 3.71 mmol), prepared as in 98(C), and triethylamine (0.77 mL; 5.57 mmol) in dry DCM (15 mL). The reaction was stirred at room temperature for 16 hours and then diluted with DCM, washed with water, 1N HCl, 10% K₂CO₃, and brine. The organic layer was dried over sodium sulphate, filtered and evaporated under reduced pressure. The crude compound was dissolved in DCM (15 mL) and treated with TFA (2 mL) at room temperature for 1 hour. The reaction was diluted with DCM, washed with 10% K₂CO₃, dried (Na₂SO₄), filtered and evaporated to dryness. The crude was purified by flash chromatography [SiO₂, Petroleum ether/EtOAc (9/1 to 6/4)] to afford the title compound as a white powder (0.65 g; 50% yield).

¹H NMR (300 MHz, CDCl3) δ(ppm): 7.82 (d, 1H), 7.77 (br. s., 1H), 7.60 (dd, 1H), 7.50 (d, 1H), 7.47 (d, 1H), 7.39 (dd, 1H), 6.94 (d, 1H), 3.98 (s, 3H), 3.97 (s, 3H), 1.89 (s, 6H).

LCMS (RT): 2.30 min (Method G); MS (ES+) gave m/z: 359.12 (MH+).

### Example 99

### N-[4-(Cyano-dimethyl-methyl)-3-trifluoromethyl-phenyl]-3,4-dimethoxy-benzamide

### 99(A) (4-Nitro-2-trifluoromethyl-phenyl)-acetonitrile

A mixture of 1-chloro-4-nitro-2-trifluoromethyl-benzene (0.50 mL; 3.38 mmol), K₂CO₃ (0.65 mg; 4.74 mmol), KI (0.006g; 0.034 mmol) and ethyl-cyanoacetate (0.40 mL; 3.72 mmol) in DMF (5 mL) was stirred at room temperature for 72 hours. The reaction was quenched with 10% citric acid and extracted twice with EtOAc. The combined organic layers were washed with brine, dried over Na₂SO₄, filtered and evaporated under vacuum. The resulting crude compound was dissolved in water/acetic acid (2.5 mL/1 mL) and then 37% HCl (0.35 mL) was added. The reaction was heated at 100° C. for 30 hours and then quenched with 10% K₂CO₃ and extracted three times with diethyl ether. The combined organic layers were washed with brine, dried over Na₂SO₄, filtered and concentrated under reduced pressure. The crude was purified by chromatography [SiO₂, Petroleum ether to petroleum ether/EtOAc (9/1)] to give the title compound as an orange oil (0.35 g, 45% yield).

LCMS (RT): 5.65 min (Method B).

### 99(B) 2-Methyl-2-(4-nitro-2-trifluoromethyl-phenyl)-propionitrile

Prepared according to Example 75(B), starting from (4-nitro-2-trifluoromethyl-phenyl)-acetonitrile (0.35 g; 1.51 mmol), and using tetrabuthylammonium bromide (0.08 g; 0.25 mmol), NaOH (0.61g; 15.1 mmol) and iodomethane (0.47 mL; 7.56 mmol). The crude product was used in the next step without any further purification.

LCMS (RT): 5.43 min (Method D).

### 99(C) 2-(4-Amino-2-trifluoromethyl-phenyl)-2-methyl-propionitrile

Prepared according to Example 1(B), starting from 2-methyl-2-(4-nitro-2-trifluoromethyl-phenyl)-propionitrile (1.50 mmol; 3.88 mg), prepared as in 99(B), and using 10% Pd/C (60 mg) in MeOH (20 mL). The catalyst was filtered off and the filtrate was evaporated under vacuum to give 0.33 g of dark oil, which was used in the next step without any further purification.

LCMS (RT): 1.99 min (Method E); MS (ES+) gave m/z: 229.1 (MH+).

### 99(D) N-[4-(Cyano-dimethyl-methyl)-3-trifluoromethyl-phenyl]-3,4-dimethoxy-benzamide

3,4-Dimethoxy-benzoyl chloride (296 mg; 1.45 mmol) was added portionwise to a solution of 2-(4-amino-2-trifluoromethyl-phenyl)-2-methyl-propionitrile (330 mg; 1.45 mmol, crude compound), prepared as in 99(C), and triethylamine (241 uL; 1.74 mmol) in dry DCM (10 mL). The reaction was stirred at room temperature for 74 hours and then diluted with DCM, washed with water, 10% K₂CO₃ and brine. The organic layer was dried over sodium sulphate, filtered and evaporated under reduced pressure. The crude compound was purified by flash chromatography [SiO₂, Petroleum ether/EtOAc (9/1 to 8/2)]. The solid that was recovered from this purification was purified again by preparative HPLC (Method Q) to afford the title compound as a white solid (15 mg; 3% yield over three steps).

¹H NMR (300 MHz, CDCl3) δ(ppm): 8.02 (dd, 1H), 7.97 (d, 1H), 7.90 (s, 1H), 7.73 (d, 1H), 7.51 (d, 1H), 7.42 (dd, 1H), 6.94 (d, 1H), 3.98 (s, 3H), 3.97 (s, 3H), 1.89 (s, 6H).

LCMS (RT): 2.38 min (Method G); MS (ES+) gave m/z: 393.18 (MH+).

### Example 100

### N-[4-(Cyano-dimethyl-methyl)-3-methoxy-phenyl]-3,4-dimethoxy-benzamide

### 100(A) (2-Methoxy-4-nitro-phenyl)-acetonitrile

Prepared according to Example 75(A), starting from 2-methoxy-1-methyl-4-nitro-benzene (0.50 g; 2.99 mmol), and using tert-butoxy-bis(dimethylamino)-methane (1.18 mL; 5.74 mmol) and then hydroxylamine-O-sulfonic acid (1.01 g; 8.97 mmol). The title compound was collected by filtration as a white compound (0.17 g; 30% yield).

LCMS (RT): 4.10 min (Method B).

### 100(B) 2-(2-Methoxy-4-nitro-phenyl)-2-methyl-propionitrile

Prepared according to Example 75(B), starting from (2-methoxy-4-nitro-phenyl)-acetonitrile (175 mg; 0.91 mmol), prepared as described in 100(A), and using tetrabuthylammonium bromide (50.0 mg; 0.15 mmol), NaOH (365 mg; 9.11 mmol) water (4 mL) and iodomethane (282 uL; 4.56 mmol). The crude product was purified by chromatography [SiO₂, Petroleum ether/EtOAc (8/2)] to give the title compound as a yellow solid (145 mg, 72% yield).

LCMS (RT): 2.75 min (Method B); MS (ES+) gave m/z: 221.1 (MH+).

### 100(C) 2-(4-Amino-2-methoxy-phenyl)-2-methyl-propionitrile

Prepared according to Example 1(B), starting from 2-(2-methoxy-4-nitro-phenyl)-2-methyl-propionitrile (145 mg; 0.66 mmol), prepared as in 100(B), and using 10% Pd/C (20 mg) in MeOH (20 mL). The catalyst was filtered off and the filtrate was evaporated under vacuum to give the title compound (114 mg; 90% yield).

LCMS (RT): 2.75 min (Method D); MS (ES+) gave m/z: 191.14 (MH+).

### 100(D) N-[4-(Cyano-dimethyl-methyl)-3-methoxy-phenyl]-3,4-dimethoxy-benzamide

Prepared according to Example 1(C), starting from 2-(4-amino-2-methoxy-phenyl)-2-methyl-propionitrile (154 mg; 0.81 mmol), prepared as in 100(C), and using 3,4-dimethoxy-benzoyl chloride (179 mg; 0.89 mmol), and triethylamine (135 uL; 0.97 mmol) in dry DCM (5 mL). The crude was purified by preparative HPLC (Method Q), to afford the title compound as a white solid (16 mg; 29% yield).

¹H NMR (300 MHz, CDCl3) δ(ppm): 7.78 (br. s., 1H), 7.70 (d, 1H), 7.52 (d, 1H), 7.40 (dd, 1H), 7.32 (d, 1H), 6.89-7.01 (m, 2H), 3.98 (s, 6H), 3.97 (s, 3H), 1.78 (s, 6H).

LCMS (RT): 2.19 min (Method G); MS (ES+) gave m/z: 355.1 (MH+).

### Example 101

### 1H-Indole-3-carboxylic acid{2-[4-(3,4-dimethoxy-benzoylamino)-phenyl]-2-methyl-propyl}-amide

A mixture of 1H-indole-3-carboxylic acid (44.0 mg; 0.27 mmol), HOBt (48.0 mg; 0.36 mmol), TEA (38.0 uL; 0.27 mmol) and EDC (68.0 mg; 0.36 mmol) in dioxane (6 mL) was stirred at room temperature under inert atmosphere for 30 min. Then a solution of N-[4-(2-amino-1,1-dimethyl-ethyl)-phenyl]-3,4-dimethoxy-benzamide (90.0 mg; 0.27 mmol), prepared as described in 26(A), in dioxane (3 mL) was added and the stirring was maintained for 16 hours at room temperature. After this period the solvent was evaporated under vacuum, the residue was taken up with DCM and washed with 2M K₂CO₃, and then water. The organic phase was dried over Na₂SO₄, filtered and evaporated to dryness. The crude was purified by chromatography [SiO₂, DCM to DCM/MeOH (98/2)] to afford the title compound as a white amorphous solid (0.80 mg; 62% yield).

¹H NMR (300 MHz, DMSO-d6, 353 K) δ(ppm): 11.25 (br. s., 1H), 9.76 (s, 1H), 7.93-7.98 (m, 1H), 7.90 (d, 1H), 7.66-7.75 (m, 2H), 7.62 (dd, 1H), 7.57 (d, 1H), 7.34-7.48 (m, 3H), 6.94-7.17 (m, 4H), 3.87 (s, 3H), 3.86 (s, 3H), 3.54 (d, 2H), 1.36 (s, 6H).

LCMS (RT): 3.28 min (Method G); MS (ES+) gave m/z: 472.17 (MH+).

### Example 102

### 1-Methyl-1H-indazole-3-carboxylic acid{1-[3-(3,4-dimethoxy-benzoylamino)-phenyl]-cyclopentylmethyl}-amide

### 102(A) N-[3-(1-Aminomethyl-cyclopentyl)-phenyl]-3,4-dimethoxy-benzamide

Prepared according to Example 13(A), staring from N-[3-(1-cyano-cyclopentyl)-phenyl]-3,4-dimethoxy-benzamide (700 mg; 2.00 mmol), prepared as in 14(C), and using 10% Pd/C (20 mg) in 37% HCl (2 mL) and MeOH (20 mL). After work-up and purification, the title compound was achieved as a yellow oil (312 mg; 44% yield).

LCMS (RT): 2.7 min (Method A); MS (ES+) gave m/z: 355.15 (MH+).

### 102(B) 1-Methyl-1H-indazole-3-carboxylic acid{1-[3-(3,4-dimethoxy-benzoylamino)-phenyl]-cyclopentylmethyl}-amide

Prepared according to Example 50, starting from N-[3-(1-aminomethyl-cyclopentyl)-phenyl]-3,4-dimethoxy-benzamide (100 mg; 0.28 mmol), prepared as in 102(A), and using 1-methyl-1H-indazole-3-carboxylic acid (59.0 mg; 0.34 mmol), HOBt (57.0 mg; 0.42 mmol), EDC (108 mg; 0.56 mmol), TEA (79 uL; 0.56 mmol) in DCM (10 mL). The crude product was purified by preparative HPLC (Method Q) to give the title compound as a pale yellow solid (18 mg; 13% yield).

¹H NMR (300 MHz, DMSO-d6) δ(ppm): 10.03 (br. s., 1H), 8.13 (dt, 1H), 7.66-7.80 (m, 3H), 7.63 (dd, 1H), 7.54 (d, 1H), 7.38-7.49 (m, 2H), 7.33 (d, 1H), 7.21-7.30 (m, 1H), 7.04-7.14 (m, 2H), 4.04 (s, 3H), 3.84 (s, 6H), 3.54 (d, 2H), 1.96-2.07 (m, 2H), 1.60-1.93 (m, 6H).

LCMS (RT): 2.63 min (Method G); MS (ES+) gave m/z: 513.3 (MH+).

### Example 103

### 1H-Indazole-3-carboxylic acid{1-[3-(3,4-dimethoxy-benzoylamino)-phenyl]-cyclopentylmethyl}-amide

Prepared according to Example 50, starting from N-[3-(1-aminomethyl-cyclopentyl)-phenyl]-3,4-dimethoxy-benzamide (100 mg; 0.28 mmol), prepared as in 102(A), and using 1H-indazole-3-carboxylic acid (54.0 mg; 0.34 mmol), HOBt (57.0 mg; 0.42 mmol) and EDC (108 mg; 0.56 mmol) in DCM (10 mL). The crude product was purified by preparative HPLC (Method Q) to give the title compound as a pale yellow solid (22 mg; 16% yield).

¹H NMR (300 MHz, DMSO-d6) δ(ppm): 13.46 (br. s., 1H), 10.03 (s, 1H), 8.13 (dt, 1H), 7.68-7.81 (m, 2H), 7.63 (dd, 1H), 7.58 (dt, 1H), 7.54 (d, 1H), 7.35-7.47 (m, 2H), 7.31 (t, 1H), 7.22 (ddd, 1H), 7.04-7.15 (m, 2H), 3.84 (s, 6H), 3.56 (d, 2H), 1.97-2.07 (m, 2H), 1.61-1.93 (m, 6H).

LCMS (RT): 2.44 min (Method G); MS (ES+) gave m/z: 499.29 (MH+).

### Example 104

### 1-Acetyl-1H-indole-3-carboxylic acid{2-[4-(3,4-dimethoxy-benzoylamino)-phenyl]-2-methyl-propyl}-amide

A mixture of 1H-indole-3-carboxylic acid{2-[4-(3,4-dimethoxy-benzoylamino)-phenyl]-2-methyl-propyl}-amide (20 mg; 40 umol), prepared as in 101, 4-dimethylamino pyridine (6.0 mg; 80 umol) and acyl chloride (6.0 uL; 80 umol) in dry DCM (1,1 mL) was heated at 70° C. under microwave irradiation for 1 hour. The solvent was removed under vacuum and the residue was directly subjected to chromatography purification [SiO₂, DCM/MeOH (99/1)] to afford the title compound as a white amorphous solid (10 mg; 97% yield).

¹H NMR (300 MHz, DMSO-d6 353K) δ(ppm): 9.76 (s, 1H), 8.40 (s, 1H), 8.24-8.37 (m, 1H), 7.99-8.13 (m, 1H), 7.71 (m, 2H), 7.53-7.68 (m, 3H), 7.43 (m, 2H), 7.24-7.39 (m, 2H), 7.07 (d, 1H), 3.86 (s, 3H), 3.86 (s, 3H), 3.56 (d, 2H), 2.69 (s, 3H), 1.38 (s, 6H).

LCMS (RT): 2.37 min (Method G); MS (ES+) gave m/z: 514.31 (MH+).

### Example 107

### 1H-Indazole-3-carboxylic acid{2-[3-(3,4-dimethoxy-benzoylamino)-phenyl]-2-methyl-propyl}-amide

### 107(A) N-[3-(2-Amino-1,1-dimethyl-ethyl)-phenyl]-3,4-dimethoxy-benzamide

Prepared according to Example 13(A), staring from N-[3-(cyano-dimethyl-methyl)-phenyl]-3,4-dimethoxy-benzamide (968 mg; 2.99 mmol), prepared as in 79(C), and using 10% Pd/C (20 mg) and 37% HCl (2 mL) in MeOH (20 mL). The catalyst was filtered off and the filtrate was concentrated under reduced pressure. The crude was dissolved in DCM and loaded onto an ion-exchange (SCX) cartridge. The un-reacted starting material was recovered by eluting with DCM/MeOH (1/1) (426 mg), and then the title compound was recovered by eluting with MeOH/NH₄OH (9/1). The title compound was obtained as a yellow solid (318 mg; 32% yield).

LCMS (RT): 3.1 min (Method A); MS (ES+) gave m/z: 329.16 (MH+).

### 107(B) 1H-Indazole-3-carboxylic acid{2-[3-(3,4-dimethoxy-benzoylamino)-phenyl]-2-methyl-propyl}-amide

Prepared according to Example 50, starting from N43-(2-amino-1,1-dimethyl-ethyl)-phenyl]-3,4-dimethoxy-benzamide (85.0 mg; 0.26 mmol), prepared as in 107(A), and using 1H-indazole-3-carboxylic acid (59.0 mg; 0.31 mmol), HOBt (52.0 mg; 0.40 mmol) and EDC (99.0 mg; 0.52 mmol) in DCM (10 mL). The crude product was purified by preparative HPLC (Method Q) to give the title compound as a white powder (39 mg; 32% yield).

¹H NMR (300 MHz, DMSO-d6) δ(ppm): 13.48 (br. s., 1H), 10.03 (br. s., 1H), 8.15 (dt, 1H), 7.82 (t, 1H), 7.71-7.78 (m, 1H), 7.57-7.69 (m, 3H), 7.55 (d, 1H), 7.36-7.44 (m, 1H), 7.33 (t, 1H), 7.15-7.28 (m, 1H), 7.08 (d, 1H), 5.75 (s, 1H), 3.85 (s, 3H), 3.84 (s, 3H), 3.57 (d, 2H), 1.34 (s, 6H).

LCMS (RT): 2.22 min (Method G); MS (ES+) gave m/z: 473.17 (MH+).

### Example 109

### 1H-Indazole-3-carboxylic acid{2-[4-(3,4-dimethoxy-benzoylamino)-2-methyl-phenyl]-2-methyl-propyl}-amide

### 109(A) N-[4-(2-Amino-1,1-dimethyl-ethyl)-3-methyl-phenyl]-3,4-dimethoxy-benzamide

Prepared according to Example 13(A), staring from N-[4-(cyano-dimethyl-methyl)-3-methyl-phenyl]-3,4-dimethoxy-benzamide (260 mg; 0.77 mmol), prepared as in 95(C), and using 10% Pd/C (30 mg) in 37% HCl (2 mL) and MeOH (20 mL). After work-up and purification, the title compound was achieved as a white foam (210 mg; 79% yield).

LCMS (RT): 0.98 min (Method D); MS (ES+) gave m/z: 343.1 (MH+).

### 109(B) 1H-Indazole-3-carboxylic acid{2-[4-(3,4-dimethoxy-benzoylamino)-2-methyl-phenyl]-2-methyl-propyl}-amide

Prepared according to Example 50, starting from N-[4-(2-amino-1,1-dimethyl-ethyl)-3-methyl-phenyl]-3,4-dimethoxy-benzamide (70.0 mg; 0.20 mmol), prepared as in 109(A), and using 1H-indazole-3-carboxylic acid (34.0 mg; 0.20 mmol), HOBt (36.0 mg; 0.27 mmol) and EDC (52.0 mg; 0.27 mmol) in DCM (10 mL). The crude was purified by chromatography [SiO₂, DCM/MeOH (50/1)] to afford the title compound as a white solid (84.9 mg; 87% yield).

¹H NMR (300 MHz, DMSO-d6) δ(ppm): 13.44 (br. s., 1H), 9.94 (s, 1H), 8.15 (dt, 1H), 7.69 (t, 1H), 7.48-7.66 (m, 5H), 7.40 (ddd, 1H), 7.33 (d, 1H), 7.23 (ddd, 1H), 7.08 (d, 1H), 3.84 (s, 3H), 3.84 (s, 3H), 3.68 (d, 2H), 2.60 (s, 3H), 1.42 (s, 6H).

LCMS (RT): 2.27 min (Method G); MS (ES+) gave m/z: 487.24 (MH+).

### Example 110

### 1-Methyl-1H-indazole-3-carboxylic acid{2-[4-(3,4-dimethoxy-benzoylamino)-2-methyl-phenyl]-2-methyl-propyl}-amide

Prepared according to Example 50, starting from N-[4-(2-amino-1,1-dimethyl-ethyl)-3-methyl-phenyl]-3,4-dimethoxy-benzamide (70.0 mg; 0.20 mmol), prepared as in 109(A), and using 1-methyl-1H-indazole-3-carboxylic acid (37.0 mg; 0.20 mmol), HOBt (36.0 mg; 0.27 mmol) and EDC (52.0 mg; 0.27 mmol) in DCM (10 mL). The crude was purified by chromatography [SiO₂, DCM/MeOH (100/1)] to afford the title compound as a white solid (84.2 mg; 84,2% yield).

¹H NMR (300 MHz, DMSO-d6) δ(ppm): 9.94 (s, 1H), 8.15 (dt, 1H), 7.67-7.81 (m, 2H), 7.50-7.67 (m, 4H), 7.45 (ddd, 1H), 7.32 (d, 1H), 7.26 (ddd, 1H), 7.07 (d, 1H), 4.09 (s, 3H), 3.84 (s, 3H), 3.84 (s, 3H), 3.68 (d, 2H), 2.61 (s, 3H), 1.41 (s, 6H).

LCMS (RT): 2.45 min (Method G); MS (ES+) gave m/z: 501.25 (MH+).

### Example 111

### N-[3-Bromo-4-(cyano-dimethyl-methyl)-phenyl]-3,4-dimethoxy-benzamide

### 111(A) (2-Bromo-4-nitro-phenyl)-acetonitrile

A suspension of NaH (60% dispersion in mineral oil; 4.18 g; 110 mmol) in dioxane (40 mL) was chilled at 0° C. A solution of ethyl-cyanoacetate (11.6 mL; 0.11 mmol) in dioxane (10 mL) was added by means of a dropping funnel under inert atmosphere over 30 min. After the reaction was stirred at 0° C. for additional 10 min, 2-bromo-1-fluoro-4-nitro-benzene (8.00 g; 36 3 mmol) was added portionwise and the resulting dark solution was allowed to warm to room temperature and stirred for 16 hours. The reaction was carefully quenched with 1N HCl and the solvent was removed under vacuum. The residue was taken up with EtOAc and washed twice with water. The organic phase was dried over Na₂SO₄, filtered and evaporated to dryness under vacuum. The resulting crude compound was dissolved in dioxane (30 mL) and then 37% HCl (10 mL) was added. The resulting yellow solution was refluxed for one day, then concentrated under reduced pressure. The resulting residue was taken up with ethyl ether, washed twice with 10% K₂CO₃ and extracted three times with diethyl ether. The ethereal phase was dried over Na₂SO₄, filtered and concentrated under reduced pressure. The crude compound was used in the next step without any further purification.

LCMS (RT): 1.37 min (Method D).

### 111(B) 2-(2-Bromo-4-nitro-phenyl)-2-methyl-propionitrile

Prepared according to Example 75(B), starting from (2-bromo-4-nitro-phenyl)-acetonitrile (8.82 g; 36.3 mmol), prepared as in 111(A), and using tetrabuthylammonium bromide (2.34 g; 7.20 mmol), NaOH (14.5 g; 36.3 mmol) and iodomethane (9 mL; 145 mmol). The crude product was purified by chromatography [SiO₂, Petroleum ether/EtOAc (9/1 to 7/3)] to give the title compound as a yellow solid (5.20 g, 53% yield over two steps).

LCMS (RT): 4.1 min (Method A); MS (ES+) gave m/z: 269.01; 271.01 (M; M+2).

### 111(C) 2-(4-Amino-2-bromo-phenyl)-2-methyl-propionitrile

Prepared according to Example 98(C), starting from 2-(2-bromo-4-nitro-phenyl)-2-methyl-propionitrile (2.00 g; 7.43 mmol), prepared as described in 111(B), and using PtO₂ (66 mg) in MeOH (25 mL). After removing the catalyst by filtration and methanol by evaporation under vacuum, the title compound was achieved as a deep yellow oil (1.75 g; quantitative yield).

LCMS (RT): 3.5 min (Method A); MS (ES+) gave m/z: 239.0; 241.0 (M; M+2).

### 111(D) N-[3-Bromo-4-(cyano-dimethyl-methyl)-phenyl]-3,4-dimethoxy-benzamide

A solution of 2-(4-amino-2-bromo-phenyl)-2-methyl-propionitrile (500 mg; 2.09 mmol), prepared as described in 111(C), and 3,4-dimethoxy-benzoyl chloride (502 mg; 2.51 mmol) in pyridine (10 mL) was heated at 100° C. under microwave irradiation for 1 hour. Pyridine was evaporated under high vacuum and the residue was portioned between DCM and 1N HCl. The organic phase was dried (Na₂SO₄), filtered and evaporated under reduced pressure to dryness. The title compound was isolated by flash chromatography [SiO₂, Petroleum ether to petroleum ether/EtOAc (7/3)] as a white solid (672 mg; 65% yield).

¹H NMR (300 MHz, CDCl3) δ(ppm): 7.99 (d, 1H), 7.77 (br. s., 1H), 7.70 (dd, 1H), 7.50 (d, 1H), 7.47 (d, 1H), 7.39 (dd, 1H), 6.94 (d, 1H), 3.98 (s, 3H), 3.97 (s, 3H), 1.91 (s, 6H).

LCMS (RT): 2.26 min (Method G); MS (ES+) gave m/z: 403.08; 405.08 (M; M+2).

### Example 112

### 5-Methoxy-1H-indazole-3-carboxylic acid{2-[4-(3,4-dimethoxy-benzoylamino)-phenyl]-2-methyl-propyl}-amide

A mixture of 5-methoxy-1H-indazole-3-carboxylic acid (58.0 mg; 0.30 mmol) (Prepared following the procedure reported in Chem. Pharm. Bull. 43/11 (1995) 1912-1930), HOBt (46.0 mg; 0.30 mmol) and EDC (86.0 mg; 0.45 mmol) in dioxane (5 mL) was stirred at 45° C. for about 1 hour. Then, the reaction was cooled to room temperature and N-[4-(2-amino-1,1-dimethyl-ethyl)-phenyl]-3,4-dimethoxy-benzamide (100 mg; 0.30 mmol), prepared as described in 26(A), and TEA (42 uL; 0.30 mmol) were added. After stirring at room temperature for 16 hours, the solvent was removed under vacuum and the residue was taken up with DCM, which was washed sequentially with 1M NaOH (twice), 1N HCl (twice) and brine. The organic phase was dried over Na₂SO₄, filtered and evaporated to dryness. The crude compound was purified by trituration with EtOAc to provide the title compound as a yellow solid (86.0 mg; 57% yield).

¹H NMR (300 MHz, DMSO-d6) δ(ppm): 13.37 (br. s., 1H), 10.03 (s, 1H), 7.73 (m, 2H), 7.62 (dd, 1H), 7.47-7.58 (m, 4H), 7.42 (m, 2H), 6.97-7.15 (m, 2H), 3.85 (s, 3H), 3.84 (s, 3H), 3.80 (s, 3H), 3.50-3.57 (m, 2H), 1.33 (s, 6H).

LCMS (RT): 2.21 min (Method G); MS (ES+) gave m/z: 503.2 (MH+).

### Example 113

### 1H-Indazole-3-carboxylic acid{2-[2-chloro-4-(3,4-dimethoxy-benzoylamino)-phenyl]-2-methyl-propyl}-amide

### 113(A) 2-(2-Chloro-4-nitro-phenyl)-2-methyl-propylamine

To a solution of 2-(2-chloro-4-nitro-phenyl)-2-methyl-propionitrile (150 mg; 0.67 mmol), prepared as described in 98(B), in dry THF (1 mL), borane-THF complex (1M solution in THF; 2.7 mL) was added dropwise while stirring under nitrogen atmosphere. The reaction was refluxed for 1 hour, cooled at room temperature and quenched by adding methanol dropwise. Then, 37% HCl was added (1 mL) and the solution was heated to reflux for 30 min. The solvent was removed under vacuum, the residue was portioned between EtOAc and 2M K₂CO₃. The organic phase was dried over Na₂SO₄, filtered and evaporated to dryness under vacuum. The crude compound was purified by ion-exchange chromatography [SCX, DCM/MeOH (1/1) to MeOH/NH₄OH (9/1)] to give the title compound (105 mg; 68% yield).

LCMS (RT): 0.97 min (Method D); MS (ES+) gave m/z: 229.1 (MH+).

### 113(B) 1H-Indazole-3-carboxylic acid[2-(2-chloro-4-nitro-phenyl)-2-methyl-propyl]-amide

Prepared according to Example 50, starting from 2-(2-chloro-4-nitro-phenyl)-2-methyl-propylamine (257 mg; 1.13 mmol), prepared as in 113(A), and using 1H-indazole-3-carboxylic acid (183 mg; 1.13 mmol), HOBt (198 mg; 1.47 mmol), EDC (281 mg; 1.47 mmol) and TEA (0.73 mL; 2.47 mmol) in DCM (10 mL). The crude product was purified by chromatography [SiO₂, Petroleum ether/EtOAc (9/1 to 7/3)] to give the title compound as a white solid (185 mg; 44% yield).

LCMS (RT): 1.58 min (Method D); MS (ES+) gave m/z: 373.1 (MH+).

### 113(C) 1H-Indazole-3-carboxylic acid[2-(4-amino-2-chloro-phenyl)-2-methyl-propyl]-amide

Prepared according to Example 98(C), starting from 1H-indazole-3-carboxylic acid [2-(2-chloro-4-nitro-phenyl)-2-methyl-propyl]-amide (185 mg; 0.50 mmol), prepared as described in 113(B), and using PtO₂ (20 mg) in MeOH (30 mL). After removing the catalyst by filtration and methanol by evaporation under vacuum, the title compound was achieved as yellow oil (160 mg; 93% yield).

LCMS (RT): 1.19 min (Method D); MS (ES+) gave m/z: 343.1 (MH+).

### 113(D) 1H-Indazole-3-carboxylic acid{2-[2-chloro-4-(3,4-dimethoxy-benzoylamino)-phenyl]-2-methyl-propyl}-amide

Prepared according to Example 1(C), starting from 1H-indazole-3-carboxylic acid[2-(4-amino-2-chloro-phenyl)-2-methyl-propyl]-amide (55.0 mg; 0.16 mmol), prepared as in 113(C), and using 3,4-dimethoxy-benzoyl chloride (59.0 mg; 0.29 mmol), and triethylamine (40 uL; 0.29 mmol) in DCM (3 mL) The crude was purified by chromatography [SiO₂, DCM to DCM/MeOH (99.3/0.7)] to afford the title compound as a white amorphous solid (27 mg; 33% yield).

¹H NMR (300 MHz, DMSO-d6) δ(ppm): 13.46 (br. s., 1H), 10.15 (s, 1H), 8.13 (dt, 1H), 7.94 (d, 1H), 7.52-7.74 (m, 5H), 7.48 (d, 1H), 7.39 (ddd, 1H), 7.22 (ddd, 1H), 7.09 (d, 1H), 3.88 (d, 2H), 3.85 (s, 3H), 3.84 (s, 3H), 1.49 (s, 6H).

LCMS (RT): 2.39 min (Method G); MS (ES+) gave m/z: 507.24 (MH+).

### Example 114

### N-[4-(2-Cyano-1,1-dimethyl-ethyl)-phenyl]-3,4-dimethoxy-benzamide

### 114(A) 1-(2-Chloro-1,1-dimethyl-ethyl)-4-nitro-benzene

A mixture of (2-chloro-1,1-dimethyl-ethyl)-benzene (500 uL; 3.10 mmol), 65% HNO₃ (393 uL) and conc. H₂SO₄ (684 uL) was stirred at room temperature for 1 hour, then was neutralized with sat. NaHCO₃ and extracted twice with DCM. The organic layers were combined, dried over Na₂SO₄, filtered and evaporated to afford the title compound as a yellow oil (616 mg; 93% yield).

LCMS (RT): 6.03 min (Method B); MS (ES+) gave m/z: 214.1 (MH+).

### 114(B) 3-Methyl-3-(4-nitro-phenyl)-butyronitrile

1-(2-Chloro-1,1-dimethyl-ethyl)-4-nitro-benzene (616 mg; 2.89 mmol), prepared as described in 114(A), trimethylsilyl cyanide (600 uL; 4.34 mmol) and tetrabutylammonium fluoride (1M solution in dry THF; 4.34 mmol) were introduced into a vessel and dissolved with acetonitrile (5 mL). The vessel was sealed and exposed to MW irradiation for 6 hours at 150° C. Then the solvent was evaporated and the crude was directly applied to chromatography purification [SiO₂, Petroleum ether to petroleum ether/EtOAc (9/1)] to give the title compound as a yellow oil (70.0 mg; 12% yield).

LCMS (RT): 2.16 min (Method E).

### 114(C) 3-(4-Amino-phenyl)-3-methyl-butyronitrile

Prepared according to Example 98(C), starting from 3-methyl-3-(4-nitro-phenyl)-butyronitrile (68.0 mg; 0.33 mmol), prepared as described in 114(B), and using PtO₂ (10 mg) in MeOH (20 mL) After removing the catalyst by filtration and methanol by evaporation under vacuum, the title compound was collected as yellow oil (56.0 mg; 97% yield).

LCMS (RT): 2.19 min (Method B); MS (ES+) gave m/z: 175.21 (MH+).

### 114(D) N-[4-(2-Cyano-1,1-dimethyl-ethyl)-phenyl]-3,4-dimethoxy-benzamide

Prepared according to Example 98(D), starting from 3-(4-amino-phenyl)-3-methyl-butyronitrile (56.0 mg; 0.32 mmol), prepared as described in 114(C), and using 3,4-dimethoxy-benzoyl chloride (71.0 mg; 0.36 mmol), and triethylamine (55 uL; 0.39 mmol) in DCM (5 mL) The crude was purified by chromatography [SiO₂, Petroleum ether to petroleum ether/EtOAc (6/4)] to give the title compound as a pale yellow amorphous solid (55.0 mg; 50% yield).

¹H NMR (300 MHz, CDCl3) δ(ppm): 7.74 (br. s., 1H), 7.63 (m, 2H), 7.51 (d, 1H), 7.36-7.42 (m, 3H), 6.93 (d, 1H), 3.98 (s, 3H), 3.96 (s, 3H), 2.63 (s, 2H), 1.54 (s, 6H).

LCMS (RT): 2.09 min (Method G); MS (ES+) gave m/z: 339.19 (MH+).

### Example 118

### N-[6-(Cyano-dimethyl-methyl)-biphenyl-3-yl]-3,4-dimethoxy-benzamide

### 118(A) 2-(5-Amino-biphenyl-2-yl)-2-methyl-propionitrile

A solution of 2-(4-amino-2-bromo-phenyl)-2-methyl-propionitrile (80.0 mg; 0.33 mmol), prepared as described in 111(C), phenylboronic acid (49.0 mg; 0.40 mmol), 2M K₂CO₃ (334 uL; 0.67 mmol) in 1,2-dimethoxyethane (3 mL) was purged with nitrogen for 30 min. Tetrakis(triphenylphospine) palladium(0) was added and the vessel was sealed and heated in a microwave oven at 80° C. for 1 hour. The solvent was removed under vacuum and the residue was portioned between water and DCM. The organic phase was dried over Na₂SO₄, filtered and evaporated to dryness. The crude compound was purified by ion-exchange chromatography [SCX, DCM/MeOH (1/1) to MeOH/NH₄OH (9/1)] to give the title compound as a yellow oil (52.0 mg; 66% yield).

LCMS (RT): 3.3 min (Method A); MS (ES+) gave m/z: 237.13 (MH+).

### 118(B) N-[6-(Cyano-dimethyl-methyl)-biphenyl-3-yl]-3,4-dimethoxy-benzamide

Prepared according to Example 1(C), starting from 2-(5-amino-biphenyl-2-yl)-2-methyl-propionitrile (52.0 mg; 0.22 mmol), prepared as in 118(A), and using 3,4-dimethoxy-benzoyl chloride (48.0 mg; 0.24 mmol), and triethylamine (37 uL; 0.26 mmol) in DCM (3 mL). The crude was purified by chromatography [SiO₂, Petroleum ether to petroleum ether/EtOAc (6/4)] to afford the title compound as a white powder (41 mg; 46% yield).

¹H NMR (300 MHz, CDCl3) δ(ppm): 7.80 (dd, 1H), 7.75 (br. s., 1H), 7.63 (d, 1H), 7.49 (d, 1H), 7.30-7.47 (m, 7H), 6.92 (d, 1H), 3.96 (s, 3H), 3.96 (s, 3H), 1.62 (s, 6H).

LCMS (RT): 2.46 min (Method G); MS (ES+) gave m/z: 401.20 (MH+).

### Example 120

### N-[3,5-Dichloro-4-(cyano-dimethyl-methyl)-phenyl]-3,4-dimethoxy-benzamide

### 120(A) (2,6-Dichloro-4-nitro-phenyl)-acetonitrile

To a cold suspension of NaH (60% dispersion in mineral oil; 306 mg; 7.95 mmol) in DMSO (5 mL), was added ethyl-cyanoacetate (847 uL; 7.95 mmol) dropwise under nitrogen atmosphere. After the reaction was stirred at room temperature for 30 min, 1,2,3-trichloro-5-nitro-benzene (600 mg; 2.65 mmol) was added and the stirring was maintained for additional 16 hours. The reaction was quenched with water and then 1N hydrochloric acid was added until the pH was about 1. The white precipitate was collected by suction filtration and dried under vacuum for one night. This intermediate was dissolved in DMSO/H₂O (2 mL/0,8 mL) in presence of LiCl (102 mg; 2.41 mmol) and the resulting dark-violet solution was stirred at 165° C. for 30 min in a preheated oil bath. Then the reaction was poured into ice-water and extracted several times with diisopropyl ether. The extracts were combined, dried over Na₂SO₄, filtered and concentrated under reduced pressure to afford the title compound as a pale yellow solid (600 mg; 98% yield).

LCMS (RT): 5.49 min (Method B).

### 120(B) 2-(2,6-Dichloro-4-nitro-phenyl)-2-methyl-propionitrile

To a solution of (2,6-dichloro-4-nitro-phenyl)-acetonitrile (600 mg; 2.60 mmol), prepared as described in 120(A), iodomethane (495 uL; 7.95 mmol) and benzyl-triethylammonium chloride (60.0 mg; 265 mmol) in THF (6 mL), was added 50% NaOH (1 mL, 2.60 mmol) dropwise. The resulting mixture was heated at 50° C. for 12 hours and then at room temperature for 72 hours. The reaction was poured into ice-water and extracted with diisopropyl ether. The ethereal phase was dried (Na₂SO₄), filtered and evaporated under reduced pressure. Flash chromatography of the crude [SiO₂, Petroleum ether to petroleum ether/EtOAc (9/1)] yielded the title compound as yellow oil (486 mg; 71% yield).

LCMS (RT): 5.69 min (Method B).

### 120(C) 2-(4-Amino-2,6-dichloro-phenyl)-2-methyl-propionitrile

Prepared according to Example 98(C), starting from 2-(2,6-dichloro-4-nitro-phenyl)-2-methyl-propionitrile (386 mg; 1.50 mmol), prepared as described in 120(B), and using PtO₂ (50 mg) in MeOH (20 mL). After removing the catalyst by filtration and methanol by evaporation under vacuum, the title compound was achieved as yellow oil (333 mg; 93% yield).

LCMS (RT): 4.99 min (Method B); MS (ES+) gave m/z: 229.04 (MH+).

### 120(D) N-[3,5-Dichloro-4-(cyano-dimethyl-methyl)-phenyl]-3,4-dimethoxy-benzamide

To a solution of 2-(4-amino-2,6-dichloro-phenyl)-2-methyl-propionitrile (67.0 mg; 0.29 mmol), prepared as described in 120(C), in acetonitrile (5 mL), was added NaH (60% dispersion in mineral oil; 20 mg; 0.88 mmol) under nitrogen atmosphere. After the reaction was stirred at room temperature for 1 h, 3,4-dimethoxy-benzoyl chloride (71.0 mg; 0.35 mmol) was added and the resulting mixture was stirred at room temperature for 4 hours. The reaction was quenched by adding water, the solvent was removed under vacuum and the residue was taken up with DCM and washed sequentially with 10% K₂CO₃, 1N HCl and brine. The organic phase was dried over Na₂SO₄, filtered and evaporated under reduced pressure. Flash chromatography of the crude [SiO₂, Petroleum ether to petroleum ether/EtOAc (7/3)] yielded the title compound as a yellow solid (65.0 mg; 49% yield).

¹H NMR (300 MHz, CDCl3) δ(ppm): 7.75 (s, 2H), 7.71 (br. s., 1H), 7.48 (d, 1H), 7.37 (dd, 1H), 6.94 (d, 1H), 3.98 (s, 3H), 3.97 (s, 3H), 2.10 (s, 6H).

LCMS (RT): 2.51 min (Method G); MS (ES+) gave m/z: 393.12 (MH+).

### Example 121

### 1-Methyl-1H-indazole-3-carboxylic acid{2-[2-chloro-4-(3,4-dimethoxy-benzoylamino)-phenyl]-2-methyl-propyl}-amide

### 121(A) 1-Methyl-1H-indazole-3-carboxylic acid[2-(2-chloro-4-nitro-phenyl)-2-methyl-propyl]amide

Prepared according to Example 50, starting from 2-(2-chloro-4-nitro-phenyl)-2-methyl-propylamine (80.0 mg; 0.35 mmol), prepared as in 113(A), and using 1-methyl-1H-indazole-3-carboxylic acid (52.0 mg; 0.35 mmol), HOBt (62.0 mg; 0.45 mmol), EDC (87.0 mg; 0.45 mmol) and TEA (107 uL; 0.77 mmol) in DCM (5 mL). After the work-up, 120 mg of title compound were collected. This crude product was used in the next step without any further purification.

LCMS (RT): 1.72 min (Method D); MS (ES+) gave m/z: 387.0 (MH+).

### 121(B) 1-Methyl-1H-indazole-3-carboxylic acid[2-(4-amino-2-chloro-phenyl)-2-methyl-propyl]-amide

Prepared according to Example 98(C), starting from 1-methyl-1H-indazole-3-carboxylic acid[2-(2-chloro-4-nitro-phenyl)-2-methyl-propyl]-amide (120 mg; 0.31 mmol), prepared as described in 121(A), and using PtO₂ (20 mg) in MeOH (20 mL) After removing the catalyst by filtration and methanol by evaporation under vacuum, 105 mg of the title compound were achieved. This product was used as such in the following step.

LCMS (RT): 1.31 min (Method D); MS (ES+) gave m/z: 357.1 (MH+).

### 121(C) 1-Methyl-1H-indazole-3-carboxylic acid{2-[2-chloro-4-(3,4-dimethoxy-benzoylamino)-phenyl]-2-methyl-propyl}-amide

Prepared according to Example 1(C), starting from 1-methyl-1H-indazole-3-carboxylic acid[2-(4-amino-2-chloro-phenyl)-2-methyl-propyl]-amide (105 mg; 0.29 mmol), prepared as in 121(B), and using 3,4-dimethoxy-benzoyl chloride (88.0 mg; 0.43 mmol), and triethylamine (62 uL; 0.43 mmol) in DCM (3 mL). The crude was purified by chromatography [SiO₂, Petroleum ether/EtOAc (6/4)] to afford the title compound as a pale yellow amorphous solid (40 mg; 22% yield over three steps).

¹H NMR (300 MHz, DMSO-d6) δ(ppm): 10.15 (s, 1H), 8.13 (ddd, 1H), 7.94 (d, 1H), 7.65-7.74 (m, 3H), 7.62 (dd, 1H), 7.53 (d, 1H), 7.37-7.51 (m, 2H), 7.26 (ddd, 1H), 7.09 (d, 1H), 4.08 (s, 3H), 3.88 (d, 2H), 3.84 (s, 3H), 3.84-3.84 (m, 3H), 1.48 (s, 6H).

LCMS (RT): 2.58 min (Method G); MS (ES+) gave m/z: 521.18 (MH+).

### Example 122

### N-[4-(2-Acetylamino-1,1-dimethyl-ethyl)-3-chloro-phenyl]-3,4-dimethoxy-benzamide

### 122(A) N-[2-(2-Chloro-4-nitro-phenyl)-2-methyl-propyl]-acetamide

Prepared according to Example 54(B), starting from 2-(2-chloro-4-nitro-phenyl)-2-methyl-propylamine (80.0 mg; 0.35 mmol), prepared as in 113(A), and using acetyl chloride (47 uL; 0.66 mmol) and triethylamine (93 uL; 0.66 mmol) in DCM (3 mL) The crude compound was purified by ion-exchange chromatography [SCX, DCM/MeOH (1/1) to MeOH/NH₄OH (9/1)] to give the title compound as a yellow solid (80.0 mg; 84% yield).

LCMS (RT): 1.33 min (Method D); MS (ES+) gave m/z: 271.1 (MH+).

### 122(B) N-[2-(4-Amino-2-chloro-phenyl)-2-methyl-propyl]-acetamide

Prepared according to Example 98(C), starting from N-[2-(2-chloro-4-nitro-phenyl)-2-methyl-propyl]-acetamide (80.0 mg; 0.30 mmol), prepared as described in 122(A), and using PtO₂ (20 mg) in MeOH (20 mL). After removing the catalyst by filtration and methanol by evaporation under vacuum, the title compound was collected (65.0 mg; 90% yield).

LCMS (RT): 0.84 min (Method D); MS (ES+) gave m/z: 241.1 (MH+).

### 122(C) N-[4-(2-Acetylamino-1,1-dimethyl-ethyl)-3-chloro-phenyl]-3,4-dimethoxy-benzamide

Prepared according to Example 1(C), starting from N-[2-(4-amino-2-chloro-phenyl)-2-methyl-propyl]-acetamide (65.0 mg; 0.27 mmol), prepared as in 122(B), and using 3,4-dimethoxy-benzoyl chloride (81.0 mg; 0.40 mmol), and triethylamine (57 uL; 0.40 mmol) in DCM (3 mL) and in presence of some drops of DMF. The crude was purified by chromatography [SiO₂, Petroleum ether/EtOAc (8/2 to 2/8)] to yield the title compound as a white amorphous solid (30 mg; 27% yield).

¹H NMR (300 MHz, CDCl3) δ(ppm):7.80 (br. s., 1H), 7.74 (d, 1 11), 7.53 (dd, 1H), 7.50 (d, 1H), 7.40 (dd, 1H), 7.37 (d, 1H), 6.93 (d, 1H), 5.09 (br. s., 1H), 3.97 (s, 3H), 3.96 (s, 3H), 3.84 (d, 2H), 1.89 (s, 3H), 1.48 (s, 6H).

LCMS (RT): 1.98 min (Method G); MS (ES+) gave m/z: 405.17 (MH+).

### Example 125

### N-[4-Chloro-3-(cyano-dimethyl-methyl)-phenyl]-3,4-dimethoxy-benzamide

### l125(A) 2-Bromomethyl-1-chloro-4-nitro-benzene

A solution of 1-chloro-2-methyl-4-nitro-benzene (3.00 g; 17.5 mmol), N-bromosuccinimide (2.50 g; 14.1 mmol) and benzoyl peroxide (0.20 g; 0.83 mmol) in carbon tetrachloride (20 mL) was refluxed for 8 hours. The insoluble salts were filtered off and the filtrate was concentrated under vacuum to dryness. The crude compound was purified by double crystallization from hexane to afford the title compound as a yellow solid (1.98 g; 45% yield).

LCMS (RT): 4.3 min (Method A).

### 125(B) (2-Chloro-5-nitro-phenyl)-acetonitrile

A solution of 2-bromomethyl-1-chloro-4-nitro-benzene (1.00 g; 4.00 mmol), prepared as described in 125(A), in ethanol (6.5 mL) was mixed to a solution of potassium cyanide (0.26 g; 4.00 mmol) in water (1.5 mL). The mixture was heated for 16 hours under a reflux condenser. After cooling, the reaction was diluted with DCM and washed with water. The organic phase was dried over Na₂SO₄, filtered and evaporated under reduced pressure. Crystallization (three times) of the crude compound from petroleum ether yielded the title compound as a white solid (0.61 mg; 78% yield).

LCMS (RT): 3.8 min (Method A).

### 125(C) 2-(2-Chloro-5-nitro-phenyl)-propionitrile

Prepared according to Example 75(B), starting from (2-chloro-5-nitro-phenyl)-acetonitrile (0.78 g; 4.00 mmol), prepared as in 125(B), and using tetrabuthylammonium bromide (0.26 mg; 0.80 mmol), NaOH (1.60 g; 40.0 mmol) and iodomethane (0.99 mL; 16.0 mmol). The crude product was purified by chromatography [SiO₂, Petroleum ether/EtOAc (8/2)] to give the title compound as a white solid (332 mg, 39% yield).

LCMS (RT): 4.0 min (Method A)

### 125(D) 2-(2-Chloro-5-nitro-phenyl)-2-methyl-propionitrile

2-(2-Chloro-5-nitro-phenyl)-propionitrile (132 mg; 0.63 mmol), prepared as described in 125(C), was dissolved in dry DMF (5 mL), under nitrogen atmosphere. The solution was chilled to 0° C. (ice-bath) and Nail (60% dipersion in mineral oil; 24.0 mg; 0.63 mmol) was added. After about 10 min, iodomethane (39 uL; 0.63 mmol) was added and the resulting dark solution was stirred at room temperature for 30 min. After this time, the solvent was evaporated under vacuum and the residue was taken up with DCM and washed twice with 1N HCl. The organic phase was dried over Na₂SO₄, filtered and evaporated under reduced pressure. The crude product was purified by filtration thought a silica pad eluting with petroleum ether/EtOAc (8/2). The title compound was collected as a pale yellow solid (115 mg; 82% yield). LCMS (RT): 1.40 min (Method D).

### 125(E) 2-(5-Amino-2-chloro-phenyl)-2-methyl-propionitrile

Prepared according to Example 98(C), starting from 2-(2-chloro-5-nitro-phenyl)-2-methyl-propionitrile (83.0 mg; 0.37 mmol), prepared as described in 125(D), and using PtO₂ (10 mg) in MeOH (20 mL). After removing the catalyst by filtration and methanol by evaporation under vacuum, the title compound was obtained as a white solid (68.0 mg; 94% yield).

LCMS (RT): 3.2 min (Method A); MS (ES+) gave m/z: 195.18 (MH+).

### 125(F) N-[4-Chloro-3-(cyano-dimethyl-methyl)-phenyl]-3,4-dimethoxy-benzamide

Prepared according to Example 1(C), starting from 2-(5-amino-2-chloro-phenyl)-2-methyl-propionitrile (68.0 mg; 0.35 mmol), prepared as in 125(E), and using 3,4-dimethoxy-benzoyl chloride (77.0 mg; 0.38 mmol), and triethylamine (59 uL; 0.42 mmol) in DCM (10 mL) The crude was purified by preparative HPLC (Method Q) to yield the title compound as a pale yellow solid (17.9 mg; 14% yield).

¹H NMR (300 MHz, CDCl3) δ(ppm): 7.91 (d, 1H), 7.82 (br. s., 1H), 7.58 (dd, 1H), 7.50 (d, 1H), 7.44 (d, 1H), 7.41 (dd, 1H), 6.94 (d, 1H), 3.98 (s, 3H), 3.97 (s, 3H), 1.92 (s, 6H).

LCMS (RT): 2.29 min (Method G); MS (ES+) gave m/z: 359.19 (MH+).

### Example 131

### 1-Methyl-1H-indazole-3-carboxylic acid{2-[2-chloro-5-(3,4-dimethoxy-benzoylamino)-phenyl]-2-methyl-propyl}-amide

### 131(A) 2-(2-Chloro-5-nitro-phenyl)-2-methyl-propylamine

Borane-THF complex (1M solution in THF; 2.6 mL) was added dropwise to a solution of 2-(5-amino-2-chloro-phenyl)-2-methyl-propionitrile (201 mg; 0.90 mmol), prepared a described in 125(E), in dry THF (10 mL) under nitrogen atmosphere. The resulting solution was refluxed for 1 hour and then cooled to 0° C. by an ice-bath. Then 1N HCl was carefully added until the pH was 1 and the resulting solution was refluxed for 1 hour. After this time the solvent was evaporated and the crude portioned between DCM and 1M Na₂CO₃. The organic phase was dried over Na₂SO₄, filtered and evaporated under reduced pressure to give the title compound as a pale yellow solid (193 mg; 94% yield).

LCMS (RT): 2.8 min (Method A); MS (ES+) gave m/z: 229.14 (MH+).

### 131(B) 1-Methyl-1H-indazole-3-carboxylic acid[2-(2-chloro-5-nitro-phenyl)-2-methyl-propyl]-amide

A mixture of 1-methyl-1H-indazole-3-carboxylic acid (46.0 mg; 0.26 mmol), HOBt (44.4 mg; 0.33 mmol), EDC (84.0 mg; 0.44 mmol) in DCM (10 mL) was stirred at room temperature for 30 min. 2-(2-chloro-5-nitro-phenyl)-2-methyl-propylamine (50.0 mg; 0.22 mmol), prepared as described in 131(A), was added and the stirring was maintained at the same temperature for additional 16 hours. The reaction was diluted with DCM and washed in sequence with 0.5M K₂CO₃ (twice), 1N HCl and brine. The organic phase was dried (Na₂SO₄), filtered and evaporated under reduced pressure to furnish the title compound as a white solid. This compound was used as such in the next step.

LCMS (RT): 4.5 min (Method A); MS (ES+) gave m/z: 387.09 (MH+).

### 131(C) 1-Methyl-1H-indazole-3-carboxylic acid[2-(5-amino-2-chloro-phenyl)-2-methyl-propyl]-amide

Prepared according to Example 98(C), starting from 1-methyl-1H-indazole-3-carboxylic acid[2-(2-chloro-5-nitro-phenyl)-2-methyl-propyl]-amide (74.0 mg; 0.20 mmol), prepared as described in 131(B), and using PtO₂ (10 mg) in MeOH (20 mL). The catalyst was filtered off and the filtrate was evaporated under vacuum to dryness. The resulting brown solid was used in the next step without any further purification.

LCMS (RT): 3.6 min (Method A); MS (ES+) gave m/z: 357.09 (MH+).

### 131(D) 1-Methyl-1H-indazole-3-carboxylic acid{2-[2-chloro-5-(3,4-dimethoxy-benzoylamino)-phenyl]-2-methyl-propyl}-amide

3,4-Dimethoxy-benzoyl chloride (41.0 g; 0.20 mmol) was added portionwise to a solution of 1-methyl-1H-indazole-3-carboxylic acid[2-(5-amino-2-chloro-phenyl)-2-methyl-propyl]-amide (61.0 mg; 0.17 mmol), prepared as in 131(C), in triethylamine (36 uL; 0.26 mmol) and dry DCM (10 mL). The mixture was heated at 70° C. for 1 hour using a microwave oven. The reaction was diluted with DCM, washed with 1N HCl and then with 1M NaHCO₃. The organic phase was dried over sodium sulphate, filtered and evaporated under reduced pressure. The crude compound was purified by preparative HPLC (Method Q) to afford the title compound as a pale yellow solid (6.9 mg; 8% yield over three steps).

¹H NMR (300 MHz, DMSO-d6, 353 K) δ(ppm): 13.65 (br. s., 1H), 10.01 (s, 1H), 7.77 (dd, 1H), 7.72 (m, 2H), 7.58-7.69 (m, 3H), 7.54 (d, 1H), 7.42 (m, 2H), 7.31 (td, 1H), 7.08 (d, 1H), 3.85 (s, 3H), 3.84 (s, 3H), 3.55 (d, 2H), 1.33 (s, 6H).

LCMS (RT): 2.60 min (Method G); MS (ES+) gave m/z: 521.20 (MH+).

### Example 132 and 133

### N-[4-(Cyano-dimethyl-methyl)-3-pyridin-3-yl-phenyl]-3,4-dimethoxy-benzamide and N-[4-(Cyano-dimethyl-methyl)-3-ethyl-phenyl]-3,4-dimethoxy-benzamide

### 132(A) 2-(4-Amino-2-pyridin-3-yl-phenyl)-2-methyl-propionitrile

### 133(A) 2-(4-Amino-2-ethyl-phenyl)-2-methyl-propionitrile

A mixture of 2-(4-amino-2-bromo-phenyl)-2-methyl-propionitrile (157 mg; 0.66 mmol), prepared as described in 111(C), diethyl-(3-pyridyl)-borane (290 mg; 1.96 mmol), 2M K₂CO₃ (657 uL; 130 mmol) and tetrakis(triphenylphospine)palladium(0) (30 mg; 0.03 mmol) in dioxane (10 mL) was heated at 110° C. for 20 hours. The solvent was removed under vacuum and the residue taken up with DCM and washed twice with water. The organic phase was dried over Na₂SO₄, filtered and evaporated to dryness. The crude compound was used in the next step without any further purification.

### 132(A) LCMS (RT): 1.5 min (Method A); MS (ES+) gave m/z: 238.13 (MH+)

### 133(A) LCMS (RT): 2.8 min (Method A); MS (ES+) gave m/z: 189.13 (MH+)

### 132(B) N-[4-(Cyano-dimethyl-methyl)-3-pyridin-3-yl-phenyl]-3,4-dimethoxy-benzamide

### 133(B) N-[4-(Cyano-dimethyl-methyl)-3-ethyl-phenyl]-3,4-dimethoxy-benzamide

3,4-Dimethoxy-benzoyl chloride (144 mg; 0.72 mmol) was added portionwise to a mixture of 2-(4-amino-2-pyridin-3-yl-phenyl)-2-methyl-propionitrile and 2-(4-amino-2-ethyl-phenyl)-2-methyl-propionitrile, prepared as in 132(A)/133(B), and triethylamine (184 uL; 1.31 mmol) in DCM (10 mL). The mixture was stirred at room temperature for 16 hours and then heated under microwave irradiation at 70° C. for 1 hour. The reaction was diluted with DCM, washed with 1M K₂CO₃ and then with brine. The organic phase was dried over sodium sulphate, filtered and evaporated under reduced pressure. The crude was dissolved in DCM and loaded onto an ion-exchange (SCX) cartridge. Compound 133(B) was recovered by eluting with DCM/MeOH (1/1), and then compound 132(B) was recovered by eluting with MeOH/NH₄OH (9/1). Both the crude products were purified by preparative HPLC (Method Q), to afford 132(B) as a pale yellow powder (34.8 mg; 13% yield over two steps) and 133(B) as a white powder (7.8 mg; 7% yield over two steps).

132(B) ¹H NMR (300 MHz, DMSO-d6) δ(ppm): 10.16 (s, 1H), 8.63 (dd, 1H), 8.57 (dd, 1H), 7.95 (dd, 1H), 7.81 (ddd, 1H), 7.62 (dd, 1H), 7.61 (d, 1H), 7.56 (d, 1H), 7.53 (d, 1H), 7.47 (ddd, 1H), 7.08 (d, 1H), 3.83 (s, 3H), 3.83 (s, 3H), 1.62 (s, 6H),

LCMS (RT): 1.55 min (Method G); MS (ES+) gave m/z: 402.21 (MH+).

133(B) ¹H NMR (300 MHz, DMSO-d6) δ(ppm): 10.06 (s, 1H), 7.73 (d, 1H), 7.66 (dd, 1H), 7.63 (dd, 1H), 7.54 (d, 1H), 7.31 (d, 1H), 7.09 (d, 1H), 3.85 (s, 3H), 3.84 (s, 3H), 2.91 (q, 2H), 1.73 (s, 6H), 1.30 (t, 3H).

LCMS (RT): 2,29 min (Method G); MS (ES+) gave m/z: 353.19 (MH+).

### Example 151

### N-[4-(2-Acetylamino-1,1-dimethyl-ethyl)-3-fluoro-phenyl]-3,4-dimethoxy-benzamide

### 151(A) 2-(2-Fluoro-4-nitro-phenyl)-2-methyl-propylamine

Prepared according to Example 113(A), starting from 2-(2-fluoro-4-nitro-phenyl)-2-methyl-propionitrile (868 mg; 4.17 mmol), prepared as described in 96(B), and using borane-THF complex (1M solution in THF; 16.7 mL) in dry THF (15 mL). The crude compound was purified by ion-exchange chromatography [SCX, DCM/MeOH (1/1) to MeOH/NH₄OH (9/1)] to give the title compound as an orange oil (610 mg; 69% yield).

LCMS (RT): 0.91 min (Method D); MS (ES+) gave m/z: 213.1 (MH+).

### 151(B) N-[2-(2-Fluoro-4-nitro-phenyl)-2-methyl-propyl]-acetamide

Prepared according to Example 54(B), starting from 2-(2-fluoro-4-nitro-phenyl)-2-methyl-propylamine (150 mg; 0.71 mmol), prepared as in 151(A), and using acetyl chloride (100 uL; 1.41 mmol) and triethylamine (200 uL; 1.41 mmol) in DCM (8 mL). The crude product was purified by chromatography [SiO₂, DCM/MeOH (98.5/1.5)] to give the title compound as a yellow oil (136 mg, 75% yield).

LCMS (RT): 1.25 min (Method D); MS (ES+) gave m/z: 255.1 (MH+).

### 151(C) N-[2-(4-Amino-2-fluoro-phenyl)-2-methyl-propyl]-acetamide

A solution of N-[2-(2-fluoro-4-nitro-phenyl)-2-methyl-propyl]-acetamide (130 mg; 0.51 mmol) in MeOH (15 mL) was hydrogenated at 1 bar and 40° C. using a H-cube instrument (Thales nanotechology) and a Pd/C cartridge. After evaporation of the solvent, the title compound was collected as colourless crystals (110 mg; 96% yield).

LCMS (RT): 0.76 min (Method D); MS (ES+) gave m/z: 225.1 (MH+).

### 151(D) N-[4-(2-Acetylamino-1,1-dimethyl-ethyl)-3-fluoro-phenyl]-3,4-dimethoxy-benzamide

Prepared according to Example 98(D) starting from N-[2-(4-amino-2-fluoro-phenyl)-2-methyl-propyl]-acetamide (110 mg; 0.49 mmol), prepared as in 151(C), and using 3,4-dimethoxy-benzoyl chloride (128 mg; 0.64 mmol), and triethylamine (136 uL; 0.98 mmol) in DCM (8 mL). Chromatography [SiO₂, DCM to DCM/MeOH (98/2)], followed by trituration from isopropyl ether yielded the title compound as a white solid (54.0 mg; 28% yield).

¹H NMR (300 MHz, DMSO-d6) δ(ppm): 10.15 (s, 1H), 7.56-7.70 (m, 3H), 7.52 (d, 1H), 7.47 (dd, 1H), 7.25 (dd, 1H), 7.09 (d, 1H), 3.85 (s, 3H), 3.84 (s, 3H), 3.36 (d, 2H), 1.77 (s, 3H), 1.27 (s, 6H)

LCMS (RT): 1.94 min (Method G); MS (ES+) gave m/z: 389.21 (MH+).

MP: 186-188° C.

### Example 152

### N-[6-(Cyano-dimethyl-methyl)-4′-trifluoromethyl-biphenyl-3-yl]-3,4-dimethoxy-benzamide

A mixture of N-[3-bromo-4-(cyano-dimethyl-methyl)-phenyl]-3,4-dimethoxy-benzamide (70.0 mg; 0.17 mmol), prepared as in 111(D), 4-(trifluoromethyephenylboronic acid (42.0 mg; 0.22 mmol) and KF (20.0 mg; 0.34 mmol) in methanol (4 mL) was purged with nitrogen for 5 min. Palladium (II) acetate was added and the vessel was sealed and heated in a microwave oven at 110° C. for 1.5 hour. The solvent was removed under vacuum and the residue was portioned between water and DCM. The organic phase was dried over Na₂SO₄, filtered and evaporated to dryness. The crude compound was purified by chromatography [SiO₂, DCM/MeOH (99/1)]. The resulting compound was further purified by crystallization from ethanol to afford the title compound as a white solid (32.0 mg; 40% yield).

¹H NMR (300 MHz, DMSO-d6) δ(ppm): 10.15 (s, 1H), 7.94 (dd, 1H), 7.80 (m, 2H), 7.58-7.68 (m, 4H), 7.56 (d, 1H), 7.53 (d, 1H), 7.08 (d, 1H), 3.84 (s, 3H), 3.83 (s, 3H), 1.61 (s, 6H)

LCMS (RT): 4.31 min (Method I); MS (ES+) gave m/z: 469.10 (MH+).

### Example 153

### 2-Chloro-N-[4-(cyano-dimethyl-methyl)-phenyl]-3,4-dimethoxy-benzamide

A mixture of 2-(4-amino-phenyl)-2-methyl-propionitrile (60.0 mg; 0.37 mmol), prepared as in 1(B), 2-chloro-3,4-dimethoxy-benzoic acid (81.0 mg, 0.37 mmol), HOBt (60.0 mg; 0.37 mmol) and EDC (107 mg; 0.56 mmol) in DCM (5 mL) was stirred at room temperature for 56 hours. Then TEA (100 ul; 0.75 mmol) was added and the resulting solution was heated at reflux for 6 hours. The reaction was diluted with DCM and washed with 2N HCl, sat. NaHCO₃ and brine. The organic phase was collected, dried (Na₂SO₄), filtered and evaporated to dryness. The crude product was purified by chromatography [SiO₂, Petroleum ether/EtOAc (2/1)] to give the title compound as a white solid (22.0 mg; 17% yield).

1H NMR (300 MHz, CDCl3-d) δ(ppm): 8.06 (br. s., 1H), 7.69 (m, 2H), 7.62 (d, 1H), 7.49 (m, 2H), 6.95 (d, 1H), 3.96 (s, 3H), 3.92 (s, 3H), 1.75 (s, 6H).

LCMS (RT): 2.33 min (Method G); MS (ES+) gave m/z: 359.12 (MH+).

MP: 195-198° C.

### Example 154

### N-[4-(Cyano-dimethyl-methyl)-phenyl]-2,4,5-trimethoxy-benzamide

Prepared according to Example 153, starting from 2-(4-amino-phenyl)-2-methyl-propionitrile (60.0 mg; 0.37 mmol), prepared as in 1(B) and using 2,4,5-trimethoxy-benzoic acid (80.0 mg, 0.37 mmol), HOBt (60.0 mg; 0.37 mmol), EDC (107 mg; 0.56 mmol) and TEA (100 uL; 0.75 mmol) in DCM (5 mL). The crude compound was purified by preparative HPLC (Method Q) to furnish the title compound as a white solid (22 mg; 16% yield).

¹H NMR (300 MHz, CDCl3-d) δ(ppm): 9.88 (m, 1H), 7.82 (s, 1H), 7.70 (m, 2H), 7.46 (m, 2H), 6.59 (s, 1H), 4.07 (s, 3H), 3.98 (s, 3H), 3.94 (s, 3H), 1.74 s, 6H)

LCMS (RT): 2.37 min (Method G); MS (ES+) gave m/z: 355.15 (MH+).

### Example 155

### 2-Chloro-N-[4-(cyano-dimethyl-methyl)-phenyl]-4,5-dimethoxy-benzamide

A mixture of 2-(4-amino-phenyl)-2-methyl-propionitrile (160 mg; 0.50 mmol), prepared as in 1(B), 2-chloro-4,5-dimethoxy-benzoic acid (108 mg, 0.50 mmol), HOBt (77.0 mg; 0.50 mmol), EDC (144 mg; 0.75 mmol) and TEA (140 uL; 1.00 mmol) in dioxane (6 mL) was warmed at 100° C. for 2 hours. Then, the reaction was diluted with DCM and washed with 1N HCl and 5% NaHCO₃. The organic phase was collected, dried (Na₂SO₄), filtered and evaporated to dryness. The crude product was purified by chromatography [SiO₂, Petroleum ether/EtOAc (1/1)] to give the title compound as a white solid (30.0 mg; 17% yield).

1H NMR (300 MHz, CDCl3-d) δ(ppm): 8.33 (br. s., 1H), 7.70 (m, 2H), 7.50 (m, 2H), 7.48 (s, 1H), 6.91 (s, 1H), 3.94-3.97 (m, 3H), 3.95 (d, 3H), 1.76 (s, 6H).

LCMS (RT): 2.24 min (Method G); MS (ES+) gave m/z: 359.18 (MH+).

MP: 142-144° C.

### Example 156

### N-[2′-Chloro-6-(cyano-dimethyl-methyl)-biphenyl-3-yl]-3,4-dimethoxy-benzamide

A mixture of N-[3-bromo-4-(cyano-dimethyl-methyl)-phenyl]-3,4-dimethoxy-benzamide (80.0 mg; 0.20 mmol), prepared as in 111(D), 2-chlorophenylboronic acid (40.0 mg; 0.26 mmol) and 2M K₂CO₃ (200 ul; 0.40 mmol) in 1,2-dimethoxyethane (4 mL) was purged with nitrogen for 30 min. Tetrakis(triphenylphospine) palladium(0) (11 mg; 0.01 mmol) was added and the vessel was sealed and heated in a microwave oven at 100° C. for 4 hours. The solvent was removed under vacuum and the residue was portioned between water and DCM. The organic phase was dried over Na₂SO₄, filtered and evaporated to dryness. The crude compound was purified by preparative HPLC (Method Q) to afford the title compound as a white solid (14.0 mg; 16% yield).

¹H NMR (300 MHz, CDCl3-d) δ(ppm): 7.84 (dd, 1H), 7.79 (s, 1H), 7.66 (d, 1H), 7.46-7.53 (m, 2H), 7.30-7.44 (m, 5H), 6.93 (d, 1H), 3.97 (s, 3H), 3.96 (s, 3H), 1.67 (s, 3H), 1.63 (s, 3H).

LCMS (RT): 3.94 min (Method I); MS (ES+) gave m/z: 435.13 (MH+).

### Example 157

### N-[3′-Chloro-6-(cyano-dimethyl-methyl)-biphenyl-3-yl]-3,4-dimethoxy-benzamide

Prepared according to Example 156, staring from N-[3-bromo-4-(cyano-dimethyl-methyl)-phenyl]-3,4-dimethoxy-benzamide (80.0 mg; 0.20 mmol), prepared as in 111(D), and using 3-chlorophenylboronic acid (40.0 mg; 0.26 mmol), 2M K₂CO₃ (200 ul; 0.40 mmol) and tetrakis(triphenylphospine) palladium(0) (11 mg; 0.01 mmol) in 1,2-dimethoxyethane (4 mL). Purification by chromatography [SiO₂, Petroleum ether/EtOAc (3/1)] afforded the title compound as a white solid (52.0 mg; 59% yield).

¹H NMR (300 MHz, CDCl3-d) δ(ppm): 7.80 (s, 1H), 7.78 (dd, 1H), 7.62 (d, 1H), 7.50 (d, 1H), 7.33-7.44 (m, 5H), 7.28-7.32 (m, 1H), 6.93 (d, 1H), 3.97 (s, 6H), 1.65 (s, 6H)

LCMS (RT): 2.64 min (Method G); MS (ES+) gave m/z: 435.19 (MH+).

MP: 144-146° C.

### Example 158

### N-[4-(Cyano-dimethyl-methyl)-3-pyridin-4-yl-phenyl]-3,4-dimethoxy-benzamide

### 158(A) N-[4-(Cyano-dimethyl-methyl)-3-(4,4,5,5-tetramethyl-[1,3,2]dioxaborolan-2-yl)-phenyl]-3,4-dimethoxy-benzamide

A mixture of N-[3-bromo-4-(cyano-dimethyl-methyl)-phenyl]-3,4-dimethoxy-benzamide (600 mg; 1.49 mmol), prepared as in 111(D), bis(pinacolato)diboron (1926 mg; 7.59 mmol), 1,1′-bis(diphenylphosphino)ferrocenedichloropalladium (II) (122 mg; 0.15 mmol) and K₂CO₃ (638 mg; 4.62 mmol) in DMSO (6 mL) was stirred at 95° C. for 1.30 hour. After this time, the reaction was diluted with EtOAc and filtered. The solution was washed with brine, dried over Na₂SO₄, filtered and evaporated to dryness. The crude was purified by chromatography [SiO₂, DCM/MeOH (99/1)] and the resulting pale yellow solid was triturated with ethanol to obtain the title compound as a white solid (247 mg; 37% yield).

LCMS (RT): 1.70 min (Method D); MS (ES+) gave m/z: 451.2 (MH+).

### 158(B) N-[4-(Cyano-dimethyl-methyl)-3-pyridin-4-yl-phenyl]-3,4-dimethoxy-benzamide

A mixture of N-[4-(cyano-dimethyl-methyl)-3-(4,4,5,5-tetramethyl-[1,3,2]dioxaborolan-2-yl)-phenyl]-3,4-dimethoxy-benzamide (90.0 mg; 0.20 mmol), prepared as in 158(A), 4-bromopyridine hydrochloride (39.0 mg; 0.20 mmol) and 2M K₂CO₃ (350 ul; 0.70 mmol) in 1,2-dimethoxyethane (4 mL) was purged with nitrogen for 30 min. Tetrakis(triphenylphospine) palladium(0) (11 mg; 0.01 mmol) was added and the vessel was sealed and heated in a microwave oven at 100° C. for 8 hours. The solvent was removed under vacuum and the residue was portioned between water and DCM. The organic phase was dried over Na₂SO₄, filtered and evaporated to dryness. The crude compound was purified by chromatography [SiO₂, EtOAc] and the resulting compound was triturated with DCM to obtain the title compound as a white solid (48.0 mg; 60% yield).

¹H NMR (300 MHz, DMSO-d6) δ(ppm): 10.17 (s, 1H), 8.54-8.71 (m, 2H), 7.95 (dd, 1H), 7.58-7.69 (m, 2H), 7.53 (d, 1H), 7.53 (d, 1H), 7.35-7.49 (m, 2H), 7.08 (d, 1H), 3.84 (s, 3H), 3.83 (s, 3H), 1.63 (s, 6H).

LCMS (RT): 2.27 min (Method G); MS (ES+) gave m/z: 402.20 (MH+).

MP: 196-199° C.

### Example 159

### N-[4-(3-Acetylamino-1,1-dimethyl-propyl)-phenyl]-3,4-dimethoxy-benzamide

### Method A

### 159(A) 3-Methyl-3-(4-nitro-phenyl)-butylamine

Prepared according to Example 113(A), starting from 3-methyl-3-(4-nitro-phenyl)-butyronitrile (160 mg; 0.78 mmol), prepared as described in 114(B), and using borane-THF complex (1M solution in THF; 3.14 mL) in dry THF (3 mL). The crude compound was purified by ion-exchange chromatography [SCX, DCM/MeOH (1/1) to MeOH/NH₄OH (9/1)] to give the title compound as a yellow oil (84.0 mg; 52% yield).

LCMS (RT): 0.95 min (Method D); MS (ES+) gave m/z: 209.1 (MH+).

### 159(B) N-[3-Methyl-3-(4-nitro-phenyl)-butyl]-acetamide

A mixture of 3-methyl-3-(4-nitro-phenyl)-butylamine (84.0 mg; 0.40 mmol), prepared as in 159(A), acetyl chloride (45 uL; 0.61 mmol) and triethylamine (84 uL; 0.61 mmol) in DCM (8 mL) was stirred at room temperature for 16 hours. The reaction was then diluted with DCM and washed three times with water. The organic layer was separated, dried over Na₂SO₄, filtered and evaporated to dryness to give the title compound (74.0 mg), which was used in the next step without any further purification.

LCMS (RT): 1.94 min (Method E); MS (ES+) gave m/z: 251.1 (MH+).

### Method B

### 159(C) 3-Methyl-3-phenylbutanoic acid

A solution of 3-methylbut-2-enoic acid (10.0 g; 0.10 mol) in dry benzene (25 mL) was stirred in an ice-bath while anhydrous aluminum chloride (16.0 g; 0.12 mol) was added in small portions over 1 h and the temperature was kept below 5° C. The reaction mixture was cooled and stirred for twenty minutes, then the cooling bath was removed and the mixture was vigorously stirred and allowed to attain room temperature. After being stirred for 16 hours, the reaction was poured over a large quantity of ice and the excess of benzene was removed in vacuo. The water was removed by filtration and the brown gum was triturated in ethanol-water (1:1 ratio). The resulting off-white solid was collected by suction filtration and dried at 50° C. under vacuum overnight (13.7 g; 77% yield).

¹H NMR (300 MHz, DMSO-d6) δ(ppm): 11.78 (br. s., 1H), 7.33-7.43 (m, 2H), 7.21-7.33 (m, 2H), 7.07-7.21 (m, 1H), 2.56 (s, 2H), 1.37 (s, 6H).

### 159(D) 3-Methyl-3-phenylbutanamide

To a solution of 3-methyl-3-phenylbutanoic acid (10.0 g; 56.0 mmol), prepared as in 159(C), in DCM (400 mL) and in presence of some drops of DMF, was added oxalyl chloride (9.5 mL; 112 mmol) at 0° C. under nitrogen atmosphere. After being stirred at room temperature for 1 hour, the solvent was evaporated under vacuum. The resulting acyl chloride was taken up with DCM (200 mL) and treated with ammonium hydroxide (200 mL) at 0° C. The reaction was vigorously stirred at room temperature for 1 hour, then the phases were separated and the organic one was diluted with EtOAc, washed with 1N HCl, dried over Na₂SO₄, filtered and evaporated to dryness. The resulting crude compound was filtered through a silica pad eluting with EtOAc/MeOH (9/1) to afford the title compound as a white solid (6.42 g; 65% yield).

LCMS (RT): 1.11 min (Method D); MS (ES+) gave m/z: 178.2 (MH+).

### 159(E) 3-Methyl-3-(4-nitrophenyl)butanamide

To a solution of 3-methyl-3-phenylbutanamide (6.42 g; 36.3 mmol), prepared as in 159(D), and KNO₃ (3.66 g; 36.3 mmol) in DCM (200 mL), was added conc. H₂SO₄ (10 mL) and the resulting reaction was stirred at 50° C. for 16 hours. After being cooled, the reaction was poured onto ice. The mixture was diluted with water, the layers were separated and the organic one was dried over Na₂SO₄, filtered and evaporated to dryness. The resulting crude compound was filtered through a silica pad eluting with EtOAc/MeOH (98/2) to afford the title compound as a pale yellow solid (4.23 g; 52% yield).

¹H NMR (300 MHz, CDCl3-d) δ(ppm): 8.08-8.31 (m, 2H), 7.58 (m, 2H), 2.66 (s, 2H), 1.55 (s, 6H).

LCMS (RT): 1.16 min (Method D); MS (ES+) gave m/z: 223.2 (MH+).

### 159(B) N-(3-methyl-3-(4-nitrophenyl)butyl)acetamide

To a solution of 3-methyl-3-(4-nitrophenyl)butanamide (670 mg; 2.41 mmol), prepared as described in 159(E), in dry THF (20 mL), was added dropwise borane-THF complex (1M solution in THF; 7.3 mL) over 15 min while stirring under nitrogen atmosphere. The resulting solution was heated at 80° C. for 16 hours, cooled at room temperature and quenched by adding methanol dropwise. The solvent was removed under vacuum, the residue was taken up with THF (30 mL) and treated with 1N HCl (3 mL). The resulting reaction was refluxed for 2 hours and then concentrated under vacuum. The residue was portioned between DCM and water, the organic phase was collected, dried over Na₂SO₄, filtered and evaporated to dryness. The crude was dissolved in dry DCM (20 mL) and TEA (676 uL; 4.82 mmol) and cooled at 0° C. by ice-bath. Acetyl chloride (206 uL; 2.89 mmol) was added and the reaction was stirred at room temperature for two hours. Then it was diluted with DCM and washed sequentially with 2M K₂CO₃, 1N HCl and brine. The organic phase was dried over sodium sulphate, filtered and evaporated under reduced pressure to give the title compound as a yellow oil (613 mg; 76% yield).

¹H NMR (300 MHz, CDCl3) δ(ppm): 8.18 (m, 2H), 7.53 (m, 2H), 5.23 (br. s., 1H), 2.98-3.14 (m, 2H), 1.88-1.98 (m, 2H), 1.86 (s, 3H), 1.40 (s, 6H)

LCMS (RT): 1.23 min (Method D); MS (ES+) gave m/z: 251.1 (MH+).

### Method C

### 159(F) 2-Methyl-2-(4-nitrophenyl)propanal

To a solution of 2-methyl-2-(4-nitro-phenyl)-propan-1-ol (0.71 g; 3.66 mmol), prepared as in 80(A), in DCM (20 mL), was added Dess-Martin periodinane (1.55 g; 3.66 mmol) and the resulting mixture was stirred at room temperature for 40 min. The reaction was diluted with DCM and washed with sat. sodium thiosulfate and then with NaHCO₃. The organic layer was dried over Na₂SO₄, filtered and evaporated to dryness. The crude was purified by flash chromatography [SiO₂, Petroleum ether/EtOAc (99/1 to 98/2)] to give the title compound as a light yellow oil (0.42 g; 59% yield).

¹H NMR (300 MHz, CDCl3) δ(ppm): 9.56 (s, 1H), 8.24 (m, 2H), 7.47 (m, 2H), 1.54 (s, 6H).

### 159(G) 3-Methyl-3-(4-nitrophenyl)butanal

To a suspension of (methoxymethyl)triphenylphosphonium chloride (2.65 g; 7.75 mmol) in dry THF (60 mL), was added potassium bis(trimethylsilyl)amide (0.5 M solution in toluene; 15.5 mL). The red mixture was stirred at room temperature for 15 min and then 2-methyl-2-(4-nitrophenyl)propanal (880 mg; 4.56 mmol), prepared as in 159(F), was added. After being stirred for 2 hours at room temperature, the reaction was quenched with water and extracted three times with DCM. The combined organic layers were dried over Na₂SO₄, filtered and concentrated in vacuo. The crude was quickly filtered through a silica pad eluting with Petroleum ether/EtOAc (99/1). The resulting product was dissolved in DCM (20 mL) and treated with a H₂O/TFA (1/1 ratio; 4.4 mL). The reaction was stirred at room temperature for 1 h, then it was diluted with DCM and pH was adjusted to about 7 by adding 5% NaHCO₃. The organic phase was collected, dried over Na₂SO₄, filtered and evaporated to dryness. Purification of the resulting crude by flash chromatography [SiO₂, Petroleum ether/EtOAc (99/1 to 95/5)] yielded the title compound as a light yellow oil (0.66 g; 70% yield).

¹H NMR (300 MHz, CDCl3-d) δ(ppm): 9.59 (s, 1H), 8.22 (m, 2H), 7.56 (m, 2H), 2.79 (d, 2H), 1.53 (s, 6H).

### 159(B) N-(3-methyl-3-(4-nitrophenyl)butyl)acetamide

Ammonium acetate was added until saturation to a solution of 3-methyl-3-(4-nitrophenyl)butanal (660 mg; 3.19 mmol), prepared as in 159(G), in MeOH. Sodium cyanoborohydride (200 mg; 3.19 mmol) was added and the resulting reaction was stirred at room temperature overnight. Then it was diluted with DCM and washed with water. The aqueous solution was extracted three times with DCM and the combined organic phases were dried over Na₂SO₄, filtered and evaporated to dryness. The crude was dissolved in DCM (12 mL) and TEA (667 uL; 4.79 mmol) and treated with acyl chloride (340 uL; 4.79 mmol). After being stirred at room temperature for one hour, the reaction was diluted with DCM and washed with 5% NaHCO₃, dried over Na₂SO₄, filtered and evaporated to dryness. The crude was purified by flash chromatography [SiO₂, Petroleum ether/EtOAc (6/4 to 1/9)] to afford the title compound as a light yellow oil (175 mg; 22% yield).

¹H NMR (300 MHz, CDCl3) δ(ppm): 8.18 (m, 2H), 7.53 (m, 2H), 5.21 (br. s., 1H), 2.93-3.22 (m, 2H), 1.88-1.97 (m, 2H), 1.86 (s, 3H), 1.40 (s, 6H)

LCMS (RT): 1.23 min (Method D); MS (ES+) gave m/z: 251.1 (MH+).

### 159(H) N-(3-(4-aminophenyl)-3-methylbutyl)acetamide

10% Pd/C (180 mg) was added to a solution of N-(3-methyl-3-(4-nitrophenyl)butyl)acetamide (1.81 g; 7.30 mmol), prepared as in 159(B) (prepared according to Method A, B or C), in MeOH (70 mL). The mixture was hydrogenated at 1.3 bar at room temperature for 2 hours, then the catalyst was filtered off and the filtrate was concentrated under reduced pressure to give the title compound as a pale yellow oil (1.54 g; 96% yield).

¹H NMR (300 MHz, CDCl3) δ(ppm): 7.14 (m, 2H), 6.66 (m, 2H), 4.97 (br. s., 1H), 3.59 (br. s., 2H), 3.04-3.16 (m, 2H), 1.78 (s, 3 1-1), 1.72-1.85 (m, 2H), 1.31 (s, 6H)

LCMS (RT): 0.73 min (Method D); MS (ES+) gave m/z: 221.1 (MH+).

### 159(I) N-(4-(4-acetamido-2-methylbutan-2-yl)phenyl)-3,4-dimethoxybenzamide

3,4-Dimethoxy-benzoyl chloride (822 mg; 4.10 mmol) was added to a solution of N-(3-(4-aminophenyl)-3-methylbutyl)acetamide (752 mg; 3.40 mmol), prepared as in 159(H), in pyridine (10 mL). The reaction was heated under microwave irradiation at 100° C. for 1 hour and then the pyridine was removed by rotary evaporator. The residue was taken up with DCM, washed sequentially with 5% NaHCO₃, 1N HCl and brine. The organic layer was dried over sodium sulphate, filtered and evaporated under reduced pressure. The crude compound was purified by flash chromatography [SiO₂, DCM to DCM/MeOH (99/1)] to afford the title compound as a white amorphous solid (660 mg; 50% yield).

¹H NMR (300 MHz, CDCl3) δ(ppm): 7.80 (s, 1H), 7.57 (m, 2H), 7.52 (d, 1H), 7.41 (dd, 1H) 7.35 (m, 2H), 6.93 (d, 1H), 5.13 (br. s., 1H), 3.97 (s, 3H), 3.96 (s, 3H), 3.03-3.15 (m, 2H), 1.84-1.90 (m, 2H), 1.82 (s, 3H), 1.36 (s, 6H)

LCMS (RT): 1.84 min (Method P); MS (ES+) gave m/z: 385.12 (MH+).

### Example 160

### N-[4-(Cyano-dimethyl-methyl)-phenyl]-3,4-diethoxy-benzamide

Prepared according to Example 153, starting from 2-(4-amino-phenyl)-2-methyl-propionitrile (60.0 mg; 0.37 mmol), prepared as in 1(B), and using 3,4-diethoxy-benzoic acid (79.0 mg, 0.37 mmol), HOBt (60.0 mg; 0.45 mmol), EDC (107 mg; 0.56 mmol) and TEA (105 uL; 0.75 mmol) in DCM (5 mL). The crude product was purified by chromatography [SiO₂, Petroleum ether/EtOAc (95/5 to 8/2)], followed by crystallization from EtOAc to give the title compound as a white solid (16.0 mg; 12% yield).

¹H NMR (300 MHz, CDCl3-d) δ(ppm): 7.77 (s, 1H), 7.66 (m, 2H), 7.44-7.53 (m, 3H), 7.38 (dd, 1H), 6.92 (d, 1H), 4.19 (q, 2H), 4.17 (q, 2H), 1.74 (s, 6H), 1.50 (t, 3H), 1.49 (t, 3H).

LCMS (RT): 2.50 min (Method G); MS (ES+) gave m/z: 353.19 (MH+).

MP: 138-140° C.

### Example 161

### Imidazo[1,2-a]pyridine-3-carboxylic acid {2-[4-(3,4-dimethoxy-benzoylamino)-phenyl]-2-methyl-propyl}-amide

A mixture of imidazo[1,2-a]pyridine-3-carboxylic acid (35.0 mg; 0.21 mmol), HOBt (37.0 mg; 0.28 mmol) and EDC (53.0 mg; 0.28 mmol) in DCM/dioxane/DMF (3/2/1 ratio; 6 mL) was stirred at room temperature for about 15 minutes. Then, N-[4-(2-amino-1,1-dimethyl-ethyl)-phenyl]-3,4-dimethoxy-benzamide (100 mg; 0.30 mmol), prepared as described in 26(A), and TEA (65 uL; 0.47 mmol) were added. After stirring at room temperature for 16 hours, the solvent was removed under vacuum and the residue was taken up with DCM, which was washed sequentially with sat.NaHCO₃ and water. The organic phase was dried over Na₂SO₄, filtered and evaporated to dryness. The crude compound was purified by crystallization from DCM/isopropyl ether (1/1) to give the title compound as a white solid (48.0 mg; 48% yield).

¹H NMR (300 MHz, CDCl3-d) δ(ppm): 10.00 (s, 1H), 9.39 (dt, 1H), 8.34 (s, 1H), 8.25 (t, 1H), 7.65-7.75 (m, 3H), 7.61 (dd, 1H), 7.53 (d, 1H), 7.36-7.48 (m, 3H), 7.03-7.14 (m, 2H), 3.84 (s, 3H), 3.83 (s, 3H), 3.49 (d, 2H), 1.33 (s, 6H).

LCMS (RT): 1.85 min (Method G); MS (ES+) gave m/z: 473.17 (MH+).

MP: 181-183° C.

### Example 162

### 1H-Benzoimidazole-2-carboxylic acid {2-[4-(3,4-dimethoxy-benzoylamino)-phenyl]-2-methyl-propyl}-amide

To a cold (0° C.) solution of 1H-benzoimidazole-2-carboxylic acid (42.0 mg; 0.23 mmol) in dry DCM (2 mL) were added oxalyl chloride (90.0 uL; 0.92 mmol) and few drops of DMF. The mixture was allowed to warm to room temperature and the stirring was maintained for 3 hours. The solvent was evaporated under vacuum and the resulting compound was dissolved in DCM (2 mL). This solution was added dropwise to a stirred mixture of N-[4-(2-amino-1,1-dimethyl-ethyl)-phenyl]-3,4-dimethoxy-benzamide (70.0 mg; 0.21 mmol), prepared as described in 26(A) and triethylamine (79 uL; 0.51 mmol) in DCM (2 mL) at 0° C. After stirring at room temperature for 16 hours, the precipitate was collected by filtration, re-dissolved in DCM and washed with sat. NaHCO₃. The organic phase was dried over Na₂SO₄, filtered and evaporated to dryness to afford the title compound as a white solid (35.0 mg; 32% yield).

¹H NMR (300 MHz, DMSO-d6) δ(ppm): 13.23 (br. s., 1H), 10.02 (s, 1H), 8.20 (t, 1H), 7.72 (m, 2H), 7.66-7.77 (m, 1H), 7.62 (dd, 1H), 7.48-7.59 (m, 1H), 7.54 (d, 1H), 7.43 (d, 2H), 7.18-7.37 (m, 2H), 7.08 (d, 1H), 3.85 (s, 3H), 3.84 (s, 3H), 3.57 (d, 2H), 1.34 (s, 6H)

LCMS (RT): 2.10 min (Method G); MS (ES+) gave m/z: 473.27 (MH+).

MP: 223-226° C.

### Example 163

### N-[4-(Cyano-dimethyl-methyl)-3-isopropenyl-phenyl]-3,4-dimethoxy-benzamide

A mixture of N-[3-bromo-4-(cyano-dimethyl-methyl)-phenyl]-3,4-dimethoxy-benzamide (100 mg; 0.25 mmol), prepared as in 111(D), and 2M K₂CO₃ (248 uL; 0.50 mmol) in xylene (3 mL) was purged with nitrogen for about 10 min. 1,1′-bis(diphenylphosphino)ferrocenedichloropalladium (II) and 2-isopropenyl-4,4,5,5-tetramethyl-[1,3,2]dioxaborolane (0.14 mL; 0.74 mg) were added, the tube was sealed and microwave-heated at 140° C. for 2 hours. Then, the reaction was portioned between water and EtOAc. The organic phase was dried over Na₂SO₄, filtered and evaporated to dryness. The crude compound was purified by chromatography [SiO₂, Petroleum ether/EtOAc (95/5 to 7/3)] to provide the title compound as a white sticky solid (45.0 mg; 50% yield).

¹H NMR (300 MHz, DMSO-d6) δ(ppm): 10.09 (s, 1H), 7.76 (dd, 1H), 7.63 (dd, 1H), 7.54 (d, 1H), 7.52 (d, 1H), 7.46 (d, 1H), 7.09 (d, 1H), 5.34 (t, 1H), 4.99 (dd, 1H), 3.85 (s, 3H), 3.84 (s, 3H), 2.14 (s, 3H), 1.77 (s, 6H).

LCMS (RT): 2.37 min (Method G); MS (ES+) gave m/z: 365.24 (MH+).

### Example 164

### N-[4-(Cyano-dimethyl-methyl)-3-hydroxy-phenyl]-3,4-dimethoxy-benzamide

Hydrogen peroxide (35%; 800 uL) was added to a solution of N-[4-(cyano-dimethyl-methyl)-3-(4,4,5,5-tetramethyl-[1,3,2]dioxaborolan-2-yl)-phenyl]-3,4-dimethoxy-benzamide (100 mg; 0.22 mmol), prepared as in 158(A), in dioxane (10 mL). The resulting solution was heated at 40° C. for 2 hours, and then an additional portion of hydrogen peroxide (35%; 200 uL) was added and the stirring was maintained for further 5 hours while heating at 40° C. After this time, the reaction was portioned between water and DCM, the organic layer was separated, dried over Na₂SO₄, filtered and evaporated to dryness. The crude compound was purified by preparative HPLC (Method S) to afford the title compound as a white solid (8.0 mg; 11% yield).

¹H NMR (300 MHz, CDCl3-d) δ(ppm): 9.15 (s, 1H), 8.31 (s, 1H), 7.49 (d, 1H), 7.47 (d, 1H), 7.44 (dd, 1H), 7.26 (d, 1H), 7.03 (dd, 1H), 6.90 (d, 1H), 3.95 (s, 3H), 3.93 (s, 3H), 1.99 (s, 6H).

LCMS (RT): 1.95 min (Method G); MS (ES+) gave m/z: 341.26 (MH+).

### Example 165

### N-[4-(Cyano-dimethyl-methyl)-3-cyclopropyl-phenyl]-3,4-dimethoxy-benzamide

### 165(A) 2-(4-Amino-2-cyclopropyl-phenyl)-2-methyl-propionitrile

A mixture of 2-(4-amino-2-bromo-phenyl)-2-methyl-propionitrile (80.0 mg; 0.33 mol), prepared as in 111(C), cyclopropylboronic acid (28.0 mg; 1.00 mmol), KF (77.0 mg; 1.33 mmol) and and tetrakis(triphenylphospine)palladium(0) (19 mg; 0.02 mmol) in toluene (3 mL) was heated under microwave irradiation at 80° C. for 1 hour. The reaction was diluted with EtOAc and washed with water. The organic phase was dried over Na₂SO₄, filtered and evaporated to dryness. The crude compound was purified by ion-exchange chromatography [SCX, DCM/MeOH (1/1) to MeOH/NH₄OH (9/1)] to give the title compound as a yellow oil (60.6 mg; 91% yield).

LCMS (RT): 0.95 min (Method D); MS (ES+) gave m/z: 201.2 (MH+).

### 165(B) N-[4-(Cyano-dimethyl-methyl)-3-cyclopropyl-phenyl]-3,4-dimethoxy-benzamide

Prepared according to Example 1(C) starting from 2-(4-amino-2-cyclopropyl-phenyl)-2-methyl-propionitrile (60.6 mg; 0.30 mmol), prepared as in 165(A), and using 3,4-dimethoxy-benzoyl chloride (72.6 mg; 0.36 mmol), and triethylamine (63 uL; 0.45 mmol) in DCM (10 mL). The crude was purified by preparative HPLC (Method Q) to give the title compound as a white powder (16 mg; 15% yield).

¹H NMR (300 MHz, CDCl3-d) δ(ppm): 7.71 (s, 1H) 7.50 (d, 1H) 7.47 (dd, 1H) 7.39 (dd, 1H) 7.32 (d, 1H) 7.21 (d, 1H) 6.93 (d, 1H) 3.97 (s, 3H) 3.96 (s, 3H) 2.35-2.62 (m, 1H) 1.89 (s, 6H) 1.07-1.30 (m, 2H) 0.86-0.97 (m, 2H).

LCMS (RT): 2.41 min (Method G); MS (ES+) gave m/z: 365.18 (MH+).

### Example 166

### N-[3-Cyano-4-(cyano-dimethyl-methyl)-phenyl]-3,4-dimethoxy-benzamide

### 166(A) 5-Amino-2-(cyano-dimethyl-methyl)-benzonitrile

A mixture of 2-(4-amino-2-bromo-phenyl)-2-methyl-propionitrile (80.0 mg; 0.33 mol), prepared as in 111(C), bis(triphenylphosphine)palladium(II) chloride (117 mg; 0.17 mmol), tributyltin cyanide (158 mg; 0.50 mmol), tetrabutylammonium bromide (107 mg; 0.33 mmol) and K₂CO₃ (46.0 mg; 0.33 mmol) in DMF (3 mL) was heated under microwave irradiation at 100° C. for 2 hours. The solvent was removed under vacuum and the residue portioned between DCM and water. The organic phase was collected, dried over Na₂SO₄, filtered and evaporated to dryness. The crude compound was partially purified by ion-exchange chromatography [SCX, DCM/MeOH (1/1) to MeOH/NH₄OH (9/1)] to give the title compound, which was used as such in the next step.

LCMS (RT): 1.12 min (Method D); MS (ES+) gave m/z: 186.1 (MH+), 208.1 (M+Na).

### 166(B) N43-Cyano-4-(cyano-dimethyl-methyl)-phenyl]-3,4-dimethoxy-benzamide

Prepared according to Example 1(C) starting from_{—}5-amino-2-(cyano-dimethyl-methyl)-benzonitrile (61 mg; 0.33 mmol), prepared as in 166(A), and using 3,4-dimethoxy-benzoyl chloride (80.0 mg; 0.40 mmol), and triethylamine (70 uL; 0.50 mmol) in DCM (10 mL) The crude was purified by preparative HPLC (Method Q) to give the title compound as a pale yellow solid (14 mg; 12% yield over two steps).

¹H NMR (300 MHz, CDCl3-d) δ (ppm): 8.18 (d, 1H) 7.87 (br. s., 1H) 7.87 (dd, 1H) 7.77 (d, 1H) 7.51 (d, 1H) 7.41 (dd, 1H) 6.96 (d, 1H) 4.00 (s, 3H) 3.99 (s, 3H) 1.99 (s, 6H).

LCMS (RT): 2.20 min (Method G); MS (ES+) gave m/z: 350.16 (MH+).

### Example 167

### N-[6-(Cyano-dimethyl-methyl)-4′-methyl-biphenyl-3-yl]-3,4-dimethoxy-benzamide

### 167(A) 2-(5-Amino-4′-methyl-biphenyl-2-yl)-2-methyl-propionitrile

A mixture of 2-(4-amino-2-bromo-phenyl)-2-methyl-propionitrile (70.0 mg; 0.30 mol), prepared as in 111(C), 4-methylbenzene boronic acid (47.0 mg; 0.35 mmol), 2 M K₂CO₃ (292 uL; 0.58 mmol) and tetrakis(triphenylphospine)palladium(0) (17 mg; 0.02 mmol) in 1,2-dimethoxyethane (3 mL) was heated under microwave irradiation at 80° C. for 1 hour. The reaction was diluted with EtOAc and washed with water. The organic phase was dried over Na2SO₄, filtered and evaporated to dryness. The crude compound was purified by ion-exchange chromatography [SCX, DCM/MeOH (1/1) to MeOH/NH₄OH (9/1)] to give the title compound as a yellow oil (31 mg; 42% yield).

LCMS (RT): 1.25 min (Method D); MS (ES+) gave m/z: 251.0 (MH+).

### 167(B) N-[6-(Cyano-dimethyl-methyl)-4′-methyl-biphenyl-3-yl]-3,4-dimethoxy-benzamide

Prepared according to Example 1(C) starting from 2-(5-amino-4′-methyl-biphenyl-2-yl)-2-methyl-propionitrile (31.0 mg; 0.12 mmol), prepared as in 167(A), and using 3,4-dimethoxy-benzoyl chloride (29.0 mg; 0.14 mmol), and triethylamine (26 uL; 0.18 mmol) in DCM (3 mL) The crude was purified by preparative HPLC (Method Q) to give the title compound as a white powder (25 mg; 49% yield).

¹H NMR (300 MHz, CDCl3-d) δ(ppm): 7.81 (dd, 1H) 7.74 (s, 1H) 7.62 (d, 1H) 7.49 (d, 1H) 7.37 (dd, 1H) 7.28 (d, 1H) 7.19-7.25 (m, 4H) 6.91 (d, 1H) 3.96 (s, 3H) 3.95 (s, 3H) 2.42 (s, 3H) 1.62 (s, 6H).

LCMS (RT): 2.69 min (Method G); MS (ES+) gave m/z: 415.13 (MH+).

### Example 168

### N-[6-(Cyano-dimethyl-methyl)-4′-methoxy-biphenyl-3-yl]-3,4-dimethoxy-benzamide

### 168(A) 2-(5-Amino-4′-methoxy-biphenyl-2-yl)-2-methyl-propionitrile

Prepared according to Example 167(A), starting from 2-(4-amino-2-bromo-phenyl)-2-methyl-propionitrile (70.0 mg; 0.30 mol), prepared as in 111(C), and using 4-methoxyphenylboronic acid (47.0 mg; 0.35 mmol), 2 M K₂CO₃ (292 uL; 0.58 mmol) and and tetrakis(triphenylphospine)palladium(0) (17 mg; 0.02 mmol) in 1,2-dimethoxyethane (3 mL). The crude compound was purified by ion-exchange chromatography [SCX, DCM/MeOH (1/1) to MeOH/NH₄OH (9/1)] to give the title compound as a yellow oil (33 mg; 42% yield).

LCMS (RT): 1.14 min (Method D); MS (ES+) gave m/z: 267.1 (MH+).

### 168(B) N-[6-(Cyano-dimethyl-methyl)-4′-methoxy-biphenyl-3-yl]-3,4-dimethoxy-benzamide

Prepared according to Example 1(C) starting from 2-(5-amino-4′-methoxy-biphenyl-2-yl)-2-methyl-propionitrile (33.0 mg; 0.12 mmol), prepared as in 167(A), and using 3,4-dimethoxy-benzoyl chloride (30.0 mg; 0.14 mmol), and triethylamine (26 uL; 0.18 mmol) in DCM (3 mL) The crude was purified by preparative HPLC (Method Q) to give the title compound as a white powder (25 mg; 49% yield).

¹H NMR (300 MHz, CDCl3-d) δ(ppm):7.81 (dd, 1H) 7.75 (br. s., 1H) 7.62 (d, 1H) 7.50 (d, 1H) 7.38 (dd, 1H) 7.29-7.32 (m, 3H) 6.86-7.01 (m, 3H) 3.97 (s, 3H) 3.97 (s, 3H) 3.88 (s, 3H) 1.64 (s, 6H).

LCMS (RT): 2.52 min (Method G); MS (ES+) gave m/z: 431.16 (MH+).

### Example 169

N-[4′-Chloro-6-(cyano-dimethyl-methyl)-biphenyl-3-yl]-3,4-dimethoxy-benzamide

### 169(A) 2-(5-Amino-4′-chloro-biphenyl-2-yl)-2-methyl-propionitrile

Prepared according to Example 167(A), starting from 2-(4-amino-2-bromo-phenyl)-2-methyl-propionitrile (70.0 mg; 0.30 mol), prepared as in 111(C), and using 4-chlorophenylboronic acid (55.0 mg; 0.35 mmol), 2 M K₂CO₃ (292 uL; 0.58 mmol) and tetrakis(triphenylphospine)palladium(0) (17 mg; 0.02 mmol) in 1,2-dimethoxyethane (3 mL). The crude compound was purified by ion-exchange chromatography [SCX, DCM/MeOH (1/1) to MeOH/NH₄OH (9/1)] to give the title compound as a yellow oil (31 mg; 39% yield).

LCMS (RT): 1.32 min (Method D); MS (ES+) gave m/z: 271.1 (MH+).

### 169(B) N-[4′-Chloro-6-(cyano-dimethyl-methyl)-biphenyl-3-yl]-3,4-dimethoxy-benzamide

Prepared according to Example 1(C) starting from 2-(5-amino-4′-chloro-biphenyl-2-yl)-2-methyl-propionitrile (31.0 mg; 0.11 mmol), prepared as in 167(A), and using 3,4-dimethoxy-benzoyl chloride (28.0 mg; 0.13 mmol), and triethylamine (24 uL; 0.17 mmol) in DCM (3 mL). The crude was purifed by preparative HPLC (Method Q) to give the title compound as a white powder (24 mg; 48% yield).

¹H NMR (300 MHz, CDCl3-d) δ(ppm): 7.75-7.80 (m, 2H) 7.61 (d, 1H) 7.50 (d, 1H) 7.30-7.46 (m, 6H) 6.93 (d, 1H) 3.97 (s, 6H) 1.64 (s, 6H).

LCMS (RT): 2.71 min (Method G); MS (ES+) gave m/z: 435.06 (MH+).

### Example 170

### N-[4-(Cyano-dimethyl-methyl)-3-thiophen-3-yl-phenyl]-3,4-dimethoxy-benzamide

### 170(A) 2-(4-Amino-2-thiophen-3-yl-phenyl)-2-methyl-propionitrile

Prepared according to Example 167(A), starting from 2-(4-amino-2-bromo-phenyl)-2-methyl-propionitrile (70.0 mg; 0.30 mol), prepared as in 111(C), and using thiophene-3-boronic acid (45.0 mg; 0.35 mmol), 2 M K₂CO₃ (292 uL; 0.58 mmol) and tetrakis(triphenylphospine)palladium(0) (17 mg; 0.02 mmol) in 1,2-dimethoxyethane (3 mL). The crude compound was purified by ion-exchange chromatography [SCX, DCM/MeOH (1/1) to MeOH/NH₄OH (9/1)] to give the title compound as a yellow oil (29 mg; 41% yield).

LCMS (RT): 1.11 min (Method D); MS (ES+) gave m/z: 243.1 (MH+).

### 170(B) N-[4-(Cyano-dimethyl-methyl)-3-thiophen-3-yl-phenyl]-3,4-dimethoxy-benzamide

Prepared according to Example 1(C) starting from 2-(4-amino-2-thiophen-3-yl-phenyl)-2-methyl-propionitrile (29.0 mg; 0.12 mmol), prepared as in 167(A), and using 3,4-dimethoxy-benzoyl chloride (28.0 mg; 0.13 mmol), and triethylamine (24 uL; 0.17 mmol) in DCM (3 mL) The crude was purified by preparative HPLC (Method Q) to give the title compound as a pale yellow powder (19 mg; 39% yield).

¹H NMR (300 MHz, CDCl3-d) δ(ppm): 7.79 (dd, 2H) 7.76 (br. s., 1H) 7.59 (d, 1H) 7.50 (d, 1H) 7.35-7.41 (m, 3H) 7.31 (dd, 1H) 7.15 (dd, 1H) 6.92 (d, 1H) 3.96 (s, 3H) 3.96 (s, 3H) 1.66 (s, 6H)

LCMS (RT): 2.50 min (Method G); MS (ES+) gave m/z: 407.10 (MH+).

### Example 173

### N-(4-{2-[2-(2-Methanesulfonylamino-phenyl)-acetylamino]-1,1-dimethyl-ethyl}-phenyl)-3,4-dimethoxy-benzamide

### 173(A) N-(4-{1,1-Dimethyl-2-[2-(2-nitro-phenyl)-acetylamino]-ethyl}-phenyl)-3,4-dimethoxy-benzamide

Prepared according to Example 50, starting from N-[4-(2-amino-1,1-dimethyl-ethyl)-phenyl]-3,4-dimethoxy-benzamide (50.0 mg; 0.15 mmol), prepared as described in 26(A), and using (2-nitro-phenyl)-acetic acid (29.0 mg; 0.16 mmol), HOBt (24.3 mg; 0.18 mmol), EDC (44.1 mg; 0.23 mmol), TEA (32 uL; 0.23 mmol) in DCM (5 mL) After the work up, the title compound was collected as a white solid (70 mg; 95% yield), which was used in the next step without any further purification.

LCMS (RT): 1.42 min (Method D); MS (ES+) gave in/z: 492.1 (MH+).

### 173(B) N-(4-{2-[2-(2-Amino-phenyl)-acetylamino]-1,1-dimethyl-ethyl}-phenyl)-3,4-dimethoxy-benzamide

10% Pd/C (7 mg) was added to a solution of N-(4-{1,1-dimethyl-2-[2-(2-nitro-phenyl)-acetylamino]-ethyl}-phenyl)-3,4-dimethoxy-benzamide (70.0 mg; 0.14 mmol), prepared as in 173(A), in MeOH (20 mL). The mixture was hydrogenated at 1 bar at room temperature for 2 hours, the catalyst was filtered off and the filtrate was concentrated under reduced pressure to give the title compound as a pale yellow solid (66 mg; quantitative yield).

LCMS (RT): 1.17 min (Method D); MS (ES+) gave m/z: 462.1 (MH+).

### 173(C) N-(4-{2-[2-(2-Methanesulfonylamino-phenyl)-acetylamino]-1,1-dimethyl-ethyl}-phenyl)-3,4-dimethoxy-benzamide

To a solution of N-(4-{2-[2-(2-amino-phenyl)-acetylamino]-1,1-dimethyl-ethyl}-phenyl)-3,4-dimethoxy-benzamide (30.0 mg; 0.07 mmol), prepared as in 173(B), in DCM (3 mL), was added methanesulfonyl chloride (8.6 uL; 0.11 mmol) and then TEA (14.8 uL; 0.11 mmol). The reaction was stirred at room temperature for 4 days and during this time two new portions of methanesulfonyl chloride (8.6 uL; 0.11 mmol) and TEA (14.8 uL; 0.11 mmol) were added. After this the reaction was heated at 40° C. for 6 hours and then diluted with DCM and washed with 0.5 M NaHCO₃ and 1N HCl. The organic layer was dried over Na₂SO₄, filtered and concentrated under vacuum. The crude compound was purified by chromatography [SiO₂, Petroleum ether/EtOAc (8/2 to 1/1)] to provide the title compound as a pale yellow amorphous solid (20.0 mg; 53% yield).

¹H NMR (300 MHz, DMSO-d6+TFA) δ(ppm): 9.98 (s, 1H), 9.75 (s, 1H), 8.03 (t, 1H), 7.67 (m, 2H), 7.62 (dd, 1H), 7.53 (d, 1H), 7.34-7.43 (m, 1H), 7.02-7.34 (m, 6H), 3.85 (s, 3H), 3.84 (s, 3H), 3.59 (s, 2H), 3.28 (d, 2H), 3.00 (s, 3H), 1.21 (s, 6H).

LCMS (RT): 3.38 min (Method G); MS (ES+) gave m/z: 540.17 (MH+).

### Example 174

### N-[4-(Cyano-dimethyl-methyl)-3-(6-methoxy-pyridin-3-yl)-phenyl]-3,4-dimethoxy-benzamide

Prepared according to Example 152, starting from N-[3-bromo-4-(cyano-dimethyl-methyl)-phenyl]-3,4-dimethoxy-benzamide (70.0 mg; 0.17 mmol), prepared as in 111(D), and using 2-methoxypyridine-5-boronic acid (24.5 mg; 0.16 mmol), KF (19.8 mg; 0.34 mmol), palladium (II) acetate (catalytic amount) in methanol (5 mL). The crude compound was purified by chromatography [SiO₂, Petroleum ether/EtOAc (85/15 to 7/3)] to afford the title compound as a white amorphous solid (23.0 mg; 44% yield).

¹H NMR (300 MHz, CDCl3) δ(ppm): 7.81 (dd, 1H) 7.75 (br. s., 1H) 7.62 (d, 1H) 7.50 (d, 1H) 7.38 (dd, 1H) 7.29-7.32 (m, 3H) 6.86-7.01 (m, 3H) 3.97 (s, 3H) 3.97 (s, 3H) 3.88 (s, 3H) 1.64 (s, 6H).

LCMS (RT): 2.52 min (Method G); MS (ES+) gave m/z: 431.16 (MH+).

### Example 177

### N-[4-(Cyano-dimethyl-methyl)-phenyl]-2-methanesulfonylamino-4,5-dimethoxy-benzamide

### 177(A) N-[4-(Cyano-dimethyl-methyl)-phenyl]-4,5-dimethoxy-2-nitro-benzamide

A mixture of 4,5-dimethoxy-2-nitro-benzoic acid (255 mg, 1.12 mmol), HOBt (182 mg; 1.35 mmol), EDC (322 mg; 1.68 mmol) in DCM (10 mL) was stirred at room temperature for 1 hour. Then, 2-(4-amino-phenyl)-2-methyl-propionitrile (180 mg; 1.12 mmol), prepared as in 1(B), and TEA (114 uL; 1.12 mmol) were added and the resulting reaction was stirred at room temperature overnight. The reaction was diluted with DCM and washed sequentially with 0.5N NaHCO₃ and 1N HCl. The organic layer was dried over Na₂SO₄, filtered and evaporated to dryness. The crude product was purified by chromatography [SiO₂, Petroleum ether/EtOAc (8/2 to 1/1)], followed by crystallization from EtOAc/iPr₂O (1/1) to give the title compound as a white amorphous solid (15.0 mg; 138% yield).

LCMS (RT): 2.20 min (Method G); MS (ES+) gave m/z: 370.23 (MH+).

### 177(B) 2-Amino-N-[4-(cyano-dimethyl-methyl)-phenyl]-4,5-dimethoxy-benzamide

10% Pd/C (13 mg) was added to a solution of N-[4-(cyano-dimethyl-methyl)-phenyl]-4,5-dimethoxy-2-nitro-benzamide (130 mg; 0.14 mmol), prepared as in 177(A), in MeOH (20 mL). The mixture was hydrogenated at 1 bar at room temperature for 2 hours, the catalyst was filtered off and the filtrate was concentrated under reduced pressure. The crude was purified by crystallization from EtOH/iPr₂O (1/1) to give the title compound as a grey amorphous solid (66 mg; quantitative yield).

LCMS (RT): 1.78 min (Method G); MS (ES+) gave m/z: 340.15 (MH+).

### 177(C) N-[4-(Cyano-dimethyl-methyl)-phenyl]-2-methanesulfonylamino-4,5-dimethoxy-benzamide

To a solution of 2-amino-N-[4-(cyano-dimethyl-methyl)-phenyl]-4,5-dimethoxy-benzamide (34.5 mg; 0.10 mmol), prepared as in 177(B), in DCM (4 mL), was added methanesulfonyl chloride (17.5 mg; 0.15 mmol) and then TEA (15.2 mg; 0.15 mmol). The reaction was stirred at room temperature for 4 days and during this time three new portions of methanesulfonyl chloride (17.5 mg; 0.15 mmol) and TEA (15.2 mg; 0.15 mmol) were added. The reaction was diluted with DCM and washed with 0.5 M NaHCO₃ and 1N HCl. The organic layer was dried over Na₂SO₄, filtered and concentrated under vacuum. The crude was dissolved in MeOH (7.5 mL) and K₂CO₃ (28.0 mg; 0.20 mmol) was added. The resulting solution was heated to reflux for 15 min, then solvent was removed under vacuum. The residue was portioned between 2N HCl and DCM, the organic phase was collected, dried (Na₂SO₄), filtered and evaporated to dryness. The crude compound was triturated with EtOH/Et₂O (1/1) to afford the title compound as a white amorphous solid (14 mg; 34% yield).

¹H NMR (300 MHz, DMSO-d6) δ(ppm): 10.35 (br. s., 2H), 7.72 (m, 2H), 7.52 (m, 2H), 7.46 (s, 1H), 7.12 (s, 1H), 3.86 (s, 3H), 3.84 (s, 3H), 3.06 (s, 3H), 1.69 (s, 6H).

LCMS (RT): 3.27 min (Method I); MS (ES+) gave m/z: 418.14 (MH+).

### Example 183

### N-[4-(Cyano-dimethyl-methyl)-3-pyridin-2-yl-phenyl]-3,4-dimethoxy-benzamide

Prepared according to Example 158(B), starting from 2 N-[4-(cyano-dimethyl-methyl)-3-(4,4,5,5-tetramethyl-[1,3,2]dioxaborolan-2-yl)-phenyl]-3,4-dimethoxy-benzamide (60.0 mg; 0.13 mmol), prepared as in 158(A), and using 2-bromo-pyridine (12.0 ul; 0.13 mmol), 2M K₂CO₃ (160 uL; 0.32 mmol) and tetrakis(triphenylphospine)palladium(0) (7 mg; cat. amount) in 1,2-dimethoxyethane (4 mL). The crude product was purified by chromatography [SiO₂, Petroleum ether/EtOAc (1/1)], followed by crystallization from EtOH to give the title compound as a white solid (10.0 mg; 19% yield).

¹H NMR (300 MHz, CDCl3) δ(ppm): 8.67 (ddd, 1H), 7.77-7.85 (m, 3H), 7.58 (d, 1H), 7.56 (d, 1H), 7.48-7.53 (m, 2H), 7.38 (dd, 1H), 7.34 (ddd, 1H), 6.92 (d, 1H), 3.96 (s, 3H), 3.96 (s, 3H), 1.80 (s, 6H).

LCMS (RT): 2.20 min (Method M); MS (ES+) gave m/z: 402.20 (MH+).

### Example 184

### N-[4-(Cyano-dimethyl-methyl)-3-pyrimidin-5-yl-phenyl]-3,4-dimethoxy-benzamide

Prepared according to Example 156, staring from N-[3-bromo-4-(cyano-dimethyl-methyl)-phenyl]-3,4-dimethoxy-benzamide (80.0 mg; 0.20 mmol), prepared as in 111(D), and using pyrimidine-5-boronic acid (30.0 mg; 0.24 mmol), 2M K₂CO₃ (200 ul; 0.40 mmol) and tetrakis(triphenylphospine) palladium(0) (11 mg; 0.01 mmol) in 1,2-dimethoxyethane (4 mL). Purification by chromatography [SiO₂, Petroleum ether/EtOAc (1/4)] afforded the title compound as a white solid (36.0 mg; 45% yield).

¹H NMR (300 MHz, DMSO-d6) δ(ppm): 9.19 (d, 1H), 8.74 (d, 1H), 8.73 (d, 1H), 8.61 (d, 1H), 8.45 (d, 1H), 8.39 (dd, 1H), 7.93 (dd, 1H), 3.69 (dd, 1H), 3.27 (s, 3H), 1.81-2.06 (m, 1H), 0.82 (d, 3H), 0.80 (d, 3H).

LCMS (RT): 1.80 min (Method M); MS (ES+) gave m/z: 403.21 (MH+).

MP: 198-200° C.

### Example 191

### 5-Fluoro-1-(2-methoxy-ethyl)-1H-indole-3-carboxylic acid {2-[4-(3,4-dimethoxy-benzoylamino)-phenyl]-2-methyl-propyl}-amide

### 191(A) 5-Fluoro-1-(2-methoxy-ethyl)-1H-indole-3-carbaldehyde

To a suspension of NaH (60% dispersion in mineral oil; 60.0 mg; 1.50 mmol) in dry DMF (1.75 mL) cooled at 0° C., was added a solution of 5-fluoro-1H-indole-3-carbaldehyde (163 mg; 1.00 mmol) in dry DMF (1.75 mL). The reaction was stirred at 0° C. for 30 min and then 1-bromo-2-methoxy-ethane (122 uL; 1.30 mmol) was added. The reaction was allowed to warm to room temperature and the stirring was maintained for 16 hours. After this period, the reaction was portioned between water and EtOAc. The organic phase was washed several times with brine, dried over Na₂SO₄, filtered and evaporated under vacuum to give a yellow oil which crystallized on standing into a white solid (175 mg; 79% yield).

LCMS (RT): 1.22 min (Method D); MS (ES+) gave m/z: 222.0 (MH+).

### 191(B) 5-Fluoro-1-(2-methoxy-ethyl)-1H-indole-3-carboxylic acid

Sulfamic acid (383 mg; 3.95 mmol) was added to a solution of 5-fluoro-1-(2-methoxy-ethyl)-1H-indole-3-carbaldehyde, prepared as in 191(A), and sodium chlorite (92.0 mg; 1.03 mmol) in dioxane (9 mL) and water (3 mL). The solution was stirred at room temperature for 16 hours, and then the solvent was evaporated under vacuum. The residue was taken up with water and treated with sodium metabisulfite (180 mg; 0.95 mmol) and sodium bicarbonate (till basic solution). The aqueous phase was washed with DCM, acidified with 2N HCl (till acidic solution) and extracted with DCM. The organic layer was collected, dried over Na₂SO₄, filtered and evaporated under vacuum to give a brown solid, which was used in the next step without any further purification.

LCMS (RT): 3.0 min (Method E); MS (ES+) gave m/z: 238.1 (MH+).

### 191(C) 5-Fluoro-1-(2-methoxy-ethyl)-1H-indole-3-carboxylic acid {2-[4-(3,4-dimethoxy-benzoylamino)-phenyl]-2-methyl-propyl}-amide

A mixture of 5-fluoro-1-(2-methoxy-ethyl)-1H-indole-3-carboxylic acid (47.0 mg; 0.20 mmol), prepared as in 191(B), N-[4-(2-amino-1,1-dimethyl-ethyl)-phenyl]-3,4-dimethoxy-benzamide (65.0 mg; 0.20 mmol), prepared as in 26(A), HOBt (31.0 mg; 0.20 mmol), EDC (60.0 mg; 0.30 mmol) and TEA (83 uL; 0.6 mmol) in dioxane (8 mL) was stirred at room temperature overnight. The solvent was removed in vacuo, the residue taken up with DCM and washed with 1N NaHCO₃, 0.5N HCl and finally brine. The organic phase was dried (Na₂SO₄), filtered and concentrated under reduced pressure. The crude product was purified by chromatography [SiO₂, EtOAc], followed by trituration with MeOH to afford the title compound as a white amorphous solid (40.0 mg; 36% yield).

¹H NMR (300 MHz, DMSO-d6) δ(ppm): 9.99 (s, 1H), 8.12 (s, 1H), 7.72-7.78 (m, 1H), 7.71 (m, 2H), 7.50-7.67 (m, 4H), 7.41 (m, 2H), 6.95-7.10 (m, 2H), 4.36 (dd, 2H), 3.84 (s, 3H), 3.84 (s, 3H), 3.67 (dd, 2H), 3.47 (d, 2H), 3.22 (s, 3H), 1.32 (s, 6H).

LCMS (RT): 2.30 min (Method M); MS (ES+) gave m/z: 548.28 (MH+).

### Example 192

### 1-(3-Dimethylamino-propyl)-5-fluoro-1H-indole-3-carboxylic acid {2-[4-(3,4-dimethoxy-benzoylamino)-phenyl]-2-methyl-propyl}-amide

### 192(A) 1-[3-(tert-Butyl-dimethyl-silanyloxy)-propyl]-5-fluoro-1H-indole-3-carbaldehyde

Prepared according to Example 191(A), starting from 5-fluoro-1H-indole-3-carbaldehyde (175 mg; 1.07 mmol), and using (3-bromo-propoxy)-tert-butyl-dimethyl-silane (352 mg; 1.60 mmol), NaH (60% dispersion in mineral oil; 38.5 mg; 1.00 mmol) in dry DMF (3.30 mL). The crude product was purified by chromatography [SiO₂, Petroleum ether/EtOAc (9/1)] to give the title compound as a yellow oil (208 mg; 58% yield).

LCMS (RT): 1.83 min (Method D); MS (ES+) gave m/z: 336.1 (MH+).

### 192(B) 5-Fluoro-1-(3-hydroxy-propyl)-1H-indole-3-carboxylic acid

Prepared according to Example 191(B), starting from 1-[3-(tert-butyl-dimethyl-silanyloxy)-propyl]-5-fluoro-1H-indole-3-carbaldehyde (208 mg; 0.62 mmol), prepared as in 192(A), and using sulfamic acid (342 mg; 3.53 mmol) and sodium chlorite (72.9 mg; 0.81 mmol) in dioxane (6.76 mL) and water (2.25 mL). The title compound was collected as a light brown solid (35.0 mg; 24% yield).

LCMS (RT): 1.00 min (Method D); MS (ES+) gave m/z: 238.0 (MH+).

### 192(C) 5-Fluoro-1-(3-hydroxy-propyl)-1H-indole-3-carboxylic acid {2-[4-(3,4-dimethoxy-benzoylamino)-phenyl]-2-methyl-propyl}-amide

Prepared according to Example 191(C), starting from 5-fluoro-1-(3-hydroxy-propyl)-1H-indole-3-carboxylic acid (30.0 mg; 0.12 mmol), prepared as in 192(B), and using N-[4-(2-amino-1,1-dimethyl-ethyl)-phenyl]-3,4-dimethoxy-benzamide (41.0 mg; 0.12 mmol), prepared as in 26(A), HOBt (23.0 mg; 0.15 mmol), EDC (36.0 mg; 0.19 mmol) and TEA (53 uL; 0.4 mmol) in dioxane (7 mL). The title compound was collected as a brown solid (63.0 mg; 92% yield).

LCMS (RT): 1.36 min (Method D); MS (ES+) gave m/z: 548.1 (MH+).

### 192(D) 1-(3-Dimethylamino-propyl)-5-fluoro-1H-indole-3-carboxylic acid {2-[4-(3,4-dimethoxy-benzoylamino)-phenyl]-2-methyl-propyl}-amide

TEA (30 uL; 0.22 mmol) was added under nitrogen atmosphere to a solution of 5-fluoro-1-(3-hydroxy-propyl)-1H-indole-3-carboxylic acid {2-[4-(3,4-dimethoxy-benzoylamino)-phenyl]-2-methyl-propyl}-amide (63.0 mg; 0.11 mmol), prepared as in 192(C), in dry DCM (5 mL). The resulting solution was cooled down at 0° C. and methanesulphonyl chloride was added. After being stirred at room temperature for 16 hours, the reaction was diluted with DCM and washed with NaHCO₃ and then with brine. The organic phase was dried (Na₂SO₄), filtered and concentrated in reduced pressure. The brown oil was dissolved in dry THF (4 mL) and treated with dimethyl amine (2M solution in THF). The reaction was stirred for one day and then the volatiles were removed under vacuum. The residue was dissolved in DCM and washed with water. The organic phase was dried (Na₂SO₄), filtered and concentrated in reduced pressure. The crude compound was partially purified by ion-exchange chromatography [SCX, DCM/MeOH (1/1) to MeOH/NH₄OH (9/1)] and then by chromatography [SiO₂, DCM/MeOH+0.5% NH₄OH (99.5/0.5 to 98/2)], to give the title compound as a pale yellow amorphous solid (17.0 mg; 27% yield).

¹H NMR (300 MHz, DMSO-d6) δ(ppm): 9.98 (s, 1H), 8.14 (s, 1H), 7.66-7.80 (m, 3H), 7.49-7.65 (m, 4H), 7.41 (m, 2H), 7.07 (d, 1H), 6.98-7.07 (m, 1H), 4.21 (dd, 2H), 3.84 (s, 3H), 3.84 (s, 3H), 3.47 (d, 2H), 2.12 (s, 6H), 2.07-2.19 (m, 2H), 1.89 (quin, 2H), 1.31 (s, 6H).

LCMS (RT): 1.88 min (Method M); MS (ES+) gave m/z: 575.36 (MH+).

### Example 193

### 6-Fluoro-1H-benzoimidazole-2-carboxylic acid {2-[4-(3,4-dimethoxy-benzoylamino)-phenyl]-2-methyl-propyl}-amide

Prepared according to Example 162, starting from 5-fluoro-1H-benzoimidazole-2-carboxylic acid (46.0 mg; 0.25 mmol), and using oxalyl chloride (87.0 uL; 1.02 mmol) in DCM (2.5 mL) and then N-[4-(2-amino-1,1-dimethyl-ethyl)-phenyl]-3,4-dimethoxy-benzamide (70.0 mg; 0.21 mmol), prepared as described in 26(A), and TEA (71 uL; 0.51 mmol) in DCM (5 mL). Trituration of the crude product with EtOAc/MeOH (1/1) provided the title compound as a white solid (36.0 mg; 34% yield).

¹H NMR (300 MHz, DMSO-d6) δ(ppm): 13.19 (br. s., 1H), 10.01 (s, 1H), 8.20 (t, 1H), 7.72 (m, 2H), 7.62 (dd, 1H), 7.53 (d, 1H), 7.42 (m, 2H), 7.11-7.36 (m, 3H), 7.08 (d, 1H), 3.85 (s, 3H), 3.84 (s, 3H), 3.56 (d, 2H), 1.33 (s, 6H).

LCMS (RT): 2.18 min (Method M); MS (ES+) gave m/z: 491.20 (MH+).

MP: 222-224° C.

### Example 194

### Imidazo[1,2-a]pyridine-3-carboxylic acid {3-[4-(3,4-dimethoxy-benzoylamino)-phenyl]-3-methyl-butyl}-amide

### 194(A) {3-[4-(3,4-Dimethoxy-benzoylamino)-phenyl]-3-methyl-butyl}-carbamic acid tert-butyl ester

To a solution of 3-methyl-3-(4-nitro-phenyl)-butylamine (140 mg; 0.67 mmol), prepared as in 159(A), in dioxane (5 mL) and water (2 mL), was added NaOH (32.0 mg; 0.81 mmol), followed by di-tert-buthyldicarbonate (176 mg; 0.81 mmol). The reaction mixture was stirred at room temperature for 64 hours, then dioxane was removed in vacuo and the residue was portioned between DCM and water. The organic phase was dried (Na₂SO₄), filtered and evaporated to dryness. The residue was dissolved in MeOH (20 mL) and hydrogenated at 1 bar in presence of 10% Pd/C (20 mg) for 1.5 hour. The catalyst was removed by filtration and the solution evaporated to dryness. The crude was dissolved in DCM (8 mL) and TEA (120 uL; 0.86 mmol) and treated with 3,4-dimethoxybenzoyl chloride (172 mg; 0.86 mmol). After being stirred at RT for 16 hours, the mixture was washed with 5% NaHCO₃, dried over Na₂SO₄ and evaporated to dryness. The crude product was eventually purified by chromatography [SiO₂, DCM to DCM/MeOH (98/2)] to give the title compound as a white amorphous solid (255 mg; 86% yield).

LCMS (RT): 1.60 min (Method D); MS (ES+) gave m/z: 443.1 (MH+).

### 194(B) N-[4-(3-Amino-1,1-dimethyl-propyl)-phenyl]-3,4-dimethoxy-benzamide

{3-[4-(3,4-dimethoxy-benzoylamino)-phenyl]-3-methyl-butyl}-carbamic acid tert-butyl ester (65 mg; 0.15 mmol), prepared as in 194(A), was dissolved in DCM (2 mL) and TFA (130 uL) and the resulting solution was stirred at room temperature for 16 hours. The reaction was then quenched with 5% NaHCO₃, the layers were separated and the organic one was dried (Na₂SO₄) and evaporated to give the title compound as a yellow oil (43.0 mg; 86% yield).

LCMS (RT): 1.01 min (Method D); MS (ES+) gave m/z: 343.1 (MH+).

### 194(C) Imidazo[1,2-a]pyridine-3-carboxylic acid {3-[4-(3,4-dimethoxy-benzoylamino)-phenyl]-3-methyl-butyl}-amide

Prepared according to Example 50, starting from N-[4-(3-amino-1,1-dimethyl-propyl)-phenyl]-3,4-dimethoxy-benzamide (31 mg; 0.09 mmol), prepared as in 194(B), and using imidazo[1,2-a]pyridine-3-carboxylic acid (15.0 mg; 0.09 mmol), HOBt (16.0 mg; 0.12 mmol), EDC (23.0 mg; 0.12 mmol), TEA (28 uL; 0.2 mmol) in DCM (3 mL). The crude product was purified by chromatography [SiO₂, DCM/TEA (99.5/0.5) to DCM/MeOH/TEA (99/0.5/0.5] to afford the title compound as a white amorphous solid (36 mg; 81% yield).

¹H NMR (300 MHz, DMSO-d6) δ(ppm): 10.00 (br. s., 1H), 9.44 (ddd, 1H), 8.32 (t, 1H), 8.24 (s, 1H), 7.65-7.74 (m, 3H), 7.62 (dd, 1H), 7.53 (d, 1H), 7.31-7.47 (m, 3H), 7.01-7.14 (m, 2H), 3.85 (s, 3H), 3.84 (s, 3H), 2.99-3.17 (m, 2H), 1.79-2.01 (m, 2H), 1.34 (s, 6H).

LCMS (RT): 1.76 min (Method M); MS (ES+) gave m/z: 487.26 (MH+).

### Example 195

### N-[4-(2-Acetylamino-1,1-dimethyl-ethyl)-3-pyridin-3-yl-phenyl]-3,4-dimethoxy-benzamide

### 195(A) 2-Methyl-2-(4-nitro-2-pyridin-3-yl-phenyl)-propionitrile

A mixture of 2-(2-bromo-4-nitro-phenyl)-2-methyl-propionitrile (400 mg; 1.49 mmol), prepared as described in 111(B), diethyl-(3-pyridyl)-borane (328 mg; 2.24 mmol), 2M K₂CO₃ (1.49 mL; 2.98 mmol) and tetrakis(triphenylphospine)palladium(0) (34 mg; 0.03 mmol) in dioxane (10 mL) was heated at 100° C. for 16 hours. The solvent was removed under vacuum and the residue taken up with DCM and washed with water. The organic phase was dried over Na₂SO₄, filtered and evaporated to dryness. The crude compound was purified by ion-exchange chromatography [SCX, DCM/MeOH (1/1) to MeOH/NH₄OH (9/1)], to give the title compound as a yellow solid (234 mg; 58% yield).

LCMS (RT): 0.96 min (Method D); MS (ES+) gave m/z: 268.0 (MH+).

### 195(B) 2-Methyl-2-(4-nitro-2-pyridin-3-yl-phenyl)-propylamine

Prepared according to Example 54(A), starting from 2-methyl-2-(4-nitro-2-pyridin-3-yl-phenyl)-propionitrile (232 mg; 0.87 mmol), prepared as in 195(A), and using borane-THF complex (1M solution in THF; 3.48 mL) in dry THF (3 mL). The title compound was collected as a yellow oil (235 mg, quantitative yield).

LCMS (RT): 0.74 min (Method D); MS (ES+) gave m/z: 272.0 (MH+).

### 195(C) N-[2-Methyl-2-(4-nitro-2-pyridin-3-yl-phenyl)-propyl]-acetamide

Prepared according to Example 54(B), starting from 2-methyl-2-(4-nitro-2-pyridin-3-yl-phenyl)-propylamine (235 mg; 0.29 mmol), prepared as in 195(B), and using acetyl chloride (31.0 uL; 0.43 mmol) and triethylamine (61.0 uL; 0.43 mmol) in DCM (2 mL). The title compound (75 mg; 79% yield) was used in the next step without any purification.

LCMS (RT): 2.89 min (Method A); MS (ES+) gave m/z: 314.1 (MH+).

### 195(D) N-[2-(4-Amino-2-pyridin-3-yl-phenyl)-2-methyl-propyl]-acetamide

Prepared according to Example 1(B) starting from N-[2-Methyl-2-(4-nitro-2-pyridin-3-yl-phenyl)-propyl]-acetamide (75 mg; 0.23 mmol), prepared as in 195(C), and using 10% Pd/C (10 mg) in MeOH (30 mL). The catalyst was filtered off and the filtrate was concentrated under reduced pressure to give the title compound as a yellow oil (67 mg; quantitative yield).

LCMS (RT): 0.72 min (Method A); MS (ES+) gave m/z: 284.15 (MH+).

### 195(E) N-[4-(2-Acetylamino-1,1-dimethyl-ethyl)-3-pyridin-3-yl-phenyl]-3,4-dimethoxy-benzamide

Prepared according to Example 1(C) starting from N-[2-(4-amino-2-pyridin-3-yl-phenyl)-2-methyl-propyl]-acetamide (67 mg; 0.24 mmol), prepared as in 195(D), and using 3,4-dimethoxy-benzoyl chloride (52 mg; 0.26 mmol) and triethylamine (49 uL; 0.35 mmol) in dry DCM (5 mL). The crude product was purified by chromatography [SiO₂, DCM to DCM/MeOH (97/3)], followed by trituration with DCM to afford the title compound as a white solid (11 mg; 10% yield).

¹H NMR (300 MHz, DMSO-d6) δ(ppm): 10.02 (s, 1H), 8.53-8.67 (m, 2H), 7.75-7.90 (m, 2H), 7.61 (dd, 1H), 7.40-7.57 (m, 4H), 7.38 (d, 1H), 7.07 (d, 1H), 3.83 (s, 3H), 3.83 (s, 3H), 3.15 (d, 2H), 1.76 (s, 3H), 1.04 (s, 6H).

LCMS (RT): 1.40 min (Method M); MS (ES+) gave m/z: 448.20 (MH+).

MP: 234-237° C.

### Example 196

### 3H-Imidazo[4,5-b]pyridine-2-carboxylic acid {3-[4-(3,4-dimethoxy-benzoylamino)-phenyl]-3-methyl-butyl}-amide

Prepared according to Example 162, starting from 3H-imidazo[4,5-b]pyridine-2-carboxylic acid hydrochloride (28.0 mg; 0.14 mmol), and using oxalyl chloride (47.0 uL; 0.56 mmol) in DCM (35 mL) and then N-[4-(3-amino-1,1-dimethyl-propyl)-phenyl]-3,4-dimethoxy-benzamide (43.0 mg; 0.12 mmol), prepared as described in 194(B), and TEA (38 uL; 0.28 mmol) in DCM (2 mL). The crude compound was purified by chromatography [SiO₂, Petroleum ether/EtOAc (1/1) to EtOAc] and then by ion-exchange chromatography [SCX, DCM/MeOH (1/1) to MeOH/NH₄OH (9/1)], to give the title compound as a white solid (16.0 mg; 18% yield).

¹H NMR (300 MHz, DMSO-d6 353K) δ(ppm): 13.05 (br. s., 1H), 9.72 (s, 1H), 8.45 (br. s., 2H), 7.98 (br. s., 1H), 7.69 (m, 2H), 7.61 (dd, 1H), 7.57 (d, 1H), 7.39 (m, 2H), 7.29 (dd, 1H), 7.07 (d, 1H), 3.87 (s, 3H), 3.87 (s, 3H), 3.16-3.28 (m, 2H), 1.93-2.06 (m, 2H), 1.37 (s, 6H).

LCMS (RT): 2.64 min (Method N); MS (ES+) gave m/z: 488.20 (MH+).

### Example 198

### 3H-Imidazo[4,5-b]pyridine-6-carboxylic acid {2-[4-(3,4-dimethoxy-benzoylamino)-2-ethyl-phenyl]-2-methyl-propyl}-amide

### 198(A) N-[4-(2-Amino-1,1-dimethyl-ethyl)-3-ethyl-phenyl]-3,4-dimethoxy-benzamide

Prepared according to Example 98(C), starting from N-[4-(cyano-dimethyl-methyl)-3-ethyl-phenyl]-3,4-dimethoxy-benzamide (40 mg; 0.11 mmol), prepared as described in 133(B), and using PtO₂ (10 mg) in MeOH (10 mL). The title compound was achieved as a pale yellow oil (20 mg; 49% yield).

LCMS (RT): 1.06 min (Method D); MS (ES+) gave m/z: 357.1 (MH+).

### 198(B) 3H-Imidazo[4,5-b]pyridine-6-carboxylic acid {2-[4-(3,4-dimethoxy-benzoylamino)-2-ethyl-phenyl]-2-methyl-propyl}-amide

Prepared according to Example 50, starting from N-[4-(2-amino-1,1-dimethyl-ethyl)-3-ethyl-phenyl]-3,4-dimethoxy-benzamide (20.0 mg; 0.06 mmol), prepared as in 198(A), and using 3H-imidazo[4,5-b]pyridine-6-carboxylic acid (9.0 mg; 0.06 mmol), HOBt (10.0 mg; 0.07 mmol), TEA (25 uL; 0.18 mmol) and EDC (14.0 mg; 0.07 mmol) in DCM (5 mL) The crude product was purified by chromatography [SiO₂, DCM/MeOH (98/2 to 95/5)] to give the title compound as a white amorphous solid (12 mg; 43% yield).

¹H NMR (300 MHz, DMSO-d6) δ(ppm): 13.10 (br. s., 1H), 9.94 (s, 1H), 8.82 (d, 1H), 8.54 (s, 1H), 8.49 (t, 1H), 8.42 (d, 1H), 7.50-7.67 (m, 4H), 7.31 (d, 1H), 7.08 (d, 1H), 3.85 (s, 3H), 3.84 (s, 3H), 3.61 (d, 2H), 3.01 (q, 2H), 1.40 (s, 6H), 1.28 (t, 3H).

LCMS (RT): 1.81 min (Method M); MS (ES+) gave m/z: 502.28 (MH+).

### Example 199

### 3H-Imidazo[4,5-b]pyridine-6-carboxylic acid {3-[4-(3,4-dimethoxy-benzoylamino)-phenyl]-3-methyl-butyl}-amide

Prepared according to Example 50, starting from N-[4-(3-amino-1,1-dimethyl-propyl)-phenyl]-3,4-dimethoxy-benzamide (49.0 mg; 0.14 mmol), prepared as in 194(B), and using 3H-imidazo[4,5-b]pyridine-6-carboxylic acid (23.0 mg; 0.14 mmol), HOBt (25.0 mg; 0.19 mmol), EDC (36.0 mg; 0.19 mmol), TEA (64 uL; 0.46 mmol) in DCM (7 mL). The crude product was purified by chromatography [SiO₂, DCM/MeOH (98/2 to 94/6)] to afford the title compound as a pale yellow amorphous solid (16 mg; 23% yield).

¹H NMR (300 MHz, DMSO-d6) δ(ppm): 13.04 (br. s., 1H), 9.99 (s, 1H), 8.80 (s, 1H), 8.28-8.61 (m, 3H), 7.71 (m, 2H), 7.62 (dd, 1H), 7.53 (d, 1H), 7.39 (m, 2H), 7.08 (d, 1H), 3.85 (s, 3H), 3.84 (s, 3H), 3.03-3.16 (m, 2H), 1.86-1.99 (m, 2H), 1.34 (s, 6H).

LCMS (RT): 1.69 min (Method M); MS (ES+) gave m/z: 488.34 (MH+).

### Example 200

### N-[4-(2-Acetylamino-1,1-dimethyl-ethyl)-3-ethyl-phenyl]-3,4-dimethoxy-benzamide

Prepared according to Example 54(B), starting from N-[4-(2-amino-1,1-dimethyl-ethyl)-3-ethyl-phenyl]-3,4-dimethoxy-benzamide (55.0 mg; 0.15 mmol), prepared as in 198(A), and using acetyl chloride (25 uL; 0.35 mmol) and triethylamine (47 uL; 0.33 mmol) in DCM (3 mL). The crude compound was purified by preparative HPLC (Method Q) to give the title compound as a yellow solid (36.0 mg; 60% yield).

¹H NMR (300 MHz, DMSO-d6) δ(ppm): 9.93 (s, 1H), 7.68 (t, 1H), 7.62 (dd, 1H), 7.58 (d, 1H), 7.51-7.57 (m, 2H), 7.23 (d, 1H), 7.08 (d, 1H), 3.85 (s, 3H), 3.84 (s, 3H), 3.35 (d, 2H), 2.89 (q, 2H), 1.82 (s, 3H), 1.30 (s, 6H), 1.23 (t, 3H).

LCMS (RT): 1.92 min (Method M); MS (ES+) gave m/z: 399.25 (MH+).

MP: 203-206° C.

### Example 201

### 1H-Pyrrolo[2,3-b]pyridine-5-carboxylic acid {2-[4-(3,4-dimethoxy-benzoylamino)-phenyl]-2-methyl-propyl}-amide

Prepared according to Example 162, starting from 6-fluoro-1H-pyrazolo[3,4-b]pyridine-3-carboxylic acid (50.0 mg; 0.28 mmol), and using oxalyl chloride (94.0 uL; 1.12 mmol) in DCM (5 mL) and then N-[4-(2-amino-1,1-dimethyl-ethyl)-phenyl]-3,4-dimethoxy-benzamide (82.0 mg; 0.25 mmol), prepared as described in 26(A), and TEA (85 uL; 0.61 mmol) in DCM (6 mL). The crude product was purified by chromatography [SiO₂, DCM/MeOH (99/1 to 98.5/1.5)], followed by trituration with DCM to afford the title compound as a white amorphous solid (33.0 mg; 24% yield).

¹H NMR (300 MHz, DMSO-d6) δ(ppm): 14.17 (br. s., 1H), 10.01 (s, 1H), 8.60 (dd, 1H), 7.81 (t, 1H), 7.72 (m, 2H), 7.62 (dd, 1H), 7.53 (d, 1H), 7.42 (m, 2H), 7.01-7.16 (m, 2H), 3.85 (s, 3H), 3.84 (s, 3H), 3.55 (d, 2H), 1.33 (s, 6H)

LCMS (RT): 2.29 min (Method O); MS (ES+) gave m/z: 492.51 (MH+).

MP: 256-258° C.

### Example 203

### Imidazo[1,2-a]pyridine-3-carboxylic acid {2-[3-(3,4-dimethoxy-benzoylamino)-phenyl]-2-methyl-propyl}-amide

Prepared according to Example 50, starting from N-[3-(2-amino-1,1-dimethyl-ethyl)-phenyl]-3,4-dimethoxy-benzamide (53.0 mg; 0.16 mmol), prepared as in 88(A), and using imidazo[1,2-a]pyridine-3-carboxylic acid (26.0 mg; 0.16 mmol), HOBt (28.0 mg; 0.21 mmol), EDC (40.0 mg; 0.21 mmol), TEA (49 uL; 0.35 mmol) in DCM (5 mL) The crude product was purified by chromatography [SiO₂, DCM to DCM/MeOH (98/2)] to afford the title compound as a white amorphous solid (57 mg; 74% yield).

¹H NMR (300 MHz, DMSO-d6) δ(ppm): 10.02 (s, 1H), 9.40 (dt, 1H), 8.36 (s, 1H), 8.30 (t, 1H), 7.84 (t, 1H), 7.66-7.74 (m, 2H), 7.63 (dd, 1H), 7.54 (d, 1H), 7.39-7.48 (m, 1H), 7.30 (dd, 1H), 7.14-7.22 (m, 1H), 7.03-7.13 (m, 2H), 3.85 (s, 3H), 3.84 (s, 3H), 3.50 (d, 2H), 1.34 (s, 6H).

LCMS (RT): 1.73 min (Method M); MS (ES+) gave m/z: 473.32 (MH+).

### Example 204

### N-[4-(Cyano-dimethyl-methyl)-3-morpholin-4-yl-phenyl]-3,4-dimethoxy-benzamide

A mixture of N-[3-bromo-4-(cyano-dimethyl-methyl)-phenyl]-3,4-dimethoxy-benzamide (100 mg; 0.25 mmol), prepared as in 111(D), morpholine (26 uL; 0.30 mmol), potassium tert-butoxide (12 mg; 0.37 mmol), (R)-(+)-2,2′-bis(diphenylphosphino)-1,1′-binaphtyl (BINAP; 31 mg; 0.05 mmol) and tris(dibenzylidenacetone) palladium (0) (23 mg; 0.02 mmol) in DMF (4 mL) was heated at 90° C. for 16 hours. The solvent was removed under vacuum and the residue was portioned between water and DCM. The organic phase was dried over Na₂SO₄, filtered and evaporated to dryness. The crude compound was purified by preparative HPLC (Method Q) to afford the title compound as a colourless amorphous solid (4.5 mg; 5% yield).

¹H NMR (300 MHz, CDCl3) δ(ppm): 7.68 (s, 1H), 7.52 (d, 1H), 7.44 (dd, 1H), 7.39 (dd, 1H), 7.23 (d, 1H), 7.07 (d, 1H), 6.94 (d, 1H), 3.98 (s, 3H), 3.97 (s, 3H), 3.81 (s, 8H), 1.54 (s, 6H).

LCMS (RT): 1.47 min (Method M); MS (ES+) gave m/z: 410.22 (MH+).

### Example 205

### N-[4-(Cyano-dimethyl-methyl)-3-(1-methyl-1H-pyrazol-4-yl)-phenyl]-3,4-dimethoxy-benzamide

### 205(A) 2-[4-Amino-2-(1-methyl-1H-pyrazol-4-yl)-phenyl]-2-methyl-propionitrile

A mixture of 2-(4-amino-2-bromo-phenyl)-2-methyl-propionitrile (67.5 mg; 0.28 mol), prepared as in 111(C), 1-methyl-4-(4,4,5,5-tetramethyl-1,3,2-dioxaborolan-2-yl)-1H-pyrazole (87.0 mg; 0.42 mmol), cesium carbonate (137 mg; 0.42 mmol) and 1,1′-bis(diphenylphosphino)ferrocenedichloropalladium (II) (11 mg; 0.01 mmol) in DMF (4 mL) was heated at 80° C. for 8 hours. The reaction was diluted with EtOAc and washed with water. The organic phase was dried over Na₂SO₄, filtered and evaporated to dryness. The crude compound was purified by trituration with to give the title compound as a yellow solid (43.0 mg; 64% yield).

LCMS (RT): 0.80 min (Method D); MS (ES+) gave m/z: 241.2 (MH+).

### 205(B) N-[4-(Cyano-dimethyl-methyl)-3-(1-methyl-1H-pyrazol-4-yl)-phenyl]-3,4-dimethoxy-benzamide

Prepared according to Example 1(C), starting from 2-[4-amino-2-(1-methyl-1H-pyrazol-4-yl)-phenyl]2-methyl-propionitrile (43.0 mg; 0.18 mmol), prepared as in 205(A), and using 3,4-dimethoxy-benzoyl chloride (43.0 mg; 0.22 mmol) and TEA (35 uL; 0.25 mmol) in DCM (30 mL). The crude compound was purified by preparative HPLC (Method Q) to give the title compound as an off-white solid (31.2 mg; 35% yield).

¹H NMR (300 MHz, DMSO-d6) δ(ppm): 10.09 (s, 1H), 7.84 (dd, 1H), 7.81 (s, 1H), 7.62 (dd, 1H), 7.59 (d, 1H), 7.49-7.55 (m, 2H), 7.49 (d, 1H), 7.08 (d, 1H), 3.91 (s, 3H), 3.84 (s, 3H), 3.84 (s, 3H), 1.65 (s, 6H).

LCMS (RT): 1.89 min (Method M); MS (ES+) gave m/z: 405.24 (MH+).

### Example 206

### N-[4-(Cyano-dimethyl-methyl)-3-thiophen-2-yl-phenyl]-3,4-dimethoxy-benzamide

### 206(A) 2-(4-Amino-2-thiophen-2-yl-phenyl)-2-methyl-propionitrile

A mixture of 2-(4-amino-2-bromo-phenyl)-2-methyl-propionitrile (90.0 mg; 0.37 mol), prepared as in 111(C), 2-thiopheneboronic acid (71.0 mg; 0.56 mmol), cesium carbonate (182 mg; 0.56 mmol) and 1,1′-bis(diphenylphosphino)ferrocenedichloropalladium (II) (15 mg; 0.02 mmol) in DMF (4 mL) was heated at 100° C. for 4 hours. The reaction was concentrated under vacuum and the residue portioned between water and DCM. The organic phase was dried over Na₂SO₄, filtered and evaporated to dryness. The resulting compound was used as such in the next step.

LCMS (RT): 3.4 min (Method A); MS (ES+) gave m/z: 243.11 (MH+).

### 206(B) N-[4-(Cyano-dimethyl-methyl)-3-thiophen-2-yl-phenyl]-3,4-dimethoxy-benzamide

Prepared according to Example 1(C), starting from 2-(4-amino-2-thiophen-2-yl-phenyl)-2-methyl-propionitrile (89.5 mg; 0.37 mmol), prepared as in 206(A), and using 3,4-dimethoxy-benzoyl chloride (81.0 mg; 0.41 mmol) and TEA (63 uL; 0.44 mmol) in DCM (5 mL). The crude compound was purified by preparative HPLC (Method Q) to give the title compound as a yellow solid (48.8 mg; 29% yield over two steps).

¹H NMR (300 MHz, CDCl3) δ(ppm): 7.87 (dd, 1H), 7.77 (s, 1H), 7.60 (d, 1H), 7.47-7.54 (m, 2H), 7.43 (dd, 1H), 7.39 (dd, 1H), 7.19 (dd, 1H), 7.10 (dd, 1H), 6.93 (d, 1H), 3.98 (s, 3H), 3.97 (s, 3H), 1.74 (s, 6H).

LCMS (RT): 2.34 min (Method M); MS (ES+) gave m/z: 407.19 (MH+).

### Example 212

### Imidazo[1,2-a]pyridine-3-carboxylic acid {2-[4-(3,4-dimethoxy-benzoylamino)-phenyl]-3-hydroxy-2-methyl-propyl}-amide

### 212(A) Cyano-(4-nitro-phenyl)-acetic acid ethyl ester

NaH (60% dispersion in mineral oil; 340 mg; 8.51 mmol) was added portionwise to a solution of ethyl-cyanoacetate (961 uL; 8.51 mmol) in dry DMF (10 mL), cooled at 0° C. and under inert atmosphere. After 1 hour, a solution of 1-fluoro-4-nitro-benzene (1.00 mL; 7.09 mmol) in dioxane (10 mL) was added and the reaction was allowed to warm to room temperature and stirred for 2 hours. After this period, the solvent was removed under vacuum, the residue was taken up with DCM and washed with 1N HCl and then water. The organic phase was dried (Na₂SO₄), filtered and concentrated by rotary evaporator. The crude product was purified by chromatography [SiO₂, Petroleum ether/EtOAc (8/2 to 9/1)] to afford the title compound as an orange solid (978 mg; 60% yield).

LCMS (RT): 3.90 min (Method A); MS (ES+) gave m/z: 235.08 (MH+).

### 212(B) Cyano-methyl-(4-nitro-phenyl)-acetic acid ethyl ester

To a solution of cyano-(4-nitro-phenyl)-acetic acid ethyl ester (500 mg; 2.13 mmol), prepared as in 212(A), in dry DMF (10 mL), was added portionwise NaH (60% dispersion in mineral oil; 127 mg; 3.19 mmol). The red solution was stirred at room temperature for 20 minutes and then treated with iodomethane (159 uL; 2.56 mmol). The reaction was stirred at room temperature for 64 hours and then the solvent was removed in vacuo. The residue was taken up with DCM, washed with water, dried over Na₂SO₄, filtered and evaporated to dryness. The crude was purified by chromatography [SiO₂, Petroleum ether/EtOAc (8/2 to 1/1)] to give the title compound as a red solid (381 mg; 72% yield).

LCMS (RT): 4.09 min (Method A); MS (ES+) gave m/z: 249.08 (MH+).

### 212(C) (4-Amino-phenyl)-cyano-methyl-acetic acid ethyl ester

Prepared according to Example 1(B) starting from cyano-methyl-(4-nitro-phenyl)-acetic acid ethyl ester (381 mg; 1.54 mmol), prepared as in 212(B), and using 10% Pd/C (10 mg) in MeOH (30 mL). The catalyst was filtered off and the filtrate was evaporated under vacuum to give the title compound as a yellow oil (229 mg; 82% yield).

LCMS (RT): 3.3 min (Method A); MS (ES+) gave m/z: 219.15 (MH+).

### 212(D) Cyano-[4-(3,4-dimethoxy-benzoylamino)-phenyl]-methyl-acetic acid ethyl ester

A mixture of 3,4-dimethoxy-benzoyl chloride (252 mg; 1.26 mmol), (4-amino-phenyl)-cyano-methyl-acetic acid ethyl ester (229 mg; 1.05 mmol), prepared as in 212(B), and triethylamine (294 uL; 2.10 mmol) in dry DCM (15 mL) was stirred at 80° C. for 24 hours. Then the reaction was diluted with DCM, washed sequentially with 1N NaHCO₃, 1N HCl and water. The organic layer was dried over sodium sulphate, filtered and evaporated under reduced pressure. The crude compound was purified by preparative HPLC (Method Q) to provide the title compound as an orange sticky oil (112 mg; 30% yield).

LCMS (RT): 4.0 min (Method A); MS (ES+) gave m/z: 383.08 (MH+).

### 212(E) 2-[4-(3,4-Dimethoxy-benzoylamino)-phenyl]-3-[(imidazo[1,2-a]pyridine-3-carbonyl)-amino]-2-methyl-propionic acid ethyl ester

To a solution of cyano-[4-(3,4-dimethoxy-benzoylamino)-phenyl]-methyl-acetic acid ethyl ester (90 mg; 0.23 mmol), prepared as in 212(D), in MeOH (50 mL) and in presence of few drops of 37% HCl, was added 10% Pd/C (10 mg) and the reaction was hydrogenated at 3.3 bar at room temperature for 16 hours. Then the catalyst was filtered off and the filtrate was concentrated under reduced pressure. The residue was dissolved in DCM (10 mL) and TEA (100 uL; 031 mmol), HOBt (38.0 mg; 0.28 mmol), EDC (68.0 mg; 0.35 mmol) and imidazo[1,2-a]pyridine-3-carboxylic acid (38.0 mg; 0.23 mmol) were added. The mixture was stirred at room temperature for 16 hours, diluted with DCM, washed with 2M K₂CO₃, dried over Na₂SO₄, filtered and evaporated to dryness. The crude was purified by chromatography [SiO₂, DCM to DCM/MeOH (9/1)] to give the title compound as an orange solid (51.0 mg; 41% yield).

LCMS (RT): 3.54 min (Method A); MS (ES+) gave m/z: 531.10 (MH+).

### 212(F) Imidazo[1,2-a]pyridine-3-carboxylic acid {2-[4-(3,4-dimethoxy-benzoylamino)-phenyl]-3-hydroxy-2-methyl-propyl}-amide

Lithium borohydride (10 mg; 0.19 mmol) was added to a solution of 2-[4-(3,4-dimethoxy-benzoylamino)-phenyl]-3-[(imidazo[1,2-a]pyridine-3-carbonyl)-amino]-2-methyl-propionic acid ethyl ester (51.0 mg; 0.09 mmol), prepared as in 212(E), in dry THF (5 mL). The reaction was heated at 50° C. for 4 hours, then cooled down to room temperature and quenched with water. The volatiles were removed under vacuum, the residue was taken up with THF (5 mL) and 1N HCl (5 mL) and boiled for 1 hour. The reaction was concentrated in vacuo, portioned between DCM and 2M K₂CO₃. The organic phase was dried over Na₂SO₄, filtered and evaporated to dryness. The crude compound was purified by preparative HPLC (Method Q) to provide the title compound as a white solid (9.0 mg; 19% yield).

¹H NMR (300 MHz, CDCl3) δ(ppm): 9.53 (d, 1H), 7.97-8.08 (m, 2H), 7.67-7.79 (m, 1H), 7.59 (m, 2H), 7.48-7.55 (m, 2H), 7.39 (m, 2H), 7.32-7.47 (m, 2H), 7.05 (dd, 1H), 6.86-7.00 (m, 2H), 3.95 (s, 6H), 3.79-3.92 (m, 2H), 3.53-3.75 (m, 2H), 1.37 (s, 3H).

LCMS (RT): 1.67 min (Method P); MS (ES+) gave m/z: 489.61 (MH+).

### Example 220

### N-[4-(Cyano-dimethyl-methyl)-3-vinyl-phenyl]-3,4-dimethoxy-benzamide

### 220(A) 2-(4-Amino-2-vinyl-phenyl)-2-methyl-propionitrile

Tetrakis(triphenylphospine)palladium(0) (9.2 mg; cat.amount) was added to a degassed mixture of 2-(4-amino-2-bromo-phenyl)-2-methyl-propionitrile (100 mg; 0.42 mol), prepared as in 111(C), 2,4,6-trivinylcyclotriboroxane pyridine complex (15 mg; 0.63 mmol) and K₂CO₃ (58.0 mg; 0.42 mmol) in 1,2-dimethoxyethane (18 mL) and water (450 uL). The reaction was heated under microwave irradiation at 130° C. for 1 hour. DCM and 5% NaHCO₃ were added, the phases were separated and the aqueous one was extracted again with DCM. The combined organic layers were dried over Na₂SO₄, filtered and evaporated. The crude was purified by ion-exchange chromatography [SCX, DCM/MeOH (1/1) to MeOH/NH₄OH (9/1)] to give the title compound as an orange oil (74.4 mg; 95% yield).

LCMS (RT): 0.94 min (Method D); MS (ES+) gave m/z: 187.1 (MH+).

### 220(B) N-[4-(Cyano-dimethyl-methyl)-3-vinyl-phenyl]-3,4-dimethoxy-benzamide

A mixture of 2-(4-amino-2-vinyl-phenyl)-2-methyl-propionitrile (74.0 mg; 0.40 mmol) and 3,4-dimethoxybenzoyl chloride (120 mg; 0.60 mmol) in pyridine (3 mL) was heated under microwave irradiation at 110° C. for 1 hour. 3,4-Dimethoxybenzoyl chloride (120 mg; 0.60 mmol) was added again and the reaction was heated as before (procedure repeated twice). The reaction was diluted with DCM and washed sequentially with 5% NaHCO₃, 2N HCl and water. The organic phase was dried over Na₂SO₄, filtered and evaporated to dryness. The crude was purified by chromatography [SiO₂, Petroleum ether/EtOAc (9/1 to 7/3)] to give the title compound as a light yellow amorphous solid (78.0 mg; 56% yield).

¹H NMR (300 MHz, CDCl₃) δ(ppm): 7.81 (s, 1H), 7.64-7.73 (m, 2H), 7.46-7.59 (m, 2H), 7.41 (dd, 1H), 7.35 (d, 1H), 6.93 (d, 1H), 5.69 (dd, 1H), 5.48 (dd, 1H), 3.97 (s, 3H), 3.96 (s, 3H), 1.81 (s, 6H).

LCMS (RT): 2.25 min (Method M); MS (ES+) gave m/z: 351.23 (MH+).

### Example 221

### N-(4-(4-acetamido-2-methylbutan-2-yl)-3-(pyridin-3-yl)-phenyl)-3,4-dimethoxybenzamide

### 221(A) 3-(2-bromo-4-nitrophenyl)-3-methylbutanamide

To a mixture of 3-methyl-3-(4-nitrophenyl)butanamide (683 mg; 3.08 mmol), prepared as in 159(C), and silver trifluoromethansulfonate (791 mg; 3.08 mmol) in conc. H₂SO₄ (6.55 mL) and H₂O (723 uL), was added bromine (388 uL) and the mixture was stirred at room temperature for 3 hours. Then aqueous sodium sulfite was added and the precipitate was filtered off, the clean solution was extracted twice with DCM and the combined organic layers were dried (Na₂SO₄), filtered and concentrated in vucuum. Purification by flash chromatography [SiO₂, Petroleum ether/EtOAc (1/1 to 4/6)] afforded the title compound as a white solid (335 mg; 36% yield).

¹H NMR (300 MHz, DMSO-d6) δ(ppm): 8.46 (d, 1H), 8.12 (dd, 1H), 7.68 (d, 1H), 4.98-5.29 (m, 2H), 3.06 (s, 2H), 1.68 (s, 6H).

LCMS (RT): 1.27 min (Method D); MS (ES+) gave m/z: 301.0; 303.0 (M; M+2).

### 221(B) 3-(2-Bromo-4-nitrophenyl)-3-methylbutan-1-amine

To a solution of 3-(2-bromo-4-nitrophenyl)-3-methylbutanamide (310 mg; 1.03 mmol), prepared as described in 221(A), in dry THF (5 mL), borane-THF complex (1M solution in THF; 4.12 mL) was added dropwise while stirring under nitrogen atmosphere. The reaction was refluxed for 6.5 hours, cooled at room temperature and quenched by adding methanol dropwise. Then, 37% HCl was added (1 mL) and the solution was heated at reflux for 2 hours. The solvent was removed under vacuum, the residue was portioned between EtOAc and 2M K₂CO₃. The organic phase was dried over Na₂SO₄, filtered and evaporated to dryness under vacuum. The crude compound was purified by ion-exchange chromatography [SCX, DCM/MeOH (1/1) to MeOH/NH₄OH (98/2)] to give the title compound as a pale yellow amorphous solid (240 mg; 81% yield).

LCMS (RT): 1.06 min (Method D); MS (ES+) gave m/z: 287.1; 289.1 (M; M+2).

### 221(C) N-(3-(2-bromo-4-nitrophenyl)-3-methylbutyl)-acetamide

A mixture of 3-(2-bromo-4-nitrophenyl)-3-methylbutan-1-amine (144 mg; 0.50 mmol), prepared as in 221(B), acetyl chloride (53 uL; 0.75 mmol) and triethylamine (104 uL; 0.75 mmol) in DCM (7 mL) was stirred at room temperature for 1 hour. The reaction was then diluted with DCM and washed with 5% NaHCO₃. The organic layer was separated, dried over Na₂SO₄, filtered and evaporated to dryness. The crude compound was purified by flash chromatography [SiO₂, Petroleum ether/EtOAc (7/3 to 3/7)] to afford the title compound as a pale yellow solid (116 mg; 71% yield).

LCMS (RT): 1.37 min (Method D); MS (ES+) gave m/z: 329.0; 331.0 (M; M+2).

### 221(D) N-(3-(4-amino-2-bromophenyl)-3-methylbutyl)acetamide

PtO₂ (10 mg) was added to a solution of N-(3-(2-bromo-4-nitrophenyl)-3-methylbutyl)-acetamide (116 mg; 0.35 mmol), prepared as in 221(C), in MeOH (30 mL). The mixture was hydrogenated at 1.6 bar at room temperature for 2 hours, then the catalyst was removed by filtration and the filtrate was concentrated under reduced pressure to give the title compound as a pale yellow amorphous solid (102 mg; 97% yield).

LCMS (RT): 0.92 min (Method D); MS (ES+) gave m/z: 299.2; 301.2 (M; M+2).

### 221(E) N-(4-(4-acetamido-2-methylbutan-2-yl)-3-(pyridin-3-yl)-phenyl)-3,4-dimethoxybenzamide

A mixture of N-(3-(4-amino-2-bromophenyl)-3-methylbutyl)-acetamide (102 mg; 0.34 mmol), prepared as described in 221(D), diethyl-(3-pyridyl)-borane (75.2 mg; 0.51 mmol), 2M K₂CO₃ (340 uL; 0.68 mmol) and tetrakis(triphenylphospine)palladium(0) (8.1 mg; 0.007 mmol) in previously degassed dioxane (10 mL) was heated at 100° C. for 16 hours. The solvent was removed under vacuum and the residue was taken up with DCM and washed with water. The organic phase was dried over Na₂SO₄, filtered and evaporated to dryness. The crude compound was partially purified by ion-exchange chromatography [SCX, DCM/MeOH (1/1) to MeOH/NH₄OH (98/2)]. The resulting compound was dissolved in pyridine (2 mL) and 3,4-dimethoxy-benzoyl chloride (74.8 mg; 0.37 mmol) was added. The reaction was heated under microwave irradiation at 100° C. for 1 hour, then a second portion of 3,4-dimethoxy-benzoyl chloride (74.8 mg; 0.37 mmol) was added and the reaction was heated again as described before. This procedure was repeated twice and then the pyridine was removed by rotary evaporator. The residue was taken up with DCM and washed with 2M K₂CO₃, The organic layer was dried over sodium sulphate, filtered and evaporated under reduced pressure. The crude compound was first purified by ion-exchange chromatography [SCX, DCM/MeOH (1/1) to MeOH/NH₄OH (98/2)] and then by flash chromatography [SiO₂, DCM to DCM/MeOH (97/3)] to afford the title compound as a pale yellow amorphous solid (24 mg; 15% yield).

¹H NMR (300 MHz, CDCl3) δ(ppm): 8.60 (dd, 1H), 8.49-8.58 (m, 1H), 7.90 (s, 1H), 7.64-7.73 (m, 2H) 7.45-7.54 (m, 2H), 7.41 (dd, 1H), 7.29-7.36 (m, 1H), 7.25-7.28 (m, 1H), 6.92 (d, 1H), 5.09-5.28 (m, 1H), 3.95 (s, 6H), 2.91-3.23 (m, 2H), 1.89 (s, 3H), 1.51-1.75 (m, 2H), 1.23 (s, 6H)

LCMS (RT): 1.47 min (Method P); MS (ES+) gave m/z: 462.19 (MH+).

The compounds reported in Table 2 were prepared following the synthetic procedure described for Example 26(B), using the suitable acyl chloride.

TABLE 2
amide derivatives prepared according to Example 26.

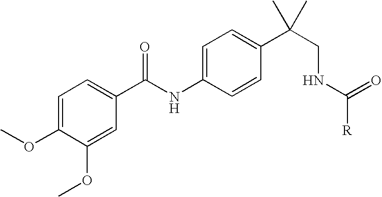

| | | | | LCMS | | | | |
|---|---|---|---|---|---|---|---|---|
| | | | MP | | | RT | | |
| R | Example | Yield^{a} | (C) | Met | [MH+] | (min) | Appearance | Purification |
| 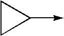 | 30 | 53 | | G | 397.1 | 1.98 | White amorphous solid | SCX^{b} |
| 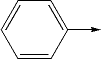 | 31 | 60 | | G | 433.1 | 2.20 | White amorphous solid | SCX^{b} |
| 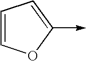 | 32 | 40 | | G | 423.1 | 2.05 | White amorphous solid | SCX^{b} |
| 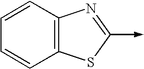 | 33 | 55 | | G | 490.1 | 2.56 | White amorphous solid | SCX^{b} |
| 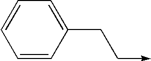 | 34 | 45 | 149-151 | G | 461.2 | 2.29 | White Powder | SCX^{b} and then crystallization from EtOAc |
| 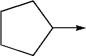 | 35 | 33 | 143-146 | G | 425.2 | 2.20 | White Powder | SCX^{b} and then crystallization from EtOAc |
| 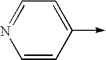 | 36 | 49 | 221-225 | G | 434.2 | 1.64 | White Powder | SCX^{b} |
| 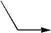 | 37 | 86 | | G | 385.2 | 1.88 | Vitreous solid | SCX^{b} |
| 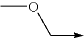 | 38 | 64 | 183-185 | G | 401.2 | 1.90 | White solid | Crystallization from EtOAc |
| 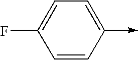 | 40 | 87 | 191-194 | G | 451.2 | 2.28 | White solid | Trituration with Et₂O |
| 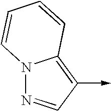 | 41 | 40 | | G | 473.2 | 2.05 | White amorphous solid | Crystallization from EtOAc |
| 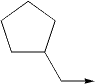 | 42 | 37 | 180-183 | G | 439.2 | 2.32 | White solid | Trituration with MeOH |
| 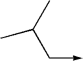 | 46 | 19 | 76-79 | G | 413.2 | 2.15 | White solid | SCX^{b} |
| 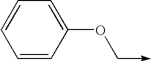 | 47 | 23 | 159-162 | G | 463.2 | 2.31 | White solid | SCX^{b} |
| 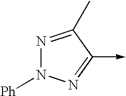 | 49 | 37 | 175-178 | G | 514.2 | 2.72 | Off-white solid | Preparative HPLC (Method S) |
| 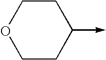 | 56 | 56 | 169-171 | G | 441.11 | 1.84 | White solid | Crystallization from EtOAc |
| 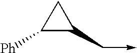 | 57 | 67 | 197-200 | G | 473.08 | 2.38 | White solid | Trituration with EtOAc |
| 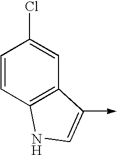 | 58 | 27 | 224-226 | G | 506.03 | 2.52 | Pale brown powder | SCX^{b} |
| 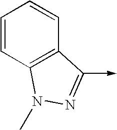 | 59 | 34 | | G | 487.09 | 2.38 | White amorphous solid | Preparative HPLC (Method M) |
| 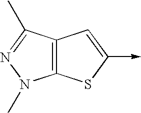 | 61 | 70 | 212-213 | G | 507.16 | 2.16 | White solid | Crystallization from EtOAc |
| 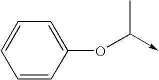 | 67 | 35 | | G | 477.20 | 2.39 | White amorphous solid | SCX^{b} |
| 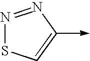 | 71 | 51 | | G | 440.97 | 3.17 | Yellow amorphous solid | SCX^{b} |
| 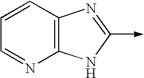 | 213^{c} | 44 | 204-206 | M | 474.21 | 1.72 | White solid | Trituration with EtOAc |
| 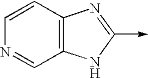 | 214 | 34 | | M | 474.25 | 1.59 | Light yellow amorphous solid | Chromatography [SiO₂, DCM/MeOH/TEA (99/0.5/0.5 to 96/2/2/)] Followed by SCX^{b} |

^{a}isolated yield of analytically pure product
^{b}eluted with DCM—MeOH (1:1) to MeOH—NH4OH (9:1)
^{c}The needed carboxylic acid was prepared according to the procedure reported in Pharmazie., (1988), 43, 315-317.

TABLE 3
NMR data of the compounds reported in Table 2.

| R | Example | NMR-data |
|---|---|---|
| 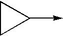 | 30 | ′1H NMR (300 MHz, CHLOROFORM-d) d ppm 7.77 (s, 1 H), 7.58-7.67 (m, 2 H), 7.52 (d, 1 H), 7.33-7.44 (m, 3 H), 6.93 (d, 1 H), 5.28 (br. s., 1 H), 3.98 (s, 3 H), 3.96 (s, 3 H), 3.50 (d, 2 H), 1.35 (s, 6 H), 1.15-1.26 (m, 1 H), 0.88- 0.98 (m, 2 H), 0.61-0.73 (m, 2 H) |
| 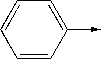 | 31 | ′1H NMR (300 MHz, CHLOROFORM-d) d ppm 7.77 (s, 1 H), 7.57-7.71 (m, 4 H), 7.52 (d, 1 H), 7.33-7.49 (m, 6 H), 6.93 (d, 1 H), 5.80 (br. s., 1 H), 3.98 (s, 3 H), 3.96 (s, 3 H), 3.68 (d, 2 H), 1.43 (s, 6 H) |
| 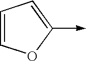 | 32 | ′1H NMR (300 MHz, CHLOROFORM-d) d ppm 7.78 (s, 1 H), 7.59-7.70 (m, 2 H), 7.52 (d, 1 H), 7.38-7.45 (m, 3 H), 7.37 (dd, 1 H), 7.07 (dd, 1 H), 6.93 (d, 1 H), 6.46 (dd, 1 H), 6.10 (br. s., 1 H), 3.97 (d, 6 H), 3.63 (d, 2 H), 1.41 (s, 6 H) |
| 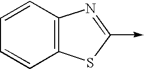 | 33 | ′1H NMR (300 MHz, CHLOROFORM-d) d ppm 8.02-8.08 (m, 1 H), 7.93- 8.01 (m, 1 H), 7.79 (s, 1 H), 7.61-7.71 (m, 2 H), 7.56 (d, 1 H), 7.53 (d, 1 H), 7.51 (dd, 1 H), 7.45-7.49 (m, 2 H), 7.42 (dd, 1 H), 7.30-7.35 (m, 1 H), 6.94 (d, 1 H), 3.99 (s, 3 H), 3.97 (s, 3 H), 3.72 (d, 2 H), 1.47 (s, 6 H) |
| 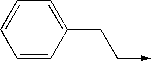 | 34 | ′1H NMR (300 MHz, CHLOROFORM-d) d ppm 7.77 (s, 1 H), 7.54-7.61 (m, 2 H), 7.52 (d, 1 H), 7.40 (dd, 1 H), 7.28-7.33 (m, 1 H), 7.11-7.26 (m, 6 H), 6.93 (d, 1 H), 5.01 (br. s., 1 H), 3.97 (s, 3 H), 3.96 (s, 3 H), 3.43 (d, 2 H), 2.91 (t, 2 H), 2.39 (t, 2 H), 1.25 (s, 6 H) |
| 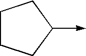 | 35 | ′1H NMR (300 MHz, CHLOROFORM-d) d ppm 7.77 (s, 1 H), 7.57-7.66 (m, 2 H), 7.52 (d, 1 H), 7.41 (dd, 1 H), 7.36 (m, 2 H), 6.93 (d, 1 H), 5.11 (br. s., 1 H), 3.98 (s, 3 H), 3.96 (s, 3 H), 3.47 (d, 2 H), 2.27-2.48 (m, 1 H), 1.62-1.82 (m, 4 H), 1.48-1.61 (m, 4 H), 1.34 (s, 6 H) |
| 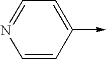 | 36 | ′1H NMR (300 MHz, CHLOROFORM-d) d ppm 8.59-8.74 (m, 2 H), 8.29 1 H), 7.64-7.73 (m, 2 H), 7.52 (d, 1 H), 7.47 (d, 1 H), 7.36-7.45 (m, 4 H), 6.91 (d, 1 H), 5.99 (br. s., 1 H), 3.95 (s, 3 H), 3.94 (s, 3 H), 3.65 (d, 2 H), 1.40 (s, 6 H) |
| 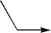 | 37 | ′1H NMR (300 MHz, CHLOROFORM-d) d ppm 7.78 (s, 1 H), 7.56-7.68 (m, 2 H), 7.52 (d, 1 H), 7.41 (dd, 1 H), 7.33-7.38 (m, 2 H), 6.93 (d, 1 H), 5.10 (br. s., 1 H), 3.98 (s, 3 H), 3.96 (s, 3 H), 3.48 (d, 2 H), 2.11 (q, 2 H), 1.34 (s, 6 H), 1.09 (t, 3 H) |
| 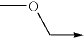 | 38 | ′1H NMR (300 MHz, CHLOROFORM-d) d ppm 8.32 (s, 1 H), 7.59-7.68 (m, 2 H), 7.51 (d, 1 H), 7.45 (dd, 1 H), 7.30-7.38 (m, 2 H), 6.90 (d, 1 H), 6.31 (br. s., 1 H), 3.94 (s, 3 H), 3.93 (s, 3 H), 3.81 (s, 2 H), 3.47 (d, 2 H), 3.28 (s, 3 H), 1.33 (s, 6 H) |
| 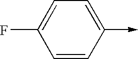 | 40 | ′1H NMR (300 MHz, DMSO-d6) d ppm 9.99 (s, 1 H), 8.22 (t, 1 H), 7.79-7.93 (m, 2 H), 7.66-7.74 (m, 2 H), 7.62 (dd, 1 H), 7.53 (d, 1 H), 7.34-7.44 (m, 1 H), 7.20-7.32 (m, 3 H), 7.08 (d, 1 H), 3.85 (s, 3 H), 3.84 (s, 3 H), 3.46 (d, 2 H), 1.31 (s, 6 H) |
| 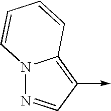 | 41 | ′1H NMR (300 MHz, CHLOROFORM-d) d ppm 8.37-8.53 (m, 1 H), 8.11- 8.22 (m, 1 H), 7.92 (s, 1 H), 7.79 (s, 1 H), 7.66 (m, 2 H), 7.52 (d, 1 H), 7.45 (m, 2 H), 7.40 (d, 1 H), 7.29-7.37 (m, 1 H), 6.94 (d, 1 H), 6.86-6.93 (m, 1 H), 5.53 (t, 1 H), 3.98 (s, 3 H), 3.97 (s, 3 H), 3.68 (d, 2 H), 1.44 (s, 6 H) |
| 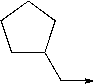 | 42 | ′1H NMR (300 MHz, CHLOROFORM-d) d ppm 7.77 (s, 1 H), 7.61 (m, 2 H), 7.52 (d, 1 H), 7.40 (dd, 1 H), 7.36 (m, 2 H), 6.93 (d, 1 H), 5.10 (t, 1 H), 3.98 (s, 3 H), 3.96 (s, 3 H), 3.49 (d, 2 H), 2.09 (s, 2 H), 2.04-2.21 (m, 1 H), 1.68-1.84 (m, 2 H), 1.45-1.66 (m, 4 H), 1.35 (s, 6 H), 0.91-1.18 (m, 2 H) |
| 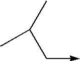 | 46 | ′1H NMR (300 MHz, CHLOROFORM-d) d ppm 7.74 (s, 1 H), 7.62 (m, 2 H), 7.52 (d, 1 H), 7.42 (dd, 1 H), 7.32-7.41 (m, 2 H), 6.94 (d, 1 H), 5.06 (br. s., 1 H), 3.98 (s, 3 H), 3.97 (s, 3 H), 3.49 (d, 2 H), 1.97-2.15 (m, 1 H), 1.87-1.97 (m, 2 H), 1.35 (s, 6 H), 0.90 (d, 6 H) |
| 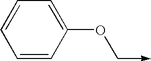 | 47 | ′1H NMR (300 MHz, CHLOROFORM-d) d ppm 7.74 (s, 1 H), 7.57 (m, 2 H), 7.53 (d, 1 H), 7.41 (dd, 1 H), 7.32-7.35 (m, 1 H), 7.31 (d, 1 H), 7.25 (m, 2 H), 6.99-7.05 (m, 1 H), 6.94 (d, 1 H), 6.81 (d, 2 H), 6.31 (br. s., 1 H), 4.46 (s, 2 H), 3.98 (s, 3 H), 3.97 (s, 3 H), 3.50 (d, 2 H), 1.32 (s, 6 H) |
| 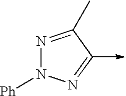 | 49 | ′1H NMR (300 MHz, CHLOROFORM-d) d ppm 7.93-8.02 (m, 2 H), 7.76 (s, 1 H), 7.61-7.71 (m, 2 H), 7.52 (d, 1 H), 7.49-7.51 (m, 1 H), 7.44-7.48 (m, 2 H), 7.41 (dd, 1 H), 7.36-7.38 (m, 1 H), 7.31-7.36 (m, 1 H), 6.94 (d, 1 H), 6.69 (t, 1 H), 3.98 (s, 3 H), 3.97 (s, 3 H), 3.65 (d, 2 H), 2.64 (s, 3 H), 1.44 (s, 6 H) |
| 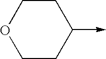 | 56 | ′1H NMR (300 MHz, CHLOROFORM-d) d ppm 8.72 (s, 1 H), 7.58-7.73 (m, 2 H), 7.45-7.51 (m, 2 H), 7.28-7.33 (m, 2 H), 6.88 (d, 1 H), 5.32 (t, 1 H), 3.95-4.03 (m, 2 H), 3.91 (s, 3 H), 3.89 (s, 3 H), 3.40 (d, 2 H), 3.30 (td, 2 H), 2.08-2.27 (m, 1 H), 1.49-1.76 (m, 4 H), 1.27 (s, 6 H) |
| 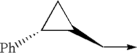 | 57 | ′1H NMR (300 MHz, CHLOROFORM-d) d ppm 8.58 (s, 1 H), 7.62 (m, 2 H), 7.50 (d, 1 H), 7.46 (dd, 1 H), 7.31 (m, 2 H), 7.06-7.23 (m, 3 H), 6.96-7.04 (m, 2 H), 6.88 (d, 1 H), 5.58 (t, 1 H), 3.92 (s, 3 H), 3.90 (s, 3 H), 3.37-3.55 (m, 2 H), 2.34-2.43 (m, 1 H), 1.45-1.57 (m, 2 H), 1.30 (d, 6 H), 1.06-1.16 (m, 1 H) |
| 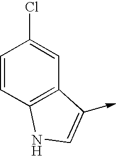 | 58 | ′1H NMR (300 MHz, CHLOROFORM-d) d ppm 9.84 (br. s., 1 H), 8.45 (s, 1 H), 7.64-7.73 (m, 2 H), 7.53 (d, 2 H), 7.48 (dd, 1 H), 7.35-7.42 (m, 2 H), 7.32 (d, 1 H), 7.16 (dd, 1 H), 6.91 (d, 1 H), 6.57 (d, 1 H), 6.09 (t, 1 H), 3.95 (s, 3 H), 3.93 (s, 3 H), 3.63 (d, 2 H), 1.39 (s, 6 H) |
| 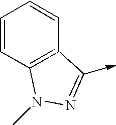 | 59 | 1H NMR (300 MHz, CHLOROFORM-d) d ppm 8.33-8.40 (m, 1 H), 7.77 (s, 1 H), 7.60-7.69 (m, 2 H), 7.52 (d, 1 H), 7.44-7.50 (m, 2 H), 7.35-7.43 (m, 3 H), 6.93 (d, 1 H), 6.84-6.90 (m, 1 H), 4.05 (s, 3 H), 3.97 (s, 3 H), 3.96 (s, 3 H), 3.71 (d, 2 H), 1.45 (s, 6 H) |
| 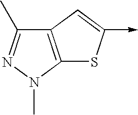 | 61 | 1H NMR (300 MHz, CHLOROFORM-d) d ppm 7.80 (s, 1 H), 7.62-7.71 (m, 2 H), 7.52 (d, 1 H), 7.41-7.46 (m, 2 H), 7.39-7.43 (m, 1 H), 7.12 (s, 1 H), 6.94 (d, 1 H), 5.62 (t, 1 H), 3.98 (s, 3 H), 3.96 (s, 3 H), 3.89 (s, 3 H), 3.65 (d, 2 H), 2.42 (s, 3 H), 1.42 (s, 6 H) |
| 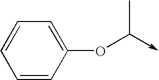 | 67 | ′1H NMR (300 MHz, CHLOROFORM-d) d ppm 7.72 (s, 1 H), 7.46-7.60 (m, 3 H), 7.41 (dd, 1 H), 7.28-7.35 (m, 2 H), 7.15 (m, 2 H), 6.97-7.06 (m, 1 H), 6.94 (d, 1 H), 6.72-6.85 (m, 2 H), 6.15 (t, 1 H), 4.62 (q, 1 H), 3.98 (s, 3 H), 3.97 (s, 3 H), 3.57 (dd, 1 H), 3.28 (dd, 1 H), 1.51 (d, 3 H), 1.30 (s, 3 H), 1.19 (s, 3H) |
| 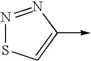 | 71 | ′1H NMR (300 MHz, CHLOROFORM-d) d ppm 9.16 (s, 1 H), 8.67 (s, 1 H), 7.62-7.70 (m, 2 H), 7.49-7.52 (m, 1 H), 7.47 (d, 1 H), 7.29-7.41 (m, 3 H), 6.88 (d, 1 H), 3.92 (s, 3 H), 3.90 (s, 3 H), 3.68 (d, 2 H), 1.39 (s, 6 H) |
| 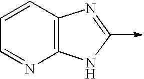 | 213^{c} | 1H NMR (300 MHz, DMSO-d6 353K) d ppm 9.78 (s, 1 H), 8.37-8.56 (m, 1 H), 7.99 (br. s., 1 H), 7.72 (m, 2 H), 7.62 (dd, 1 H), 7.57 (d, 1 H), 7.44 (m, 2 H), 7.31 (dd, 1 H), 7.07 (d, 1 H), 3.87 (d, 6 H), 3.62 (d, 2 H), 1.39 (s, 6 H) |
| 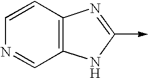 | 214 | 1H NMR (300 MHz, DMSO-d6 353K) d ppm 13.09 (s, 1 H), 9.77 (s, 1 H), 8.98 (s, 1 H), 8.36 (d, 1 H), 8.01 (t, 1 H), 7.72 (m, 2 H), 7.62 (dd, 1 H), 7.57 (d, 1 H), 7.51-7.59 (m, 1 H), 7.43 (m, 2 H), 7.07 (d, 1 H), 3.87 (s, 3 H), 3.87 (s, 3 H), 3.63 (d, 2 H), 1.38 (s, 6 H) |

The compounds reported in Table 4 were prepared following the synthetic procedure described for Example 50, using the suitable carboxylic acids.

TABLE 4
amide derivatives prepared according to Example 50.

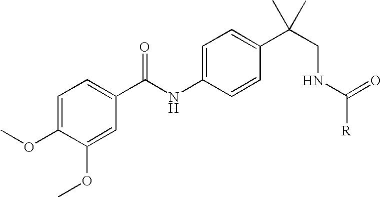

| | | | | LCMS | | | | |
|---|---|---|---|---|---|---|---|---|
| | | | MP | | | RT | | |
| R | Example | Yield^{a} | (° C.) | Met | [MH+] | (min) | Appearance | Purification |
| 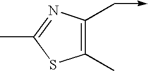 | 51 | 33 | 162- 165 | G | 482.1 | 1.79 | White solid | Preparative HPLC (Method S) |
| 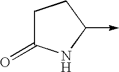 | 55 | 12 | 112- 115 | G | 440.1 | 1.66 | White solid | Preparative HPLC (Method S) |
| 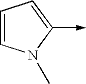 | 62 | 64 | 183- 137 | G | 436.20 | 2.22 | White powder | Crystallization from EtOAc |
| 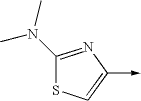 | 63 | 22 | | G | 483.07 | 2.18 | Pale yellow amorphous solid | Preparative HPLC (Method Q) |
| 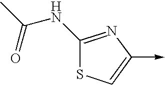 | 64 | 42 | | G | 497.06 | 1.98 | White amorphous solid | SCX^{b} |
| 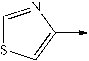 | 65 | 17 | | G | 440.10 | 2.06 | Pale yellow amorphous solid | Preparative HPLC (Method S) |
| 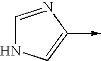 | 66 | 7 | | G | 423.14 | 1.59 | White amorphous solid | Preparative HPLC (Method S) |
| 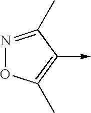 | 68 | 9 | | G | 452.15 | 2.06 | White solid | Preparative HPLC (Method S) |
| 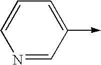 | 69 | 21 | | G | 434.18 | 1.64 | White solid | Preparative HPLC (Method S) |
| 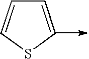 | 70 | 31 | | G | 439.09 | 2.17 | White solid | Preparative HPLC (Method S) |
| 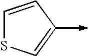 | 72 | 67 | 189- 190 | G | 439.09 | 2.16 | White solid | Trituration with EtOAc |
| 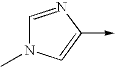 | 73 | 73 | 210- 212 | G | 437.14 | 1.64 | White solid | Crystallization from EtOAc |
| 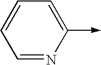 | 74 | 35 | | G | 434.18 | 2.18 | Yellow vitreous solid | Prepartive HPLC (Method T) |
| 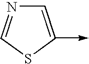 | 76 | 53 | | G | 440.10 | 1.94 | White amorphous solid | SCX^{b} |
| 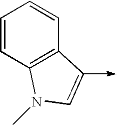 | 81 | 30 | | G | 486.15 | 2.27 | White powder | Chromatography [SiO₂, DCM/ MeOH (97/3)] Followed by Preparative HPLC (Method Q) |
| 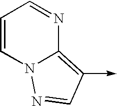 | 82 | 51 | 224- 226 | G | 474.31 | 2.01 | White powder | Chromatography [SiO₂, DCM/ MeOH (98/2)] |
| 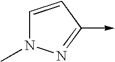 | 83 | 76 | 175- 176 | G | 437.35 | 1.96 | White powder | Chromatography [SiO₂, DCM/ MeOH (98/2)] |
| 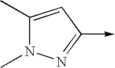 | 85 | 64 | 96- 98 | G | 451.36 | 2.03 | White powder | Chromatography [SiO₂, DCM/ MeOH (98/2)] |
| 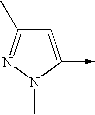 | 86 | 37 | 210- 211 | G | 451.36 | 2.03 | White powder | Chromatography [SiO₂, DCM/ MeOH (98/2)] |
| 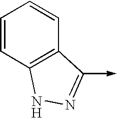 | 87 | 42 | 214- 216 | G | 473.30 | 2.23 | White powder | Chromatography [SiO₂, DCM/ MeOH (98/2)] |
| 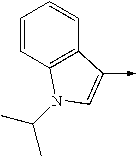 | 105 | 49 | | G | 514.31 | 2.45 | White amorphous solid | Chromatography [SiO₂, DCM to DCM/MeOH (99/1)] |
| 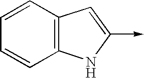 | 106 | 71 | 234- 236 | G | 472.16 | 2.32 | White solid | Chromatography [SiO₂, DCM to DCM/MeOH (99/1)] Followed by crystallization from EtOAc |
| 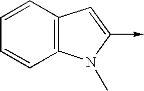 | 108 | 17 | 176- 178 | G | 486.30 | 2.51 | White solid | Trituration with DCM |
| 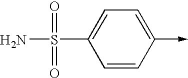 | 115 | 95 | 237- 239 | G | 512.22 | 1.89 | White solid | Filtration from reaction system |
| 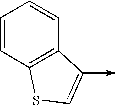 | 116 | 70 | 145- 147 | G | 489.19 | 2.44 | White solid | Trituration with EtOAc |
| 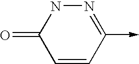 | 117 | 22 | 241- 243 | G | 451.22 | 1.79 | Pale yellow solid | Trituration with EtOAc |
| 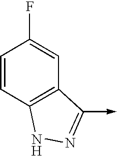 | 119^{c} | 26 | | G | 491.21 | 2.29 | Yellow solid | Chromatography [SiO₂, DCM/ MeOH (50/1)] |
| 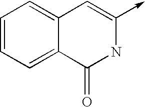 | 124 | 61 | 228- 229 | G | 500.24 | 2.07 | White solid | Trituration with EtOAc/Et₂O (1/1 ratio) |
| 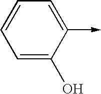 | 129 | 64 | 140- 141 | G | 449.20 | 2.35 | White solid | Trituration with EtOAc |
| 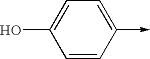 | 130 | 21 | | G | 449.20 | 1.93 | White amourphous solid | Preparative HPLC (Method S) |
| 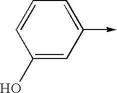 | 134 | 15 | | G | 449.27 | 1.97 | White amourphous solid | Preparative HPLC (Method R) |
| 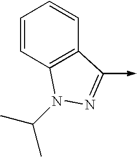 | 135 | 99 | | G | 515.25 | 2.64 | White powder | Chromatography [SiO₂, Hexane/ EtOAc (9/1 to 6/4)] |
| 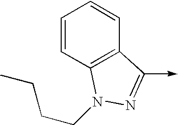 | 136 | 92 | | G | 529.26 | 2.77 | White powder | Chromatography [SiO₂, Hexane/ EtOAc (9/1 to 6/4)] |
| 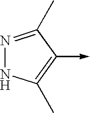 | 137 | 31 | | G | 451.29 | 1.74 | White amorphous solid | Chromatography [SiO₂, DCM to DCM/MeOH (95/5)] |
| 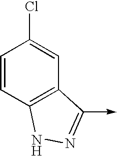 | 138^{c} | 30 | | G | 507.24 | 2.41 | Pale yellow amorphous solid | Chromatography [SiO₂, Petroleum ether to petroleum ether/EtOAc (1/1)] |
| 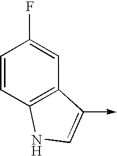 | 139^{d} | 66 | 134- 135 | G | 490.14 | 2.21 | White solid | Trituration with EtOAc |
| 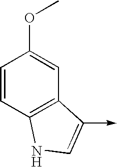 | 140^{d} | 28 | 208- 209 | G | 502.12 | 2.13 | Pale yellow solid | Trituration with EtOAc |
| 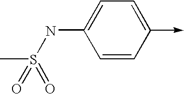 | 141 | 10 | | G | 526.23 | 2.98 | Off-white solid | Precipitation from acqueous phase |
| 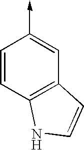 | 142 | 51 | | G | 472.23 | 2.13 | White solid | Chromatography [SiO₂, DCM/ MeOH (98/2)] |
| 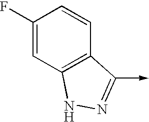 | 143^{c} | 19 | | G | 491.15 | 2.31 | Yellow solid | Chromatography [SiO₂, DCM/MeOH (99/1] |
| 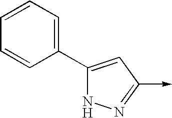 | 144 | 46 | | G | 499.16 | 2.28 | Pale yellow solid | Chromatography [SiO₂, DCM/ MeOH (98/2 to 95/5)] |
| 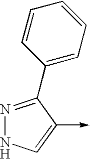 | 145 | 16 | | G | 499.16 | 2.27 | White powder | Chromatography [SiO₂, DCM/ MeOH (98/2 to 95/5)] |
| 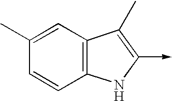 | 146 | 51 | | G | 500.24 | 2.59 | Yellow powder | Chromatography [SiO₂, Petroleum ether/ EtOAc (8/2 to 7/3)] |
| 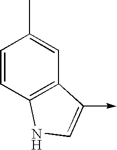 | 171^{c} | 37 | | G | 487.24 | 2.28 | Yellow amorphous solid | Chromatography [SiO₂, Petroleum ether/ EtOAc (95/5 to 1/1)] |
| 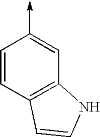 | 172 | 84 | | G | 472.23 | 2.13 | White amorphous solid | Trituration with EtOAc/Et₂O (1/1) |
| 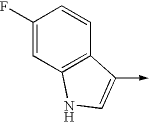 | 175^{e} | 56 | 206- 208 | G | 490.07 | 2.24 | White solid | Chromatography [SiO₂, Petroleum ether/ EtOAc (6/4)] |
| 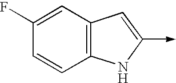 | 176 | 83 | 230- 232 | G | 490.14 | 2.47 | White solid | Filtration from reaction system |
| 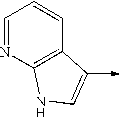 | 178^{f} | 31 | | G | 473.32 | 1.73 | Off-white amophous solid | Chromatography [SiO₂, DCM to DCM/MeOH (9/1)] Followed by Trituration EtOAc |
| 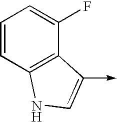 | 179^{e} | 43 | | G | 490.32 | 221 | White amophous solid | Trituration with EtOAc |
| 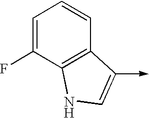 | 180^{e} | 54 | 155- 158 | G | 490.23 | 2.21 | White solid | Filtration from reaction system |
| 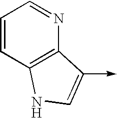 | 181^{f} | 48 | | G | 473.26 | 1.69 | Light yellow amourphous solid | Chromatography [SiO₂, Petroleum ether/ EtOAc (7/3) to EtOAc] |
| 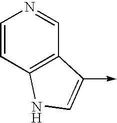 | 182^{f} | 13 | | G | 473.21 | 1.62 | Light yellow amourphous solid | Chromatography [SiO₂, DCM/ MeOH/TEA (97/2.5/0.5 to 97/2.5/1] |
| 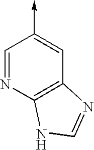 | 189 | 23 | | M | 488.34 | 1.69 | White solid | Chromayography [SiO₂, DCM/ MeOH (98/2 to 94/6] |
| 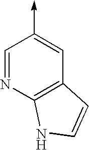 | 202 | 62 | | O | 473.54 | 1.99 | White powder | Filtration from reaction system |
| 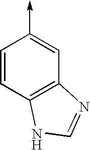 | 207 | 8 | | M | 473.27 | 1.61 | White solid | Preparative HPLC (Method Q) |
| 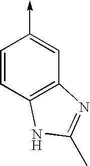 | 208 | 17 | | M | 487.26 | 1.62 | White solid | Preparative HPLC (Method Q) |
| 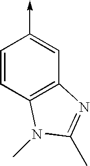 | 209 | 61 | | M | 501.31 | 1.64 | Pale yellow solid | Preparative HPLC (Method Q) |
| 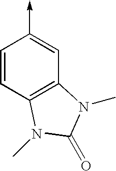 | 210 | 32 | | M | 517.30 | 1.91 | White solid | Preparative HPLC (Method Q) |
| 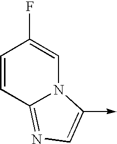 | 211 | 50 | | M | 491.24 | 1.81 | Off-white solid | Chromatography [SiO₂, DCM to DCM/MeOH (95/5)] |
| 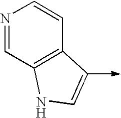 | 215^{f} | 31 | | M | 473.26 | 1.61 | White amorphous solid | Chromatography [SiO₂, EtOAc/MeOH + 0.5% NH₄OH (99/1 to 96/5)] Followed by SCX^{b} |
| 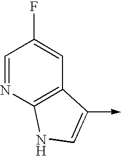 | 216^{f} | 71 | | M | 491.24 | 1.99 | Pale yellow amorphous solid | Trituration with EtOAc/Et₂O (1/1 ration) |
| 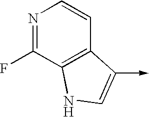 | 217^{g} | 35 | | M | 491.24 | 1.94 | Pale yellow amorphous solid | Chromatography [SiO₂, DCM/MeOH + 0.5% NH₄OH (99.5/0.5 to 98/2)] |
| 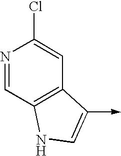 | 218^{h} | 46 | | M | 507.24 | 1.92 | Pale yellow amorphous solid | Chromatography [SiO₂, Petroleum ether/EtOAc (9/1 to 8/2)] Followed by SCX^{b} |
| 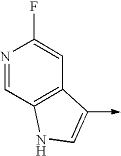 | 219^{h} | 59 | | O | 491.52 | 2.09 | Off-white amorphous solid | Chromatography [SiO₂, Petroleum ether/EtOAc (9/1 to 8/2)] Followed by SCX^{b} |

^{a}isolated yield of analytically pure product
^{b}eluted with DCM-MeOH (1:1) to MeOH-NH4OH (9:1)
^{c}The needed carboxylic acid was prepared according to the procedure reported in J. Med. Chem, (2000), 43, 1 pg 41.
^{d}The needed carboxylic acid was prepared according to the procedure reported in Chem. Pharm. Bull., (1995), 43, 11, 1912-1930.
^{e}The needed carboxylic acid was prepared according to the procedure reported in J. Med. Chem, (1990), 33, 2777-2784 and Eur. J. Med. Chem Chim. Ther. (1999), 34, 2, 93-106.
^{f}The needed carboxylic acid was prepared according to the procedure reported in Heterocycles (1999), 50, 2, 1065-1080 and Synthesis (2000), 4, 549-556.
^{g}The needed carboxylic acid was prepared according to the procedure reported in JOC (2002), 67, 2345-2347, Biorg. Med. Chem. (1999), 7, 921-932 and Synthesis (2000), 4, 549-556.
^{h}The needed carboxylic acid was prepared according to the procedure reported in J. Heterocyclic Chem (1992), 29, 359-367, Biorg. Med. Chem. (1999), 7, 921-932 and Synthesis (2000), 4, 549-556.

TABLE 5
NMR data of the compounds reported in Table 4.

| R | Example | NMR-data |
|---|---|---|
| 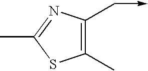 | 51 | 1H NMR (300 MHz, DMSO-d6 +TFA ) d ppm 10.00 (s, 1 H), 7.68 (d, 2 H), 7.62 (dd, 1 H), 7.54-7.60 (m, 1 H), 7.53 (d, 1 H), 7.30 (d, 2 H), 7.08 (d, 1 H), 3.84 (s, 3 H), 3.84 (s, 3 H), 3.48 (s, 2 H), 3.28 (d, 2 H), 2.57 (s, 3 H), 2.27 (s, 3 H), 1.22 (s, 6 H) |
| 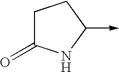 | 55 | 1H NMR (300 MHz, CHLOROFORM-d) d ppm 7.93 (br. s., 1 H), 7.59 (m, 2 H), 7.49- 7.54 (m, 1 H), 7.43 (d, 1 H), 7.31 (m, 2 H), 6.93 (d, 1 H), 6.00 (br. s., 1 H), 5.76 (br. s., 1 H), 3.99-4.09 (m, 1 H), 3.97 (br. s., 6 H), 3.29-3.62 (m, 2 H), 2.33-2.54 (m, 1 H), 2.20- 2.33 (m, 2 H), 1.93-2.15 (m, 1 H), 1.34 (s, 6 H) |
| 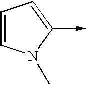 | 62 | 1H NMR (300 MHz, CHLOROFORM-d) d ppm 7.77 (s, 1 H), 7.60-7.70 (m, 2 H), 7.52 (d, 1 H), 7.37-7.47 (m, 3 H), 6.93 (d, 1 H), 6.65-6.72 (m, 1 H), 6.27 (dd, 1 H), 6.01 (dd, 1 H), 5.58 (t, 1 H), 3.98 (s, 3 H), 3.96 (s, 3 H), 3.92 (s, 3 H), 3.57 (d, 2 H), 1.40 (s, 6 H) |
| 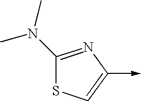 | 63 | ′1H NMR (300 MHz, CHLOROFORM-d) d ppm 7.77 (s, 1 H), 7.62 (m, 2 H), 7.56 (br. s., 1 H), 7.51 (d, 1 H), 7.44 (m, 2 H), 7.40 (dd, 1 H), 7.29 (s, 1 H), 6.93 (d, 1 H), 3.97 (s, 3 H), 3.96 (s, 3 H), 3.58 (d, 2 H), 3.12 (s, 6 H), 1.42 (s, 6 H) |
| 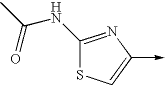 | 64 | ′1H NMR (300 MHz, CHLOROFORM-d) d ppm 10.84 (s, 1 H), 8.70 (s, 1 H), 7.63 (s, 1 H), 7.56-7.62 (m, 2 H), 7.45-7.53 (m, 2 H), 7.30-7.42 (m, 2 H), 6.91 (d, 1 H), 6.72-6.81 (m, 1 H), 3.93 (s, 6 H), 3.56 (d, 2 H), 2.23 (s, 3 H), 1.37 (s, 6 H) |
| 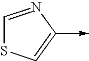 | 65 | 1H NMR (300 MHz, CHLOROFORM-d) d ppm 8.68 (d, 1 H), 8.14 (d, 1 H), 7.75 (s, 1 H), 7.59-7.68 (m, 2 H), 7.52 (d, 1 H), 7.40-7.47 (m, 2 H), 7.41(dd, 1 H), 7.28 (br. s., 1 H), 6.93 (d, 1 H), 3.98 (s, 3 H), 3.96 (s, 3 H), 3.67 (d, 2 H), 1.42 (s, 6 H) |
| 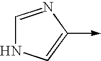 | 66 | 1H NMR (300 MHz, CHLOROFORM-d) d ppm 8.44 (s, 1 H), 7.62 (m, 2 H), 7.55 (d, 1 H), 7.49-7.54(m, 2 H), 7.46(dd, 1 H), 7.38 (m, 2 H), 7.01 (br. s., 1 H), 6.89 (d, 1 H), 3.93 (s, 3 H), 3.91 (s, 3 H), 3.58 (d, 2 H), 1.36 (s, 6 H) |
| 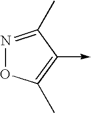 | 68 | ′1H NMR (300 MHz, CHLOROFORM-d) d ppm 8.69 (s, 1 H), 7.67 (m, 2 H), 7.44-7.51 (m, 2 H), 7.33 (m, 2 H), 6.88 (d, 1 H), 5.44 (t, 1 H), 3.92 (s, 3 H), 3.90 (s, 3 H), 3.57 (d, 2 H), 2.38 (s, 3 H), 2.12 (s, 3 H), 1.35 (s, 6 H) |
| 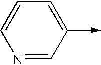 | 69 | ′1H NMR (300 MHz, CHLOROFORM-d) d ppm 9.13 (s, 1 H), 8.71 (s, 1 H), 8.51 (dd, 1 H), 7.79-7.92 (m, 1 H), 7.60 (d, 2 H), 7.37-7.53 (m, 2 H), 7.22-7.26 (m, 2 H), 7.16-7.22 (m, 1 H), 6.79 (d, 1 H), 6.59 (t, 1 H), 3.81 (s, 3 H), 3.79 (s, 3 H), 3.49 (d, 2 H), 1.26 (s, 6 H) |
| 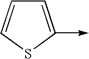 | 70 | ′1H NMR (300 MHz, CHLOROFORM-d) d ppm 8.86 (s, 1 H), 7.65 (m, 2 H), 7.44-7.53 (m, 2 H), 7.35 (dd, 1 H), 7.32 (m, 2 H), 7.25 (dd, 1 H), 6.94 (dd, 1 H), 6.86 (d, 1 H), 5.83- 5.97 (m, 1 H), 3.89 (s, 3 H), 3.87 (s, 3 H), 3.53 (d, 2 H), 1.32 (s, 6 H) |
| 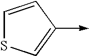 | 72 | ′1H NMR (300 MHz, CHLOROFORM-d) d ppm 7.80 (s, 1 H), 7.70 (dd, 1 H), 7.62-7.68 (m, 2 H), 7.52 (d, 1 H), 7.36-7.48 (m, 3 H), 7.30 (dd, 1 H), 7.21 (dd, 1 H), 6.93 (d, 1 H), 5.62 (t, 1 H), 3.98 (s, 3 H), 3.96 (s, 3 H), 3.63 (d, 2 H), 1.41 (s, 6 H) |
| 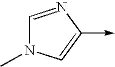 | 73 | ′1H NMR (300 MHz, CHLOROFORM-d) d ppm 7.84 (s, 1 H), 7.57-7.66 (m, 2 H), 7.51 (d, 1 H), 7.48 (d, 1 H), 7.37-7.44 (m, 3 H), 7.34 (d, 1 H), 7.04 (t, 1 H), 6.92 (d, 1 H), 3.97 (s, 3 H), 3.96 (s, 3 H), 3.71 (s, 3 H), 3.61 (d, 2 H), 1.40 (s, 6 H) |
| 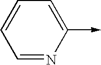 | 74 | ′1H NMR (300 MHz, CHLOROFORM-d) d ppm 8.48 (ddd, 1 H), 8.12-8.25 (m, 1 H), 7.92- 8.07 (m, 1 H), 7.79-7.86 (m, 1 H), 7.56-7.71 (m, 2 H), 7.52 (d, 2 H), 7.42-7.47 (m, 2 H), 7.36-7.43 (m, 2 H), 6.93 (d, 1 H), 3.97 (s, 3 H), 3.96 (s, 3 H), 3.68 (d, 2 H), 1.43 (s, 6 H) |
| 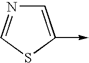 | 76 | ′1H NMR (300 MHz, CHLOROFORM-d) d ppm 8.88 (s, 1 H), 8.09 (s, 1 H), 7.83 (s, 1 H), 7.59-7.73 (m, 2 H), 7.53 (d, 1 H), 7.44 (d, 1 H), 7.38-7.43 (m, 2 H), 6.94 (d, 1 H), 5.80 (t, 1 H), 3.99 (s, 3 H), 3.97 (s, 3 H), 3.65 (d, 2 H), 1.43 (s, 6 H) |
| 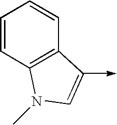 | 81 | ′1H NMR (300 MHz, DMSO-d6) d ppm 9.99 (s, 1 H), 7.94-8.07 (m, 2 H), 7.66-7.78 (m, 2 H), 7.62 (dd, 1 H), 7.53 (d, 1 H), 7.34-7.51 (m, 4 H), 7.10-7.23 (m, 2 H), 7.07 (d, 1 H), 3.84 (s, 3 H), 3.84 (s, 3 H), 3.81 (s, 3 H), 3.48 (d, 2 H), 1.32 (s, 6 H) |
| 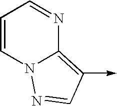 | 82 | ′1H NMR (300 MHz, DMSO-d6) ppm 10.01 (s, 1 H), 9.27 (dd, 1 H), 8.66 (dd, 1 H), 8.53 (s, 1 H), 7.84 (t, 1 H), 7.71 (m, 2 H), 7.63 (dd, 1 H), 7.54 (d, 1 H), 7.43 (m, 2 H), 7.22 (dd, 1 H), 7.08 (d, 1 H), 3.85 (s, 3 H), 3.84 (s, 3 H), 3.58 (d, 2 H), 1.36 (s, 6 H) |
| 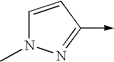 | 83 | ′1H NMR (300 MHz, DMSO-d6) ppm 10.02 (s, 1 H), 7.74 (d, 1 H), 7.71 (m, 2 H), 7.62 (dd, 1 H), 7.54 (d, 1 H), 7.39 (m, 2 H), 7.31 (t, 1 H), 7.08 (d, 1 H), 6.59 (d, 1 H), 3.86 (s, 3 H), 3.85 (s, 3 H), 3.84 (s, 3 H), 3.47 (d, 2 H), 1.28 (s, 6 H) |
| 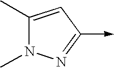 | 85 | 1H NMR (300 MHz, DMSO-d6) ppm 10.01 (s, 1 H), 7.70 (m, 2 H), 7.62 (dd, 1 H), 7.53 (d, 1 H), 7.37 (m, 2 H), 7.21 (t, 1 H), 7.08 (d, 1 H), 6.38 (d, 1 H), 3.84 (d, 6 H), 3.72 (s, 3 H), 3.45 (d, 2 H), 2.24 (s, 3 H), 1.26 (s, 6 H) |
| 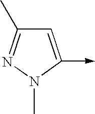 | 86 | ′1H NMR (300 MHz, DMSO-d6) ppm 9.99 (s, 1 H), 8.09 (t, 1 H), 7.69 (m, 2 H), 7.61 (dd, 1 H), 7.53 (d, 1 H), 7.37 (m, 2 H), 7.07 (d, 1 H), 6.54 (s, 1 H), 3.88 (s, 3 H), 3.84 (s, 3 H), 3.84 (s, 3 H), 3.36-3.42 (m, 2 H), 2.13 (s, 3 H), 1.28 (s, 6 H) |
| 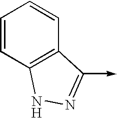 | 87 | 1H NMR (300 MHz, DMSO-d6) d ppm 13.47 (s, 1 H), 10.02 (s, 1 H), 8.14 (d, 1 H), 7.67- 7.81 (m, 2 H), 7.49-7.67 (m, 4 H), 7.33-7.49 (m, 3 H), 7.23 (ddd, 1 H), 7.08 (d, 1 H), 3.85 (s, 3 H), 3.84 (s, 3 H), 3.56 (d, 2 H), 1.33 (s, 6 H) |
| 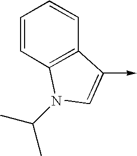 | 105 | ′1H NMR (300 MHz, DMSO-d6) ppm 10.00 (s, 1 H), 8.19 (s, 1 H), 8.04 (dt, 1 H), 7.71 (m, 2 H), 7.62 (dd, 1 H), 7.48-7.58 (m, 3 H), 7.42 (m, 2 H), 7.04-7.21 (m, 3 H), 4.77 (spt, 1 H), 3.84 (s, 3 H), 3.84 (s, 3 H), 3.48 (d, 2 H), 1.48 (d, 6 H), 1.33 (s, 6 H) |
| 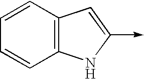 | 106 | ′1H NMR (300 MHz, DMSO-d6) ppm 11.49 (br. s., 1 H), 9.99 (br. s., 1 H), 8.14 (t, 1 H), 7.70 (m, 2 H), 7.57-7.65 (m, 2 H), 7.53 (d, 1 H), 7.30-7.48 (m, 3 H), 7.11-7.21 (m, 2 H), 7.07 (d, 1 H), 7.02 (ddd, 1 H), 3.84 (s, 3 H), 3.83 (s, 3 H), 3.50 (d, 2 H), 1.33 (s, 6 H) |
| 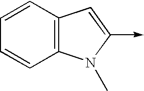 | 108 | ′1H NMR (300 MHz, DMSO-d6) ppm 9.99 (s, 1 H), 8.23 (t, 1 H), 7.71 (m, 2 H), 7.58- 7.67 (m, 2 H), 7.53 (d, 1 H), 7.50 (dd, 1 H), 7.41 (m, 2 H), 7.26 (ddd, 1 H), 7.04-7.15 (m, 2 H), 7.00 (s, 1 H), 3.90 (s, 3 H), 3.85 (s, 3 H), 3.84 (s, 3 H), 3.47 (d, 2 H), 1.34 (s, 6 H) |
| 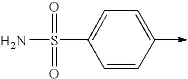 | 115 | ′1H NMR (300 MHz, DMSO-d6) ppm 9.99 (s, 1 H), 8.38 (t, 1 H), 7.81-7.99 (m, 4 H), 7.69 (m, 2 H), 7.61 (dd, 1 H), 7.53 (d, 1 H), 7.36-7.44 (m, 4 H), 7.07 (d, 1 H), 3.84 (s, 3 H), 3.84 (s, 3 H), 3.47 (d, 2 H), 1.32 (s, 6 H) |
| 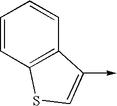 | 116 | ′1H NMR (300 MHz, DMSO-d6) d ppm 9.99 (s, 1 H), 8.21-8.27 (m, 2 H), 8.19 (t, 1 H), 7.90-8.11 (m, 1 H), 7.71 (m, 2 H), 7.62 (dd, 1 H), 7:53 (d, 1 H), 7.28-7.50 (m, 4 H), 7.07 (d, 1 H), 3.84 (s, 3 H), 3.84 (s, 3 H), 3.50 (d, 2 H), 1.35 (s, 6 H) |
| 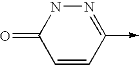 | 117 | ′1H NMR (300 MHz, DMSO-d6) d ppm 13.29 (br. s., 1 H), 10.01 (s, 1 H), 7.81 (d, 1 H), 7.66-7.76 (m, 3 H), 7.62 (dd, 1 H), 7.53 (d, 1 H), 7.38 (m, 2 H), 7.08 (d, 1 H), 6.94 (d, 1 H), 3.85 (s, 3 H), 3.84 (s, 3 H), 3.49 (d, 2 H), 1.28 (s, 6 H) |
| 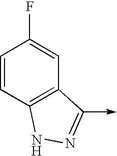 | 119 | ′1H NMR (300 MHz, DMSO-d6) d ppm 13.65 (br. s., 1 H), 10.01 (s, 1 H), 7.77 (dd, 1 H), 7.72 (m, 2 H), 7.58-7.69 (m, 3 H), 7.54 (d, 1 H), 7.42 (m, 2 H), 7.31 (td, 1 H), 7.08 (d, 1 H), 3.85 (s, 3 H), 3.84 (s, 3 H), 3.55 (d, 2 H), 1.33 (s, 6 H) |
| 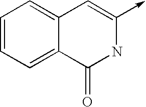 | 124 | ′1H NMR (300 MHz, DMSO-d6) d ppm 10.73 (s, 1 H), 10.00 (s, 1 H), 8.52 (t, 1 H), 8.22 (d, 1 H), 7.66-7.81 (m, 4 H), 7.56-7.65 (m, 2 H), 7.53 (d, 1 H), 7.40 (d, 2 H), 7.27 (s, 1 H), 7.07 (d, 1 H), 3.84 (s, 3 H), 3.83 (s, 3 H), 3.49 (d, 2 H), 1.33 (s, 6 H) |
| 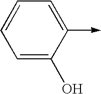 | 129 | ′1H NMR (300 MHz, DMSO-d6 353 K) d ppm 11.70 (br. s., 1 H), 9.76 (s, 1 H), 8.22 (t, 1 H), 7.77-7.90 (m, 1 H), 7.66-7.77 (m, 2 H), 7.61 (dd, 1 H), 7.56 (d, 1 H), 7.37-7.43 (m, 2 H), 7.34 (ddd, 1 H), 7.07 (d, 1 H), 6.78-6.96 (m, 2 H), 3.86 (s, 3 H), 3.86 (s, 3 H), 3.57 (d, 2 H), 1.35 (s, 6 H) |
| 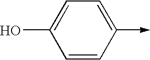 | 130 | ′1H NMR (300 MHz, DMSO-d6 353 K) d ppm 9.75 (s, 1 H), 7.66-7.71 (m, 2 H), 7.55- 7.66 (m, 4 H), 7.44 (td, 1 H), 7.35-7.41 (m, 2 H), 7.07 (d, 1 H), 6.70-6.83 (m, 2 H), 3.86 (s, 3 H), 3.86 (s, 3 H), 3.49 (d, 2 H), 1.32 (s, 6 H) |
| 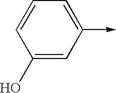 | 134 | ′1H NMR (300 MHz, CHLOROFORM-d 328 K) d ppm 8.06 (br. s., 1 H), 7.61 (m, 2 H), 7.52 (d, 1 H), 7.46 (dd, 1 H), 7.41 (m, 2 H), 7.19 (t, 1 H), 7.03-7.13 (m, 1 H), 6.95-6.99 (m, 1 H), 6.90-6.95 (m, 2 H), 5.68 (t, 1 H), 3.96 (s, 3 H), 3.94 (s, 3 H), 3.62 (d, 2 H), 1.42 (S, 6 H) |
| 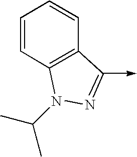 | 135 | ′1H NMR (300 MHz, DMSO-d6) d ppm 10.02 (s, 1 H), 8.06-8.19 (m, 1 H), 7.68-7.83 (m, 3 H), 7.63 (dd, 1 H), 7.54 (d, 1 H), 7.38-7.50 (m, 4 H), 7.26 (ddd, 1 H), 7.08 (d, 1 H), 4.91- 5.15 (m, 1 H), 3.85 (s, 3 H), 3.84 (s, 3 H), 3.55 (d, 2 H), 1.50 (d, 6 H), 1.34 (s, 6 H) |
| 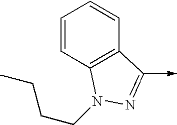 | 136 | ′1H NMR (300 MHz, DMSO-d6) d ppm 10.01 (s, 1 H), 8.14 (dt, 1 H), 7.69-7.79 (m, 3 H), 7.62 (dd, 1 H), 7.49-7.56 (m, 2 H), 7.38-7.48 (m, 3 H), 7.25 (ddd, 1 H), 7.08 (d, 1 H), 4.44 (t, 2 H), 3.85 (s, 3 H), 3.84 (s, 3 H), 3.55 (d, 2 H), 1.70-1.91 (m, 2 H), 1.33 (s, 6 H), 1.14-1.31 (m, 2 H), 0.87 (t, 3 H) |
| 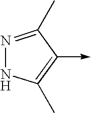 | 137 | ′1H NMR (300 MHz, CHLOROFORM-d) d ppm 8.41 (s, 1 H), 7.66 (m, 2 H), 7.42-7.56 (m, 2 H), 7.36 (m, 2 H), 6.90 (d, 1 H), 5.30-5.41 (m, 1 H), 3.94 (s, 3 H), 3.93 (s, 3 H), 3.61 (d, 2 H), 2.26 (s, 6 H), 1.38 (s, 6 H) |
| 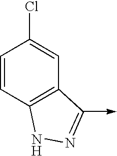 | 138 | ′1H NMR (300 MHz, CHLOROFORM-d) d ppm 8.49 (s, 1 H), 8.29-8.37 (m, 1 H), 7.60- 7.71 (m, 2 H), 7.52 (d, 1 H), 7.48 (dd, 1 H), 7.37-7.45 (m, 3 H), 7.26-7.31 (m, 1 H), 6.91 (d, 1 H), 6.79-6.88 (m, 1 H), 3.95 (s, 3 H), 3.93 (s, 3 H), 3.67 (d, 2 H), 1.41 (s, 6 H) |
| 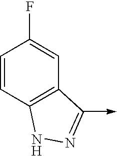 | 139 | ′1H NMR (300 MHz, DMSO-d6) d ppm 11.60 (d, 1 H), 9.99 (s, 1 H), 8.10 (d, 1 H), 7.66- 7.80 (m, 3 H), 7.61 (dd, 1 H), 7.60 (t, 1 H), 7.53 (d, 1 H), 7.31-7.48 (m, 3 H), 7.07 (d, 1 H), 6.98 (ddd, 1 H), 3.84 (s, 3 H), 3.84 (s, 3 H), 3.47 (d, 2 H), 1.32 (s, 6 H) |
| 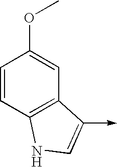 | 140 | ′1H NMR (300 MHz, DMSO-d6) d ppm 11.36 (d, 1 H), 9.99 (s, 1 H), 7.96 (d, 1 H), 7.71 (m, 2 H), 7.62 (dd, 1 H), 7.52-7.57 (m, 2 H), 7.47 (t, 1 H), 7.41 (m, 2 H), 7.29 (d, 1 H), 7.07 (d, 1 H), 6.76 (dd, 1 H), 3.84 (s, 3 H), 3.84 (s, 3 H), 3.74 (s, 3 H), 3.47 (d, 2 H), 1.32 (s, 6H) |
| 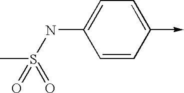 | 141 | ′1H NMR (300 MHz, DMSO-d6) d ppm 10.02 (s, 1 H), 9.99 (s, 1 H), 8.06 (t, 1 H), 7.76 (m, 2 H), 7.69 (m, 2 H), 7.61 (dd, 1 H), 7.53 (d, 1 H), 7.38 (m, 2 H), 7.22 (m, 2 H), 7.07 (d, 1 H), 3.84 (s, 3 H), 3.84 (s, 3 H), 3.45 (d, 2 H), 3.04 (s, 3 H), 1.30 (s, 6 H) |
| 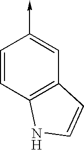 | 142 | 1H NMR (300 MHz, DMSO-d6) d ppm 11.25 (br. s., 1 H), 9.99 (s, 1 H), 8.05 (d, 1 H), 7.96 (t, 1 H), 7.70 (m, 2 H), 7.61 (dd, 1 H), 7.56 (dd, 1 H), 7.53 (d, 1 H), 7.31-7.47 (m, 4 H), 7.07 (d, 1 H), 6.51 (td, 1 H), 3.84 (s, 3 H), 3.83 (s, 3 H), 3.49 (d, 2 H), 1.32 (s, 6 H) |
| 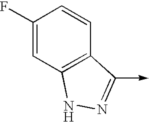 | 143 | ′1H NMR (300 MHz, DMSO-d6) d ppm 11.37 (br. s, 1 H), 10.01 (s, 1 H), 8.14 (ddd, 1 H), 7.72 (m, 2 H), 7.57-7.67 (m, 2 H), 7.53 (d, 1 H), 7.42 (m, 2 H), 7.39 (dd, 1 H), 7.12 (ddd, 1 H), 7.08 (d, 1 H), 3.84 (s, 3 H), 3.84 (s, 3 H), 3.55 (d, 2 H), 1.33 (s, 6 H) |
| 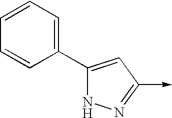 | 144 | ′1H NMR (300 MHz, DMSO-d6) d ppm 13.57 (br. s., 1 H), 10.01 (s, 1 H), 7.67-7.84 (m, 4 H), 7.62 (dd, 1 H), 7.53 (d, 1 H), 7.29-7.51 (m, 6 H), 7.11 (br. s., 1 H), 7.08 (d, 1 H), 3.84 (s, 3 H), 3.84 (s, 3 H), 3.50 (d, 2 H), 1.31 (s, 6 H) |
| 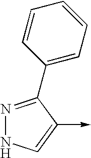 | 145 | ′1H NMR (300 MHz, DMSO-d6) d ppm 13.57 (br. s., 1 H), 10.01 (s, 1 H), 7.68-7.83 (m, 4 H), 7.62 (dd, 1 H), 7.53 (d, 1 H), 7.29-7.50 (m, 6 H), 7.11 (br. s., 1 H), 7.08 (d, 1 H), 3.84 (s, 3 H), 3.84 (s, 3 H), 3.50 (d, 2 H), 1.31 (s, 6 H) |
| 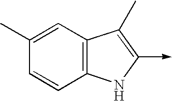 | 146 | ′1H NMR (300 MHz, DMSO-d6) d ppm 11.03 (s, 1 H), 10.00 (s, 1 H), 7.67-7.77 (m, 2 H), 7.62 (dd, 1 H), 7.53 (d, 1 H), 7.42 (d, 2 H), 7.32-7.38 (m, 1 H), 7.31-7.34 (m, 1 H), 7.25 (d, 1 H), 7.07 (d, 1 H), 7.01 (dd, 1 H), 3.84 (s, 3 H), 3.84 (s, 3 H), 3.54 (d, 2 H), 2.37 (s, 3 H), 2.36 (s, 3 H), 1.34 (s, 6 H) |
| 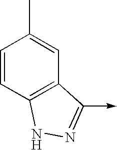 | 171 | 1H NMR (300 MHz, DMSO-d6) d ppm 13.34 (br. s., 1 H), 10.01 (s, 1 H), 7.92 (s, 1 H), 7.72 (m, 2 H), 7.62 (dd, 1 H), 7.46-7.56 (m, 3 H), 7.42 (m, 2 H), 7.23 (dd, 1 H), 7.08 (d, 1 H), 3.85 (s, 3 H), 3.84 (s, 3 H), 3.56 (d, 2 H), 2.42 (s, 3 H), 1.33 (s, 6 H) |
| 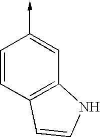 | 172 | 1H NMR (300 MHz, DMSO-d6) d ppm 11.30 (br. s., 1 H), 9.99 (s, 1 H), 8.02 (t, 1 H), 7.82- 7.90 (m, 1 H), 7.70 (m, 2 H), 7.50-7.65 (m, 3 H), 7.43-7.49 (m, 2 H), 7.41 (m, 2 H), 7.07 (d, 1 H), 6.47 (ddd, 1 H), 3.84 (s, 3 H), 3.83 (s, 3 H), 3.49 (d, 2 H), 1.32 (s, 6 H) |
| 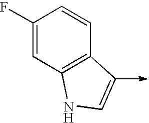 | 175 | 1H NMR (300 MHz, CHLOROFORM-d) d ppm 8.54 (br. s., 1 H), 7.82 (s, 1 H), 7.67 (m, 2 H), 7.57 (dd, 1 H), 7.52-7.55 (m, 2 H), 7.46 (m, 2 H), 7.43 (dd, 1 H), 7.07 (dd, 1 H), 6.87- 7.01 (m, 2 H), 5.58 (t, 1 H), 3.99 (s, 3 H), 3.98 (s, 3 H), 3.72 (d, 2 H), 1.46 (s, 6 H) |
| 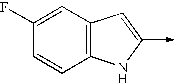 | 176 | 1H NMR (300 MHz, DMSO-d6) d ppm 11.61 (d, 1 H), 9.99 (s, 1 H), 8.20 (t, 1 H), 7.70 (m, 2 H), 7.61 (dd, 1 H), 7.53 (d, 1 H), 7.31-7.46 (m, 4 H), 7.13 (d, 1 H), 7.07 (d, 1 H), 7.02 (td, 1 H), 3.84 (s, 3 H), 3.84 (s, 3 H), 3.50 (d, 2 H), 1.32 (s, 6 H) |
| 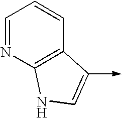 | 178 | 1H NMR (300 MHz, DMSO-d6) d ppm 12.02 (d, 1 H), 9.99 (s, 1 H), 8.35 (dd, 1 H), 8.25 (dd, 1 H), 8.16 (d, 1 H), 7.71 (m, 2 H), 7.65-7.71 (m, 1 H), 7.62 (dd, 1 H), 7.53 (d, 1 H), 7.41 (m, 2 H), 7.15 (dd, 1 H), 7.08 (d, 1 H), 3.85 (s, 3 H), 3.84 (s, 3 H), 3.47 (d, 2 H), 1.32 (s, 6 H) |
| 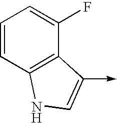 | 179 | 1H NMR (300 MHz, DMSO-d6) d ppm 11.89 (br. s., 1 H), 10.00 (s, 1 H), 7.88 (d, 1 H), 7.71 (m, 2 H), 7.62 (dd, 1 H), 7.53 (d, 1 H), 7.41 (m, 2 H), 7.22-7.36 (m, 2 H), 7.13 (td, 1 H), 7.08 (d, 1 H), 6.87 (dd, 1 H), 3.84 (s, 3 H), 3.84 (s, 3 H), 3.54 (d, 2 H), 1.34 (s, 6 H) |
| 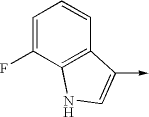 | 180 | 1H NMR (300 MHz, DMSO-d6) d ppm 12.00 (d, 1 H), 9.99 (s, 1 H), 8.09 (d, 1 H), 7.86 (d, 1 H), 7.71 (m, 2 H), 7.61-7.66 (m, 1 H), 7.62 (dd, 1 H), 7.53 (d, 1 H), 7.41 (m, 2 H), 6.88- 7.16 (m, 3 H), 3.85 (s, 3 H), 3.84 (s, 3 H), 3.48 (d, 2 H), 1.32 (s, 6 H) |
| 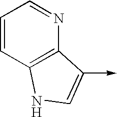 | 181 | 1H NMR (300 MHz, DMSO-d6) d ppm 11.86 (br. s., 1 H), 10.00 (s, 1 H), 8.84 (t, 1 H), 8.34 (dd, 1 H), 8.11 (d, 1 H), 7.88 (dd, 1 H), 7.71 (m, 2 H), 7.63 (dd, 1 H), 7.54 (d, 1 H), 7.44 (m, 2 H), 7.20 (dd, 1 H), 7.08 (d, 1 H), 3.85 (s, 3 H), 3.84 (s, 3 H), 3.60 (d, 2 H), 1.37 (s, 6 H) |
| 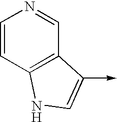 | 182 | 1H NMR (300 MHz, Me0D) d ppm 9.19 (d, 1 H), 8.25 (d, 1 H), 8.00 (s, 1 H), 7.66 (m, 2 H), 7.62 (dd, 1 H), 7.54-7.59 (m, 2 H), 7.51 (m, 2 H), 7.08 (d, 1 H), 3.93 (s, 3 H), 3.93 (s, 3 H), 3.65 (s, 2 H), 1.45 (s, 6 H) |
| 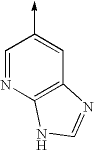 | 189 | 1H NMR (300 MHz, DMSO-d6) ppm 13.04 (br. s., 1 H), 9.99 (s, 1 H), 8.80 (s, 1 H), 8.28- 8.61 (m, 3 H), 7.71 (m, 2 H), 7.62 (dd, 1 H), 7.53 (d, 1 H), 7.39 (m, 2 H), 7.08 (d, 1 H), 3.85 (s, 3 H), 3.84 (s, 3 H), 3.03-3.16 (m, 2 H), 1.86-1.99 (m, 2 H), 1.34 (s, 6 H) |
| 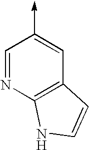 | 202 | 1H NMR (300 MHz, DMSO-d6) ppm 11.83 (br. s., 1 H), 9.99 (s, 1 H), 8.64 (d, 1 H), 8.36 (d, 1 H), 8.21 (t, 1 H), 7.70 (m, 2 H), 7.61 (dd, 1 H), 7.50-7.56 (m, 2 H), 7.41 (m, 2 H), 7.07 (d, 1 H), 6.54 (ddd, 1 H), 3.84 (s, 3 H), 3.84 (s, 3 H), 3.50 (d, 2 H), 1.33 (s, 6 H) |
| 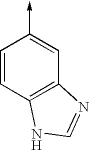 | 207 | 1H NMR (300 MHz, DMSO-d6) d ppm 10.00 (s, 1 H), 8.27 (s, 1 H), 8.01-8.19 (m, 2 H), 7.49-7.76 (m, 6 H), 7.41 (m, 2 H), 7.08 (d, 1 H), 3.84 (s, 3 H), 3.84 (s, 3 H), 3.49 (d, 2 H), 1.33 (s, 6 H) |
| 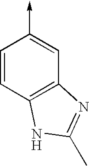 | 208 | 1H NMR (300 MHz, DMSO-d6) d ppm 12.33 (br. s., 1 H), 9.99 (s, 1 H), 8.07 (t, 1 H), 7.91 (s, 1 H), 7.70 (m, 2 H), 7.56-7.65 (m, 2 H), 7.53 (d, 1 H), 7.44 (d, 1 H), 7.41 (m, 2 H), 7.08 (d, 1 H), 3.85 (s, 3 H), 3.84 (s, 3 H), 3.48 (d, 2 H), 2.54 (s, 3 H), 1.32 (s, 6 H) |
| 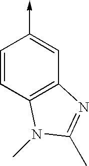 | 209 | 1H NMR (300 MHz, DMSO-d6) d ppm 9.99 (s, 1 H), 8.09 (t, 1 H), 8.00 (d, 1 H), 7.71 (m, 2 H), 7.68 (dd, 1 H), 7.62 (dd, 1 H), 7.53 (d, 1 H), 7.49 (d, 1 H), 7.41 (m, 2 H), 7.08 (d, 1 H), 3.85 (s, 3 H), 3.84 (s, 3 H), 3.75 (s, 3 H), 3.49 (d, 2 H), 2.54 (s, 3 H), 1.33 (s, 6 H) |
| 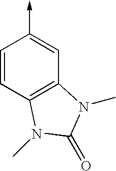 | 210 | 1H NMR (300 MHz, DMSO-d6) d ppm 9.99 (s, 1 H), 8.06 (t, 1 H), 7.70 (m, 2 H), 7.56- 7.66 (m, 3 H), 7.53 (d, 1 H), 7.40 (m, 2 H), 7.18 (d, 1 H), 7.08 (d, 1 H), 3.85 (s, 3 H), 3.84 (s, 3 H), 3.48 (d, 2 H), 3.36 (s, 3 H), 3.35 (s, 3 H), 1.32 (s, 6 H) |
| 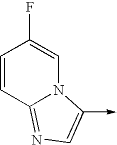 | 211 | 1H NMR (300 MHz, DMSO-d6) ppm 9.99 (s, 1 H), 9.35-9.45 (m, 1 H), 8.40 (s, 1 H), 8.25-8.37 (m, 1 H), 7.74-7.81 (m, 1 H), 7.70 (m, 2 H), 7.60 (td, 1 H), 7.49-7.56 (m, 2 H), 7.41 (m, 2 H), 7.07 (d, 1 H), 3.84 (d, 6 H), 3.49 (d, 2 H), 1.33 (s, 6 H) |
| 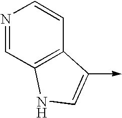 | 215 | 1H NMR (300 MHz, DMSO-d6) ppm 11.97 (br. s., 1 H), 9.99 (s, 1 H), 8.77 (d, 1 H), 8.11- 8.26 (m, 2 H), 7.90 (dd, 1 H), 7.65-7.75 (m, 3 H), 7.62 (dd, 1 H), 7.53 (d, 1 H), 7.41 (m, 2 H), 7.07 (d, 1 H), 3.84 (s, 3 H), 3.84 (s, 3 H), 3.48 (d, 2 H), 1.32 (s, 6 H) |
| 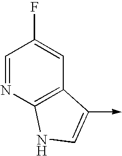 | 216 | 1H NMR (300 MHz, DMSO-d6) d ppm 12.21 (br. s., 1 H), 9.99 (s, 1 H), 8.28 (d, 1 H), 8.25 (dd, 1 H), 8.10 (dd, 1 H), 7.77 (t, 1 H), 7.70 (m, 2 H), 7.61 (dd, 1 H), 7.53 (d, 1 H), 7.41 (m, 2 H), 7.07 (d, 1 H), 3.84 (s, 3 H), 3.84 (s, 3 H), 3.47 (d, 2 H), 1.32 (s, 6 H) |
| 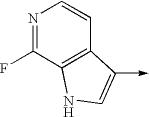 | 217^{h} | 1H NMR (300 MHz, DMSO-d6) ppm 12.54 (br. s., 1 H), 9.99 (s, 1 H), 8.30 (s, 1 H), 7.87 (dd, 1 H), 7.80 (t, 1 H), 7.66-7.76 (m, 3 H), 7.62 (dd, 1 H), 7.53 (d, 1 H), 7.41 (m, 2 H), 7.07 (d, 1 H), 3.84 (s, 3 H), 3.84 (s, 3 H), 3.48 (d, 2 H), 1.32 (s, 6 H) |
| 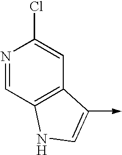 | 218ⁱ | 1H NMR (300 MHz, DMSO-d6) ppm 12.16 (s, 1 H), 9.99 (s, 1 H), 8.60 (d, 1 H), 8.34 (s, 1 H), 7.93 (d, 1 H), 7.80 (t, 1 H), 7.71 (m, 2 H), 7.62 (dd, 1 H), 7.53 (d, 1 H), 7.41 (m, 2 H), 7.08 (d, 1 H), 3.85 (s, 3 H), 3.84 (s, 3 H), 3.48 (d, 2 H), 1.32 (s, 6 H) |
| 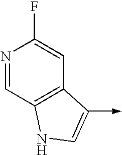 | 219ⁱ | 1H NMR (300 MHz, DMSO-d6) ppm 12.08 (br. s., 1 H), 9.99 (s, 1 H), 8.40 (t, 1 H), 8.36 (s, 1 H), 7.74-7.82 (m, 1 H), 7.70 (m, 2 H), 7.61 (dd, 1 H), 7.53 (d, 1 H), 7.49 (dd, 1 H), 7.41 (m, 2 H), 7.07 (d, 1 H), 3.84 (s, 6 H), 3.47 (d, 2 H), 1.32 (s, 6 H) |

The compounds reported in Table 6 were prepared following the synthetic procedure described for Example 112, using the suitable carboxylic acids.

TABLE 6
amide derivatives prepared according to Example 112.

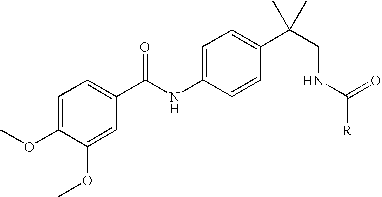

| | | | | LCMS | | | | |
|---|---|---|---|---|---|---|---|---|
| | | | MP | | | RT | | |
| R | Example | Yield^{a} | (° C.) | Met | [MH+] | (min) | Appearance | Purification |
| 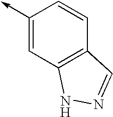 | 123^{d} | 15 | | G | 473.24 | 1.96 | White solid | Chromatography [SiO₂, EtOAc] |
| 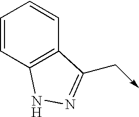 | 126 | 99 | 94- 96 | G | 486.23 | 2.16 | White solid | Not required |
| 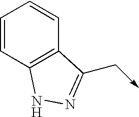 | 127 | 44 | | G | 437.28 | 1.88 | White amorphous solid | Chromatography [SiO₂, DCM/MeOH (97/3)] |
| 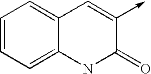 | 128 | 29 | 260- 262 | G | 500.24 | 2.12 | White solid | Trituration with MeOH |
| 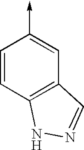 | 147^{d} | 38 | | G | 473.24 | 1.92 | Light yellow solid | Trituration with iPr₂O Followed by Chromatography [SiO₂, DCM/MeOH (95/5)] |
| 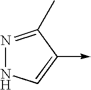 | 148 | 58 | | G | 437.15 | 1.73 | White solid | Chromatography [SiO₂, EtOAc/MeOH (98/2)] |
| 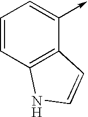 | 149 | 51 | | G | 472.16 | 2.13 | White solid | Chromatography [SiO₂, DCM/MeOH (98/2)] |
| 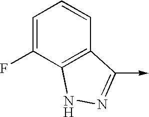 | 150^{c} | 8 | | G | 491.15 | 2.13 | Yellow sticky solid | Chromatography [SiO₂, DCM/MeOH (98/2)] |
| 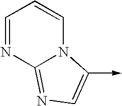 | 185^{e} | 26 | | M | 474.20 | 1.68 | White solid | Preparative HPLC (Method Q) |
| 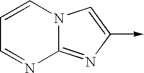 | 186^{e} | 8 | | N | 474.18 | 2.61 | White solid | Preparative HPLC (Method Q) |
| 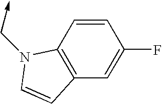 | 187 | 43 | | M | 504.23 | 2.13 | White solid | Crystallization from MeOH |
| 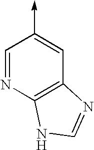 | 189 | 25 | | M | 474.20 | 1.61 | White solid | Preparative HPLC (Method Q) |
| 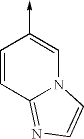 | 190 | 38 | 194- 196 | M | 473.19 | 1.64 | Off-white solid | Crystallization from EtOH/MeOH (9/1) |
| 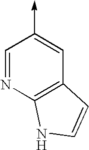 | 202 | 61 | | M | 473.12 | 1.80 | White solid | Crystallization from MeOH |

^{a}isolated yield of analytically pure product
^{b}eluted with DCM-MeOH (1:1) to MeOH-NH4OH (9:1)
^{c}The needed carboxylic acid was prepared according to the procedure reported in J. Med. Chem, (2000), 43/1 pg 41.
^{d}The needed carboxylic acid was prepared according to the procedure reported in Chem. Pharm. Bull., (1995), 43, 11, 1912-1930.
^{e}The needed carboxylic acid was prepared according to the procedure reported in Eur. J. Med. Chem. (1991), 26 pg 13.

TABLE 7
NMR data of the compounds reported in Table 6.

| R | Example | NMR-data |
|---|---|---|
| 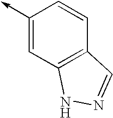 | 123 | 1H NMR (300 MHz, DMSO-d6) d ppm 13.28 (s, 1 H), 10.00 (s, 1 H), 8.30 (t, 1 H), 8.12 (s, 1 H), 7.95 (s, 1 H), 7.79 (d, 1 H), 7.70 (d, 2 H), 7.61 (dd, 1 H), 7.48-7.57 (m, 2 H), 7.41 (d, 2 H), 7.07 (d, 1 H), 3.84 (s, 3 H), 3.83 (s, 3 H), 3.50 (d, 2 H), 1.33 (s, 6 H) |
| 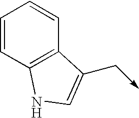 | 126 | 1H NMR (300 MHz, DMSO-d6 353 K) d ppm 10.56 (br. s., 1 H), 9.73 (s, 1 H), 7.59-7.69 (m, 3 H), 7.57 (d, 1 H), 7.45-7.53 (m, 1 H), 7.34 (dt, 1 H), 7.16-7.29 (m, 2 H), 7.02-7.14 (m, 3 H), 6.90-6.98 (m, 1 H), 6.97 (ddd, 1 H), 3.87 (s, 3 H), 3.86 (s, 3 H), 3.53 (d, 2 H), 3.30 (d, 2 H), 1.20 (s, 6 H) |
| 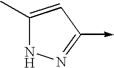 | 127 | 1H NMR (300 MHz, DMSO-d6) d ppm 12.80 (br. s., 1 H), 10.01 (s, 1 H), 7.70 (m, 2 H), 7.62 (dd, 1 H), 7.53 (d, 1 H), 7.38 (m, 2 H), 7.18 (br. s., 1 H), 7.08 (d, 1 H), 6.34 (br. s., 1 H), 3.85 (s, 3 H), 3.84 (s, 3 H), 3.45 (d, 2 H), 2.22 (s, 3 H), 1.28 (s, 6 H) |
| 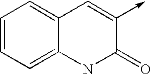 | 128 | 1H NMR (300 MHz, DMSO-d6) d ppm 12.33 (br. s., 1 H), 10.00 (br. s., 1 H), 9.82 (t, 1 H), 8.81 (s, 1 H), 7.92 (dd, 1 H), 7.71 (d, 2 H), 7.58-7.67 (m, 2 H), 7.53 (d, 1 H), 7.34-7.46 (m, 3 H), 7.21-7.33 (m, 1 H), 7.07 (d, 1 H), 3.84 (s, 3 H), 3.84 (s, 3 H), 3.59 (d, 2 H), 1.34 (s, 6 H) |
| 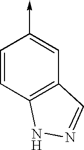 | 147 | 1H NMR (300 MHz, DMSO-d6) d ppm 13.22 (br. s., 1 H), 10.00 (s, 1 H), 8.08-8.35 (m, 3 H), 7.78 (dd, 1 H), 7.70 (m, 2 H), 7.61 (dd, 1 H), 7.49-7.58 (m, 2 H), 7.41 (m, 2 H), 7.07 (d, 1 H), 3.84 (s, 3 H), 3.83 (s, 3 H), 3.49 (d, 2 H), 1.33 (s, 6 H) |
| 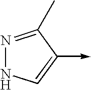 | 148 | 1H NMR (300 MHz, DMSO-d6) d ppm 12.58-12.81 (m, 1 H), 9.99 (s, 1 H), 7.81 (br. s., 1 H), 7.69 (m, 2 H), 7.62 (dd, 1 H), 7.43-7.56 (m, 2 H), 7.37 (m, 2 H), 7.07 (d, 1 H), 3.84 (s, 3 H), 3.84 (s, 3 H), 3.38 (d, 2 H), 2.14-2.44 (m, 3 H), 1.27 (s, 6 H) |
| 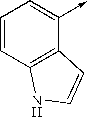 | 149 | 1H NMR (300 MHz, DMSO-d6) d ppm 11.23 (br. s., 1 H), 10.00 (s, 1 H), 7.68-7.78 (m, 3 H), 7.62 (dd, 1 H), 7.53 (d, 1 H), 7.50 (dt, 1 H), 7.43 (m, 2 H), 7.38 (t, 1 H), 7.31 (dd, 1 H), 7.03-7.16 (m, 2 H), 6.55-6.65 (m, 1 H), 3.84 (s, 3 H), 3.84 (s, 3 H), 3.53 (d, 2 H), 1.35 (s, 6 H) |
| 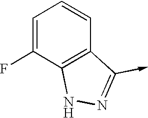 | 150 | 1H NMR (300 MHz, METHANOL-d4) d ppm 7.98 (d, 1 H), 7.63-7.71 (m, 2 H), 7.60 (dd, 1 H), 7.56 (d, 1 H), 7.46-7.53 (m, 2 H), 7.16 -.7.24 (m, 1 H), 7.13 (ddd, 1 H), 7.06 (d, 1 H), 3.91 (s, 6 H), 3.67 (s, 2 H), 1.44 (s, 6 H) |
| 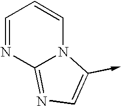 | 185 | 1H NMR (300 MHz, DMSO-d6) d ppm 9.99 (s, 1 H), 9.68 (dd, 1 H), 8.68 (dd, 1 H), 8.50 (s, 1 H), 8.38 (t, 1 H), 7.70 (m, 2 H), 7.61 (dd, 1 H), 7.53 (d, 1 H), 7.41 (m, 2 H), 7.24 (dd, 1 H), 7.07 (d, 1 H), 3.84 (s, 3 H), 3.84 (s, 3 H), 3.49 (d, 2 H), 1.34 (s, 6 H) |
| 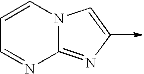 | 186 | 1H NMR (300 MHz, DMSO-d6) d ppm 10.02 (s, 1 H), 8.97 (dd, 1 H), 8.62 (dd, 1 H), 8.29 (s, 1 H), 7.76 (t, 1 H), 7.72 (d, 2 H), 7.62 (dd, 1 H), 7.54 (d, 1 H), 7.42 (d, 2 H), 7.12 (dd, 1 H), 7.08 (d, 1 H), 3.85 (s, 3 H), 3.84 (s, 3 H), 3.55 (d, 2 H), 1.32 (s, 6 H) |
| 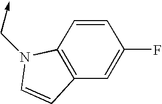 | 187 | 1H NMR (300 MHz, DMSO-d6) ppm 10.91 (br. s., 1 H) 9.99 (s, 1 H) 7.46-7.78 (m, 5 H) 7.24-7.36 (m, 4 H) 7.20 (d, 1 H) 7.08 (d, 1 H) 6.89 (td, 1 H) 3.85 (s, 3 H) 3.84 (s, 3 H) 3.48 (s, 2 H) 3.26 (d, 2 H) 1.18 (s, 6 H) |
| 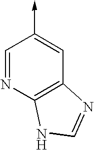 | 189 | 1H NMR (300 MHz, DMSO-d6) ppm 12.84 (br. s., 1 H) 9.99 (s, 1 H) 8.77 (d, 1 H) 8.53 (s, 1 H) 8.26-8.45 (m, 2 H) 7.70 (m, 2 H) 7.61 (dd, 1 H) 7.53 (d, 1 H) 7.41 (m, 2 H) 7.07 (d, 1 H) 3.84 (s, 3 H) 3.84 (s, 3 H) 3.51 (d, 2 H) 1.34 (s, 6 H) |
| 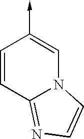 | 190 | 1H NMR (300 MHz, DMSO-d6) d ppm 10.24 (t, 1 H), 9.98 (s, 1 H), 8.73 (dd, 1 H), 8.08 (d, 1 H), 7.98 (dd, 1 H), 7.69 (m, 2 H), 7.63 (d, 1 H), 7.61 (dd, 1 H), 7.53 (d, 1 H), 7.43 (m, 2 H), 7.07 (d, 1 H), 7.05 (d, 1 H), 3.84 (s, 3 H), 3.84 (s, 3 H), 3.66 (d, 2 H), 1.39 (s, 6 H) |
| 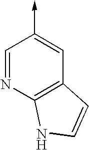 | 202 | 1H NMR (300 MHz, DMSO-d6) ppm 11.83 (br. s., 1 H) 9.99 (s, 1 H) 8.64 (d, 1 H) 8.36 (d, 1 H) 8.20 (t, 1 H) 7.70 (m, 2 H) 7.61 (dd, 1 H) 7.49-7.57 (m, 2 H) 7.41 (m, 2 H) 7.07 (d, 1 H) 6.54 (d, 1 H) 3.84 (s, 3 H) 3.84 (s, 3 H) 3.50 (d, 2 H) 1.34 (s, 6 H) |

### FORMULATION EXAMPLES

Typical examples of recipes for the formulation of the invention are as follows:

1) Tablets

| | | |
|---|---|---|
| Compounds of the general formula I | 5 to 50 | mg |
| Di-calcium phosphate | 20 | mg |
| Lactose | 30 | mg |
| Talcum | 10 | mg |
| Magnesium stearate | 5 | mg |
| Potato starch | ad 200 | mg |

2) Suspension

An aqueous suspension is prepared for oral administration so that each 1 milliliter contains 1 to 5 mg of one of the described example, 50 mg of sodium carboxymethyl cellulose, 1 mg of sodium benzoate, 500 mg of sorbitol and water ad 1 ml.

3) Injectable

A parenteral composition is prepared by stirring 1.5% by weight of active ingredient of the invention in 10% by volume propylene glycol and water.

4) Ointment

| | | |
|---|---|---|
| Compounds of the general formula I | 5 to 1000 | mg |
| Stearyl alcohol | 3 | g |
| Lanoline | 5 | g |
| White petroleum | 15 | g |
| Water | ad 100 | g |

Reasonable variations are not to be regarded as a departure from the scope of the invention. It will be obvious that the thus described invention may be varied in many ways by those skilled in the art.

1-33. (canceled)

34. A compound having the formula I-A 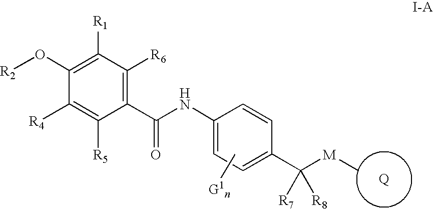
or a pharmaceutically acceptable salt, hydrate or solvate of such compound
wherein:
represents independently O—(C₁-C₆)alkyl, O—(C₂-C₆)alkynyl, O—(C₂-C₆)alkenyl, O—(C₃-C₇)cycloalkyl and O-alkylcycloalkyl;
R₂ represents independently hydrogen, OH, an optionally substituted (C₁-C₆)alkyl, (C₂-C₆)alkynyl, (C₂-C₆)alkenyl, (C₃-C₇)cycloalkyl, (C₄-C₁₀)alkylcycloalkyl, (C₁-C₆)heterocycloalkyl, (C₁-C₆)alkyl-heteroaryl, (C₁-C₆)alkyl-aryl or (C₁-C₆)alkyl-CN;
R₁ and R₂ according to the above definitions can be combined to form a heterocycloalkyl ring;
R₄ is independently selected from the group consisting of hydrogen, OH, (C₀-C₆)alkyl-CN, (C₁-C₆)alkyl, (C₀-C₆)alkylhalo, (C₃-C₆)cycloalkyl, (C₁-C₆)alkyl-(C₃-C₈)cycloalkyl, (C₂-C₆)alkenyl, (C₂-C₆)alkynyl, (C₀-C₆)alkyl-OR₉, (C₁-C₆)alkyl-NR_{g}R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O) R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀) R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-) cycloalkyl) or N((C₀-C₆)alkyl)(aryl) substituents;
R₅, R₆ are each independently selected from the group consisting of hydrogen, (C₀-C₆)alkyl-CN, (C₁-C₆)alkyl, (C₀-C₆)alkylhalo, (C₃-C₆)cycloalkyl, (C₁-C₆)alkyl-(C₃-C₈)cycloalkyl, (C₂-C₆)alkenyl, (C₂-C₆)alkynyl, (C₀-C₆)alkyl-OR₉, (C₁-C₆)alkyl-NR_{g}R₁₀, (C₀-C₆)-alkyl-NR₉C0R₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O) R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀) R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-) cycloalkyl) or N((C₀-C₆)alkyl)(aryl) substituents;
G¹ is independently selected from the group consisting of hydrogen, OH, (C₀-C₆)alkyl-CN, (C₁-C₆)alkyl, (C₀-C₆)alkylhalo, (C₃-C₆)cycloalkyl, (C₀-C₆)alkyl-(C₃-C₈)cycloalkyl, (C₂-C₆)alkenyl, (C₂-C₆)alkynyl, (C₀-C₆)alkyl-OR₉, (C₀-C₆)alkyl-NR₉R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O) R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀) R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-) cycloalkyl) or N((C₀-C₆)alkyl)(aryl) substituents, wherein optionally two substituents are combined to the intervening atoms to form a bicyclic heterocycloalkyl, aryl or heteroaryl ring; wherein each ring is optionally further substituted with 1-5 independent halogen, CN, (C₁-C₆)alkyl, O—(C₀-C₆)alkyl, O-alkylcycloalkyl, O(aryl), O(heteroaryl), O-arylalkyl, O-heteroarylalkyl, N((-C₀-C₆)alkyl)((C₀-C₃)arylalkyl) or N((C₀-C₆)alkyl)(heteroarylalkyl) groups;
R₉, R₁₀, R₁₁ each independently is hydrogen, (C₁-C₆)alkyl, (C₃-C₆)cycloalkyl, (C₁-C₆)alkyl-(C₃-C₈)cycloalkyl, (C₂-C₆)alkenyl, (C₂-C₆)alkynyl, (C₁-C₆)alkylhalo, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), N(C₀-C₆-alkyl)₂, —N((C₀-C₆)alkyl)((C₃-C₇-)cycloalkyl) or N((C₀-C₆)alkyl)(aryl) substituents;
n is an integer from 1 to 4, provided that when n>1, the G¹ groups may be the same or different from each other;
R₇ and R₈ represent independently an optionally substituted (C₁-C₄)alkyl, (C₁-C₆)alkylhalo, (C₀-C₆)alkyl-aryl, (C₁-C₆)alkyl-O—(C₀-C₆)-alkyl, (C₀-C₆)alkyl-heteroaryl, (C₀-C₆)alkyl-heterocycloalkyl, (C₀-C₆)alkyl-(C₃-C₇)cycloalkyl or R⁷ and R⁸ can together form a (C₃-C₆)cycloalkyl or an heterocycloalkyl group of formula: 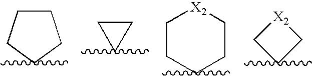
X₂ is independently selected from the group consisting of CH₂, O, S, SO₂;
M is independently selected from the group of consisting of a bond, an optionally substituted (C₁-C₆)alkyl, (C₂-C₆)alkynyl, (C₁-C₆)alkylhalo, (C₂-C₆)alkenyl, (C₁-C₆)alkyl-O—(C₀-C₆)alkyl, (C₀-C₆)alkyl-S(═O)₂NR₁₂—(C₀-C₆)alkyl, (C₀-C₆)alkyl-NR₁₂—S(═O)₂—(C₀-C₆)alkyl, (C₀-C₆)alkyl-C(═O)—NR₁₂-(C₀-C₆)alkyl, (C₀-C₆)alkyl-NR₁₂C(═O)—(C₀-C₆)alkyl, (C₀-C₆)alkyl-NR₁₂—C(═O)—O—(C₀-C₆)alkyl, (C₀-C₆)alkyl-O—C(═O)—NR₁₂—(C₀-C₆)alkyl or (C₀-C₆)alkyl-NR₁₂—C(═O)—NR₁₃—(C₀-C₆)alkyl substituents;
wherein optionally two substituents are combined with the intervening atoms to form a cycloalkyl or heterocycloalkyl ring;
R₁₂ and R₁₃ are each independently selected from the group consisting of hydrogen, an optionally substituted (C₁-C₆)alkyl;
Q represents independently H, an optionally substituted (C₁-C₆)alkyl, (C₀-C₆)alkyl-CN, (C₁-C₆)alkylhalo, (C₃-C₇)cycloalkyl, (C₃-C₇) heterocycloalkyl or one of the following aryl or heteroaryl: 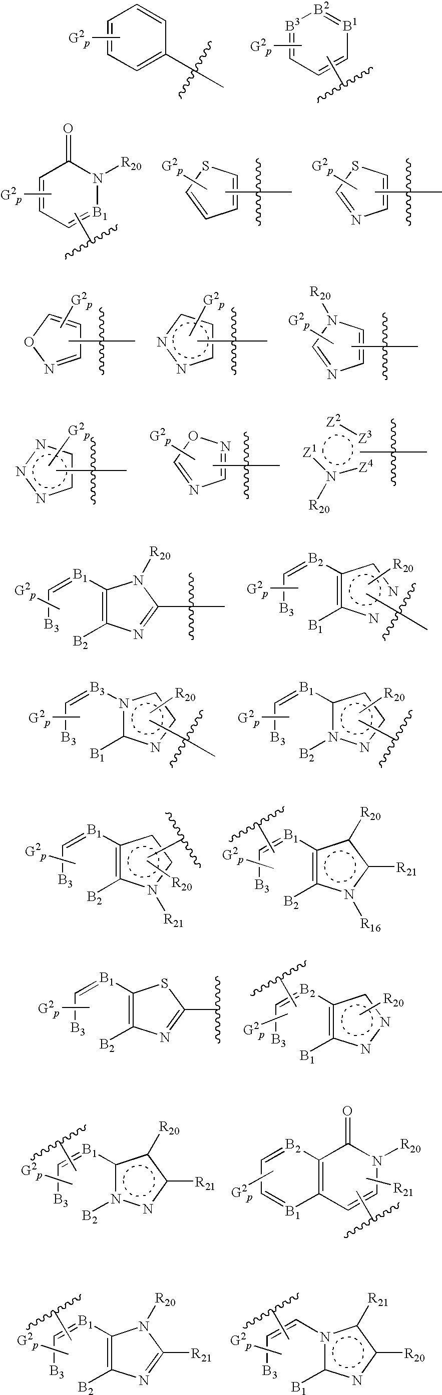
G² groups are each independently selected from the group consisting of hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₂-C₆)alkyl-OR₁₄, O—(C₃-C₇)cycloalkyl, O—(C₁-C₆)alkyl-heteroaryl, O—(C₁-C₆)alkyl-aryl, (C₀-C₆)alkyl-OR₁₄, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl, (C₁-C₆)alkyl-aryl, (C₁-C₆)alkylhalo-OR₁₇, (C₃-C₆)alkynyl-OR₁₇, (C₃-C₆)alkenyl-OR₁₇, (C₀-C₆)alkyl-S—R₁₇, O—(C₂-C₆)alkyl-S—R₁₇, (C₀-C₆)alkyl-S(═O)—R₁₇, O—(C₂-C₆)alkyl-S(═O)—R₁₇, (C₀-C₆)alkyl-S(═O)₂—R₁₇, O—(C₁-C₆)alkyl-S(═O)₂—R₁₇, (C₀-C₆)alkyl-NR₁₇R₁₈, O—(C₂-C₆)alkyl-NR₁₇R₁₈, (C₀-C₆)alkyl-S(═O)₂NR₁₇R₁₈, (C₀-C₆)alkyl-NR₁₇—S(═O)₂R₁₈, O—(C₁-C₆)alkyl-S(═O)₂NR₁₇R₁₈, O—(C₂-C₆)alkyl-NR₁₇—S(═O)₂R₁₈, (C₀-C₆)alkyl-C(═O)—NR₁₇R₁₈, (C₀-C₆)alkyl-NR₁₇C(═O)—R₁₈, O—(C₁-C₆)alkyl-C(═O)—NR₁₇R₁₈, O—(C₂-C₆)alkyl-NR₁₇C(═O)—R₁₈, (C₀-C₆)alkyl-OC(═O)—R₁₇, (C₀-C₆)alkyl-C(═O)—OR₁₇, O—(C₂-C₆)alkyl-OC(═O)—R₁₇, O—(C₁-C₆)alkyl-C(═O)—OR₁₇, (C₀-C₆)alkyl-C(═O)—R₁₇, O—(C₁-C₆)alkyl-C(═O)—R₁₇, (C₀-C₆)alkyl-NR₁₇—C(═O)—OR₁₈, (C₀-C₆)alkyl-O—C(═O)—NR₁₇R₁₈ or (C₀-C₆)alkyl-NR₁₇—C(═O)—NR₁₈R₁₉ substituents;
wherein optionally two substituents are combined to the intervening atoms to form a bicyclic aryl, cycloalkyl, heterocycloalkyl or heteroaryl ring; wherein each ring is optionally further substituted with 1-5 independent hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₃-C₇)cycloalkyl, O—(C₁-C₆)alkyl-heteroaryl, O—(C₁-C₆)alkylaryl, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl;
p is an integer that is selected from the group consisting of 1, 2, 3, 4 and 5 provided that when p>1, the G² groups may be the same or different from each other;
R₁₆, R₁₇, R₁₈, R₁₉, R₂₀ and R₂₁ are each independently selected from the group consisting of hydrogen, an optionally substituted (C₁-C₆)alkyl, (C₁-C₆)alkylhalo, (C₁-C₆)alkyl-CN, (C₁-C₆)alkyl-O—(C₀-C₆)alkyl, (C₁-C₆)alkyl-N((C₀-C₆)alkyl)₂, (C₁-C₆)alkyl-C(═O)—O—(C₀-C₆)alkyl, (C₁-C₆)alkyl-heterocycloalkyl, (C₂-C₆)alkynyl, (C₂-C₆)alkenyl, (C₃-C₇)cycloalkyl, (C₃-C₇)cycloalkyl-(C₁-C₆)alkyl, heteroaryl, (C₁-C₆)alkyl-heteroaryl, aryl;
Z¹, Z², Z³ and Z⁴ are each independently selected from the group consisting of bond, —C═, —C═C—, —C(═O)—, —C(═S)—, —C—, —O—, —N═, —N— or —S— which may further be substituted by G²ₚ groups;
B¹, B² and B³ are each selected independently from —C— or —N— which may further be substituted by one G²ₚ group;
Any N or S bearing ring may be in its N-oxide, S-oxide or S-dioxide form;
And wherein the compound of formula I-A includes both possible stereoisomers and includes not only racemic compounds but the individual enantiomers as well;
With the following provisos:
When R₇ and R₈ are each independently selected from an optionally substituted (C₁-C₄)alkyl, or can together form a (C₃-C₆)cycloalkyl or an heterocycloalkyl group of formula 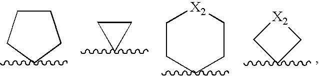
and Gⁱₙ are hydrogens, then 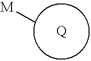
can not be 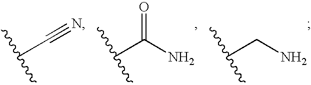
If R₇ and R₈ all represent CH₃ at the same time, then M-Q may not represent CH₃;
R₇ and R₈ may not represent at the same time (C₀-C₆)alkyl-aryl, (C₀-C₆)alkyl-heteroaryl;
When n>1, G¹ₙ groups may not represent at the same time OH;
If R₇, R₈ and M represent at the same time an optionally substituted (C₁-C₄)alkyl, then Q can not be H;
If R₇, R₈ represent 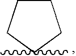
then compounds of the following list are excluded:
3,4-dimethoxy-N-[4-[1-[[(4-methoxyphenyl)amino]carbonyl]cyclopentyl]phenyl]-benzamide
N-[4-(1-cyanocyclopentyl)phenyl]-3,4-dimethoxy-benzamide.

35. A compound according to claim 34 having the formula I-A2-a 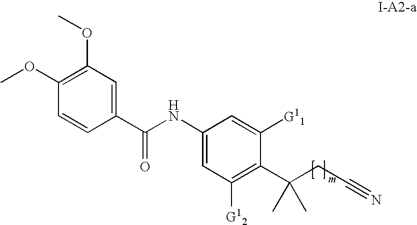
or a pharmaceutically acceptable salt, hydrate or solvate of such compound
wherein:
G¹₁ and G¹₂ are each independently selected from the group consisting of hydrogen, (C₁-C₆)alkyl, (C₀-C₆)alkylhalo, (C₀-C₆)alkyl-CN, (C₃-C₆)cycloalkyl, (C₀-C₆)alkyl-(C₃-C₈)cycloalkyl, (C₂-C₆)alkenyl, (C₂-C₆)alkynyl, (C₀-C₆)alkyl-OR₉, (C₀-C₆)alkyl-NR₉R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O) R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀) R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-) cycloalkyl) or N((C₀-C₆)alkyl)(aryl) substituents, wherein optionally two substituents are combined to the intervening atoms to form a bicyclic heterocycloalkyl, aryl or heteroaryl ring; wherein each ring is optionally further substituted with 1-5 independent halogen, CN, (C₁-C₆)alkyl, O—(C₀-C₆)alkyl, O-alkylcycloalkyl, O(aryl), O(heteroaryl), O-arylalkyl, O-heteroarylalkyl, N((—C₀-C₆)alkyl)((C₀-C₃)arylalkyl) or N((C₀-C₆)alkyl)(heteroarylalkyl) groups;
R₉, R₁₀, R₁₁ each independently is hydrogen, (C₁-C₆)alkyl, (C₃-C₆)cycloalkyl, (C₁-C₆)alkyl-(C₃-C₈)cycloalkyl, (C₂-C₆)alkenyl, (C₂-C₆)alkynyl, (C₁-C₆)alkylhalo, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), N(C₀-C₆-alkyl)₂, —N((C₀-C₆)alkyl)((C₃-C₇-)cycloalkyl) or N((C₀-C₆)alkyl)(aryl) substituents;
m is an integer from 0 to 2;
Any N or S bearing ring may be in its N-oxide, S-oxide or S-dioxide form;
And wherein the compound of formula I-A2-a1 includes both possible stereoisomers and includes not only racemic compounds but the individual enantiomers as well;
With the following provisos:
When G¹₁ and G¹₂ represent at the same time an hydrogen, then m cannot be equal to 0.

36. A compound according to claim 34 having the formula I-A2-b 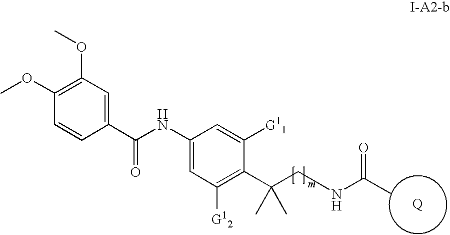
or a pharmaceutically acceptable salt, hydrate or solvate of such compound
wherein:
G¹₁ and G¹₂ are each independently selected from the group consisting of hydrogen, (C₁-C₆)alkyl, (C₀-C₆)alkylhalo, (C₀-C₆)alkyl-CN, (C₃-C₆)cycloalkyl, (C₀-C₆)alkyl-(C₃-C₈)cycloalkyl, (C₂-C₆)alkenyl, (C₂-C₆)alkynyl, (C₀-C₆)alkyl-OR₉, (C₀-C₆)alkyl-NR₉R₁₀, (C₀-C₆)-alkyl-NR₉C0R₁₀, (C₀-C₆)alkyl-NR₉SO₂R₁₀, (C₀-C₆)alkyl-NR₁₁CONR₁₀R₉, (C₀-C₆)alkyl-S R₉, (C₀-C₆)alkyl-S(═O)R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀)R₉, or (C₀-C₆)alkyl-C(═NOR₁₀) R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-) cycloalkyl) or N((C₀-C₆)alkyl)(aryl) substituents, wherein optionally two substituents are combined to the intervening atoms to form a bicyclic heterocycloalkyl, aryl or heteroaryl ring; wherein each ring is optionally further substituted with 1-5 independent halogen, CN, (C₁-C₆)alkyl, O—(C₀-C₆)alkyl, O-alkylcycloalkyl, O(aryl), O(heteroaryl), O-arylalkyl, O-heteroarylalkyl, N((—C₀-C₆)alkyl)((C₀-C₃)arylalkyl) or N((C₀-C₆)alkyl)(heteroarylalkyl) groups; R₉, R₁₀, R₁₁ each independently is hydrogen, (C₁-C₆)alkyl, (C₃-C₆)cycloalkyl, (C₁-C₆)alkyl-(C₃-C₈)cycloalkyl, (C₂-C₆)alkenyl, (C₂-C₆)alkynyl, (C₁-C₆)alkylhalo, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), N(C₀-C₆-alkyl)₂, —N((C₀-C₆)alkyl)((C₃-C₇-)cycloalkyl) or N((C₀-C₆)alkyl)(aryl) substituents;
m is an integer from 0 to 2;
Q represent independently an optionally substituted (C₁-C₆)alkyl, (C₀-C₆)alkyl-CN, (C₁-C₆)alkylhalo, (C₃-C₇)cycloalkyl, (C₃-C₇) heterocycloalkyl or one of the following aryl or heteroaryl: 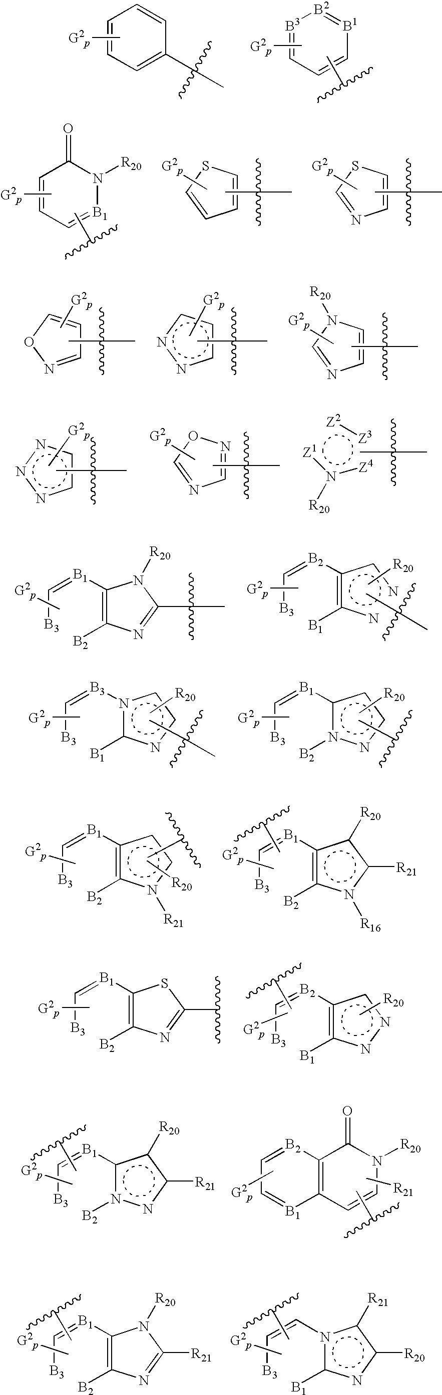
G² groups are each independently selected from the group consisting of hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₂-C₆)alkyl-OR₁₄, O—(C₃-C₇)cycloalkyl, O—(C₁-C₆)alkyl-heteroaryl, O—(C₁-C₆)alkyl-aryl, (C₀-C₆)alkyl-OR₁₄, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl, (C₁-C₆)alkyl-aryl, (C₁-C₆)alkylhalo-OR₁₇, (C₃-C₆)alkynyl-OR₁₇, (C₃-C₆)alkenyl-OR₁₇, (C₀-C₆)alkyl-S—R₁₇, C(C₂-C₆)alkyl-S—₁₇, (C₀-C₆)alkyl-S(═O)—R₁₇, (C₂-C₆)alkyl-S(═O)—R₁₇, (C₀-C₆)alkyl-S(═O)₂—R₁₇, O—(C₁-C₆)alkyl-S(═O)₂—R₁₇, (C₀-C₆)alkyl-NR₁₇R₁₈, O—(C₂-C₆)alkyl-NR₁₇R₁₈, (C₀-C₆)alkyl-S(═O)₂NR₁₇R₁₈, (C₀-C₆)alkyl-NR₁₇—S(═O)₂R₁₈, O—(C₁-C₆)alkyl-S(═O)₂NR₁₇R₁₈, O—(C₂-C₆)alkyl-NR₁₇—S(═O)₂R₁₈, (C₀-C₆)alkyl-C(═O)—NR₁₇R₁₈, (C₀-C₆)alkyl-NR₁₇C(═O)—R₁₈, O—(C₁-C₆)alkyl-C(═O)—NR₁₇R₁₈, O—(C₂-C₆)alkyl-NR₁₇C(═O)—R₁₈, (C₀-C₆)alkyl-OC(═O)—R₁₇, (C₀-C₆)alkyl-C(═O)—OR₁₇, O—(C₂-C₆)alkyl-OC(═O)—R₁₇, O—(C₁-C₆)alkyl-C(═O)—OR₁₇, (C₀-C₆)alkyl-C(═O)—R₁₇, O—(C₁-C₆)alkyl-C(═O)—R₁₇, (C₀-C₆)alkyl-N₁₇—C(═O)—OR₁₈, (C₀-C₆)alkyl-O—C(═O)—NR₁₇R₁₈ or (C₀-C₆)alkyl-NR₁₇—C(═O)—NR₁₈R₁₉ substituents;
wherein optionally two substituents are combined to the intervening atoms to form a bicyclic aryl, cycloalkyl, heterocycloalkyl or heteroaryl ring; wherein each ring is optionally further substituted with 1-5 independent hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₃-C₇)cycloalkyl, O—(C₁-C₆)alkyl-heteroaryl, O—(C₁-C₆)alkylaryl, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl;
p is an integer that is selected from the group consisting of 1, 2, 3, 4 and 5 provided that when p>1, the G² groups may be the same or different from each other;
R₁₆, R₁₇, R₁₈, R₁₉, R₂₀ and R₂₁ are each independently selected from the group consisting of hydrogen, an optionally substituted (C₁-C₆)alkyl, (C₁-C₆)alkylhalo, (C₁-C₆)alkyl-CN, (C₁-C₆)alkyl-O—(C₀-C₆)alkyl, (C₁-C₆)alkyl-N((C₀-C₆)alkyl)₂, (C₁-C₆)alkyl-C(═O)—O—(C₀-C₆)alkyl, (C₁-C₆)alkyl-heterocycloalkyl, (C₂-C₆)alkynyl, (C₂-C₆)alkenyl, (C₃-C₇)cycloalkyl, (C₃-C₇)cycloalkyl-(C₁-C₆)alkyl, heteroaryl, (C₁-C₆)alkyl-heteroaryl, aryl;
Z¹, Z², Z³ and Z⁴ are each independently selected from the group consisting of bond, —C═, —C═C—, —C(═O)—, —C(═S)—, —C—, -O—, —N═, —N— or —S— which may further be substituted by G²ₚ groups;
B¹, B² and B³ are each selected independently from —C— or —N— which may further be substituted by one G²ₚ group;
Any N or S bearing ring may be in its N-oxide, S-oxide or S-dioxide form;
And wherein the compound of formula I-A2-b1 includes both possible stereoisomers and includes not only racemic compounds but the individual enantiomers as well.

37. A compound according to claim 36 having the formula I-A2-b1 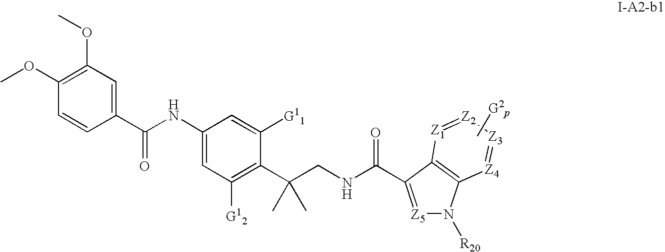
or a pharmaceutically acceptable salt, hydrate or solvate of such compound
wherein:
G¹₁ and G¹₂ are each independently selected from the group consisting of hydrogen, (C₀-C₆)alkyl-CN, (C₁-C₆)alkyl, (C₀-C₆)alkylhalo, (C₃-C₆)cycloalkyl, (C₀-C₆)alkyl-(C₃-C₈)cycloalkyl, (C₂-C₆)alkenyl, (C₂-C₆)alkynyl, (C₀-C₆)alkyl-OR₉, (C₀-C₆)alkyl-NR₉R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O)R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀)R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-) cycloalkyl) or N((C₀-C₆)alkyl)(aryl) substituents, wherein optionally two substituents are combined to the intervening atoms to form a bicyclic heterocycloalkyl, aryl or heteroaryl ring; wherein each ring is optionally further substituted with 1-5 independent halogen, CN, (C₁-C₆)alkyl, O—(C₀-C₆)alkyl, O-alkylcycloalkyl, O(aryl), O(heteroaryl), O-arylalkyl, O-heteroarylalkyl, N((—C₀-C₆)alkyl)((C₀-C₃)arylalkyl) or N((C₀-C₆)alkyl)(heteroarylalkyl) groups;
R₉, R₁₀, R₁₁ each independently is hydrogen, (C₁-C₆)alkyl, (C₃-C₆)cycloalkyl, (C₁-C₆)alkyl-(C₃-C₈)cycloalkyl, (C₂-C₆)alkenyl, (C₂-C₆)alkynyl, (C₁-C₆)alkylhalo, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), N(C₀-C₆-alkyl)₂, —N((C₀-C₆)alkyl)((C₃-C₇-)cycloalkyl) or N((C₀-C₆)alkyl)(aryl) substituents;
G² groups are each independently selected from the group consisting of hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₂-C₆)alkyl-OR₁₄, O—(C₃-C₇)cycloalkyl, O—(C₁-C₆)alkyl-heteroaryl, O—(C₁-C₆)alkyl-aryl, (C₀-C₆)alkyl-OR₁₄, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl, (C₁-C₆)alkyl-aryl, (C₁-C₆)alkylhalo-OR₁₇, (C₃-C₆)alkynyl-OR₁₇, (C₃-C₆)alkenyl-OR₁₇, (C₀-C₆)alkyl-S—R₁₇, O—(C₂-C₆)alkyl-S—R₁₇, (C₀-C₆)alkyl-S(═O)—R₁₇, O—(C₂-C₆)alkyl-S(═O)—R₁₇, (C₀-C₆)alkyl-S(═O)₂—R₁₇, O—(C₁-C₆)alkyl-S(═O)₂—R₁₇, (C₀-C₆)alkyl-NR₁₇R₁₈, O—(C₂-C₆)alkyl-NR₁₇R₁₈, (C₀-C₆)alkyl-S(═O)₂NR₁₇R₁₈, (C₀-C₆)alkyl-NR₁₇—S(═O)₂R₁₈, O—(C₁-C₆)alkyl-S(═O)₂N₁₇R₁₈, O—(C₂-C₆)alkyl-NR₁₇—S(═O)₂R₁₈, (C₀-C₆)alkyl-C(═O)—NR₁₇R₁₈, (C₀-C₆)alkyl-N₇C(═O)—R₁₈, O—(C₁-C₆)alkyl-C(═O)—N₁₇R₁₈, O—(C₂-C₆)alkyl-NR₁₇C(═O)—R₁₈, (C₀-C₆)alkyl-OC(═O)—R₁₇, (C₀-C₆)alkyl-C(═O)—OR₁₇, O—(C₂-C₆)alkyl-OC(═O)—R₁₇, O—(C₁-C₆)alkyl-C(═O)—OR₁₇, (C₀-C₆)alkyl-C(═O)—R₁₇, O—(C₁-C₆)alkyl-C(═O)—R₁₇, (C₀-C₆)alkyl-N₁₇—C(═O)—OR₈, (C₀-C₆)alkyl-O—C(═O)—NR₁₇R₁₈ or (C₀-C₆)alkyl-NR₁₇—C(═O)—NR₁₈R₁₉ substituents;
wherein optionally two substituents are combined to the intervening atoms to form a bicyclic aryl, cycloalkyl, heterocycloalkyl or heteroaryl ring; wherein each ring is optionally further substituted with 1-5 independent hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, —O(C₁-C₆)alkylhalo, O—(C₃-C₆)alkynyl, O—(C₃-C₆)alkenyl, O—(C₃-C₇)cycloalkyl, O—(C₁-C₆)alkyl-heteroaryl, O—(C₁-C₆)alkylaryl, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl;
p is an integer that is selected from the group consisting of 1, 2, 3, 4 and 5 provided that when p>1, the G² groups may be the same or different from each other;
R₁₆, R₁₇, R₁₈, R₁₉ and R₂₀ are each independently selected from the group consisting of hydrogen, an optionally substituted (C₁-C₆)alkyl, (C₁-C₆)alkylhalo, (C₁-C₆)alkyl-CN, (C₁-C₆)alkyl-O—(C₀-C₆)alkyl, (C₁-C₆)alkyl-N((C₀-C₆)alkyl)₂, (C₁-C₆)alkyl-C(═O)—O—(C₀-C₆)alkyl, (C₁-C₆)alkyl-heterocycloalkyl, (C₂-C₆)alkynyl, (C₂-C₆)alkenyl, (C₃-C₇)cycloalkyl, (C₃-C₇)cycloalkyl-(C₁-C₆)alkyl, heteroaryl, (C₁-C₆)alkyl-heteroaryl, aryl;
Z¹, Z², Z³ and Z⁴ are each independently selected from the group consisting of bond, —C═ or —N═ which may further be substituted by G²ₚ groups;
Z⁵ is independently selected from —C— or —N— which may further be substituted by one G²ₚ group;
Any N or S bearing ring may be in its N-oxide, S-oxide or S-dioxide form;
And wherein the compound of formula I-A2-b1 includes both possible stereoisomers and includes not only racemic compounds but the individual enantiomers as well.

38. A compound according to claim 36 having the formula I-A2-b2 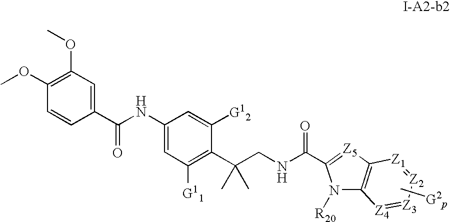
or a pharmaceutically acceptable salt, hydrate or solvate of such compound
wherein:
G¹₁ and G¹₂ are each independently selected from the group consisting of hydrogen, (C₀-C₆)alkyl-CN, (C₁-C₆)alkyl, (C₀-C₆)alkylhalo, (C₃-C₆)cycloalkyl, (C₀-C₆)alkyl-(C₃-C₈)cycloalkyl, (C₂-C₆)alkenyl, (C₂-C₆)alkynyl, (C₀-C₆)alkyl-OR₉, (C₀-C₆)alkyl-NR₉R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O)R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀)R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-) cycloalkyl) or N((C₀-C₆)alkyl)(aryl) substituents, wherein optionally two substituents are combined to the intervening atoms to form a bicyclic heterocycloalkyl, aryl or heteroaryl ring; wherein each ring is optionally further substituted with 1-5 independent halogen, CN, (C₁-C₆)alkyl, O—(C₀-C₆)alkyl, O-alkylcycloalkyl, O(aryl), O(heteroaryl), O-arylalkyl, O-heteroarylalkyl, N((—C₀-C₆)alkyl)((C₀-C₃)arylalkyl) or N((C₀-C₆)alkyl)(heteroarylalkyl) groups;
R₉, R₁₀, R₁₁ each independently is hydrogen, (C₁-C₆)alkyl, (C₃-C₆)cycloalkyl, (C₁-C₆)alkyl-(C₃-C₈)cycloalkyl, (C₂-C₆)alkenyl, (C₂-C₆)alkynyl, (C₁-C₆)alkylhalo, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), N(C₀-C₆-alkyl)₂, —N((C₀-C₆)alkyl)((C₃-C₇-)cycloalkyl) or N((C₀-C₆)alkyl)(aryl) substituents;
G² groups are each independently selected from the group consisting of hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₂-C₆)alkyl-OR₁₄, O—(C₃-C₇)cycloalkyl, O—(C₁-C₆)alkyl-heteroaryl, O—(C₁-C₆)alkyl-aryl, (C₀-C₆)alkyl-OR₁₄, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl, (C₁-C₆)alkyl-aryl, (C₁-C₆)alkylhalo-OR₁₇, (C₃-C₆)alkynyl-OR₁₇, (C₃-C₆)alkenyl-OR₁₇, (C₀-C₆)alkyl-S—R₁₇, O—(C₂-C₆)alkyl-S—R₁₇, (C₀-C₆)alkyl-S(═O)—R₁₇, O—(C₂-C₆)alkyl-S(═O)—R₁₇, (C₀-C₆)alkyl-S(═O)₂—R₁₇, O—(C₁-C₆)alkyl-S(═O)₂—R₁₇, (C₀-C₆)alkyl-NR₁₇R₁₈, O—(C₂-C₆)alkyl-NR₁₇R₁₈, (C₀-C₆)alkyl-S(═O)₂NR₁₇R₁₈, (C₀-C₆)alkyl-NR₁₇—S(═O)₂R₁₈, O—(C₁-C₆)alkyl-S(═O)₂NR₁₇R₁₈, O—(C₂-C₆)alkyl-NR₁₇—S(═O)₂R₁₈, (C₀-C₆)alkyl-C(═O)—NR₁₇R₁₈, (C₀-C₆)alkyl-NR₁₇C(═O)—R₁₈, O—(C₁-C₆)alkyl-C(═O)—N₁₇R₁₈, O—(C₂-C₆)alkyl-NR₁₇C(═O)—R₁₈, (C₀-C₆)alkyl-OC(═O)—R₁₇, (C₀-C₆)alkyl-C(═O)—OR₁₇, O—(C₂-C₆)alkyl-OC(═O)—R₁₇, O—(C₁-C₆)alkyl-C(═O)—OR₁₇, (C₀-C₆)alkyl-C(═O)—R₁₇, O—(C₁-C₆)alkyl-C(═O)—R₁₇, (C₀-C₆)alkyl-N₁₇—C(═O)—O₈, (C₀-C₆)alkyl-O—C(═O)—NR₁₇R₁₈ or (C₀-C₆)alkyl-NR₁₇—C(═O)—NR₁₈R₁₉ substituents;
wherein optionally two substituents are combined to the intervening atoms to form a bicyclic aryl, cycloalkyl, heterocycloalkyl or heteroaryl ring; wherein each ring is optionally further substituted with 1-5 independent hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₃-C₇)cycloalkyl, O—(C₁-C₆)alkyl-heteroaryl, O—(C₁-C₆)alkylaryl, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl;
p is an integer that is selected from the group consisting of 1, 2, 3, 4 and 5 provided that when p>1, the G² groups may be the same or different from each other;
R₁₆, R₁₇, R₁₈, R₁₉ and R₂₀ are each independently selected from the group consisting of hydrogen, an optionally substituted (C₁-C₆)alkyl, (C₁-C₆)alkylhalo, (C₁-C₆)alkyl-CN, (C₁-C₆)alkyl-O—(C₀-C₆)alkyl, (C₁-C₆)alkyl-N((C₀-C₆)alkyl)₂, (C₁-C₆)alkyl-C(═O)—O—(C₀-C₆)alkyl, (C₁-C₆)alkyl-heterocycloalkyl, (C₂-C₆)alkynyl, (C₂-C₆)alkenyl, (C₃-C₇)cycloalkyl, (C₃-C₇)cycloalkyl-(C₁-C₆)alkyl, heteroaryl, (C₁-C₆)alkyl-heteroaryl, aryl;
Z¹, Z², Z³ and Z⁴ are each independently selected from the group consisting of bond, —C═ or —N═ which may further be substituted by G²ₚ groups; Z⁵ is independently selected from —C═ or —N═ which may further be substituted by one G²ₚ group;
Any N or S bearing ring may be in its N-oxide, S-oxide or S-dioxide form;
And wherein the compound of formula I-A2-b2 includes both possible stereoisomers and includes not only racemic compounds but the individual enantiomers as well.

39. A compound according to claim 36 having the formula I-A2-b3 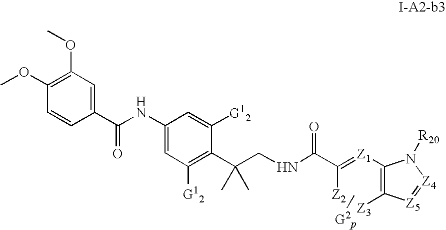
or a pharmaceutically acceptable salt, hydrate or solvate of such compound
wherein:
G¹₁ and G¹₂ are each independently selected from the group consisting of hydrogen, (C₀-C₆)alkyl-CN, (C₁-C₆)alkyl, (C₀-C₆)alkylhalo, (C₃-C₆)cycloalkyl, (C₀-C₆)alkyl-(C₃-C₈)cycloalkyl, (C₂-C₆)alkenyl, (C₂-C₆)alkynyl, (C₀-C₆)alkyl-OR₉, (C₀-C₆)alkyl-NR₉R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O)R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀)R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-) cycloalkyl) or N((C₀-C₆)alkyl)(aryl) substituents, wherein optionally two substituents are combined to the intervening atoms to form a bicyclic heterocycloalkyl, aryl or heteroaryl ring; wherein each ring is optionally further substituted with 1-5 independent halogen, CN, (C₁-C₆)alkyl, O—(C₀-C₆)alkyl, O-alkylcycloalkyl, O(aryl), O(heteroaryl), O-arylalkyl, O-heteroarylalkyl, N((—C₀-C₆)alkyl)((C₀-C₃)arylalkyl) or N((C₀-C₆)alkyl)(heteroarylalkyl) groups;
R₉, R₁₀, R₁₁ each independently is hydrogen, (C₁-C₆)alkyl, (C₃-C₆)cycloalkyl, (C₁-C₆)alkyl-(C₃-C₈)cycloalkyl, (C₂-C₆)alkenyl, (C₂-C₆)alkynyl, (C₁-C₆)alkylhalo, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), N(C₀-C₆-alkyl)₂, —N((C₀-C₆)alkyl)((C₃-C₇-)cycloalkyl) or N((C₀-C₆)alkyl)(aryl) substituents;
G² groups are each independently selected from the group consisting of hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, —O(C₁-C₆)alkylhalo, O—(C₃-C₆)alkynyl, O—(C₃-C₆)alkenyl, O—(C₂-C₆)alkyl-OR₁₄, O—(C₃-C₇)cycloalkyl, O—(C₁-C₆)alkyl-heteroaryl, O—(C₁-C₆)alkyl-aryl, (C₀-C₆)alkyl-OR₁₄, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl, (C₁-C₆)alkyl-aryl, (C₁-C₆)alkylhalo-OR₁₇, (C₃-C₆)alkynyl-OR₁₇, (C₃-C₆)alkenyl-OR₁₇, (C₀-C₆)alkyl-S—R₁₇, C(C₂-C₆)alkyl-S—₁₇, (C₀-C₆)alkyl-S(═O)—R₁₇, (C₂-C₆)alkyl-S(═O)—R₁₇, (C₀-C₆)alkyl-S(═O)₂—R₁₇, O—(C₁-C₆)alkyl-S(═O)₂—R₁₇, (C₀-C₆)alkyl-NR₁₇R₁₈, O—(C₂-C₆)alkyl-NR₁₇R₁₈, (C₀-C₆)alkyl-S(═O)₂NR₁₇R₁₈, (C₀-C₆)alkyl-NR₁₇—S(═O)₂R₁₈, O—(C₁-C₆)alkyl-S(═O)₂NR₁₇R₁₈, O—(C₂-C₆)alkyl-NR₁₇—S(═O)₂R₁₈, (C₀-C₆)alkyl-C(═O)—NR₁₇R₁₈, (C₀-C₆)alkyl-NR₁₇C(═O)—R₁₈, O—(C₁-C₆)alkyl-C(═O)—NR₁₇R₁₈, O—(C₂-C₆)alkyl-NR₁₇C(═O)—R₁₈, (C₀-C₆)alkyl-OC(═O)—R₁₇, (C₀-C₆)alkyl-C(═O)—OR₁₇, O—(C₂-C₆)alkyl-OC(═O)—R₁₇, O—(C₁-C₆)alkyl-C(═O)—OR₁₇, (C₀-C₆)alkyl-C(═O)—R₁₇, O—(C₁-C₆)alkyl-C(═O)—R₁₇, (C₀-C₆)alkyl-NR₁₇—C(═O)—OR₁₈, (C₀-C₆)alkyl-O—C(═O)—NR₁₇R₁₈ or (C₀-C₆)alkyl-NR₁₇—C(═O)—NR₁₈R₁₉ substituents;
wherein optionally two substituents are combined to the intervening atoms to form a bicyclic aryl, cycloalkyl, heterocycloalkyl or heteroaryl ring; wherein each ring is optionally further substituted with 1-5 independent hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₃-C₇)cycloalkyl, O—(C₁-C₆)alkyl-heteroaryl, O—(C₁-C₆)alkylaryl, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl;
p is an integer that is selected from the group consisting of 1, 2, 3, 4 and 5 provided that when p>1, the G² groups may be the same or different from each other;
R₁₆, R₁₇, R₁₈, R₁₉ and R₂₀ are each independently selected from the group consisting of hydrogen, an optionally substituted (C₁-C₆)alkyl, (C₁-C₆)alkylhalo, (C₁-C₆)alkyl-CN, (C₁-C₆)alkyl-O—(C₀-C₆)alkyl, (C₁-C₆)alkyl-N((C₀-C₆)alkyl)₂, (C₁-C₆)alkyl-C(═O)—O—(C₀-C₆)alkyl, (C₁-C₆)alkyl-heterocycloalkyl, (C₂-C₆)alkynyl, (C₂-C₆)alkenyl, (C₃-C₇)cycloalkyl, (C₃-C₇)cycloalkyl-(C₁-C₆)alkyl, heteroaryl, (C₁-C₆)alkyl-heteroaryl, aryl;
Z¹, Z² and Z³ are each independently selected from the group consisting of bond, —C═ or —N═ which may further be substituted by G²ₚ groups;
Z⁴ and Z⁵ are each independently selected from —C— or —N— which may further be substituted by one G²ₚ group;
Any N or S bearing ring may be in its N-oxide, S-oxide or S-dioxide form;
And wherein the compound of formula I-A2-b3 includes both possible stereoisomers and includes not only racemic compounds but the individual enantiomers as well.

40. A compound according to claim 36 having the formula I-A2-b4 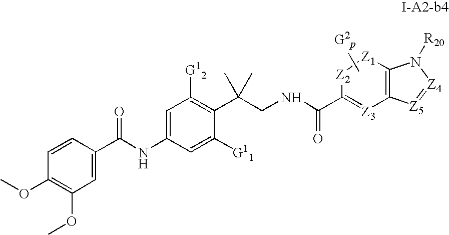
or a pharmaceutically acceptable salt, hydrate or solvate of such compound
wherein:
G¹₁ and G¹₂ are each independently selected from the group consisting of hydrogen, (C₀-C₆)alkyl-CN, (C₁-C₆)alkyl, (C₀-C₆)alkylhalo, (C₃-C₆)cycloalkyl, (C₀-C₆)alkyl-(C₃-C₈)cycloalkyl, (C₂-C₆)alkenyl, (C₂-C₆)alkynyl, (C₀-C₆)alkyl-OR₉, (C₀-C₆)alkyl-NR₉R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O) R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀)R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-)cycloalkyl) or N((C₀-C₆)alkyl)(aryl) substituents, wherein optionally two substituents are combined to the intervening atoms to form a bicyclic heterocycloalkyl, aryl or heteroaryl ring; wherein each ring is optionally further substituted with 1-5 independent halogen, CN, (C₁-C₆)alkyl, O—(C₀-C₆)alkyl, O-alkylcycloalkyl, O(aryl), O(heteroaryl), O-arylalkyl, O-heteroarylalkyl, N((—C₀-C₆)alkyl)((C₀-C₃)arylalkyl) or N((C₀-C₆)alkyl)(heteroarylalkyl) groups;
R₉, R₁₀, R₁₁ each independently is hydrogen, (C₁-C₆)alkyl, (C₃-C₆)cycloalkyl, (C₁-C₆)alkyl-(C₃-C₈)cycloalkyl, (C₂-C₆)alkenyl, (C₂-C₆)alkynyl, (C₁-C₆)alkylhalo, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), N(C₀-C₆-alkyl)₂, —N((C₀-C₆)alkyl)((C₃-C₇-)cycloalkyl) or N((C₀-C₆)alkyl)(aryl) substituents;
G² groups are each independently selected from the group consisting of hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₂-C₆)alkyl-OR₁₄, O—(C₃-C₇)cycloalkyl, O—(C₁-C₆)alkyl-heteroaryl, O—(C₁-C₆)alkyl-aryl, (C₀-C₆)alkyl-OR₁₄, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl, (C₁-C₆)alkyl-aryl, (C₁-C₆)alkylhalo-OR₁₇, (C₃-C₆)alkynyl-OR₁₇, (C₃-C₆)alkenyl-OR₁₇, (C₀-C₆)alkyl-S—R₁₇, C(C₂-C₆)alkyl-S—R₁₇, (C₀-C₆)alkyl-S(═O)—R₁₇, (C₂-C₆)alkyl-S(═O)—R₁₇, (C₀-C₆)alkyl-S(═O)₂—R₁₇, O—(C₁-C₆)alkyl-S(═O)₂—R₁₇, (C₀-C₆)alkyl-NR₁₇R₁₈, O—(C₂-C₆)alkyl-NR₁₇R₁₈, (C₀-C₆)alkyl-S(═O)₂NR₁₇R₁₈, (C₀-C₆)alkyl-NR₁₇—S(═O)₂R₁₈, O—(C₁-C₆)alkyl-S(═O)₂NR₁₇R₁₈, O—(C₂-C₆)alkyl-NR₁₇—S(═O)₂R₁₈, (C₀-C₆)alkyl-C(═O)—NR₁₇R₁₈, (C₀-C₆)alkyl-NR₁₇C(═O)—R₁₈, O—(C₁-C₆)alkyl-C(═O)—NR₁₇R₁₈, O—(C₂-C₆)alkyl-NR₁₇C(═O)—R₁₈, (C₀-C₆)alkyl-OC(═O)—R₁₇, (C₀-C₆)alkyl-C(═O)—OR₁₇, O—(C₂-C₆)alkyl-OC(═O)—R₁₇, O—(C₁-C₆)alkyl-C(═O)—OR₁₇, (C₀-C₆)alkyl-C(═O)—R₁₇, O—(C₁-C₆)alkyl-C(═O)—R₁₇, (C₀-C₆)alkyl-NR₁₇—C(═O)—OR₁₈, (C₀-C₆)alkyl-O—C(═O)—NR₁₇R₁₈ or (C₀-C₆)alkyl-NR₁₇—C(═O)—NR₁₈R₁₉ substituents;
wherein optionally two substituents are combined to the intervening atoms to form a bicyclic aryl, cycloalkyl, heterocycloalkyl or heteroaryl ring; wherein each ring is optionally further substituted with 1-5 independent hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₃-C₇)cycloalkyl, O—(C₁-C₆)alkyl-heteroaryl, O—(C₁-C₆)alkylaryl, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl;
p is an integer that is selected from the group consisting of 1, 2, 3, 4 and 5 provided that when p>1, the G² groups may be the same or different from each other;
R₁₆, R₁₇, R₁₈, R₁₉ and R₂₀ are each independently selected from the group consisting of hydrogen, an optionally substituted (C₁-C₆)alkyl, (C₁-C₆)alkylhalo, (C₁-C₆)alkyl-CN, (C₁-C₆)alkyl-O—(C₀-C₆)alkyl, (C₁-C₆)alkyl-N((C₀-C₆)alkyl)₂, (C₁-C₆)alkyl-C(O)—O—(C₀-C₆)alkyl, (C₁-C₆)alkyl-heterocycloalkyl, (C₂-C₆)alkynyl, (C₂-C₆)alkenyl, (C₃-C₇)cycloalkyl, (C₃-C₇)cycloalkyl-(C₁-C₆)alkyl, heteroaryl, (C₁-C₆)alkyl-heteroaryl, aryl;
Z¹, Z² and Z³ are each independently selected from the group consisting of bond, —C═ or —N═ which may further be substituted by G²ₚ groups;
Z⁴ and Z⁵ are each independently selected from —C— or —N— which may further be substituted by one G²ₚ group;
Any N or S bearing ring may be in its N-oxide, S-oxide or S-dioxide form;
And wherein the compound of formula I-A2-b4 includes both possible stereoisomers and includes not only racemic compounds but the individual enantiomers as well.

41. A compound according to claim 34 having the formula 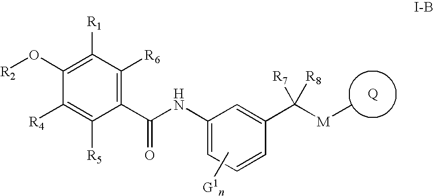
or a pharmaceutically acceptable salt, hydrate or solvate of such compound
wherein:
represents independently O—(C₁-C₆)alkyl, O—(C₂-C₆)alkynyl, O—(C₂-C₆)alkenyl, O—(C₃-C₇)cycloalkyl and O-alkylcycloalkyl;
R₂ represents independently hydrogen, an optionally substituted (C₁-C₆)alkyl, (C₂-C₆)alkynyl, (C₂-C₆)alkenyl, (C₃-C₇)cycloalkyl, (C₄-C₁₀)alkylcycloalkyl, (C₁-C₆)heterocycloalkyl, (C₁-C₆)alkyl-heteroaryl, (C₁-C₆)alkyl-aryl or (C₁-C₆)alkyl-CN;
R₁ and R₂ according to the above definitions can be combined to form a heterocycloalkyl ring;
R₄ is independently selected from the group consisting of hydrogen, OH, (C₀-C₆)alkyl-CN, (C₁-C₆)alkyl, (C₀-C₆)alkylhalo, (C₃-C₆)cycloalkyl, (C₁-C₆)alkyl-(C₃-C₈)cycloalkyl, (C₂-C₆)alkenyl, (C₂-C₆)alkynyl, (C₀-C₆)alkyl-OR₉, (C₁-C₆)alkyl-NR₉R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O)R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═N₁₀) R₉, or (C₀-C₆)alkyl-C(═NOR₁₀)R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-) cycloalkyl) or N((C₀-C₆)alkyl)(aryl) substituents;
R₅, R₆ are each independently selected from the group consisting of hydrogen, (C₀-C₆)alkyl-CN, (C₁-C₆)alkyl, (C₀-C₆)alkylhalo, (C₃-C₆)cycloalkyl, (C₁-C₆)alkyl-(C_{(C}₃-C₈)cycloalkyl, (C₂-C₆)alkenyl, (C₂-C₆)alkynyl, (C₀-C₆)alkyl-OR₉, (C₁-C₆)alkyl-NR₉R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O)R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀)R₉, or (C₀-C₆)alkyl-C(═NOR₁₀) R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-)cycloalkyl) or N((C₀-C₆)alkyl)(aryl) substituents;
G¹ is independently selected from the group consisting of hydrogen, OH, (C₀-C₆)alkyl-CN, (C₁-C₆)alkyl, (C₀-C₆)alkylhalo, (C₃-C₆)cycloalkyl, (C₀-C₆)alkyl-(C₃-C₈)cycloalkyl, (C₂-C₆)alkenyl, (C₂-C₆)alkynyl, (C₀-C₆)alkyl-OR₉, (C₀-C₆)alkyl-NR₉R₁₀, (C₀-C₆)-alkyl-NR₉COR₁₀, (C₀-C₆)alkyl-NR₉SO₂R₁₀, (C₀-C₆)alkyl-NR₁₁CONR₁₀R₉, (C₀-C₆)alkyl-SR₉, (C₀-C₆)alkyl-S(═O)R₉, (C₀-C₆)alkyl-S(═O)₂R₉, (C₀-C₆)alkyl-S(═O)₂NR₁₀R₉, (C₀-C₆)alkyl-C(═O)—(C₁-C₆), (C₀-C₆)alkyl-C(O)—O—R₉, (C₀-C₆)alkyl-C(═O)NR₁₀R₉, (C₀-C₆)alkyl-C(═NR₁₀) R₉, or (C₀-C₆)alkyl-C(═NOR₁₀) R₉, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-) cycloalkyl) or N((C₀-C₆)alkyl)(aryl) substituents, wherein optionally two substituents are combined to the intervening atoms to form a bicyclic heterocycloalkyl, aryl or heteroaryl ring; wherein each ring is optionally further substituted with 1-5 independent halogen, CN, (C₁-C₆)alkyl, O—(C₀-C₆)alkyl, O-alkylcycloalkyl, O(aryl), O(heteroaryl), O-arylalkyl, O-heteroarylalkyl, N((—C₀-C₆)alkyl)((C₀-C₃)arylalkyl) or N((C₀-C₆)alkyl)(heteroarylalkyl) groups; R₉, R₁₀, R₁₁ each independently is hydrogen, (C₁-C₆)alkyl, (C₃-C₆)cycloalkyl, (C₁-C₆)alkyl-(C₃-C₈)cycloalkyl, (C₂-C₆)alkenyl, (C₂-C₆)alkynyl, (C₁-C₆)alkylhalo, heterocycloalkyl, heteroaryl, heteroarylalkyl, arylalkyl or aryl; any of which is optionally substituted with 1-5 independent halogen, CN, (C₁-C₆)alkyl, O—(C₀-C₆)alkyl, O-alkylcycloalkyl, O(aryl), O(heteroaryl), N(C₀-C₆-alkyl)₂, —N((C₀-C₆)alkyl)((C₃-C₇-)cycloalkyl) or N((C₀-C₆)alkyl)(aryl) substituents;
n is an integer from 1 to 4, provided that when n>1, the G¹ groups may be the same or different from each other;
R₇ and R₈ represent independently an optionally substituted (C₁-C₄)alkyl, (C₁-C₆)alkylhalo, (C₀-C₆)alkyl-aryl, (C₁-C₆)alkyl-O—(C₀-C₆)-alkyl, (C₀-C₆)alkyl-heteroaryl, (C₀-C₆)alkyl-heterocycloalkyl, (C₀-C₆)alkyl-(C₃-C₇)cycloalkyl or R⁷ and R⁸ can together form a (C₃-C₆)cycloalkyl or an heterocycloalkyl group of formula: 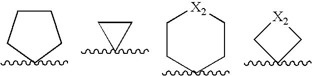
X₂ is independently selected from the group consisting of CH₂, O, S, SO₂;
M is independently selected from the group of consisting of a bond, an optionally substituted (C₁-C₆)alkyl, (C₂-C₆)alkynyl, (C₁-C₆)alkylhalo, (C₂-C₆)alkenyl, (C₁-C₆)alkyl-O—(C₀-C₆)alkyl, (C₀-C₆)alkyl-S(═O)₂NR₁₂-(C₀-C₆)alkyl, (C₀-C₆)alkyl-NR₁₂—S(═O)₂—(C₀-C₆)alkyl, (C₀-C₆)alkyl-C(═O)—NR₁₂—(C₀-C₆)alkyl, (C₀-C₆)alkyl-NR₁₂C(═O)—(C₀-C₆)alkyl, (C₀-C₆)alkyl-NR₁₂—C(═O)—O—(C₀-C₆)alkyl, (C₀-C₆)alkyl-O—C(═O)—NR₁₂—(C₀-C₆)alkyl or (C₀-C₆)alkyl-NR₁₂—C(═O)—NR₁₃—(C₀-C₆)alkyl substituents;
wherein optionally two substituents are combined with the intervening atoms to form a cycloalkyl or heterocycloalkyl ring;
R₁₂ and R₁₃ are each independently selected from the group consisting of hydrogen, an optionally substituted (C₁-C₆)alkyl;
Q represent independently H, an optionally substituted (C₁-C₆)alkyl, (C₀-C₆)alkyl-CN, (C₁-C₆)alkylhalo, (C₃-C₇)cycloalkyl, (C₃-C₇)heterocycloalkyl or one of the following aryl or heteroaryl: 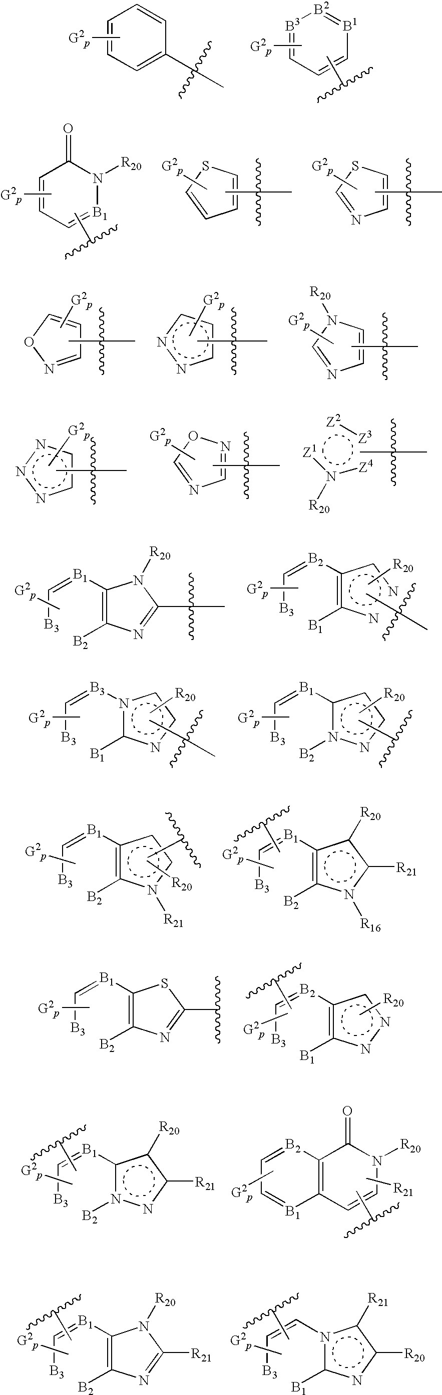
G² groups are each independently selected from the group consisting of hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₂-C₆)alkyl-OR₁₄, O—(C₃-C₇)cycloalkyl, O—(C₁-C₆)alkyl-heteroaryl, O—(C₁-C₆)alkyl-aryl, (C₀-C₆)alkyl-OR₁₄, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl, (C₁-C₆)alkyl-aryl, (C₁-C₆)alkylhalo-OR₁₇, (C₃-C₆)alkynyl-OR₁₇, (C₃-C₆)alkenyl-OR₁₇, (C₀-C₆)alkyl-S—R₁₇, O—(C₂-C₆)alkyl-S—R₁₇, (C₀-C₆)alkyl-S(═O)—R₁₇, O—(C₂-C₆)alkyl-S(═O)—R₁₇, (C₀-C₆)alkyl-S(═O)₂—R₁₇, O—(C₁-C₆)alkyl-S(═O)₂—R₁₇, (C₀-C₆)alkyl-NR₁₇R₁₈, O—(C₂-C₆)alkyl-NR₁₇R₁₈, (C₀-C₆)alkyl-S(═O)₂NR₁₇R₁₈, (C₀-C₆)alkyl-NR₁₇—S(═O)₂R₁₈, O—(C₁-C₆)alkyl-S(═O)₂NR₁₇R₁₈, O—(C₂-C₆)alkyl-NR₁₇—S(═O)₂R₁₈, (C₀-C₆)alkyl-C(═O)—NR₁₇R₁₈, (C₀-C₆)alkyl-NR₁₇C(═O)—R₁₈, O—(C₁-C₆)alkyl-C(═O)—NR₁₇R₁₈, O—(C₂-C₆)alkyl-NR₁₇C(═O)—R₁₈, (C₀-C₆)alkyl-OC(═O)—R₁₇, (C₀-C₆)alkyl-C(═O)—OR₁₇, O—(C₂-C₆)alkyl-OC(═O)—R₁₇, O—(C₁-C₆)alkyl-C(═O)—OR₁₇, (C₀-C₆)alkyl-C(═O)—R₁₇, O—(C₁-C₆)alkyl-C(═O)—R₁₇, (C₀-C₆)alkyl-NR₁₇—C(═O)—OR₁₈, (C₀-C₆)alkyl-O—C(═O)—NR₁₇R₁₈ or (C₀-C₆)alkyl-NR₁₇—C(═O)—N₁₈R₁₉ substituents;
wherein optionally two substituents are combined to the intervening atoms to form a bicyclic aryl, cycloalkyl, heterocycloalkyl or heteroaryl ring; wherein each ring is optionally further substituted with 1-5 independent hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₃-C₇)cycloalkyl, O—(C₁-C₆)alkyl-heteroaryl, O—(C₁-C₆)alkylaryl, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl;
p is an integer that is selected from the group consisting of 1, 2, 3, 4 and 5 provided that when p>1, the G² groups may be the same or different from each other;
R₁₆, R₁₇, R₁₈, R₁₉, R₂₀ and R₂₁ are each independently selected from the group consisting of hydrogen, an optionally substituted (C₁-C₆)alkyl, (C₁-C₆)alkylhalo, (C₁-C₆)alkyl-CN, (C₁-C₆)alkyl-O—(C₀-C₆)alkyl, (C₁-C₆)alkyl-N((C₀-C₆)alkyl)₂, (C₁-C₆)alkyl-C(═O)—O—(C₀-C₆)alkyl, (C₁-C₆)alkyl-heterocycloalkyl, (C₂-C₆)alkynyl, (C₂-C₆)alkenyl, (C₃-C₇)cycloalkyl, (C₃-C₇)cycloalkyl-(C₁-C₆)alkyl, heteroaryl, (C₁-C₆)alkyl-heteroaryl, aryl;
Z¹, Z², Z³ and Z⁴ are each independently selected from the group consisting of bond, —C═, —C═C—, —C(═O)—, —C(═S)—, —C—, —O—, —N═, —N— or —S— which may further be substituted by G²ₚ groups;
B¹, B² and B³ are each selected independently from —C— or —N— which may further be substituted by one G²ₚ group;
Any N or S bearing ring may be in its N-oxide, S-oxide or S-dioxide form;
And wherein the compound of formula I-B includes both possible stereoisomers and includes not only racemic compounds but the individual enantiomers as well;
with the following provisos:
If R₇ and R₈ all represent CH₃ at the same time, then M-Q may not represent CH₃;
If R₅ or R₆ are represented by (C₀-C₆)alkyl-OR₉, then R₉ may not represent a hydrogen;
R₇ and R₈ may not represent at the same time (C₀-C₆)alkyl-aryl, (C₀-C₆)alkyl-heteroaryl;
When n>1, G¹ₙ groups may not represent at the same time OH;
If R₇, R₈ and M represent at the same time an optionally substituted (C₁-C₄)alkyl, then Q can not be H.

42. A compound according to claim 41 having the formula I-B2-a 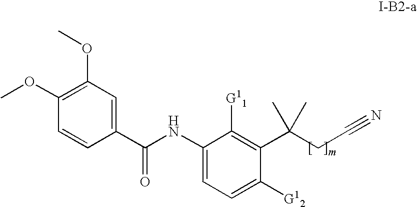
or a pharmaceutically acceptable salt, hydrate or solvate of such compound
wherein:
G¹₁ represents hydrogen, (C₁-C₆)alkyl, (C₀-C₆)alkylhalo or (C₂-C₆)alkenyl;
G¹₂ is independently selected from the group consisting of hydrogen, (C₁-C₆)alkyl, (C₀-C₆)alkylhalo, heterocycloalkyl, heteroaryl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-)cycloalkyl) or N((C₀-C₆)alkyl)(aryl) substituents;
m is an integer from 0 to 2;
Any N or S bearing ring may be in its N-oxide, S-oxide or S-dioxide form;
And wherein the compound of formula I-B2-a includes both possible stereoisomers and includes not only racemic compounds but the individual enantiomers as well.

43. A compound according to claim 41 having the formula I-B2-b 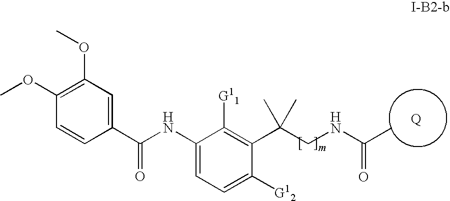
or a pharmaceutically acceptable salt, hydrate or solvate of such compound
wherein:
G¹₁ represents hydrogen, (C₁-C₆)alkyl, (C₀-C₆)alkylhalo or (C₂-C₆)alkenyl;
G¹₂ is independently selected from the group consisting of hydrogen, (C₁-C₆)alkyl, (C₀-C₆)alkylhalo, heterocycloalkyl, heteroaryl or aryl; any of which is optionally substituted with 1-5 independent halogen, CN, (C₁-C₆)alkyl, O—(C₀-C₆)alkyl, O-alkylcycloalkyl, O(aryl), O(heteroaryl), O-(heterocycloalkyl), N(C₀-C₆-alkyl)₂, N((C₀-C₆)alkyl)((C₃-C₇-)cycloalkyl) or N((C₀-C₆)alkyl)(aryl) substituents;
m is an integer from 0 to 2;
represent independently an optionally substituted (C₁-C₆)alkyl, (C₀-C₆)alkyl-CN, (C₁-C₆)alkylhalo, (C₃-C₇)cycloalkyl, (C₃-C₇) heterocycloalkyl or one of the following aryl or heteroaryl: 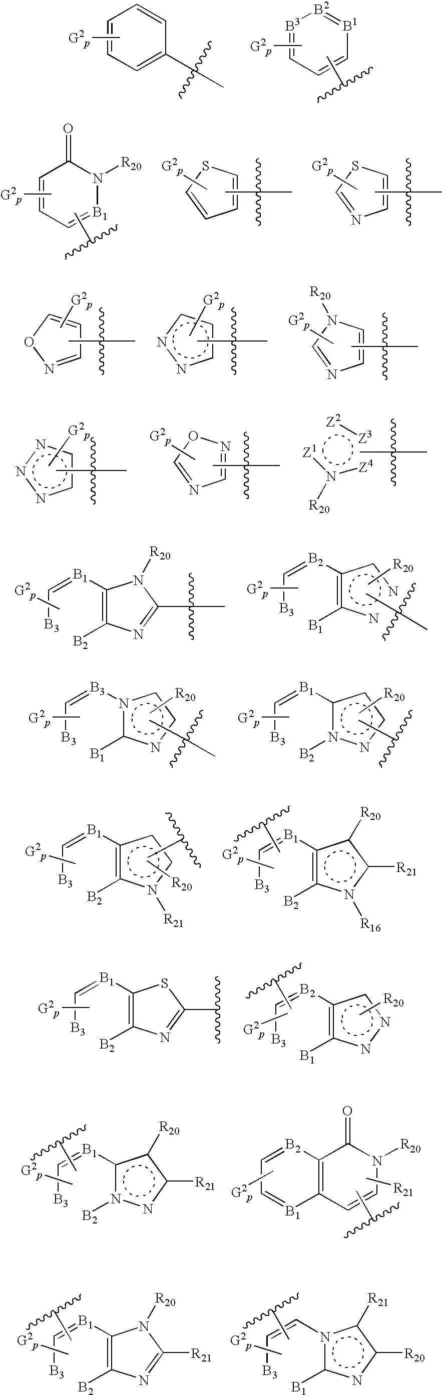
G² groups are each independently selected from the group consisting of hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₂-C₆)alkyl-OR₁₄, O—(C₃-C₇)cycloalkyl, O—(C₁-C₆)alkyl-heteroaryl, O—(C₁-C₆)alkyl-aryl, (C₀-C₆)alkyl-OR₁₄, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl, (C₁-C₆)alkyl-aryl, (C₁-C₆)alkylhalo-OR₁₇, (C₃-C₆)alkynyl-OR₁₇, (C₃-C₆)alkenyl-OR₁₇, (C₀-C₆)alkyl-S—R₁₇, C(C₂-C₆)alkyl-S—R₁₇, (C₀-C₆)alkyl-S(═O)—R₁₇, O—(C₂-C₆)alkyl-S(═O)—R₁₇, (C₀-C₆)alkyl-S(═O)₂—R₁₇, O—(C₁-C₆)alkyl-S(═O)₂—R₁₇, (C₀-C₆)alkyl-NR₁₇R₁₈, O—(C₂-C₆)alkyl-NR₁₇R₁₈, (C₀-C₆)alkyl-S(═O)₂NR₁₇R₁₈, (C₀-C₆)alkyl-NR₁₇—S(═O)₂R₁₈, O—(C₁-C₆)alkyl-S(═O)₂NR₁₇R₁₈, O—(C₂-C₆)alkyl-NR₁₇—S(═O)₂R₁₈, (C₀-C₆)alkyl-C(═O)—NR₁₇R₁₈, (C₀-C₆)alkyl-NR₁₇C(═O)—R₁₈, O—(C₁-C₆)alkyl-C(═O)—NR₁₇R₁₈, O—(C₂-C₆)alkyl-NR₁₇C(═O)—R₁₈, (C₀-C₆)alkyl-OC(═O)—R₁₇, (C₀-C₆)alkyl-C(═O)—OR₁₇, O—(C₂-C₆)alkyl-OC(═O)—R₁₇, O—(C₁-C₆)alkyl-C(═O)—OR₁₇, (C₀-C₆)alkyl-C(═O)—R₁₇, O—(C₁-C₆)alkyl-C(═O)—R₁₇, (C₀-C₆)alkyl-NR₁₇—C(═O)—OR₁₈, (C₀-C₆)alkyl-O—C(═O)—NR₁₇R₁₈ or (C₀-C₆)alkyl-NR₁₇—C(═O)—NR₁₈R₁₉ substituents;
wherein optionally two substituents are combined to the intervening atoms to form a bicyclic aryl, cycloalkyl, heterocycloalkyl or heteroaryl ring; wherein each ring is optionally further substituted with 1-5 independent hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₃-C₇)cycloalkyl, O—(C₁-C₆)alkyl-heteroaryl, O—(C₁-C₆)alkylaryl, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl;
p is an integer that is selected from the group consisting of 1, 2, 3, 4 and 5 provided that when p>1, the G² groups may be the same or different from each other;
R₁₆, R₁₇, R₁₈, R₁₉, R₂₀ and R₂₁ are each independently selected from the group consisting of hydrogen, an optionally substituted (C₁-C₆)alkyl, (C₁-C₆)alkylhalo, (C₁-C₆)alkyl-CN, (C₁-C₆)alkyl-O—(C₀-C₆)alkyl, (C₁-C₆)alkyl-N((C₀-C₆)alkyl)₂, (C₁-C₆)alkyl-C(═O)—O—(C₀-C₆)alkyl, (C₁-C₆)alkyl-heterocycloalkyl, (C₂-C₆)alkynyl, (C₂-C₆)alkenyl, (C₃-C₇)cycloalkyl, (C₃-C₇)cycloalkyl-(C₁-C₆)alkyl, heteroaryl, (C₁-C₆)alkyl-heteroaryl, aryl;
Z¹, Z², Z³, Z⁴, Z⁵, Z⁶, Z⁷, Z⁸ and Z⁹ are each independently selected from the group consisting of bond, —C═, —C═C—, —C(═O)—, —C(═S)—, —C—, —O—, —N═, —N— or —S— which may further be substituted by G²ₚ groups;
B¹, B² and B³ are each selected independently from —C—, —N—, —O— or —S— which may further be substituted by one G²ₚ group;
Any N or S bearing ring may be in its N-oxide, S-oxide or S-dioxide form;
And wherein the compound of formula I-B2-b includes both possible stereoisomers and includes not only racemic compounds but the individual enantiomers as well.

44. A compound according to claim 43 having the formula I-B2-b1 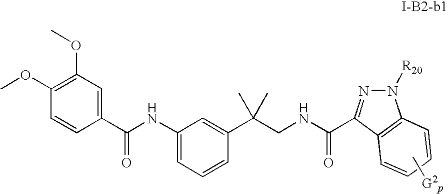
or a pharmaceutically acceptable salt, hydrate or solvate of such compound
wherein:
G² groups are each independently selected from the group consisting of hydrogen, halogen, CN, OH, nitro, an optionally substituted (C₁-C₆)alkyl, (C₁-C₆)alkylhalo, (C₂-C₆)alkynyl, (C₂-C₆)alkenyl, O—(C₁-C₆)alkyl, O—(C₁-C₆)alkylhalo, O—(C₃-C₆)alkynyl, O—(C₃-C₆)alkenyl, O—(C₃-C₇)cycloalkyl, O—(C₁-C₆)alkyl-heteroaryl, O—(C₁-C₆)alkyl-aryl, (C₃-C₇)cycloalkyl, (C₃-C₇)cycloalkyl-(C₁-C₆)alkyl, O—(C₃-C₇)cycloalkyl-(C₁-C₆)alkyl, O-heteroaryl, heteroaryl, (C₁-C₆)alkyl-heteroaryl, aryl, O-aryl or (C₁-C₆)alkyl-aryl substituents;
p is an integer that is selected from the group consisting of 1, 2, 3, 4 and 5 provided that when p>1, the G² groups may be the same or different from each other;
R₂₀ is independently selected from the group consisting of hydrogen or an optionally substituted (C₁-C₆)alkyl;
Any N or S bearing ring may be in its N-oxide, S-oxide or S-dioxide form;
And wherein the compound of formula I-B2-b2 includes both possible stereoisomers and includes not only racemic compounds but the individual enantiomers as well.

45. A compound according to claim 1, which can exist as optical isomers, wherein said compound is either the racemic mixture or one or both of the individual optical isomers.

46. A compound according to claim 1, wherein said compound is selected from:
Pyrazolo[1,5-a]pyridine-3-carboxylic acid {2-[4-(3,4-dimethoxy-benzoylamino)-phenyl]-2-methyl-propyl}-amide
Benzo[b]thiophene-3-carboxylic acid {2-[4-(3,4-dimethoxy-benzoylamino)-phenyl]-2-methyl-propyl}-amide
N-[4-(Cyano-dimethyl-methyl)-3-ethyl-phenyl]-3,4-dimethoxy-benzamide
5-Fluoro-1H-indole-3-carboxylic acid {2-[4-(3,4-dimethoxy-benzoylamino)-phenyl]-2-methyl-propyl}-amide
6-Fluoro-1H-benzoimidazole-2-carboxylic acid {2-[4-(3,4-dimethoxy-benzoylamino)-phenyl]-2-methyl-propyl}-amide
N-[4-(Cyano-dimethyl-methyl)-3-thiophen-2-yl-phenyl]-3,4-dimethoxy-benzamide
6-Fluoro-imidazo[1,2-a]pyridine-3-carboxylic acid {2-[4-(3,4-dimethoxy-benzoylamino)-phenyl]-2-methyl-propyl}-amide
N-(4-{2-[2-(5-Fluoro-indol-1-yl)-acetylamino]-1,1-dimethyl-ethyl}-phenyl)-3,4-dimethoxy-benzamide
1H-Pyrrolo[2,3-b]pyridine-5-carboxylic acid {2-[4-(3,4-dimethoxy-benzoylamino)-phenyl]-2-methyl-propyl}-amide
N-[4-(2-Acetylamino-1,1-dimethyl-ethyl)-3-ethyl-phenyl]-3,4-dimethoxy-benzamide
3H-Imidazo[4,5-b]pyridine-6-carboxylic acid {2-[4-(3,4-dimethoxy-benzoylamino)-2-ethyl-phenyl]-2-methyl-propyl}-amide
5-Fluoro-1H-pyrrolo[2,3-c]pyridine-3-carboxylic acid {2-[4-(3,4-dimethoxy-benzoylamino)-phenyl]-2-methyl-propyl}-amide
N-[4-(2-Benzoylamino-1,1-dimethyl-ethyl)-phenyl]-3,4-dimethoxy-benzamide
Furan-2-carboxylic acid {2-[4-(3,4-dimethoxy-benzoylamino)-phenyl]-2-methyl-propyl}-amide
N-{4-[1,1-Dimethyl-2-(3-phenyl-propionylamino)-ethyl]-phenyl}-3,4-dimethoxy-benzamide
1-Methyl-1H-indazole-3-carboxylic acid {2-[4-(3,4-dimethoxy-benzoylamino)-phenyl]-2-methyl-propyl}-amide
Thiophene-3-carboxylic acid {2-[4-(3,4-dimethoxy-benzoylamino)-phenyl]-2-methyl-propyl}-amide
Pyridine-2-carboxylic acid {2-[4-(3,4-dimethoxy-benzoylamino)-phenyl]-2-methyl-propyl}-amide
1H-Indazole-3-carboxylic acid {2-[4-(3,4-dimethoxy-benzoylamino)-phenyl]-2-methyl-propyl}-amide
N-[4-(Cyano-dimethyl-methyl)-3-methyl-phenyl]-3,4-dimethoxy-benzamide
N-[4-(Cyano-dimethyl-methyl)-3-trifluoromethyl-phenyl]-3,4-dimethoxy-benzamide
1H-Indole-3-carboxylic acid {2-[4-(3,4-dimethoxy-benzoylamino)-phenyl]-2-methyl-propyl}-amide
1H-Indazole-3-carboxylic acid {2-[4-(3,4-dimethoxy-benzoylamino)-2-methyl-phenyl]-2-methyl-propyl}-amide
1-Methyl-1H-Indazole-3-carboxylic acid {2-[4-(3,4-dimethoxy-benzoylamino)-2-methyl-phenyl]-2-methyl-propyl}-amide
1H-Indazole-3-carboxylic acid {2-[2-chloro-4-(3,4-dimethoxy-benzoylamino)-phenyl]-2-methyl-propyl}-amide
N-[3,5-Dichloro-4-(cyano-dimethyl-methyl)-phenyl]-3,4-dimethoxy-benzamide
1-Methyl-1H-indazole-3-carboxylic acid {2-[2-chloro-4-(3,4-dimethoxy-benzoylamino)-phenyl]-2-methyl-propyl}-amide
N-{4-[2-(2-1H-Indol-3-yl-acetylamino)-1,1-dimethyl-ethyl]-phenyl
N-[4-(Cyano-dimethyl-methyl)-3-pyridin-3-yl-phenyl]-3,4-dimethoxy-benzamide
1H-Indole-5-carboxylic acid {2-[4-(3,4-dimethoxy-benzoylamino)-phenyl]-2-methyl-propyl}-amide
1H-Benzoimidazole-5-carboxylic acid {2-[4-(3,4-dimethoxy-benzoylamino)-phenyl]-2-methyl-propyl}-amide
Imidazo[1,2-a]pyridine-3-carboxylic acid {3-[4-(3,4-dimethoxy-benzoylamino)-phenyl]-3-methyl-butyl}-amide
3H-Imidazo[4,5-c]pyridine-2-carboxylic acid {2-[4-(3,4-dimethoxy-benzoylamino)-phenyl]-2-methyl-propyl}-amide
5-Fluoro-1H-pyrrolo[2,3-b]pyridine-3-carboxylic acid {2-[4-(3,4-dimethoxy-benzoylamino)-phenyl]-2-methyl-propyl}-amide
3H-Imidazo[4,5-b]pyridine-2-carboxylic acid {3-[4-(3,4-dimethoxy-benzoylamino)-phenyl]-3-methyl-butyl}-amide
5-Fluoro-1-(2-methoxy-ethyl)-1H-indole-3-carboxylic acid {2-[4-(3,4-dimethoxy-benzoylamino)-phenyl]-2-methyl-propyl}-amide
7-Fluoro-1H-pyrrolo[2,3-c]pyridine-3-carboxylic acid {2-[4-(3,4-dimethoxy-benzoylamino)-phenyl]-2-methyl-propyl}-amide
3H-Imidazo[4,5-b]pyridine-6-carboxylic acid {3-[4-(3,4-dimethoxy-benzoylamino)-phenyl]-3-methyl-butyl}-amide
5-Chloro-1H-pyrrolo[2,3-c]pyridine-3-carboxylic acid {2-[4-(3,4-dimethoxy-benzoylamino)-phenyl]-2-methyl-propyl}-amide
1H-Pyrrolo[2,3-b]pyridine-5-carboxylic acid {2-[4-(3,4-dimethoxy-benzoylamino)-phenyl]-2-methyl-propyl}-amide
Imidazo[1,2-a]pyridine-3-carboxylic acid {2-[3-(3,4-dimethoxy-benzoylamino)-phenyl]-2-methyl-propyl}-amide
and pharmaceutically acceptable salts thereof.

47. A compound according to claim 1 wherein said compound is selected from:
1-Acetyl-1H-indole-3-carboxylic acid {2-[4-(3,4-dimethoxy-benzoylamino)-phenyl]-2-methyl-propyl}-amide
1-Methyl-1H-indole-2-carboxylic acid {2-[4-(3,4-dimethoxy-benzoylamino)-phenyl]-2-methyl-propyl}-amide
5-Methyl-1H-pyrazole-3-carboxylic acid {2-[4-(3,4-dimethoxy-benzoylamino)-phenyl]-2-methyl-propyl}-amide
3H-Imidazo[4,5-b]pyridine-2-carboxylic acid {2-[4-(3,4-dimethoxy-benzoylamino)-phenyl]-2-methyl-propyl}-amide
N-[4-(Cyano-dimethyl-methyl)-3-(1-methyl-1H-pyrazol-4-yl)-phenyl]-3,4-dimethoxy-benzamide
2-Methyl-1H-benzoimidazole-5-carboxylic acid {2-[4-(3,4-dimethoxy-benzoylamino)-phenyl]-2-methyl-propyl}-amide
1,3-Dimethyl-2-oxo-2,3-dihydro-1H-benzoimidazole-5-carboxylic acid {2-[4-(3,4-dimethoxy-benzoylamino)-phenyl]-2-methyl-propyl}-amide
1H-Pyrrolo[2,3-c]pyridine-3-carboxylic acid {2-[4-(3,4-dimethoxy-benzoylamino)-phenyl]-2-methyl-propyl}-amide
Imidazo[1,2-a]pyrimidine-3-carboxylic acid {2-[4-(3,4-dimethoxy-benzoylamino)-phenyl]-2-methyl-propyl}-amide
N-(4-(4-acetamido-2-methylbutan-2-yl)-3-(pyridin-3-yl)phenyl)-3,4-dimethoxybenzamide
1-Methyl-1H-indazole-3-carboxylic acid {1-[3-(3,4-dimethoxy-benzoylamino)-phenyl]-cyclopentylmethyl}-amide
N-[4-Chloro-3-(cyano-dimethyl-methyl)-phenyl]-3,4-dimethoxy-benzamide
N-[4-(Cyano-dimethyl-methyl)-3-pyrimidin-5-yl-phenyl]-3,4-dimethoxy-benzamide
N-[4-(Cyano-dimethyl-methyl)-3-pyridin-2-yl-phenyl]-3,4-dimethoxy-benzamide
N-[4-(Cyano-dimethyl-methyl)-3-morpholin-4-yl-phenyl]-3,4-dimethoxy-benzamide
1,2-Dimethyl-1H-benzoimidazole-5-carboxylic acid {2-[4-(3,4-dimethoxy-benzoylamino)-phenyl]-2-methyl-propyl}-amide
Imidazo[1,2-a]pyrimidine-2-carboxylic acid {2-[4-(3,4-dimethoxy-benzoylamino)-phenyl]-2-methyl-propyl}-amide
3H-Imidazo[4,5-b]pyridine-6-carboxylic acid {2-[4-(3,4-dimethoxy-benzoylamino)-phenyl]-2-methyl-propyl}-amide
N-[4-(2-Acetylamino-1,1-dimethyl-ethyl)-3-pyridin-3-yl-phenyl]-3,4-dimethoxy-benzamide
Imidazo[1,2-a]pyridine-6-carboxylic acid {2-[4-(3,4-dimethoxy-benzoylamino)-phenyl]-2-methyl-propyl}-amide
1-(3-Dimethylamino-propyl)-5-fluoro-1H-indole-3-carboxylic acid {2-[4-(3,4-dimethoxy-benzoylamino)-phenyl]-2-methyl-propyl}-amide
Imidazo[1,2-a]pyridine-3-carboxylic acid {2-[4-(3,4-dimethoxy-benzoylamino)-phenyl]-3-hydroxy-2-methyl-propyl}-amide
and pharmaceutically acceptable salts thereof.

48. A pharmaceutical composition comprising a therapeutically effective amount of a compound according to claim 1 and a pharmaceutically acceptable carrier and/or excipient.

49. A method in treating or preventing a condition in a mammal, including a human, the treatment or prevention of which is affected or facilitated by the effect of FSH receptor negative allosteric modulators, the method comprising:
administering a compound of claim 1 to the mammal in need thereof.

50. A method to provide female or male contraception, the method comprising:
administering a compound according to claim 1 to a female or male in need thereof.

51. A method to treat or prevent disorders selected from the group consisting of uterine fibroids, endometriosis, polycystic ovarian disease, dysfunctional uterine bleeding, hormono-dependent cancer, prostate cancer, uterine cancer, breast cancer and ovarian cancer; or osteoporosis, the method comprising:
administering a compound of claim 1 to a subject suffering from or susceptible to a disorder selected from the group consisting of uterine fibroids, endometriosis, polycystic ovarian disease, dysfunctional uterine bleeding, hormono-dependent cancer, prostate cancer, uterine cancer, breast cancer and ovarian cancer; or osteoporosis.

52. A method to treat or prevent disorders selected from the group consisting of uterine fibroids, endometriosis, polycystic ovarian disease, dysfunctional uterine bleeding, hormono-dependent cancer, prostate cancer, uterine cancer, breast cancer and ovarian cancer; or osteoporosis, the method comprising:
administering a compound of claim 1 to a subject suffering from or susceptible to a disorder selected from the group consisting of uterine fibroids, endometriosis, polycystic ovarian disease, dysfunctional uterine bleeding, hormono-dependent cancer, prostate cancer, uterine cancer, breast cancer and ovarian cancer; or osteoporosis.

53. A method for fertility control in a mammal including men or women, the method comprising:
administering a compound of claim 1 to a mammal including men or women in need thereof.

54. A method for treating or preventing uterine fibroids, endometriosis, polycystic ovarian disease, and/or dysfunctional uterine bleeding, the method comprising:
administering a compound of claim 1 to a subject suffering from or susceptible to uterine fibroids, endometriosis, polycystic ovarian disease, and/or dysfunctional uterine bleeding.

55. A method to treat hormono-dependent cancer, prostate cancer, uterine cancer, breast cancer and/or ovarian cancer, the method comprising:
administering a compound of claim 1 to a subject suffering from or susceptible to hormono-dependent cancer, prostate cancer, uterine cancer, breast cancer and/or ovarian cancer.

56. A method for treating or preventing osteoporosis, the method comprising:
administering a compound of claim 1 to a subject suffering from or susceptible to osteoporosis.

57. A method for preparing a tracer for imaging FSH receptors, comprising:
using a compound of claim 1 to prepare a tracer for imaging a FSH receptor.
